SEQUENCE LISTING (133 sequences)

SEQ ID NO: 1 - RNA, 1464 nt, synthetic construct
  source 1..1464: mol_type = other RNA
atggagttcggcttgagctgggtgttcctagtggccatcttgaagggcgtgcagtgcgacgagcctcctcagagcccttgggacagagtgaaggacttggccaccgtgtacgtggacgtgcttaaggacagcggcagagactacgtgagccagttcgagggcagcgccctgggcaagcagctgaacctgaagctgctggacaactgggacagcgtgaccagcaccttcagcaagctgagagagcagctgggccctgtgacccaggagttctgggacaacctggagaaggagacagagggcctgagacaggagatgagcaaggacttagaggaggtgaaggccaaggtgcagccttacctggacgacttccagaagaagtggcaggaggagatggagctgtacagacagaaggtggagcctctgagagccgagctgcaggagggcgcacggcagaagctgcacgaactgcaggagaagctgagccctctgggcgaggaaatgagagacagagccagagcccacgtggatgccctgagaacccacctggccccttactcggacgagctgcgacagagactggccgccagactggaggccctgaaggagaacggcggcgcaagactcgcagagtaccacgccaaggccaccgagcacctgagcaccctgagcgagaaggctaagcctgctctggaggacctgcggcagggcctgctgcctgtgctggagagcttcaaggtgagcttcctgtccgcactggaagaatacaccaagaagctgaacacccagggcggcggaggatcgggtggaggtggtagtggtggtggcggttccatcacctgccctcctcctatgagcgtggagcacgccgacatctgggtaaagtcttacagcctgtacagcagagagagatacatctgcaacagcggcttcaagagaaaggccggcaccagcagcctgaccgagtgtgtcctgaacaaggcaaccaacgtggcccactggaccacccctagcctgaagtgcatcagagatccagcccttgtgcaccaaagacctgcccctccatcaggaggttccggcggtggaggatcaggcggcggcagcggaggtggcggatccctgcagaactgggtaaacgtgatcagcgacctgaagaagatcgaagatctgatccagagcatgcacatcgacgccaccctgtacaccgagagcgacgtgcaccctagctgcaaggtgaccgccatgaagtgcttcctgctggaactccaggttatctccctggagagtggcgacgccagcatccacgacaccgtggagaacctgatcatcctggccaacaacagcctgagcagcaacggcaacgtgactgaaagcggctgtaaggagtgcgaggagctcgaggagaagaacatcaaggagttcctgcagagcttcgtccacatcgtgcagatgttcatcaacaccagc

SEQ ID NO: 2 - protein, 488 aa, synthetic construct
MEFGLSWVFLVAILKGVQCDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGGGGSGGGGSGGGGSITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - RNA, 1428 nt, synthetic construct
  source 1..1428: mol_type = other RNA
atggagttcggcttaagctgggtgttcctcgtggccatcttaaagggcgtgcagtgcgacgagcctcctcagagcccttgggacagagtgaaggacctcgccaccgtgtacgtggacgtgctcaaggacagcggcagagactacgtgagccagttcgagggcagcgccctgggcaagcagctgaacctgaagctgctggacaactgggacagcgtgaccagcaccttcagcaagctgagagagcagctgggccctgtgacccaggagttctgggacaacctggagaaggagactgagggcctgagacaggagatgagcaaggatctggaggaggtgaaggccaaggtgcagccttacctggacgacttccagaagaagtggcaggaggagatggagctgtacagacagaaggtggagcctctgagagccgagctgcaggagggcgcacgtcagaagctgcacgaacttcaggagaagctgagccctctgggcgaagaaatgagagacagagccagagcccatgtggacgccctgagaacccacctggccccttacagcgacgaactgagacaaagactggccgccagactggaggccctgaaggagaacggcggtgcgagattagctgagtaccacgccaaggccaccgagcacctgagcaccctgagcgagaaggctaagcctgccctcgaggacctgcgacaaggcctgctgcctgtgctggagagcttcaaggtgagcttcctgtcagcacttgaagagtacaccaagaagctgaacacccagggcggcggcggaagcggcggtggcggcagtggaggcggtggaagcatcacctgcccgcctcctatgagcgtggagcacgccgacatctgggtaaagtcctacagcctgtacagcagagagagatacatctgcaacagcggcttcaagagaaaggccggcaccagcagcctgaccgagtgcgtgcttaataaggccactaacgtggcccactggaccacccctagcctgaagtgcatcagagacggcggttcaggaggcggaggttccggtggaggctcaggtggaggtggtagcctgcagaactgggtaaacgtgatcagcgacctgaagaagatcgaagatctcatccagagcatgcacatcgacgccaccctgtacaccgagagcgacgtgcaccctagctgcaaggtgaccgccatgaagtgcttcctgctggaactgcaggtgataagcctggagtctggcgacgccagcatccacgacaccgtggagaacctgatcatcctggccaacaacagcctgagcagcaacggcaacgtaaccgagtccggctgtaaggagtgcgaggagttggaggagaagaacatcaaggagttcctgcagagcttcgttcacatcgtgcagatgttcatcaacaccagc

SEQ ID NO: 4 - protein, 476 aa, synthetic construct
MEFGLSWVFLVAILKGVQCDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGGGGSGGGGSGGGGSITCPPPMSVEHADIWVKSYSLYSRERYICNSGFKRKAGTSSLTECVLNKATNVAHWTTPSLKCIRDGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 5 - RNA, 1386 nt, synthetic construct
  source 1..1386: mol_type = other RNA
atggagttcggcttgagctgggtgttcttagtggccatcttaaagggcgtgcagtgcgagcctaagagctgcgacaagacccacacctgccctccttgcccggccccagagttgcttggcggccctagcgtcttcctcttccctcctaagcctaaggacaccctgatgatcagcagaacccctgaggtgacctgcgtggtggtggccgtgagccacgaggaccctgaagtaaagttcaactggtacgtggacggcgtggaggtgcacaacgccaagaccaagcctagagaggagcagtacaacagcacctacagagtggtgagcgtgctgaccgtgctgcaccaggactggctgaacggcaaggagtacaagtgcaaggtgagcaacaaggccctgcctgcacctatcgagaagaccatcagcaaggccaagggccaacctcgagagcctcaggtgtacaccctgcctcctagcagagacgagctgaccaagaaccaggtgagcctgacctgcctggtgaagggcttctaccctagcgacatcgccgtggagtgggagagcaacggccaaccagagaacaactacaagaccacccctcctgtgctggacagcgacggcagcttcttcctctacagcaagctgactgttgacaagagcagatggcagcagggcaacgtgttcagctgcagcgtgatgcacgaggcgctgcacaaccactacacccagaagagtctttccctgagccctggcaagataacgtgtccgcctcctatgagcgtggagcacgccgacatctgggtgaagagttacagcctgtattccagagagagatacatctgcaacagcggcttcaagagaaaggccggcaccagcagcttaaccgagtgcgtccttaacaaggctaccaacgtggcccactggacaacgccgagcctgaagtgcatccgcgaccctgccctggtgcaccaacgtccagcacctccgagtggcggcagtggtggcggaggctcaggcggtggtagtggaggaggcggaagtctgcagaattgggtgaacgtgatcagcgacctgaagaagatcgaggacctgatccagagcatgcacatcgacgccaccctgtacaccgagagcgacgtgcaccctagctgtaaggtaaccgccatgaagtgcttcctgctggagctgcaggtgattagcctggagagcggcgacgccagcatccacgacaccgtggagaacctgatcatcctggccaacaattctctcagttctaacggtaacgtgaccgaatcgggttgtaaggagtgcgaggagctggaggagaagaacatcaaggagttcctgcagagcttcgtgcacattgtgcagatgttcatcaacaccagc

SEQ ID NO: 6 - protein, 462 aa, synthetic construct
MEFGLSWVFLVAILKGVQCEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 7 - RNA, 594 nt, synthetic construct
  source 1..594: mol_type = other RNA
aaggaaggggaaggggttcaacccctggaagagaatctggaaaacggatcaaggccaagattcaagaggaagaagacgctaaggctggaggtctctgggatcaagggagcagggaagcttctgagcttcatctaagtctgcctgcaactctcttcctctccggcaaaggaccctccaatccaaagactcagaggagcagttaccagaagagaggaagggcaactattcatcagctcatacaagaaagagtatcaaactaaggaggagcagaacaattcggttgtcatcaagagcgaagggctttatatcatctacctgaagggctcctttttccaggaggtcaagattgaccttcatttccgggaggatcataatcccatctctattccaaagctgaacgaaggtcgaaggattgtcttcactgaggaggcctctttggctttcaaagataaagtttacctgactgtaaaagctcctgatactctctgcgaacacctccagataaaagaaggggagctgattgttgtccagctaacgcctggatactgagctcctgaaggatcttaccacagcactgagaaccaagtaccactg

SEQ ID NO: 8 - RNA, 1425 nt, synthetic construct
  source 1..1425: mol_type = other RNA
atggctcctcgacgcgctcggggatgccggaccctcggtttgccggcgttgctactccttctcctactccgcccacctgccactagaggcatcacgtgccctcctcccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacacctagtctcaaatgcattagagacggcggctctggaggtggaggatccggcggaggatctggaggaggaggcagcctgcagaactgggtgaatgtaataagtgatttgaagaagattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagagaatctgatcatcctagcaaacaacagtttgtcttctaatgggaatgtaacagaatctggatgcaaagaatgtgaggaactggaggagaagaacattaaagaattcttgcagagttttgtacatattgtccaaatgttcatcaacacttctggcgcgcccgatgaaccaccccagagcccctgggatcgagtgaaggacctggccactgtgtacgtggatgtgctcaaagacagcggcagagactatgtgtcccagtttgaaggctccgccttgggcaagcagctaaacctaaagctccttgacaactgggacagcgtgacctccaccttcagcaagctgcgcgaacagctcggccctgtgacccaggagttctgggataacctggagaaggagacagagggcctgaggcaggagatgagcaaggacctagaggaggtgaaggccaaggtgcagccctacctggacgacttccagaagaagtggcaggaggagatggagctctaccgccagaaggtggagccgctgcgcgcagagctccaagaaggtgccagacagaagctgcacgagctgcaagagaagctgtctccactgggcgaagagatgcgcgacagagctcgcgcgcatgtggacgcgctgcgcacgcatctggcaccttacagcgatgagctccgccagcgcttggccgcgcgccttgaggctctcaaggagaacggcggcgccagactggccgagtaccacgctaaggcgaccgagcatctgagcacgctcagcgagaaggcaaagcccgcgctcgaggacctccgacaaggactgctgcccgtgctggagagcttcaaggtcagcttcctgagcgctctcgaggagtacactaagaagctcaacacccag

SEQ ID NO: 9 - protein, 475 aa, synthetic construct
MAPRRARGCRTLGLPALLLLLLLRPPATRGITCPPPMSVEHADIWVKSYSLYSRERYICNSGFKRKAGTSSLTECVLNKATNVAHWTTPSLKCIRDGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGAPDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 10 - RNA, 1461 nt, synthetic construct
  source 1..1461: mol_type = other RNA
atggcccctcggcgggccagaggctgcagaaccttaggcctcccagcccttctattgctcttgctactcagacctccagccaccagaggcatcacctgccctcctcctatgagcgtggagcacgccgacatctgggtaaagtcctacagcctgtacagcagagagagatacatctgcaacagcggcttcaagagaaaggccggcaccagcagcctgaccgagtgcgtgctgaacaaggccaccaacgtggcccactggaccacccctagcctgaagtgcatcagggacccagctctagtgcaccagcggcctgcccctcctagcggcggctcgggtggcggtggatcgggtggtggttccggcggaggaggctctctccagaactgggttaacgtgatcagcgacctgaagaagatcgaggacctgatccagagcatgcacatcgacgccaccctgtacaccgagagcgacgtgcaccctagctgcaaggtgaccgccatgaagtgcttcctgctggagctgcaggtaatttctctggagagcggcgacgccagcatccacgacaccgtggagaacctgatcatcctggccaacaacagcctgagcagcaacggcaacgtgactgagtccggatgcaaggagtgcgaggagctggaggagaagaacatcaaggagttcctgcagagcttcgtgcatatcgtgcagatgttcatcaacaccagcggcgcccctgacgagcctcctcagagcccttgggacagagtgaaggacctggccaccgtgtacgtggacgtgttaaaggacagcggcagagactacgtgagccagttcgagggcagcgccctgggcaagcagctgaacctgaagctgctggacaactgggacagcgtgaccagcaccttcagcaagctgagagagcagctgggccctgtgacccaggagttctgggacaacctggagaaggaaactgagggcctgagacaggagatgagcaaggaccttgaagaagtgaaggccaaggtgcagccttacctggacgacttccagaagaagtggcaggaggagatggagctgtacagacagaaggtggagcctctgagagccgaacttcaggagggcgctcgccagaagctgcacgagctacaagagaagctgagccctctgggcgaagagatgcgcgaccgggctagggcccatgttgacgccctgagaacccacctggccccttacagcgatgaactgcgccagcgcctggccgccagactggaggccctgaaggagaacggcggcgctcgactagccgagtaccacgccaaggctactgagcacctgagcactctgagcgagaaggcgaagccggccctggaggatctcaggcaaggcctgctgcctgtgctggagtcattcaaggtgagcttcctgtccgcgctcgaagagtacaccaagaagttgaacacccag

SEQ ID NO: 11 - protein, 487 aa, synthetic construct
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGAPDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 12 - protein, 30 aa, Homo sapiens
MAPRRARGCRTLGLPALLLLLLLRPPATRG

SEQ ID NO: 13 - protein, 19 aa, Homo sapiens
MEFGLSWVFLVAILKGVQC

SEQ ID NO: 14 - protein, 243 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 15 - protein, 232 aa, synthetic construct
EPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 17 - protein, 78 aa, synthetic construct
ITCPPPMSVEHADIWVKSYSLYSRERYICNSGFKRKAGTSSLTECVLNKATNVAHWTTPSLKCIRDPALVHQRPAPPS

SEQ ID NO: 18 - protein, 66 aa, synthetic construct
ITCPPPMSVEHADIWVKSYSLYSRERYICNSGFKRKAGTSSLTECVLNKATNVAHWTTPSLKCIRD

SEQ ID NO: 19 - RNA, 57 nt, synthetic construct
  source 1..57: mol_type = other RNA
gggaaataagagagaaaagaagagtaagaagaaatataagaccccggcgccgccacc

SEQ ID NO: 20 - RNA, 119 nt, synthetic construct
  source 1..119: mol_type = other RNA
tgataataggctggagcctcggtggcctagcttcttgccccttgggcctccccccagcccctcctccccttcctgcacccgtacccccgtggtctttgaataaagtctgagtgggcggc

SEQ ID NO: 21 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 22 - protein, 445 aa, synthetic construct
ITCPPPMSVEHADIWVKSYSLYSRERYICNSGFKRKAGTSSLTECVLNKATNVAHWTTPSLKCIRDGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGAPDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 23 - protein, 457 aa, synthetic construct
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGAPDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 24 - RNA, 57 nt, synthetic construct
  source 1..57: mol_type = other RNA
atggagttcggcttgagctgggtgttcctagtggccatcttgaagggcgtgcagtgc

SEQ ID NO: 25 - RNA, 57 nt, synthetic construct
  source 1..57: mol_type = other RNA
atggagttcggcttaagctgggtgttcctcgtggccatcttaaagggcgtgcagtgc

SEQ ID NO: 26 - RNA, 57 nt, synthetic construct
  source 1..57: mol_type = other RNA
atggagttcggcttgagctgggtgttcttagtggccatcttaaagggcgtgcagtgc

SEQ ID NO: 27 - RNA, 90 nt, synthetic construct
  source 1..90: mol_type = other RNA
atggctcctcgacgcgctcggggatgccggaccctcggtttgccggcgttgctactccttctcctactccgcccacctgccactagaggc

SEQ ID NO: 28 - RNA, 90 nt, synthetic construct
  source 1..90: mol_type = other RNA
atggcccctcggcgggccagaggctgcagaaccttaggcctcccagcccttctattgctcttgctactcagacctccagccaccagaggc

SEQ ID NO: 29 - RNA, 234 nt, synthetic construct
  source 1..234: mol_type = other RNA
atcacctgccctcctcctatgagcgtggagcacgccgacatctgggtaaagtcttacagcctgtacagcagagagagatacatctgcaacagcggcttcaagagaaaggccggcaccagcagcctgaccgagtgtgtcctgaacaaggcaaccaacgtggcccactggaccacccctagcctgaagtgcatcagagatccagcccttgtgcaccaaagacctgcccctccatca

SEQ ID NO: 30 - RNA, 234 nt, synthetic construct
  source 1..234: mol_type = other RNA
ataacgtgtccgcctcctatgagcgtggagcacgccgacatctgggtgaagagttacagcctgtattccagagagagatacatctgcaacagcggcttcaagagaaaggccggcaccagcagcttaaccgagtgcgtccttaacaaggctaccaacgtggcccactggacaacgccgagcctgaagtgcatccgcgaccctgccctggtgcaccaacgtccagcacctccgagt

SEQ ID NO: 31 - RNA, 234 nt, synthetic construct
  source 1..234: mol_type = other RNA
atcacctgccctcctcctatgagcgtggagcacgccgacatctgggtaaagtcctacagcctgtacagcagagagagatacatctgcaacagcggcttcaagagaaaggccggcaccagcagcctgaccgagtgcgtgctgaacaaggccaccaacgtggcccactggaccacccctagcctgaagtgcatcagggacccagctctagtgcaccagcggcctgcccctcctagc

SEQ ID NO: 32 - RNA, 198 nt, synthetic construct
  source 1..198: mol_type = other RNA
atcacctgcccgcctcctatgagcgtggagcacgccgacatctgggtaaagtcctacagcctgtacagcagagagagatacatctgcaacagcggcttcaagagaaaggccggcaccagcagcctgaccgagtgcgtgcttaataaggccactaacgtggcccactggaccacccctagcctgaagtgcatcagagac

SEQ ID NO: 33 - RNA, 198 nt, synthetic construct
  source 1..198: mol_type = other RNA
atcacgtgccctcctcccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacacctagtctcaaatgcattagagac

SEQ ID NO: 34 - RNA, 729 nt, synthetic construct
  source 1..729: mol_type = other RNA
gacgagcctcctcagagcccttgggacagagtgaaggacttggccaccgtgtacgtggacgtgcttaaggacagcggcagagactacgtgagccagttcgagggcagcgccctgggcaagcagctgaacctgaagctgctggacaactgggacagcgtgaccagcaccttcagcaagctgagagagcagctgggccctgtgacccaggagttctgggacaacctggagaaggagacagagggcctgagacaggagatgagcaaggacttagaggaggtgaaggccaaggtgcagccttacctggacgacttccagaagaagtggcaggaggagatggagctgtacagacagaaggtggagcctctgagagccgagctgcaggagggcgcacggcagaagctgcacgaactgcaggagaagctgagccctctgggcgaggaaatgagagacagagccagagcccacgtggatgccctgagaacccacctggccccttactcggacgagctgcgacagagactggccgccagactggaggccctgaaggagaacggcggcgcaagactcgcagagtaccacgccaaggccaccgagcacctgagcaccctgagcgagaaggctaagcctgctctggaggacctgcggcagggcctgctgcctgtgctggagagcttcaaggtgagcttcctgtccgcactggaagaatacaccaagaagctgaacacccag

SEQ ID NO: 35 - RNA, 729 nt, synthetic construct
  source 1..729: mol_type = other RNA
gacgagcctcctcagagcccttgggacagagtgaaggacctcgccaccgtgtacgtggacgtgctcaaggacagcggcagagactacgtgagccagttcgagggcagcgccctgggcaagcagctgaacctgaagctgctggacaactgggacagcgtgaccagcaccttcagcaagctgagagagcagctgggccctgtgacccaggagttctgggacaacctggagaaggagactgagggcctgagacaggagatgagcaaggatctggaggaggtgaaggccaaggtgcagccttacctggacgacttccagaagaagtggcaggaggagatggagctgtacagacagaaggtggagcctctgagagccgagctgcaggagggcgcacgtcagaagctgcacgaacttcaggagaagctgagccctctgggcgaagaaatgagagacagagccagagcccatgtggacgccctgagaacccacctggccccttacagcgacgaactgagacaaagactggccgccagactggaggccctgaaggagaacggcggtgcgagattagctgagtaccacgccaaggccaccgagcacctgagcaccctgagcgagaaggctaagcctgccctcgaggacctgcgacaaggcctgctgcctgtgctggagagcttcaaggtgagcttcctgtcagcacttgaagagtacaccaagaagctgaacacccag

SEQ ID NO: 36 - RNA, 729 nt, synthetic construct
  source 1..729: mol_type = other RNA
gatgaaccaccccagagcccctgggatcgagtgaaggacctggccactgtgtacgtggatgtgctcaaagacagcggcagagactatgtgtcccagtttgaaggctccgccttgggcaagcagctaaacctaaagctccttgacaactgggacagcgtgacctccaccttcagcaagctgcgcgaacagctcggccctgtgacccaggagttctgggataacctggagaaggagacagagggcctgaggcaggagatgagcaaggacctagaggaggtgaaggccaaggtgcagccctacctggacgacttccagaagaagtggcaggaggagatggagctctaccgccagaaggtggagccgctgcgcgcagagctccaagaaggtgccagacagaagctgcacgagctgcaagagaagctgtctccactgggcgaagagatgcgcgacagagctcgcgcgcatgtggacgcgctgcgcacgcatctggcaccttacagcgatgagctccgccagcgcttggccgcgcgccttgaggctctcaaggagaacggcggcgccagactggccgagtaccacgctaaggcgaccgagcatctgagcacgctcagcgagaaggcaaagcccgcgctcgaggacctccgacaaggactgctgcccgtgctggagagcttcaaggtcagcttcctgagcgctctcgaggagtacactaagaagctcaacacccag

SEQ ID NO: 37 - RNA, 729 nt, synthetic construct
  source 1..729: mol_type = other RNA
gacgagcctcctcagagcccttgggacagagtgaaggacctggccaccgtgtacgtggacgtgttaaaggacagcggcagagactacgtgagccagttcgagggcagcgccctgggcaagcagctgaacctgaagctgctggacaactgggacagcgtgaccagcaccttcagcaagctgagagagcagctgggccctgtgacccaggagttctgggacaacctggagaaggaaactgagggcctgagacaggagatgagcaaggaccttgaagaagtgaaggccaaggtgcagccttacctggacgacttccagaagaagtggcaggaggagatggagctgtacagacagaaggtggagcctctgagagccgaacttcaggagggcgctcgccagaagctgcacgagctacaagagaagctgagccctctgggcgaagagatgcgcgaccgggctagggcccatgttgacgccctgagaacccacctggccccttacagcgatgaactgcgccagcgcctggccgccagactggaggccctgaaggagaacggcggcgctcgactagccgagtaccacgccaaggctactgagcacctgagcactctgagcgagaaggcgaagccggccctggaggatctcaggcaaggcctgctgcctgtgctggagtcattcaaggtgagcttcctgtccgcgctcgaagagtacaccaagaagttgaacacccag

SEQ ID NO: 38 - RNA, 342 nt, synthetic construct
  source 1..342: mol_type = other RNA
aactgggtaaacgtgatcagcgacctgaagaagatcgaagatctgatccagagcatgcacatcgacgccaccctgtacaccgagagcgacgtgcaccctagctgcaaggtgaccgccatgaagtgcttcctgctggaactccaggttatctccctggagagtggcgacgccagcatccacgacaccgtggagaacctgatcatcctggccaacaacagcctgagcagcaacggcaacgtgactgaaagcggctgtaaggagtgcgaggagctcgaggagaagaacatcaaggagttcctgcagagcttcgtccacatcgtgcagatgttcatcaacaccagc

SEQ ID NO: 39 - RNA, 342 nt, synthetic construct
  source 1..342: mol_type = other RNA
aactgggtaaacgtgatcagcgacctgaagaagatcgaagatctcatccagagcatgcacatcgacgccaccctgtacaccgagagcgacgtgcaccctagctgcaaggtgaccgccatgaagtgcttcctgctggaactgcaggtgataagcctggagtctggcgacgccagcatccacgacaccgtggagaacctgatcatcctggccaacaacagcctgagcagcaacggcaacgtaaccgagtccggctgtaaggagtgcgaggagttggaggagaagaacatcaaggagttcctgcagagcttcgttcacatcgtgcagatgttcatcaacaccagc

SEQ ID NO: 40 - RNA, 342 nt, synthetic construct
  source 1..342: mol_type = other RNA
aattgggtgaacgtgatcagcgacctgaagaagatcgaggacctgatccagagcatgcacatcgacgccaccctgtacaccgagagcgacgtgcaccctagctgtaaggtaaccgccatgaagtgcttcctgctggagctgcaggtgattagcctggagagcggcgacgccagcatccacgacaccgtggagaacctgatcatcctggccaacaattctctcagttctaacggtaacgtgaccgaatcgggttgtaaggagtgcgaggagctggaggagaagaacatcaaggagttcctgcagagcttcgtgcacattgtgcagatgttcatcaacaccagc

SEQ ID NO: 41 - RNA, 342 nt, synthetic construct
  source 1..342: mol_type = other RNA
aactgggtgaatgtaataagtgatttgaagaagattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagagaatctgatcatcctagcaaacaacagtttgtcttctaatgggaatgtaacagaatctggatgcaaagaatgtgaggaactggaggagaagaacattaaagaattcttgcagagttttgtacatattgtccaaatgttcatcaacacttct

SEQ ID NO: 42 - RNA, 342 nt, synthetic construct
  source 1..342: mol_type = other RNA
aactgggttaacgtgatcagcgacctgaagaagatcgaggacctgatccagagcatgcacatcgacgccaccctgtacaccgagagcgacgtgcaccctagctgcaaggtgaccgccatgaagtgcttcctgctggagctgcaggtaatttctctggagagcggcgacgccagcatccacgacaccgtggagaacctgatcatcctggccaacaacagcctgagcagcaacggcaacgtgactgagtccggatgcaaggagtgcgaggagctggaggagaagaacatcaaggagttcctgcagagcttcgtgcatatcgtgcagatgttcatcaacaccagc

SEQ ID NO: 43 - RNA, 696 nt, synthetic construct
  source 1..696: mol_type = other RNA
gagcctaagagctgcgacaagacccacacctgccctccttgcccggccccagagttgcttggcggccctagcgtcttcctcttccctcctaagcctaaggacaccctgatgatcagcagaacccctgaggtgacctgcgtggtggtggccgtgagccacgaggaccctgaagtaaagttcaactggtacgtggacggcgtggaggtgcacaacgccaagaccaagcctagagaggagcagtacaacagcacctacagagtggtgagcgtgctgaccgtgctgcaccaggactggctgaacggcaaggagtacaagtgcaaggtgagcaacaaggccctgcctgcacctatcgagaagaccatcagcaaggccaagggccaacctcgagagcctcaggtgtacaccctgcctcctagcagagacgagctgaccaagaaccaggtgagcctgacctgcctggtgaagggcttctaccctagcgacatcgccgtggagtgggagagcaacggccaaccagagaacaactacaagaccacccctcctgtgctggacagcgacggcagcttcttcctctacagcaagctgactgttgacaagagcagatggcagcagggcaacgtgttcagctgcagcgtgatgcacgaggcgctgcacaaccactacacccagaagagtctttccctgagccctggcaag

SEQ ID NO: 44 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 45 - RNA, 486 nt, Homo sapiens
  source 1..486: mol_type = genomic RNA
atgcgcatcagcaaacctcatttacggagtatcagcatccagtgctatctctgcctgcttctgaacagtcattttctgactgaggcgggcattcatgtctttattttaggctgcttttccgcaggtctgcccaaaacagaagcaaattgggtgaacgtgatcagcgacctgaagaagattgaggatctaattcaaagcatgcatattgatgccacactctacaccgaatccgacgtgcacccttcgtgtaaagtgactgcaatgaagtgtttcttactggaactgcaggtgatcagtctggagtccggggatgcatcaatccacgacacagtggaaaacctgattatcctggcaaacaattccctgagcagtaatggcaatgtcacggagagcggatgtaaggagtgtgaggaattagaggaaaagaatatcaaggaattccttcagtcctttgtgcacatcgtacagatgtttattaatacatcc

SEQ ID NO: 46 - RNA, 801 nt, Homo sapiens
  source 1..801: mol_type = genomic RNA
atggctccccgccgcgcgcgaggctgtcgcaccctcggacttcctgcactcttgcttttgctcctccttagaccccctgcaaccagagggataacctgtccacctccaatgagcgtcgagcacgcagacatttgggtgaaatcatacagtctgtacagtagagagcggtacatctgcaacagtgggtttaaaagaaaagcaggcacttcatctctgacagagtgcgtgctgaacaaagcaactaatgtagctcattggacaaccccatcactgaaatgcattagagatccagctctggtgcatcaaagaccagcaccaccaagtaccgtaacaaccgcaggggtgacccctcagcctgagtccctatctccctccggcaaggagccagcagcatcttcacctagctccaataacaccgcagctaccactgccgccatagtccccggaagccagctgatgcctagtaaatctccctcaacaggtaccaccgaaatttctagccatgagtcctcgcacggcaccccgtcacagactacagctaaaaactgggagctaacggcttcggcatcccaccaacctccaggcgtttatccccaaggtcactccgacactactgtggcgattagcacaagtaccgtccttctgtgtggactgagtgcagtttcattgctggcctgttatctgaaatctcgccagacccctcccctcgccagtgttgagatggaagccatggaagcacttcctgtgacttggggaacatcctcgagggacgaggacctcgagaactgctctcaccacctg

SEQ ID NO: 47 - protein, 61 aa, Homo sapiens
CPPPMSVEHADIWVKSYSLYSRERYICNSGFKRKAGTSSLTECVLNKATNVAHWTTPSLKC

SEQ ID NO: 48 - RNA, 183 nt, Homo sapiens
  source 1..183: mol_type = genomic RNA
tgtccacctccaatgagcgtcgagcacgcagacatttgggtgaaatcatacagtctgtacagtagagagcggtacatctgcaacagtgggtttaaaagaaaagcaggcacttcatctctgacagagtgcgtgctgaacaaagcaactaatgtagctcattggacaaccccatcactgaaatgc

SEQ ID NO: 49 - protein, 23 aa, Homo sapiens
VAISTSTVLLCGLSAVSLLACYL

SEQ ID NO: 50 - protein, 39 aa, Homo sapiens
KSRQTPPLASVEMEAMEALPVTWGTSSRDEDLENCSHHL

SEQ ID NO: 51 - protein, 205 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 52 - RNA, 615 nt, Homo sapiens
  source 1..615: mol_type = genomic RNA
atggctccccgccgcgcgcgaggctgtcgcaccctcggacttcctgcactcttgcttttgctcctccttagaccccctgcaaccagagggataacctgtccacctccaatgagcgtcgagcacgcagacatttgggtgaaatcatacagtctgtacagtagagagcggtacatctgcaacagtgggtttaaaagaaaagcaggcacttcatctctgacagagtgcgtgctgaacaaagcaactaatgtagctcattggacaaccccatcactgaaatgcattagagatccagctctggtgcatcaaagaccagcaccaccaagtaccgtaacaaccgcaggggtgacccctcagcctgagtccctatctccctccggcaaggagccagcagcatcttcacctagctccaataacaccgcagctaccactgccgccatagtccccggaagccagctgatgcctagtaaatctccctcaacaggtaccaccgaaatttctagccatgagtcctcgcacggcaccccgtcacagactacagctaaaaactgggagctaacggcttcggcatcccaccaacctccaggcgtttatccccaaggtcactccgacactact

SEQ ID NO: 53 - protein, 25 aa, synthetic construct
  VARIANT 1..25: note = This sequence may encompass 1-5 GGGGS repeating units
GGGGSGGGGSGGGGSGGGGSGGGGS

SEQ ID NO: 54 - protein, 10 aa, synthetic construct
GGSGGGGSGG

SEQ ID NO: 55 - protein, 8 aa, synthetic construct
GGSGGGGG

SEQ ID NO: 56 - protein, 8 aa, synthetic construct
GSGSGSGS

SEQ ID NO: 57 - protein, 7 aa, synthetic construct
GGGGGGS

SEQ ID NO: 58 - protein, 5 aa, synthetic construct
GGGGG

SEQ ID NO: 59 - protein, 6 aa, synthetic construct
GGGGGG

SEQ ID NO: 60 - protein, 7 aa, synthetic construct
GGGGGGG

SEQ ID NO: 61 - protein, 8 aa, synthetic construct
GGGGGGGG

SEQ ID NO: 62 - protein, 25 aa, synthetic construct
  VARIANT 1..25: note = This sequence may encompass 1-5 EAAAK repeating units
EAAAKEAAAKEAAAKEAAAKEAAAK

SEQ ID NO: 63 - protein, 15 aa, synthetic construct
EAAAKEAAAKEAAAK

SEQ ID NO: 64 - protein, 16 aa, synthetic construct
GGGGSLVPRGSGGGGS

SEQ ID NO: 65 - protein, 6 aa, synthetic construct
GSGSGS

SEQ ID NO: 66 - protein, 15 aa, synthetic construct
GGGGSLVPRGSGGGG

SEQ ID NO: 67 - protein, 11 aa, synthetic construct
GGSGGHMGSGG

SEQ ID NO: 68 - protein, 11 aa, synthetic construct
GGSGGSGGSGG

SEQ ID NO: 69 - protein, 5 aa, synthetic construct
GGSGG

SEQ ID NO: 70 - protein, 18 aa, synthetic construct
GGGSEGGGSEGGGSEGGG

SEQ ID NO: 71 - protein, 8 aa, synthetic construct
AAGAATAA

SEQ ID NO: 72 - protein, 5 aa, synthetic construct
GGSSG

SEQ ID NO: 73 - protein, 11 aa, synthetic construct
GSGGGTGGGSG

SEQ ID NO: 74 - protein, 12 aa, synthetic construct
GSGSGSGSGGSG

SEQ ID NO: 75 - protein, 14 aa, synthetic construct
GSGGSGSGGSGGSG

SEQ ID NO: 76 - protein, 12 aa, synthetic construct
GGGSGGGSGGGS

SEQ ID NO: 77 - protein, 17 aa, synthetic construct
GGSGGGSGGGSGGGSLQ

SEQ ID NO: 78 - protein, 14 aa, synthetic construct
GSGGSGGSGGSGGS

SEQ ID NO: 79 - protein, 40 aa, synthetic construct
  VARIANT 1..40: note = This sequence may encompass 1-40 residues
GGGGGGGGGGGGGGGGGGGGGGGGGGGGGGGGGGGGGGGG

SEQ ID NO: 80 - RNA, 54 nt, synthetic construct
  source 1..54: mol_type = other RNA
gggaaataagagagaaaagaagagtaagaagaaatataagaccccggcgccacc

SEQ ID NO: 81 - RNA, 47 nt, synthetic construct
  source 1..47: mol_type = other RNA
gggagatcagagagaaaagaagagtaagaagaaatataagagccacc

SEQ ID NO: 82 - RNA, 42 nt, synthetic construct
  source 1..42: mol_type = other RNA
gggagacaagcttggcattccggtactgttggtaaagccacc

SEQ ID NO: 83 - RNA, 47 nt, synthetic construct
  source 1..47: mol_type = other RNA
gggaattaacagagaaaagaagagtaagaagaaatataagagccacc

SEQ ID NO: 84 - RNA, 47 nt, synthetic construct
  source 1..47: mol_type = other RNA
gggaaattagacagaaaagaagagtaagaagaaatataagagccacc

SEQ ID NO: 85 - RNA, 47 nt, synthetic construct
  source 1..47: mol_type = other RNA
gggaaataagagagtaaagaacagtaagaagaaatataagagccacc

SEQ ID NO: 86 - RNA, 47 nt, synthetic construct
  source 1..47: mol_type = other RNA
gggaaaaaagagagaaaagaagactaagaagaaatataagagccacc

SEQ ID NO: 87 - RNA, 47 nt, synthetic construct
  source 1..47: mol_type = other RNA
gggaaataagagagaaaagaagagtaagaagatatataagagccacc

SEQ ID NO: 88 - RNA, 47 nt, synthetic construct
  source 1..47: mol_type = other RNA
gggaaataagagacaaaacaagagtaagaagaaatataagagccacc

SEQ ID NO: 89 - RNA, 47 nt, synthetic construct
  source 1..47: mol_type = other RNA
gggaaattagagagtaaagaacagtaagtagaattaaaagagccacc

SEQ ID NO: 90 - RNA, 47 nt, synthetic construct
  source 1..47: mol_type = other RNA
gggaaataagagagaatagaagagtaagaagaaatataagagccacc

SEQ ID NO: 91 - RNA, 47 nt, synthetic construct
  source 1..47: mol_type = other RNA
gggaaataagagagaaaagaagagtaagaagaaaattaagagccacc

SEQ ID NO: 92 - RNA, 47 nt, synthetic construct
  source 1..47: mol_type = other RNA
gggaaataagagagaaaagaagagtaagaagaaatttaagagccacc

SEQ ID NO: 93 - RNA, 41 nt, synthetic construct
  source 1..41: mol_type = other RNA
gggaaataagagagaaaagaagagtaagaagaaatataaga

SEQ ID NO: 94 - DNA, 57 bp, synthetic construct
  source 1..57: mol_type = other DNA
gggaaataagagagaaaagaagagtaagaagaaatataagaccccggcgccgccacc

SEQ ID NO: 95 - DNA, 54 bp, synthetic construct
  source 1..54: mol_type = other DNA
gggaaataagagagaaaagaagagtaagaagaaatataagaccccggcgccacc

SEQ ID NO: 96 - DNA, 47 bp, synthetic construct
  source 1..47: mol_type = other DNA
gggaaataagagagaaaagaagagtaagaagaaatataagagccacc

SEQ ID NO: 97 - DNA, 41 bp, synthetic construct
  source 1..41: mol_type = other DNA
gggaaataagagagaaaagaagagtaagaagaaatataaga

SEQ ID NO: 98 - RNA, 141 nt, synthetic construct
  source 1..141: mol_type = other RNA
tgataataggctggagcctcggtggcctagcttcttgccccttgggcctccccccagcccctcctccccttcctgcacccgtaccccccaaacaccattgtcacactccagtggtctttgaataaagtctgagtgggcggc

SEQ ID NO: 99 - skipped

SEQ ID NO: 100 - protein, 22 aa, synthetic construct
GSGATNFSLLKQAGDVEENPGP

SEQ ID NO: 101 - DNA, 66 bp, synthetic construct
  source 1..66: mol_type = other DNA
ggaagcggagctactaacttcagcctgctgaagcaggctggagacgtggaggagaaccctggacct

SEQ ID NO: 102 - DNA, 108 bp, synthetic construct
  source 1..108: mol_type = other DNA
tccggactcagatccggggatctcaaaattgtcgctcctgtcaaacaaactcttaactttgatttactcaaactggctggggatgtagaaagcaatccaggtccactc

SEQ ID NO: 103 - RNA, 30 nt, synthetic construct
  source 1..30: mol_type = other RNA
  misc_difference 1..30: note = This sequence may encompass 1-10 ccg repeating units
ccgccgccgccgccgccgccgccgccgccg

SEQ ID NO: 104 - RNA, 10 nt, synthetic construct
  source 1..10: mol_type = other RNA
ccccggcgcc

SEQ ID NO: 105-106 - skipped (2 entries)

SEQ ID NO: 107 - RNA, 47 nt, synthetic construct
  source 1..47: mol_type = other RNA
gggaaataagagagaaaagaagagtaagaagaaatataagagccacc

SEQ ID NO: 108 - RNA, 92 nt, synthetic construct
  source 1..92: mol_type = other RNA
tcaagcttttggaccctcgtacagaagctaatacgactcactatagggaaataagagagaaaagaagagtaagaagaaatataagagccacc

SEQ ID NO: 109 - RNA, 141 nt, synthetic construct
  source 1..141: mol_type = other RNA
tgataataggctggagcctcggtggccatgcttcttgccccttgggcctccccccagcccctcctccccttcctgcacccgtaccccccaaacaccattgtcacactccagtggtctttgaataaagtctgagtgggcggc

SEQ ID NO: 110 - RNA, 164 nt, synthetic construct
  source 1..164: mol_type = other RNA
tgataataggctggagcctcggtggccatgcttcttgccccttgggcccaaacaccattgtcacactccatccccccagcccctcctccccttcctccataaagtaggaaacactacatgcacccgtacccccgtggtctttgaataaagtctgagtgggcggc

SEQ ID NO: 111 - RNA, 85 nt, Homo sapiens
  source 1..85: mol_type = genomic RNA
ccttagcagagctgtggagtgtgacaatggtgtttgtgtctaaactatcaaacgccattatcacactaaatagctactgctaggc

SEQ ID NO: 112 - RNA, 22 nt, Homo sapiens
  source 1..22: mol_type = genomic RNA
aacgccattatcacactaaata

SEQ ID NO: 113 - RNA, 22 nt, Homo sapiens
  source 1..22: mol_type = genomic RNA
tatttagtgtgataatggcgtt

SEQ ID NO: 114 - RNA, 22 nt, Homo sapiens
  source 1..22: mol_type = genomic RNA
tggagtgtgacaatggtgtttg

SEQ ID NO: 115 - RNA, 22 nt, Homo sapiens
  source 1..22: mol_type = genomic RNA
caaacaccattgtcacactcca

SEQ ID NO: 116 - RNA, 22 nt, Homo sapiens
  source 1..22: mol_type = genomic RNA
tagcttatcagactgatgttga

SEQ ID NO: 117 - RNA, 21 nt, Homo sapiens
  source 1..21: mol_type = genomic RNA
caacaccagtcgatgggctgt

SEQ ID NO: 118 - RNA, 23 nt, Homo sapiens
  source 1..23: mol_type = genomic RNA
tgtagtgtttcctactttatgga

SEQ ID NO: 119 - RNA, 21 nt, Homo sapiens
  source 1..21: mol_type = genomic RNA
cataaagtagaaagcactact

SEQ ID NO: 120 - RNA, 1407 nt, synthetic construct
  source 1..1407: mol_type = other RNA
gacgagcctcctcagagcccttgggacagagtgaaggacttggccaccgtgtacgtggacgtgcttaaggacagcggcagagactacgtgagccagttcgagggcagcgccctgggcaagcagctgaacctgaagctgctggacaactgggacagcgtgaccagcaccttcagcaagctgagagagcagctgggccctgtgacccaggagttctgggacaacctggagaaggagacagagggcctgagacaggagatgagcaaggacttagaggaggtgaaggccaaggtgcagccttacctggacgacttccagaagaagtggcaggaggagatggagctgtacagacagaaggtggagcctctgagagccgagctgcaggagggcgcacggcagaagctgcacgaactgcaggagaagctgagccctctgggcgaggaaatgagagacagagccagagcccacgtggatgccctgagaacccacctggccccttactcggacgagctgcgacagagactggccgccagactggaggccctgaaggagaacggcggcgcaagactcgcagagtaccacgccaaggccaccgagcacctgagcaccctgagcgagaaggctaagcctgctctggaggacctgcggcagggcctgctgcctgtgctggagagcttcaaggtgagcttcctgtccgcactggaagaatacaccaagaagctgaacacccagggcggcggaggatcgggtggaggtggtagtggtggtggcggttccatcacctgccctcctcctatgagcgtggagcacgccgacatctgggtaaagtcttacagcctgtacagcagagagagatacatctgcaacagcggcttcaagagaaaggccggcaccagcagcctgaccgagtgtgtcctgaacaaggcaaccaacgtggcccactggaccacccctagcctgaagtgcatcagagatccagcccttgtgcaccaaagacctgcccctccatcaggaggttccggcggtggaggatcaggcggcggcagcggaggtggcggatccctgcagaactgggtaaacgtgatcagcgacctgaagaagatcgaagatctgatccagagcatgcacatcgacgccaccctgtacaccgagagcgacgtgcaccctagctgcaaggtgaccgccatgaagtgcttcctgctggaactccaggttatctccctggagagtggcgacgccagcatccacgacaccgtggagaacctgatcatcctggccaacaacagcctgagcagcaacggcaacgtgactgaaagcggctgtaaggagtgcgaggagctcgaggagaagaacatcaaggagttcctgcagagcttcgtccacatcgtgcagatgttcatcaacaccagc

SEQ ID NO: 121 - protein, 469 aa, synthetic construct
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGGGGSGGGGSGGGGSITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 122 - RNA, 1371 nt, synthetic construct
  source 1..1371: mol_type = other RNA
gacgagcctcctcagagcccttgggacagagtgaaggacctcgccaccgtgtacgtggacgtgctcaaggacagcggcagagactacgtgagccagttcgagggcagcgccctgggcaagcagctgaacctgaagctgctggacaactgggacagcgtgaccagcaccttcagcaagctgagagagcagctgggccctgtgacccaggagttctgggacaacctggagaaggagactgagggcctgagacaggagatgagcaaggatctggaggaggtgaaggccaaggtgcagccttacctggacgacttccagaagaagtggcaggaggagatggagctgtacagacagaaggtggagcctctgagagccgagctgcaggagggcgcacgtcagaagctgcacgaacttcaggagaagctgagccctctgggcgaagaaatgagagacagagccagagcccatgtggacgccctgagaacccacctggccccttacagcgacgaactgagacaaagactggccgccagactggaggccctgaaggagaacggcggtgcgagattagctgagtaccacgccaaggccaccgagcacctgagcaccctgagcgagaaggctaagcctgccctcgaggacctgcgacaaggcctgctgcctgtgctggagagcttcaaggtgagcttcctgtcagcacttgaagagtacaccaagaagctgaacacccagggcggcggcggaagcggcggtggcggcagtggaggcggtggaagcatcacctgcccgcctcctatgagcgtggagcacgccgacatctgggtaaagtcctacagcctgtacagcagagagagatacatctgcaacagcggcttcaagagaaaggccggcaccagcagcctgaccgagtgcgtgcttaataaggccactaacgtggcccactggaccacccctagcctgaagtgcatcagagacggcggttcaggaggcggaggttccggtggaggctcaggtggaggtggtagcctgcagaactgggtaaacgtgatcagcgacctgaagaagatcgaagatctcatccagagcatgcacatcgacgccaccctgtacaccgagagcgacgtgcaccctagctgcaaggtgaccgccatgaagtgcttcctgctggaactgcaggtgataagcctggagtctggcgacgccagcatccacgacaccgtggagaacctgatcatcctggccaacaacagcctgagcagcaacggcaacgtaaccgagtccggctgtaaggagtgcgaggagttggaggagaagaacatcaaggagttcctgcagagcttcgttcacatcgtgcagatgttcatcaacaccagc

SEQ ID NO: 123 - protein, 457 aa, synthetic construct
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGGGGSGGGGSGGGGSITCPPPMSVEHADIWVKSYSLYSRERYICNSGFKRKAGTSSLTECVLNKATNVAHWTTPSLKCIRDGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 124 - protein, 175 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 125 - RNA, 525 nt, Homo sapiens
  source 1..525: mol_type = genomic RNA
ataacctgtccacctccaatgagcgtcgagcacgcagacatttgggtgaaatcatacagtctgtacagtagagagcggtacatctgcaacagtgggtttaaaagaaaagcaggcacttcatctctgacagagtgcgtgctgaacaaagcaactaatgtagctcattggacaaccccatcactgaaatgcattagagatccagctctggtgcatcaaagaccagcaccaccaagtaccgtaacaaccgcaggggtgacccctcagcctgagtccctatctccctccggcaaggagccagcagcatcttcacctagctccaataacaccgcagctaccactgccgccatagtccccggaagccagctgatgcctagtaaatctccctcaacaggtaccaccgaaatttctagccatgagtcctcgcacggcaccccgtcacagactacagctaaaaactgggagctaacggcttcggcatcccaccaacctccaggcgtttatccccaaggtcactccgacactact

SEQ ID NO: 126 - RNA, 1329 nt, synthetic construct
  source 1..1329: mol_type = other RNA
gagcctaagagctgcgacaagacccacacctgccctccttgcccggccccagagttgcttggcggccctagcgtcttcctcttccctcctaagcctaaggacaccctgatgatcagcagaacccctgaggtgacctgcgtggtggtggccgtgagccacgaggaccctgaagtaaagttcaactggtacgtggacggcgtggaggtgcacaacgccaagaccaagcctagagaggagcagtacaacagcacctacagagtggtgagcgtgctgaccgtgctgcaccaggactggctgaacggcaaggagtacaagtgcaaggtgagcaacaaggccctgcctgcacctatcgagaagaccatcagcaaggccaagggccaacctcgagagcctcaggtgtacaccctgcctcctagcagagacgagctgaccaagaaccaggtgagcctgacctgcctggtgaagggcttctaccctagcgacatcgccgtggagtgggagagcaacggccaaccagagaacaactacaagaccacccctcctgtgctggacagcgacggcagcttcttcctctacagcaagctgactgttgacaagagcagatggcagcagggcaacgtgttcagctgcagcgtgatgcacgaggcgctgcacaaccactacacccagaagagtctttccctgagccctggcaagataacgtgtccgcctcctatgagcgtggagcacgccgacatctgggtgaagagttacagcctgtattccagagagagatacatctgcaacagcggcttcaagagaaaggccggcaccagcagcttaaccgagtgcgtccttaacaaggctaccaacgtggcccactggacaacgccgagcctgaagtgcatccgcgaccctgccctggtgcaccaacgtccagcacctccgagtggcggcagtggtggcggaggctcaggcggtggtagtggaggaggcggaagtctgcagaattgggtgaacgtgatcagcgacctgaagaagatcgaggacctgatccagagcatgcacatcgacgccaccctgtacaccgagagcgacgtgcaccctagctgtaaggtaaccgccatgaagtgcttcctgctggagctgcaggtgattagcctggagagcggcgacgccagcatccacgacaccgtggagaacctgatcatcctggccaacaattctctcagttctaacggtaacgtgaccgaatcgggttgtaaggagtgcgaggagctggaggagaagaacatcaaggagttcctgcagagcttcgtgcacattgtgcagatgttcatcaacaccagc

SEQ ID NO: 127 - protein, 443 aa, synthetic construct
EPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 128 - RNA, 15 nt, synthetic construct
  source 1..15: mol_type = other RNA
  misc_difference 1..15: note = This sequence may encompass 1-5 ccg repeating units
ccgccgccgccgccg

SEQ ID NO: 129 - RNA, 12 nt, synthetic construct
  source 1..12: mol_type = other RNA
ccgccgccgccg

SEQ ID NO: 130 - RNA, 15 nt, synthetic construct
  source 1..15: mol_type = other RNA
ccgccgccgccgccg

SEQ ID NO: 131 - protein, 4 aa, synthetic construct
NPGP

SEQ ID NO: 132 - protein, 420 aa, synthetic construct
  VARIANT 1..20: note = This region may encompass 1-20 residues
  VARIANT 22..41: note = This region may encompass 1-20 residues
  VARIANT 43..62: note = This region may encompass 1-20 residues
  VARIANT 64..83: note = This region may encompass 1-20 residues
  VARIANT 85..104: note = This region may encompass 1-20 residues
  VARIANT 106..125: note = This region may encompass 1-20 residues
  VARIANT 127..146: note = This region may encompass 1-20 residues
  VARIANT 148..167: note = This region may encompass 1-20 residues
  VARIANT 169..188: note = This region may encompass 1-20 residues
  VARIANT 190..209: note = This region may encompass 1-20 residues
  VARIANT 211..230: note = This region may encompass 1-20 residues
  VARIANT 232..251: note = This region may encompass 1-20 residues
  VARIANT 253..272: note = This region may encompass 1-20 residues
  VARIANT 274..293: note = This region may encompass 1-20 residues
  VARIANT 295..314: note = This region may encompass 1-20 residues
  VARIANT 316..335: note = This region may encompass 1-20 residues
  VARIANT 337..356: note = This region may encompass 1-20 residues
  VARIANT 358..377: note = This region may encompass 1-20 residues
  VARIANT 379..398: note = This region may encompass 1-20 residues
  VARIANT 400..419: note = This region may encompass 1-20 residues
  VARIANT 1..420: note = This sequence may encompass 1-20 GnS repeating units, wherein n = 1-20
GGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGS

SEQ ID NO: 133 - RNA, 24 nt, synthetic construct
  source 1..24: mol_type = other RNA
gtgcacaatgatcatcgtctagat

(19) United States
(12) Patent Application Publication
(10) Pub. No.: US 2025/0281583 A1
(43) Pub. Date: Sep. 11, 2025

(54) COMPOSITION OF MRNA-ENCODED IL15 FUSION PROTEINS AND METHODS OF USE THEREOF

(71) Applicants: ModernaTX, Inc., Cambridge, MA (US); Fundación Para La Investigación Médica Aplicada, Pamplona (ES)

(72) Inventors: Pedro Berraondo López, Pamplona (ES); Ignacio Javier Melero Bermejo, Pamplona (ES); Myriam Fernández Fernández, Pamplona (ES); Maite Álvarez Rodríguez, Pamplona (ES); Joshua P. Frederick, Cambridge, MA (US); Ailin Bai, Cambridge, MA (US)

(21) Appl. No.: 18/700,981

(22) PCT Filed: Oct. 13, 2022
(86) PCT No.: PCT/US2022/046566

§ 371 Date: Apr. 12, 2024

Related U.S. Application Data
(60) Provisional application No. 63/255,329, filed on Oct. 13, 2021.

(51) Int. Cl. A61K 39/00; A61K 9/00; A61K 9/1272; A61K 9/51; A61K 38/00; A61P 35/00; C07K 14/54; C07K 14/715; C07K 14/775

(52) U.S. Cl. CPC A61K 39/001119; A61K 9/0019; A61K 9/1272; A61K 9/5123; A61K 39/00114; A61P 35/00; C07K 14/5443; C07K 14/715; C07K 14/775; A61K 38/00; A61K 2039/53; A61K 2039/54; A61K 2039/55555; A61K 2039/572; C07K 2319/02; C12N 2830/50

(57) ABSTRACT

The present disclosure provides mRNAs encoding an IL 15 fusion protein comprising an IL 15 polypeptide, an IL 15Rα polypeptide, and an ApoA polypeptide and methods of treating cancer, including solid tumors and disseminated cancers such as myeloid malignancies, using the mRNAs described herein, optionally formulated as lipid nanoparticles.

### CROSS-REFERENCE TO RELATED APPLICATIONS

This application is a U.S. National Phase Application under 35 U.S.C. § 371 of International Application No. PCT/US2022/046566 filed Oct. 13, 2022, which claims the benefit of U.S. Provisional Application Ser. No. 63/255,329, filed on Oct. 13, 2021. The entire contents of the above-referenced applications are incorporated herein by reference.

### SEQUENCE LISTING

The instant application contains a Sequence Listing which has been submitted electronically in XML format and is hereby incorporated by reference in its entirety. Said XML copy, created on Jan. 13, 2025, is named MRNA-819-US-PCT_SL.xml and is 154,281 bytes in size.

### BACKGROUND

Cancer immunotherapy has revolutionized oncology practice due to the consolidation of checkpoint inhibitors and adoptive transfer of T lymphocytes for the treatment of various malignancies. Despite the impressive clinical results, primary and secondary resistance limits the percentage of patients that benefit from these new immunotherapies (Berraondo P, et al. British Journal of Cancer. 2019; 120(1):6-15; Sharma P, et al., Cell. 2017; 168(4):707-23). Strategies to expand the main antitumor effector immune cells such as T lymphocytes and NK cells may improve the outcome of these new immunotherapies. This goal can be achieved with cytokines of the IL2 family (Berraondo P, et al. British Journal of Cancer. 2019; 120(1):6-15). IL2 cytokine is widely used in adoptive transfer protocols to expand cultured lymphocytes and to increase the persistence of the transferred cells in cancer patients (Rosenberg S A, et al Nature Reviews Cancer. 2008; 8(4):299-308). Moreover, infusion of high doses of this cytokine is approved for the treatment of RCC and metastatic melanoma (Berraondo P, et al. British Journal of Cancer. 2019; 120(1):6-15).

Interleukin 15 (“IL-15” or “IL15”) is a cytokine that has been described as a soluble factor mimicking the activities of IL2 in vitro (Grabstein, et al (1994) Science 264:965). Both cytokines belong to the four-α-helix bundle family, and their membrane receptors share two subunits (IL2/IL15β and IL2/IL15R7 chains) responsible for signal transduction (Giri, et al (1994) EMBO J. 13:2822). High affinity IL2 and IL15 receptors incorporate a private chain (IL2Rα and IL15Rα respectively) that confer cytokine specificity and enhanced affinity for cytokine binding (Anderson, et al (1995) J. Biol Chem 270:29862). The IL15Rα and IL2R≢ subunits form a sub-family of cytokine receptors that comprise at the N-terminus of their extracellular domain a “sushi” structural domain (one in IL15Rα and two in IL2Rα) that are also found in complement and adhesion molecules (Norman, et al (1991) J. Mol Biol 219:717). In both cases the sushi domain contains the structural elements of the respective receptors that enable cytokine binding. For example, the IL15Rα sushi domain contains the major structural elements that facilitate IL15 binding (Mortier, et al (2006) J. Biol Chem 281:1612).

Despite similar functional effects in vitro, IL2 and IL15 exert complementary actions in vivo. Both cytokines contribute to innate and adaptive immunity. But whereas IL2 plays a major role in limiting continuous expansion of activation of T cells, IL15 is critical for the development of NK cells, the initiation of T cell division, and the survival of memory T cells (Kennedy, et al (2000) JExp Med 191:771; Lodolce, et al (2001) JExp Med 194: 1187; Li, et al (2001) Nat. Med. 7:114). Additionally, unlike IL2, IL15 expands T and NK cells without expanding Tregs (Steel J C, et al. Trends in Pharmacological Sciences. 2012; 33(1):35-41).

Recombinant IL15 or optimized variants are being evaluated in the clinic as a monotherapy or in combination with T cells or NK cell adoptive transfer or antibodies that induce antibody-dependent cellular cytotoxicity (ADCC) (Cooley S, et al. Blood Advances. 2019; 3(13):1970-80; Conlon K C, et al., Clinical Cancer Research. 2019; 25(16):4945-54). However, the efficacy of IL15 is limited by its short plasma half-life (Kukita, et al (2002) Br. J. Haematol. 119:467-74). Thus, the in vivo application of recombinant IL15 requires the use of high doses and frequent administration, which can result in undesirable systemic toxicity. Moreover, therapeutic use of recombinant proteins has drawbacks for long-term treatment of cancer, such as the high production cost.

Accordingly, there is a need for formulations that enable delivery of IL15 in a manner that maximizes its anti-tumor effects, while mitigating IL15-related secondary effects.

### SUMMARY

In some aspects, the disclosure provides a messenger RNA (mRNA) comprising an open reading frame (ORF) encoding a fusion protein, wherein the fusion protein comprises from N-terminus to C-terminus: (i) an apolipoprotein A (ApoA) polypeptide; (ii) an extended IL15 Receptor alpha (IL15Rα) Sushi polypeptide; and (iii) an interleukin 15 (IL15) polypeptide, wherein (i), (ii), and (iii) are operably linked, optionally via a linker. In some aspects, the extended IL15Rα Sushi polypeptide comprises the Sushi domain of a human IL15R≢ ectodomain, wherein the human IL15Rα ectodomain comprises the amino acid sequence of SEQ ID NO: 51. In some aspects, the human IL15Rα ectodomain comprises an amino acid sequence having at least about 90% identity to SEQ ID NO: 51. In some aspects, the human IL15R≢ ectodomain comprises an amino acid sequence having at least about 90%, about 91%, about 92%, about 93%, about 94%, about 95%, about 96%, about 97%, about 98%, or about 99% identity to SEQ ID NO: 51. In some aspects, the extended IL15Rα Sushi polypeptide comprises a contiguous amino acid sequence extending from the N-terminus of the Sushi domain to at least one amino acid residue after the fourth cysteine residue of the Sushi domain of a human IL15R≢ ectodomain, wherein the human IL15Rα ectodomain comprises the amino acid sequence of SEQ ID NO: 51. In some aspects, the extended IL15Rα Sushi polypeptide comprises a contiguous amino acid sequence extending from the N-terminus of the Sushi domain to at least one amino acid residue after the fourth cysteine residue of the Sushi domain of a human IL15R≢ ectodomain, wherein the human IL15Rα ectodomain comprises an amino acid sequence having at least about 90% identity to SEQ ID NO: 51. In some aspects, the extended IL15Rα Sushi polypeptide comprises a contiguous amino acid sequence extending from an amino acid residue at position 31, 32, or 33 of a human IL15Rα ectodomain to at least one amino acid residue after position 93 (e.g., extends to an amino acid residue at position 94, 95, 96, 97, 98, 99, 100, 101, 102, 103, 104, 105, 106, 107, 108, 109, or 110), wherein the human IL15Rα ectodomain comprises an amino acid sequence having at least about 90%, about 91%, about 92%, about 93%, about 94%, about 95%, about 96%, about 97%, about 98%, about 99%, or 100% identity to SEQ ID NO: 51.

In any of the foregoing or related aspects, the extended IL15Rα Sushi polypeptide is at least 62 amino acid residues in length. In some aspects, the extended IL15Rα Sushi polypeptide is 62-80 amino acid residues in length. In some aspects, the extended IL15Rα Sushi polypeptide is 62-66 amino acid residues in length. In some aspects, the extended IL15Rα Sushi polypeptide is 66 amino acid residues in length. In some aspects, the extended IL15Rα Sushi polypeptide is 66-78 amino acid residues in length. In some aspects, the extended IL15Rα Sushi polypeptide is 78 amino acid residues in length. In some aspects, the extended IL15Rα Sushi polypeptide is 78-80 amino acid residues in length.

In some aspects, the disclosure provides an mRNA comprising an ORF encoding a fusion protein, wherein the fusion protein comprises from N-terminus to C-terminus: (i) an ApoA polypeptide; (ii) an extended IL15Rα Sushi polypeptide comprising an amino acid sequence having at least about 90% identity to the amino acid sequence of SEQ ID NO: 17; and (iii) an IL15 polypeptide, wherein (i), (ii), and (iii) are operably linked, optionally via a linker. In some embodiments, the extended IL15Rα Sushi polypeptide comprises an amino acid sequence having at least about 90%, about 91%, about 92%, about 93%, about 94%, about 95%, about 96%, about 97%, about 98%, or about 99% identity to the amino acid sequence of SEQ ID NO: 17. In some embodiments, the extended IL15Rα Sushi polypeptide comprises the amino acid sequence of SEQ ID NO: 17. In some aspects, the extended IL15Rα Sushi polypeptide is encoded by a nucleotide sequence having at least about 80%, about 85%, about 90%, about 95%, or about 99% identity to a nucleotide sequence selected from SEQ ID NOs: 29-31. In some aspects, the extended IL15Rα Sushi polypeptide is encoded by a nucleotide sequence having at least about 90%, about 91%, about 92%, about 93%, about 94%, about 95%, about 96%, about 97%, about 98%, or about 99% to a nucleotide sequence selected from SEQ ID NOs: 29-31. In some aspects, the extended IL15Rα Sushi polypeptide is encoded by a nucleotide sequence selected from SEQ ID NOs: 29-31.

In some aspects, the disclosure provides an mRNA comprising an ORF encoding a fusion protein, wherein the fusion protein comprises from N‘ terminus to C’ terminus: (i) an ApoA polypeptide; (ii) an extended IL15 Receptor alpha (IL15Rα) Sushi polypeptide comprising the amino acid having at least about 90% identity to the amino acid sequence of SEQ ID NO: 18; and (iii) an IL15 polypeptide, wherein (i), (ii), and (iii) are operably linked, optionally via a linker. In some embodiments, the extended IL15Rα Sushi polypeptide comprises an amino acid sequence having at least about 90%, about 91%, about 92%, about 93%, about 94%, about 95%, about 96%, about 97%, about 98%, or about 99% identity to the amino acid sequence of SEQ ID NO: 18. In some embodiments, the extended IL15Rα Sushi polypeptide comprises the amino acid sequence of SEQ ID NO: 18. In some aspects, the extended IL15Rα Sushi polypeptide is encoded by a nucleotide sequence comprising a nucleotide sequence having at least 80%, about 85%, about 90%, about 95%, or about 99% identity to a nucleotide sequence selected from SEQ ID NOs: 32 and 33. In some aspects, the extended IL15Rα Sushi polypeptide is encoded by a nucleotide sequence having at least about 90%, about 91%, about 92%, about 93%, about 94%, about 95%, about 96%, about 97%, about 98%, or about 99% to a nucleotide sequence selected from SEQ ID NOs: 32 and 33. In some aspects, the extended IL15Rα Sushi polypeptide is encoded by a nucleotide sequence selected from SEQ ID NOs: 32 and 33.

In any of the foregoing or related aspects, the ApoA polypeptide comprises a human origin ApoA-1 polypeptide or functional derivative thereof. In some aspects, the ApoA polypeptide comprises an amino acid sequence having at least about 90%, about 91%, about 92%, about 93%, about 94%, about 95%, about 96%, about 97%, about 98%, or about 99% identity to the amino acid sequence of SEQ ID NO: 14. In some aspects, the ApoA polypeptide comprises the amino acid sequence of SEQ ID NO: 14. In some aspects, the ApoA polypeptide is encoded by a nucleotide sequence having at least about 80% sequence identity to a nucleotide sequence selected from SEQ ID NOs: 34-37. In some aspects, the ApoA polypeptide is encoded by a nucleotide sequence having at least about 80%, about 85%, about 90%, about 95%, or about 99% identity to a nucleotide sequence selected from SEQ ID NOs: 34-37. In some aspects, the ApoA polypeptide is encoded by a nucleotide sequence having at least about 90%, about 91%, about 92%, about 93%, about 94%, about 95%, about 96%, about 97%, about 98%, or about 99% identity to a nucleotide sequence selected from SEQ ID NOs: 34-37. In some aspects, the ApoA polypeptide is encoded by a nucleotide sequence selected from SEQ ID NOs: 34-37.

In any of the foregoing or related aspects, the IL15 polypeptide is a human IL15 polypeptide or functional derivative thereof. In some aspects, the IL15 polypeptide comprises an amino acid sequence having at least about 80%, about 85%, about 90%, about 95%, or about 99% identity to the amino acid sequence of SEQ ID NO: 16. In some aspects, the IL15 polypeptide comprises an amino acid sequence having at least about 90%, about 91%, about 92%, about 93%, about 94%, about 95%, about 96%, about 97%, about 98%, or about 99% identity to the amino acid sequence of SEQ ID NO: 16. In some aspects, the IL15 polypeptide comprises the amino acid sequence of SEQ ID NO: 16. In some aspects, the IL15 polypeptide is encoded by a nucleotide sequence having at least about 80%, about 85%, about 90%, about 95%, or about 99% identity to a nucleotide sequence selected from SEQ ID NOs: 38-42. In some aspects, the IL15 polypeptide is encoded by a nucleotide sequence having at least about 90%, about 91%, about 92%, about 93%, about 94%, about 95%, about 96%, about 97%, about 98%, or about 99% identity to a nucleotide sequence selected from SEQ ID NOs: 38-42. In some aspects, the IL15 polypeptide is encoded by a nucleotide sequence selected from SEQ ID NOs: 38-42.

In any of the foregoing or related aspects, the ApoA polypeptide is directly fused to the extended IL15Rα Sushi polypeptide. In some aspects, the ApoA polypeptide is operably linked to the extended IL15Rα Sushi polypeptide by a linker. In some aspects, the linker is a peptide linker. In some aspects, the IL15Rα Sushi polypeptide is directly fused to the IL15 polypeptide.

In some aspects, the IL15Rα Sushi polypeptide is operably linked to the IL15 polypeptide by a linker. In some aspects, the linker is a peptide linker. In some aspects, the peptide linker is a GlySer linker. In some aspects, the peptide linker (e.g., GlySer linker) comprises an amino acid sequence selected from SEQ ID NOs: 53-79. In some aspects, the GlySer linker comprises (GGGS)₃ (SEQ ID NO: 76).

In some aspects, the disclosure provides an mRNA comprising an ORF encoding a fusion protein, wherein the fusion protein comprises an amino acid sequence having at least about 80%, about 85%, about 90%, about 95%, or about 99% identity to the amino acid sequence of SEQ ID NO: 123. In some aspects, the fusion protein comprises an amino acid sequence having at least about 90%, about 91%, about 92%, about 93%, about 94%, about 95%, about 96%, about 97%, about 98%, or about 99% identity to the amino acid sequence of SEQ ID NO: 123. In some aspects, the fusion protein comprises the amino acid sequence of SEQ ID NO: 123. In some aspects, the fusion protein is encoded by a nucleotide sequence having at least about 80%, about 85%, about 90%, about 95%, or about 99% identity to the nucleotide sequence of SEQ ID NO: 122. In some aspects, the fusion protein is encoded by a nucleotide sequence having at least about 90%, about 91%, about 92%, about 93%, about 94%, about 95%, about 96%, about 97%, about 98%, or about 99% identity to the nucleotide sequence of SEQ ID NO: 122. In some aspects, the fusion protein is encoded by the nucleotide sequence of SEQ ID NO: 122. In some aspects, the fusion protein comprises a signal peptide at the N-terminus. In some aspects, the signal peptide is a human IgG heavy chain signal peptide. In some aspects, the signal peptide comprises the amino acid sequence of SEQ ID NO: 13.

In some aspects, the disclosure provides an mRNA comprising an ORF encoding a fusion protein, wherein the ORF comprises a nucleotide sequence having at least about 80% identity to the nucleotide sequence of SEQ ID NO: 122. In some aspects, the ORF comprises a nucleotide sequence having at least about 80%, about 85%, about 90%, about 95%, or about 99% identity to the nucleotide sequence of SEQ ID NO: 122. In some aspects, the ORF comprises a nucleotide sequence having at least about 90%, about 91%, about 92%, about 93%, about 94%, about 95%, about 96%, about 97%, about 98%, or about 99% identity to the nucleotide sequence of SEQ ID NO: 122. In some aspects, the ORF comprises the nucleotide sequence of SEQ ID NO: 122.

In some aspects, the disclosure provides an mRNA comprising an ORF encoding a fusion protein, wherein the fusion protein comprises an amino acid sequence having at least about 80%, about 85%, about 90%, about 95%, or about 99% identity to the amino acid sequence of SEQ ID NO: 121. In some aspects, the fusion protein comprises an amino acid sequence having at least about 90%, about 91%, about 92%, about 93%, about 94%, about 95%, about 96%, about 97%, about 98%, or about 99% identity to the amino acid sequence of SEQ ID NO: 121. In some aspects, the fusion protein comprises the amino acid sequence of SEQ ID NO: 121. In some aspects, the fusion protein is encoded by a nucleotide sequence having at least about 80%, about 85%, about 90%, about 95%, or about 99% identity to the nucleotide sequence of SEQ ID NO: 120. In some aspects, the fusion protein is encoded by a nucleotide sequence having at least about 90%, about 91%, about 92%, about 93%, about 94%, about 95%, about 96%, about 97%, about 98%, or about 99% identity to the nucleotide sequence of SEQ ID NO: 120. In some aspects, the fusion protein is encoded by the nucleotide sequence of SEQ ID NO: 120. In some aspects, the fusion protein comprises a signal peptide at the N-terminus. In some aspects, the signal peptide is a human IgG heavy chain signal peptide. In some aspects, the signal peptide comprises the amino acid sequence of SEQ ID NO: 13.

In some aspects, the disclosure provides an mRNA comprising an ORF encoding a fusion protein, wherein the ORF comprises a nucleotide sequence having at least about 80% identity to the nucleotide sequence of SEQ ID NO: 120. In some aspects, the ORF comprises a nucleotide sequence having at least about 80%, about 85%, about 90%, about 95%, or about 99% identity to the nucleotide sequence of SEQ ID NO: 120. In some aspects, the ORF comprises a nucleotide sequence having at least about 90%, about 91%, about 92%, about 93%, about 94%, about 95%, about 96%, about 97%, about 98%, or about 99% identity to the nucleotide sequence of SEQ ID NO: 120. In some aspects, the ORF comprises the nucleotide sequence of SEQ ID NO: 120.

In any of the foregoing or related aspects, the mRNA comprises a 5′ untranslated region (UTR). In some aspects, the 5′UTR comprises a nucleotide sequence having at least about 80%, about 85%, about 90%, about 95%, or about 99% identity to the nucleotide sequence of SEQ ID NO: 19. In some aspects, the 5′UTR comprises a nucleotide sequence having at least about 90%, about 91%, about 92%, about 93%, about 94%, about 95%, about 96%, about 97%, about 98%, or about 99% identity to the nucleotide sequence of SEQ ID NO: 19. In some aspects, the 5′UTR comprises the nucleotide sequence set forth in SEQ ID NO: 19.

In any of the foregoing or related aspects, the mRNA comprises a 3′UTR. In some aspects, the 3′UTR comprises a nucleotide sequence having at least about 80%, about 85%, about 90%, about 95%, or about 99% identity to the nucleotide sequence set forth in SEQ ID NO: 20. In some aspects, the 3′UTR comprises a nucleotide sequence having at least about 90%, about 91%, about 92%, about 93%, about 94%, about 95%, about 96%, about 97%, about 98%, or about 99% identity to the nucleotide sequence set forth in SEQ ID NO: 20. In some aspects, the 3′UTR comprises the nucleotide sequence set forth in SEQ ID NO: 20. In some aspects, the 3′UTR comprises at least one microRNA (miR) binding site.

In any of the foregoing or related aspects, the mRNA comprises at least one chemical modification. In some aspects, the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine. In some aspects, at least 95% of uridines are chemically-modified. In some aspects, at least 99% of uridines are chemically-modified. In some aspects, 100% of uridines are chemically-modified. In some aspects, at least 95% of uridines are N1-methylpseudouridine. In some aspects, at least 99% of uridines are N1-methylpseudouridine. In some aspects, 100% of uridines are N1-methylpseudouridine.

In any of the foregoing or related aspects, the mRNA comprises a polyA tail. In some aspects, the mRNA comprises a 5′Cap. In some aspects, the 5′Cap is a Cap 1 structure. In some aspects, the disclosure provides a lipid nanoparticle (LNP) comprising an mRNA described herein. In some aspects, the LNP comprises an ionizable amino lipid, a phospholipid, a structural lipid, and a polyethylene glycol (PEG)-modified lipid. In some aspects, the LNP comprises 40-60% ionizable amino lipid, 8-16% phospholipid, 30-45% sterol, and 1-5% PEG modified lipid. In some aspects, the LNP comprises a molar ratio of 45-65% ionizable amino lipid, 5-10% phospholipid, 25-40% sterol, and 0.5-5% PEG modified lipid. In some aspects, the ionizable amino lipid is Compound 1. In some aspects, the sterol is cholesterol and the PEG-modified lipid is PEG-DMG. In some aspects, the sterol is cholesterol and the PEG-modified lipid is Compound 2. In some aspects, the LNP comprises about 40-60 mol % Compound 1; about 8-16 mol % DSPC; about 30-45 mol % cholesterol; and about 1-5 mol % PEG-DMG. In some aspects, the LNP comprises about 45-65 mol % Compound 1; about 5-10 mol % DSPC; about 25-40 mol % cholesterol; and about 0.5-5 mol % PEG-DMG. In some aspects, the LNP comprises about 40-60 mol % Compound 1; about 8-16 mol % DSPC; about 30-45 mol % cholesterol; and about 1-5 mol % Compound 2. In some aspects, the LNP comprises about 45-65 mol % Compound 1; about 5-10 mol % DSPC; about 25-40 mol % cholesterol; and about 0.5-5 mol % Compound 2. In some aspects, the LNP is formulated for intravenous delivery.

In some aspects, the disclosure provides an LNP comprising an mRNA comprising an ORF encoding a fusion protein, wherein the fusion protein comprises an amino acid sequence having at least about 90%, about 91%, about 92%, about 93%, about 94%, about 95%, about 96%, about 97%, about 98%, or about 99% identity to the amino acid sequence of SEQ ID NO: 123, and wherein the LNP comprises an ionizable amino lipid, a phospholipid, a sterol, and a PEG modified lipid. In some aspects, the disclosure provides an LNP comprising an mRNA comprising an ORF encoding a fusion protein, wherein the fusion protein comprises the amino acid sequence of SEQ ID NO: 123, and wherein the LNP comprises an ionizable amino lipid, a phospholipid, a sterol, and a PEG modified lipid. In some aspects, the disclosure provides an LNP comprising an mRNA comprising an ORF encoding a fusion protein, wherein the ORF comprises a nucleotide sequence having at least about 80%, about 85%, about 90%, about 91%, about 92%, about 93%, about 94%, about 95%, about 96%, about 97%, about 98%, or about 99% identity to the nucleotide sequence of SEQ ID NO: 122, and wherein the LNP comprises an ionizable amino lipid, a phospholipid, a sterol, and a PEG modified lipid. In some aspects, the disclosure provides an LNP comprising an mRNA comprising an ORF encoding a fusion protein, wherein the ORF comprises the nucleotide sequence of SEQ ID NO: 122, and wherein the LNP comprises an ionizable amino lipid, a phospholipid, a sterol, and a PEG modified lipid. In some aspects, the ionizable amino lipid is Compound 1. In some aspects, the sterol is cholesterol and the PEG-modified lipid is PEG-DMG. In some aspects, the sterol is cholesterol and the PEG-modified lipid is Compound 2.In some aspects, the disclosure provides an LNP comprising an mRNA comprising an ORF encoding a fusion protein, wherein the fusion protein comprises an amino acid sequence having at least about 90%, about 91%, about 92%, about 93%, about 94%, about 95%, about 96%, about 97%, about 98%, or about 99% identity to the amino acid sequence of SEQ ID NO: 123, and wherein the LNP comprises 40-60% ionizable amino lipid, 8-16% phospholipid, 30-45% sterol, and 1-5% PEG modified lipid. In some aspects, the disclosure provides an LNP comprising an mRNA comprising an ORF encoding a fusion protein, wherein the fusion protein comprises the amino acid sequence of SEQ ID NO: 123, and wherein the LNP comprises 40-60% ionizable amino lipid, 8-16% phospholipid, 30-45% sterol, and 1-5% PEG modified lipid. In some aspects, the disclosure provides an LNP comprising an mRNA comprising an ORF encoding a fusion protein, wherein the ORF comprises a nucleotide sequence having at least about 80%, about 85%, about 90%, about 91%, about 92%, about 93%, about 94%, about 95%, about 96%, about 97%, about 98%, or about 99% identity to the nucleotide sequence of SEQ ID NO: 122, and wherein the LNP comprises 40-60% ionizable amino lipid, 8-16% phospholipid, 30-45% sterol, and 1-5% PEG modified lipid. In some aspects, the disclosure provides an LNP comprising an mRNA comprising an ORF encoding a fusion protein, wherein the ORF comprises the nucleotide sequence of SEQ ID NO: 122, and wherein the LNP comprises 40-60% ionizable amino lipid, 8-16% phospholipid, 30-45% sterol, and 1-5% PEG modified lipid. In some aspects, the ionizable amino lipid is Compound 1. In some aspects, the sterol is cholesterol and the PEG-modified lipid is PEG-DMG. In some aspects, the sterol is cholesterol and the PEG-modified lipid is Compound 2.

In some aspects, the disclosure provides an LNP comprising an mRNA comprising an ORF encoding a fusion protein, wherein the fusion protein comprises an amino acid sequence having at least about 90%, about 91%, about 92%, about 93%, about 94%, about 95%, about 96%, about 97%, about 98%, or about 99% identity to the amino acid sequence of SEQ ID NO: 123, and wherein the LNP comprises 45-65% ionizable amino lipid, 5-10% phospholipid, 25-40% sterol, and 0.5-5% PEG modified lipid. In some aspects, the disclosure provides an LNP comprising an mRNA comprising an ORF encoding a fusion protein, wherein the fusion protein comprises the amino acid sequence of SEQ ID NO: 123, and wherein the LNP comprises 45-65% ionizable amino lipid, 5-10% phospholipid, 25-40% sterol, and 0.5-5% PEG modified lipid.

In some aspects, the disclosure provides an LNP comprising an mRNA comprising an ORF encoding a fusion protein, wherein the ORF comprises a nucleotide sequence having at least about 80%, about 85%, about 90%, about 91%, about 92%, about 93%, about 94%, about 95%, about 96%, about 97%, about 98%, or about 99% identity to the nucleotide sequence of SEQ ID NO: 122, and wherein the LNP comprises 45-65% ionizable amino lipid, 5-10% phospholipid, 25-40% sterol, and 0.5-5% PEG modified lipid. In some aspects, the disclosure provides an LNP comprising an mRNA comprising an ORF encoding a fusion protein, wherein the ORF comprises the nucleotide sequence of SEQ ID NO: 122, and wherein the LNP comprises 45-65% ionizable amino lipid, 5-10% phospholipid, 25-40% sterol, and 0.5-5% PEG modified lipid. In some aspects, the ionizable amino lipid is Compound 1. In some aspects, the sterol is cholesterol and the PEG-modified lipid is PEG-DMG. In some aspects, the sterol is cholesterol and the PEG-modified lipid is Compound 2.

In some aspects, the disclosure provides an LNP comprising an mRNA comprising an ORF encoding a fusion protein, wherein the fusion protein comprises an amino acid sequence having at least about 90%, about 91%, about 92%, about 93%, about 94%, about 95%, about 96%, about 97%, about 98%, or about 99% identity to the amino acid sequence of SEQ ID NO: 121, and wherein the LNP comprises an ionizable amino lipid, a phospholipid, a sterol, and a PEG modified lipid. In some aspects, the disclosure provides an LNP comprising an mRNA comprising an ORF encoding a fusion protein, wherein the fusion protein comprises the amino acid sequence of SEQ ID NO: 121, and wherein the LNP comprises an ionizable amino lipid, a phospholipid, a sterol, and a PEG modified lipid. In some aspects, the disclosure provides an LNP comprising an mRNA comprising an ORF encoding a fusion protein, wherein the ORF comprises a nucleotide sequence having at least about 80%, about 85%, about 90%, about 91%, about 92%, about 93%, about 94%, about 95%, about 96%, about 97%, about 98%, or about 99% identity to the nucleotide sequence of SEQ ID NO: 120, and wherein the LNP comprises an ionizable amino lipid, a phospholipid, a sterol, and a PEG modified lipid. In some aspects, the disclosure provides an LNP comprising an mRNA comprising an ORF encoding a fusion protein, wherein the ORF comprises the nucleotide sequence of SEQ ID NO: 120, and wherein the LNP comprises an ionizable amino lipid, a phospholipid, a sterol, and a PEG modified lipid. In some aspects, the ionizable amino lipid is Compound 1. In some aspects, the sterol is cholesterol and the PEG-modified lipid is PEG-DMG. In some aspects, the sterol is cholesterol and the PEG-modified lipid is Compound 2.

In some aspects, the disclosure provides an LNP comprising an mRNA comprising an ORF encoding a fusion protein, wherein the fusion protein comprises an amino acid sequence having at least about 90%, about 91%, about 92%, about 93%, about 94%, about 95%, about 96%, about 97%, about 98%, or about 99% identity to the amino acid sequence of SEQ ID NO: 121, and wherein the LNP comprises an ionizable amino lipid, a phospholipid, a sterol, and a PEG modified lipid. In some aspects, the disclosure provides an LNP comprising an mRNA comprising an ORF encoding a fusion protein, wherein the fusion protein comprises the amino acid sequence of SEQ ID NO: 121, and wherein the LNP comprises 40-60% ionizable amino lipid, 8-16% phospholipid, 30-45% sterol, and 1-5% PEG modified lipid. In some aspects, the disclosure provides an LNP comprising an mRNA comprising an ORF encoding a fusion protein, wherein the ORF comprises a nucleotide sequence having at least about 80%, about 85%, about 90%, about 91%, about 92%, about 93%, about 94%, about 95%, about 96%, about 97%, about 98%, or about 99% identity to the nucleotide sequence of SEQ ID NO: 120, and wherein the LNP comprises 40-60% ionizable amino lipid, 8-16% phospholipid, 30-45% sterol, and 1-5% PEG modified lipid. In some aspects, the disclosure provides an LNP comprising an mRNA comprising an ORF encoding a fusion protein, wherein the ORF comprises the nucleotide sequence of SEQ ID NO: 120, and wherein the LNP comprises an ionizable amino lipid, a phospholipid, a sterol, and a PEG modified lipid. In some aspects, the ionizable amino lipid is Compound 1. In some aspects, the sterol is cholesterol and the PEG-modified lipid is PEG-DMG. In some aspects, the sterol is cholesterol and the PEG-modified lipid is Compound 2.

In some aspects, the disclosure provides an LNP comprising an mRNA comprising an ORF encoding a fusion protein, wherein the fusion protein comprises an amino acid sequence having at least about 90%, about 91%, about 92%, about 93%, about 94%, about 95%, about 96%, about 97%, about 98%, or about 99% identity to the amino acid sequence of SEQ ID NO: 121, and wherein the LNP comprises 45-65% ionizable amino lipid, 5-10% phospholipid, 25-40% sterol, and 0.5-5% PEG modified lipid. In some aspects, the disclosure provides an LNP comprising an mRNA comprising an ORF encoding a fusion protein, wherein the fusion protein comprises the amino acid sequence of SEQ ID NO: 121, and wherein the LNP comprises 45-65% ionizable amino lipid, 5-10% phospholipid, 25-40% sterol, and 0.5-5% PEG modified lipid.

In some aspects, the disclosure provides an LNP comprising an mRNA comprising an ORF encoding a fusion protein, wherein the ORF comprises a nucleotide sequence having at least about 80%, about 85%, about 90%, about 91%, about 92%, about 93%, about 94%, about 95%, about 96%, about 97%, about 98%, or about 99% identity to the nucleotide sequence of SEQ ID NO: 120, and wherein the LNP comprises 45-65% ionizable amino lipid, 5-10% phospholipid, 25-40% sterol, and 0.5-5% PEG modified lipid. In some aspects, the disclosure provides an LNP comprising an mRNA comprising an ORF encoding a fusion protein, wherein the ORF comprises the nucleotide sequence of SEQ ID NO: 120, and wherein the LNP comprises 45-65% ionizable amino lipid, 5-10% phospholipid, 25-40% sterol, and 0.5-5% PEG modified lipid. In some aspects, the ionizable amino lipid is Compound 1. In some aspects, the sterol is cholesterol and the PEG-modified lipid is PEG-DMG. In some aspects, the sterol is cholesterol and the PEG-modified lipid is Compound 2.

In some aspects, the disclosure provides a pharmaceutical composition comprising an mRNA described herein, and a pharmaceutically acceptable carrier. In some aspects, the disclosure provides a pharmaceutical composition comprising a lipid nanoparticle described herein, and a pharmaceutically acceptable carrier.

In some aspects, the disclosure provides a method of treating a cancer in a subject, comprising administering to the patient an mRNA described herein, a lipid nanoparticle described herein, or a pharmaceutical composition described herein. In some aspects, the disclosure provides a method of treating a cancer in a human subject, comprising administering to the patient an mRNA described herein, a lipid nanoparticle described herein, or a pharmaceutical composition described herein

In some aspects, the disclosure provides a method of reducing or inhibiting tumor growth in a subject, comprising administering to the patient an mRNA described herein, a lipid nanoparticle described herein, or a pharmaceutical composition described herein. In some aspects, the patient has a disseminated tumor. In some aspects, the patient has a solid tumor. In some aspects, the disclosure provides a method of reducing or inhibiting tumor growth in a human patient, comprising administering to the patient an mRNA described herein, a lipid nanoparticle described herein, or a pharmaceutical composition described herein. In some aspects, the patient has a disseminated tumor. In some aspects, the patient has a solid tumor.

In some aspects, the disclosure provides a method of inducing or enhancing an anti-tumor immune response in a subject, comprising administering to the patient an mRNA described herein, a lipid nanoparticle described herein, or a pharmaceutical composition described herein.

In some aspects, the disclosure provides a method of inducing or enhancing an anti-tumor immune response in a human patient, comprising administering to the patient an mRNA described herein, a lipid nanoparticle described herein, or a pharmaceutical composition described herein. In some aspects, the anti-tumor immune response comprises increased proliferation of CD8 T cells, NK cells, NKT cells, or a combination thereof. In some aspects, the anti-tumor immune response comprises increased activation of CD8 T cells, NK cells, NKT cells, or a combination thereof.

In some aspects, the disclosure provides use of an mRNA described herein, a lipid nanoparticle described herein, or a pharmaceutical composition described herein, for treating a cancer in a subject. In some aspects, the disclosure provides use of an mRNA described herein, a lipid nanoparticle described herein, or a pharmaceutical composition described herein, for treating a cancer in a human patient. In some aspects, the disclosure provides use of an mRNA described herein, a lipid nanoparticle described herein, or a pharmaceutical composition described herein for the manufacture of a medicament for treating a cancer in a subject. In some aspects, the disclosure provides use of an mRNA described herein, a lipid nanoparticle described herein, or a pharmaceutical composition described herein for the manufacture of a medicament for treating a cancer in a human patient.

In some aspects, the disclosure provides use of an mRNA described herein, a lipid nanoparticle described herein, or a pharmaceutical composition described herein for reducing or inhibiting tumor growth in a subject. In some aspects, the disclosure provides use of an mRNA described herein, a lipid nanoparticle described herein, or a pharmaceutical composition described herein for reducing or inhibiting tumor growth in a human patient. In some aspects, the disclosure provides use of an mRNA described herein, a lipid nanoparticle described herein, or a pharmaceutical composition described herein for the manufacture of a medicament for reducing or inhibiting tumor growth in a subject. In some aspects, the disclosure provides use of an mRNA described herein, a lipid nanoparticle described herein, or a pharmaceutical composition described herein for the manufacture of a medicament for reducing or inhibiting tumor growth in a human patient.

In some aspects, the disclosure provides use of an mRNA described herein, a lipid nanoparticle described herein, or a pharmaceutical composition described herein for inducing or enhancing an anti-tumor response in a subject. In some aspects, the disclosure provides use of an mRNA described herein, a lipid nanoparticle described herein, or a pharmaceutical composition described herein for inducing or enhancing an anti-tumor response in a human patient. In some aspects, the disclosure provides use of an mRNA described herein, a lipid nanoparticle described herein, or a pharmaceutical composition described herein for the manufacture of a medicament for inducing or enhancing an anti-tumor response in a subject. In some aspects, the disclosure provides use of an mRNA described herein, a lipid nanoparticle described herein, or a pharmaceutical composition described herein for the manufacture of a medicament for inducing or enhancing an anti-tumor response in a human patient.

In any of the foregoing or related aspects, the mRNA, the lipid nanoparticle, or the pharmaceutical composition is administered to the subject by intravenous injection. In some aspects, the mRNA, the lipid nanoparticle, or the pharmaceutical composition is administered to the human patient by intravenous injection. In some aspects, the mRNA-encoded fusion protein is expressed in the liver, the spleen, or both. In some aspects, the mRNA-encoded fusion protein is expressed in the liver. In some aspects, the mRNA-encoded fusion protein is expressed in hepatocytes. In some aspects, the mRNA-encoded fusion protein is expressed in Kupffer cells. In some aspects, the mRNA-encoded fusion protein is expressed in the spleen. In some aspects, the mRNA-encoded fusion protein is expressed in spleen macrophages. In some aspects, the ApoA polypeptide assembles to form a high-density lipoprotein (HDL) particle comprising the fusion protein following expression of the mRNA. In some aspects, the HDL particle anchors the IL15 polypeptide and extended IL15Rα Sushi polypeptide for presentation to immune cells. In some aspects, the HDL particle facilitates trafficking of the fusion protein to the tumor.

In some aspects, the disclosure provides a kit comprising a container comprising an mRNA described herein, a lipid nanoparticle described herein, or a pharmaceutical composition described herein, and a package insert comprising instructions for administering the mRNA, the lipid nanoparticle, or the pharmaceutical composition for treating a cancer in a subject. In some aspects, the disclosure provides a kit comprising a container comprising an mRNA described herein, a lipid nanoparticle described herein, or a pharmaceutical composition described herein, and a package insert comprising instructions for administering the mRNA, the lipid nanoparticle, or the pharmaceutical composition for treating a cancer in a human patient.

In some aspects, the disclosure provides a kit comprising a container comprising an mRNA described herein, a lipid nanoparticle described herein, or a pharmaceutical composition described herein, and a package insert comprising instructions for administering the mRNA, the lipid nanoparticle, or the pharmaceutical composition for reducing or inhibiting tumor growth in a subject. In some aspects, the disclosure provides a kit comprising a container comprising an mRNA described herein, a lipid nanoparticle described herein, or a pharmaceutical composition described herein, and a package insert comprising instructions for administering the mRNA, the lipid nanoparticle, or the pharmaceutical composition for reducing or inhibiting tumor growth in a human patient.

In some aspects, the disclosure provides a kit comprising a container comprising an mRNA described herein, a lipid nanoparticle described herein, or a pharmaceutical composition described herein, and a package insert comprising instructions for administering the mRNA, the lipid nanoparticle, or the pharmaceutical composition for inducing or enhancing an anti-tumor immune response in a subject. In some aspects, the disclosure provides a kit comprising a container comprising an mRNA described herein, a lipid nanoparticle described herein, or a pharmaceutical composition described herein, and a package insert comprising instructions for administering the mRNA, the lipid nanoparticle, or the pharmaceutical composition for inducing or enhancing an anti-tumor immune response in a human patient.

### BRIEF DESCRIPTION OF THE FIGURES

FIG. 1A provides schematics depicting exemplary chimeric human IL15 (hIL15) fusion proteins of the disclosure. The hIL15 fusion proteins contain a signal peptide (SP1 or SP2); a carrier protein (Fc or ApoA-1); a 66 amino acid residue or 78 amino acid residue portion of the human IL15Rα extracellular domain spanning the Sushi domain (SushiS or SushiL respectively); and hIL15. Sequences of the fusion proteins are provided in Table 1.

FIG. 1B provides a schematic depicting exemplary anti-tumor effects of mRNA encoding Apo-containing fusion proteins of FIG. 1A. Following intravenous administration of the mRNA formulated in lipid nanoparticles (LNPs), the IL15 fusion protein encoded by the mRNA is expressed in the liver. The Apo domain facilitates binding of the IL15 fusion protein to high density lipoprotein (HDL), which in turn enables trafficking of the fusion protein to tumors where activation of immune cells by IL15/Sushi-mediated signaling occurs. Figure discloses SEQ ID NO: 133.

FIGS. 2A-2B provide graphs measuring expression of human ApoA (FIG. 2A) and human IL15/IL15Rα (FIG. 2B) by HEK293T cells transfected with mRNA encoding the human IL15 fusion proteins depicted in FIG. 1A. The negative control were cells transfected with mRNA encoding non-translatable (NST) murine OX40 ligand.

FIGS. 3A-3B provide graphs depicting IL15 bioactivity as measured by cellular proliferation in CTLL2 cells that express IL15Rα (FIG. 3A) and Mo7e cells that do not express IL15Rα (FIG. 3B) following transfection with mRNA encoding the hIL15 fusion proteins depicted in FIG. 1A.

FIG. 4A provides a graph quantifying whole-body bioluminescence over time in mice that received an intravenous injection of TransIT-complexed mRNA encoding luciferase (“TransIT” refers to Mirus TransIT® Transfection Reagent). Bioluminescence measurements were performed using an in vivo imaging system (IVIS).

FIG. 4B provides a graph quantifying bioluminescence of tissues harvested from the mice at 24 hours following administration of the TransIT-complexed mRNA or from control mice administered mRNA encoding NST murine OX40 ligand and complexed to TransIT. Bioluminescence measurements were performed using IVIS.

FIG. 5A provides a graph quantifying whole-body bioluminescence in mice that received an intravenous injection of clodronate liposomes (for depletion of Kuppfer cells) followed by an intravenous injection of TransIT-complexed mRNA encoding luciferase 24 hours later.

Bioluminescence was measured at 24 hours following administration of the TransIT-complexed mRNA. Control mice (labeled “Luc”) received the injection of TransIT-complexed mRNA encoding luciferase only, but not the injection of clodronate liposomes.

FIG. 5B provides a graph quantifying bioluminescence of tissues harvested from the mice in FIG. 5A at 24 hours following administration of TransIT-complexed mRNA. Tissues from mice that received both clodronate liposomes and TransIT-complexed mRNA encoding luciferase are labeled as “Luc+clodronate” and tissues from control mice that received TransIT-complexed mRNA encoding luciferase only are labeled as “Luc.” Bioluminescence measurements were performed using IVIS.

FIG. 6 provides a graph quantifying levels of human IL15/IL15Rα measured by ELISA in serum collected from mice administered the mRNA depicted in FIG. 1A complexed with TransIT. Control mice were administered TransIT-complexed mRNA encoding NST murine OX40 ligand.

FIG. 7 provides flow cytometry cluster analysis of immune populations in livers harvested from mice that were intravenously injected with TransIT-complexed mRNA encoding the indicated chimeric hIL15 fusion proteins. Livers were harvested at 5 days following injection of the TransIT-complexed mRNA. Shown are clusters of immune cell populations based on flow cytometry (top row) and expression of Ki67 as an indicator of cellular proliferation (bottom row). Control mice received TransIT-complexed mRNA encoding NST murine OX40 ligand.

FIG. 8A provide graphs quantifying immune cell subsets in livers harvested from mice at different time points following intravenous injection of TransIT-complexed mRNA encoding the indicated chimeric hIL15 fusion proteins. Each plot shows the total cell count per liver versus day after treatment. Control mice received TransIT-complexed mRNA encoding NST murine OX40 ligand.

FIG. 8B provides graphs quantifying Ki67 expression in certain immune cell subsets identified in FIG. 8A. Shown is the percentage of Ki67 expressing NK cells over the total number of NK cells; percentage of Ki67 expressing NKT cells over the total number of NKT cells; percentage of Ki67 expressing CD8 T cells over the total number of CD8 T cells; and percentage of Ki67 expressing effector CD8 T cells (CD44high) over the total number of effector CD8 T cells.

FIG. 8C provides graphs quantifying IFN7 expression among NK cells identified in FIG. 8A. Shown is the percentage of liver NK cells expressing IFN7 (left panel) and the total number of IFN7-expressing NK cells per liver (right panel).

FIG. 9A provides a graph showing tumor growth over time in mice bearing MC38 flank tumors that were intravenously administered TransIT-complexed mRNA encoding the indicated hIL15 fusion proteins. Control mice received TransIT-complexed mRNA encoding NST murine OX40 ligand.

FIG. 9B provides a graph showing tumor growth over time in mice bearing B16-Ova flank tumors that were intravenously administered TransIT-complexed mRNA encoding the indicated hIL15 fusion proteins. Control mice received TransIT-complexed mRNA encoding NST murine OX40 ligand.

FIG. 10 provides graphs showing the effect of immune cell depletion on tumor growth in mice bearing B16-Ova-tumors that received TransIT complexed mRNA encoding SP2-ApoA-SushiL-IL15. Control mice received TransIT-complexed mRNA encoding NST murine OX40 ligand. Immune cell depletion of CD8+T cells, NK cells, and CD4+T cells was performed using anti-CD8, anti-NK1.1, and anti-CD4 respectively.

FIGS. 11A-11B provide graphs quantifying levels of hIL15/IL15Rα in livers (FIG. 11A) and tumors (FIG. 11B) harvested from mice bearing MC38 tumors at 24 hours following intravenous injection of mRNA encoding the indicated hIL15 fusion proteins. Control mice received TransIT-complexed mRNA encoding NST murine OX40 ligand. Tissue quantification of the hIL15/IL15Rα complex was measured by ELISA.

### DETAILED DESCRIPTION

### Overview

The present disclosure is based, at least in part, on the discovery that mRNA-encoded IL15 fusions proteins comprising an IL15 polypeptide, an extended sushi domain of an IL15R≢ polypeptide (referred to hereinafter as “extended sushi” or “ExtSushi”), and an ApoA polypeptide (referred to herein after as “ApoA”) elicits an effective anti-tumor immune response, e.g., as compared to an IL15 fusion protein lacking the ApoA polypeptide. As described herein, an IL15 fusion protein comprising an IL15 polypeptide operably linked to an extended sushi domain of an IL15Rα polypeptide (referred to as “an IL15/Sushi fusion protein”) elicits signal activation when contacted with IL15Rβ/γ receptor-expressing cells. However, the inclusion of an ApoA polypeptide yields an IL15/Sushi fusion protein having (i) increased circulation half-life; (ii) improved accumulation and/or penetration of target tissues (e.g., tumor tissues); and/or (iii) improved activation of one or more target cell populations (e.g., lymphatic cells, e.g., NK cells, NKT cells, T cells) following in vivo administration as compared to an IL15/Sushi fusion protein lacking the ApoA polypeptide. Moreover, it was demonstrated that an IL15/Sushi fusion protein comprising an N-terminal ApoA polypeptide is particularly effective for inducing proliferation of IL15Rβ/γ-expressing cells, e.g., as compared to an IL15/Sushi fusion protein comprising a C-terminal ApoA polypeptide.

In some aspects, an IL15/Sushi fusion protein comprising an ApoA polypeptide (“IL15/Sushi/ApoA fusion protein”) has similar or improved functional properties as compared to an IL15/Sushi fusion protein comprising an immunoglobulin constant domain (“IL15/Sushi/Fc fusion protein”) (e.g., an IgG Fc domain). For example, it was demonstrated that an mRNA-encoded IL15/Sushi/ApoA fusion protein induced activation of NK cells, NKT cells, and CD8 T cells, both in vitro and in vivo, to a similar extent as an mRNA-encoded IL15/Sushi/Fc fusion protein. However, it was discovered that an mRNA-encoded IL15/Sushi/ApoA fusion protein elicited a superior anti-tumor immune response in preclinical tumor models following systemic administration (e.g., via intravenous injection) as compared to an IL15/Sushi/Fc fusion protein.

Without being bound by theory, the ApoA polypeptide facilitates improved delivery of the fusion protein to tumor and/or tumor draining lymphatic tissues following systemic administration (e.g., intravenous administration) as compared to the Fc domain. As further described herein, and without being bound by theory, the improved delivery of an IL15/Sushi/ApoA fusion protein to tumor and/or tumor draining lymphatic tissues following in vivo administration results from assembly of ApoA into HDL particles that effectively target HDL-scavenging receptors expressed on tumor cells.

Accordingly, in some aspects, the disclosure provides an mRNA comprising an open reading frame (ORF) encoding an IL15 fusion protein described herein. In some aspects, the IL15 fusion protein comprises an IL15 polypeptide, an extended sushi domain of an IL15R≢ polypeptide, and an ApoA polypeptide. In some aspects, the IL15 polypeptide is operably linked to the extended sushi domain. In some aspects, the ApoA polypeptide is positioned at the N-terminus of the IL15 fusion protein. In some aspects, the IL15 fusion protein comprises from N-terminus to C-terminus: an ApoA polypeptide, an extended sushi domain of an IL15R≢ polypeptide, and an IL15 polypeptide. In some embodiments, the ORF encodes a signal peptide at the 5′end of the IL15 fusion protein. In some aspects, the mRNA is formulated as an LNP. In some aspects, the mRNA is complexed with a transfection reagent (e.g., TransIT).

### IL15 Fusion Protein Components

### IL15 Polypeptides

In some embodiments, the disclosure provides mRNA encoding an IL15 fusion protein comprising an IL15 polypeptide. In some embodiments, the IL15 polypeptide is a human IL15 polypeptide. In some embodiments, the IL15 polypeptide is a variant, a peptide or a polypeptide containing a substitution, and insertion and/or an addition, a deletion and/or a covalent modification with respect to a wild-type IL15 polypeptide (e.g., a human wild-type IL15 polypeptide). As referred herein, the term “IL15 polypeptide” refers to the mature IL15 polypeptide (i.e., without its signal peptide and propeptide). In some embodiments, a mature IL15 polypeptide of the disclosure comprises or consists of the amino acid sequences set forth in SEQ ID NO: 16. In some embodiments, a mature IL15 polypeptide of the disclosure comprises or consists of the amino acid sequence encoded by a nucleotide sequence set forth in SEQ ID NOs: 38-42. In one embodiment, the IL15 polypeptide comprises a signal peptide and/or propeptide. In some embodiments, the IL15 polypeptide comprises or consists of the amino acid sequence set forth in SEQ ID NO: 44, or an amino acid sequence encoded by the nucleotide sequence set forth in SEQ ID NO: 45.

In some embodiments, the IL15 polypeptide comprise an amino acid sequence encoded by the human IL15 gene. The human IL15 gene encodes a 162 amino acid preprotein having a signal peptide of 48 amino acids, with the mature protein being 114 amino acids in length. Bamford, R. N., et al., Proc. Natl. Acad. Sci. USA 93: 2897-2902 (1996). See also, e.g., GenBank Accession Numbers NM_000585 for the Homo sapiens IL15 transcript variant 3 mRNA sequence and NP_000576 for the corresponding IL15 isoform 1 preproprotein. In some embodiments, the IL15 polypeptide is the mature protein encoded by the human IL15 gene.

In some embodiments, the IL15 polypeptide is selected from:
(i) the mature human IL15 polypeptide (e.g., having the same or essentially the same length as wild-type human IL15) with or without a signal peptide;
(ii) a functional fragment of the mature human IL15 polypeptide (e.g., a truncated (e.g., deletion of carboxy, amino terminal, or internal regions) sequence shorter than an IL15 wildtype; but still retaining IL15 activity);
(iii) a functional variant of the mature human IL15 polypeptide (e.g., full-length, mature, or truncated IL15 proteins in which one or more amino acids have been replaced, e.g., variants that retain all or most of the IL15 activity of the polypeptide with respect to the wild-type IL15 polypeptide); and
(iv) a fusion protein comprising (a) a mature human IL15 wild-type, a functional fragment or a variant thereof, with or without a signal peptide and (b) a heterologous protein.

As used herein, “functional fragment of the mature human IL15 polypeptide” or “a functional variant of the mature human IL15 polypeptide” is understood to mean a polypeptide that maintains one or more functional properties of native human IL15. In some embodiments, the one or more functional properties comprise the capacity to promote the proliferation of CD8+T cells determined, for example, by the method described by Montes, et al, Clin. Exp. Immunol., 2005, 142:292-302, wherein a population of peripheral blood mononuclear cells is incubated with an antigen peptide in the presence of the functional fragment or variant of IL15 followed by the determination of the percentage of cells that can be labelled with specific antibodies against CD8. In some embodiments, the one or more functional properties comprise the capacity to promote the activation of NK cells after being presented in trans by dendritic cells. This capacity may be determined by measuring the incorporation of tritiated thymidine on the part of the CD56+NK cells in the presence of IL15 or by measuring the NK cell secretion of the GM-CSF cytokine. Methods for determining both IL15 functionalities have been described by Carson, W. et al. J. Exp. med., 1994, 180:1395-1403. In some embodiments, the one or more functional properties comprise the capacity to inhibit Fas-mediated apoptosis in B-cell precursors, as described by Demirci et al. (Cell Mol Immunol. 2004, 1:123-8.), which can be determined using standard techniques for determining apoptosis such as TUNEL or the determination of DNA fragmentation by gel electrophoresis and ethidium bromide staining.

In some embodiments, the IL15 polypeptide is a mammalian IL15 polypeptide, such as a non-human (e.g., primate) IL15, a functional fragment or a variant thereof. Non-limiting exemplary non-human mammalian IL15 polypeptides are murine IL15 (e.g., accession number NM_008357), rat IL15 (e.g., accession number NM_013129), rabbit IL15 (e.g., accession number DQ157152), sheep IL15 (e.g., accession number NM_001009734), or a pig IL15 (e.g., accession number NM_211390).

In some embodiments, a human IL15 polypeptide of the disclosure comprises an amino acid sequence having at least about 50%, at least about 60%, at least about 70%, at least about 80%, at least about 85%, at least about 90%, at least about 95%, at least about 98%, at least about 99%, or 100% identical to the amino acid sequence of SEQ ID NO: 16. In some embodiments, the human IL15 polypeptide comprises an amino acid sequence having at least about 90%, about 95%, about 98%, about 99% identity to the amino acid sequence of SEQ ID NO: 16. In some embodiments, the human IL15 polypeptide comprises an amino acid sequence encoded by a nucleotide sequence having at least about 80%, about 85%, about 90%, about 95%, about 98%, or about 99% identity to a nucleotide sequence selected from SEQ ID NOs: 38-42, wherein the human IL15 polypeptide is capable of binding to a human IL15 receptor. In some embodiments, the human IL15 polypeptide comprises an amino acid sequence encoded by a nucleotide sequence having at least about 90%, about 95%, about 98%, or about 99% identity to a nucleotide sequence selected from SEQ ID NOs: 38-42, wherein the human IL15 polypeptide is capable of binding to a human IL15 receptor. In some embodiments, the human IL15 polypeptide comprises an amino acid sequence encoded by a nucleotide sequence selected from SEQ ID NOs: 38-42, wherein the human IL15 polypeptide is capable of binding to a human IL15 receptor.

In some embodiments, the mRNA comprises a nucleotide sequence having at least about 50%, about 60%, about 70%, about 80%, about 85%, about 90%, about 95%, about 98%, about 99%, or 100% identity to a nucleic acid sequence selected from SEQ ID NOs: 38-42. In some embodiments, the mRNA comprises a nucleotide sequence having at least about 90%, about 95%, about 98%, about 99%, or 100% identity to a nucleic acid sequence selected from SEQ ID NOs: 38-42. In some embodiments, the mRNA comprises a nucleotide sequence selected from SEQ ID NOs: 38-42.

In some embodiments, a human IL15 polypeptide of the disclosure comprises an amino acid sequence listed in SEQ ID NO: 16 with one or more conservative substitutions, wherein the conservative substitutions do not significantly affect the binding activity of the IL15 polypeptide to its receptor, i.e., the IL15 polypeptide binds to the IL15 receptor after the substitutions.

In other embodiments, the disclosure provides an mRNA encoding an IL15 fusion protein comprising a IL15 polypeptide. A person of ordinary skill in the art would understand that nucleotide sequences encoding mammalian IL15 can be identified in nucleic acid repositories, and include, for example, polynucleotides whose sequences are identified by accession numbers U14407 (human IL15); U14332 (mouse IL15); U69272 (rat IL15); AF108148 (cat IL15), and U42433 (bovine IL15).

### IL15Rα Polypeptide

In some embodiments, the disclosure provides mRNA encoding an IL15 fusion protein comprising an IL15 polypeptide operably linked to an IL15Rα polypeptide. The term “IL15R≢ polypeptide” as used herein refers to a full-length IL15Rα polypeptide or a fragment thereof comprising at least the sushi domain of an IL15Rα polypeptide. As used herein, the “sushi domain of an IL15Rα polypeptide” refers to a contiguous amino acid sequence of the IL15R≢ ectodomain that begins at the first cysteine residue of the IL15Rα chain after its signal sequence and ends at the fourth cystine residue of the IL15Rα chain after its signal sequence. In some embodiments, a sushi domain of an IL15Rα polypeptide of the disclosure consists of the amino acid sequence set forth in SEQ ID NO: 47. In some embodiments, a sushi domain of an IL15R≢ polypeptide of the disclosure is an amino acid sequence having 1-10 amino acid alterations (e.g., substitution, deletion, insertion) relative to the amino acid sequence set forth in SEQ ID NO: 47. In some embodiments, a sushi domain of an IL15Rα polypeptide of the disclosure is 61 amino acid residues in length and has at least about 80%, at least about 85%, at least about 90%, at least about 95%, at least about 98%, or at least about 99% identity to the amino acid sequence set forth in SEQ ID NO: 47. In some embodiments, the sushi domain of an IL15Rα polypeptide consists of an amino acid sequence encoded by a nucleotide sequence set forth in SEQ ID NO: 48. In some embodiments, the sushi domain is 61 amino acid residues in length and encoded by a nucleotide sequence having one or more nonsynonymous mutations relative to the nucleotide sequence set forth in SEQ ID NO: 48. In some embodiments, the sushi domain is 61 amino acid residues in length and encoded by a nucleotides sequence having at least about 60%, at least about 65%, at least about 70%, at least about 75%, at least about 80%, at least about 85%, at least about 90%, at least about 95%, at least about 98%, or at least about 99% identity to the nucleotide sequence set forth in SEQ ID NO: 48.

In some embodiments, the IL15Rα polypeptide is a full-length IL15Rα. In some embodiments, a full-length IL15Rα polypeptide of the disclosure comprises or consists of the amino acid sequence set forth in SEQ ID NO: 21, e.g., with or without the signal peptide. In some embodiments, the full-length IL15Rα polypeptide of the disclosure comprises or consists of an amino acid sequence encoded by the nucleotide sequence set forth in SEQ ID NO: 46, e.g., with or without the nucleotide sequence encoding the signal peptide. In some embodiments, the IL15Rα polypeptide comprises or consists of a contiguous fragment of an IL15Rα. In some embodiments, the IL15Rα polypeptide comprises or consists of the ectodomain of an IL15Rα polypeptide. In some embodiments, an ectodomain of an IL15Rα polypeptide comprises or consists of the amino acid sequence set forth in SEQ ID NO: 51, e.g., with or without the signal peptide. In some embodiments, an ectodomain of an IL15Rα polypeptide comprises or consists of the amino acid sequence encoded by the nucleotide sequence set forth in SEQ ID NO: 52, e.g., with or without the nucleotide sequence encoding the signal peptide. In some embodiments, the IL15Rα polypeptide comprises or consists of a contiguous fragment of the ectodomain of an IL15Rα polypeptide, wherein the contiguous fragment of the ectodomain comprises the sushi domain of the IL15Rα polypeptide. In some embodiments, the contiguous fragment of the ectodomain of an IL15Rα polypeptide comprises the amino acid sequence set forth in SEQ ID NO: 47.

In some embodiments, the IL15Rα polypeptide comprises a full-length human IL15Rα polypeptide (e.g., SEQ ID NO: 21, with or without the signal peptide). In some embodiments, the human IL15Rα polypeptide comprise an amino acid sequence encoded by the human IL15Rα gene. The IL15Rα gene encodes a 267 amino acid pre-protein having a signal peptide of 30 amino acids, with the mature protein being 237 amino acids in length. In some embodiments, the signal peptide corresponds to amino acid residues 1-30 of the human IL15Rα pre-protein (e.g., amino acid residues 1-30 of SEQ ID NO: 21) and the sushi domain corresponds to amino acid residues 33-93 of the human IL15Rα pre-protein (e.g., amino acid residues 33-93 of SEQ ID NO: 21). See, e.g., GenBank Accession Numbers NM_002189 for the Homo sapiens IL15Rα transcript variant 1 mRNA and NP_002180 for the Homo sapiens IL15Rα isoform 1 precursor amino acid sequence. Exon 1 of the IL15Rα gene encodes the IL15Rα signal peptide; exon 2 of the IL15Rα gene encodes amino acid residues 31-95 of the IL15Rα pre-protein, which includes the sushi domain of IL15Rα (amino acid residues 33-93); the 5′end of exon 3 of the IL15Rα gene encodes a hinge domain of the IL15Rα polypeptide; the 3′end of exon3, exon 4, and exon 5 of the IL15Rα gene a Pro/Thr rich and glycosylated domain of IL15Rα polypeptide; exon 6 of the IL15Rα gene encodes the IL15Rα transmembrane domain; and exon 7 of the IL15Rα gene encodes the IL15Rα intracellular domain (see, e.g., Bouchaud, et al (2008) JMol Biol 382:1-12).

In some embodiments, the IL15Rα polypeptide comprises an amino acid sequence encoded by exon 2 of the IL15Rα gene. In some embodiments, the IL15Rα polypeptide comprises an amino acid sequence encoded by exon 2 of the IL15Rα gene and at least one codon (e.g., 1-15 codons) at the 5′end of exon 3 of the IL15Rα gene. In some embodiments, the IL15Rα polypeptide comprises an amino acid sequence encoded by exon 2 and exon 3 of the IL15Rα gene. In some embodiments, the IL15Rα polypeptide comprises an amino acid sequence encoded by exon 2, exon 3, and exon 4 of the IL15Rα gene. In some embodiments, the IL15Rα polypeptide comprises an amino acid sequence encoded by exon 2, exon 3, exon 4, and exon 5 of the IL15Rα gene. In some embodiments, the IL15Rα polypeptide comprises an amino acid sequence encoded by exon 2, exon 3, exon 4, exon 5, and exon 6 of the IL15Rα gene

In some embodiments, the IL15Rα polypeptide comprises a fragment of human IL15Rα comprising at least the sushi domain of human IL15Rα. As used herein, the “sushi domain of human IL15Rαα refers the portion of the human IL15Rα ectodomain beginning at the first cysteine from the signal peptide (amino acid residue 33 of human IL15Rα) and ending at the fourth cysteine from the signal peptide (amino acid residue 93 of human IL15Rα). In some embodiments, the human IL15Rα polypeptide (including signal peptide) has an amino acid sequence as set forth in SEQ ID NO: 21, or an amino acid sequence having at least 90% sequence identity to SEQ ID NO: 21, wherein the sushi domain corresponds to residues 33-93 of SEQ ID NO: 21. In some embodiments, the sushi domain of a human IL15Rα polypeptide consists of the amino acid sequence set forth in SEQ ID NO: 47, or of an amino acid sequence having at least 90% identity to SEQ ID NO: 47. In some embodiments, the sushi domain of a human IL15Rα polypeptide consists of an amino acid sequence encoded by the nucleotide sequence set forth in SEQ ID NO: 48, or of an amino acid sequence encoded by a nucleotide sequence having at least about 80%, 85%, 90%, or 95% identity to SEQ ID NO: 48.

In some embodiments, the IL15Rα polypeptide is selected from:
(i) the full-length human IL15Rα polypeptide (e.g., having the same or essentially the same length as wild-type human IL15Rα);
(ii) a functional fragment of the full-length human IL15Rα polypeptide (e.g., a truncated (e.g., deletion of carboxy, amino terminal, or internal regions) sequence shorter than full-length human IL15Rα wild-type; but still retaining IL15Rα activity) comprising at least the sushi domain of human IL15Rα;
(iii) a variant of the full-length human IL15Rα polypeptide or of a truncated human IL15Rα polypeptide comprising at least the sushi domain (e.g., full-length or truncated human IL15Rα proteins comprising one or more amino acid substitutions, wherein the variants retain all or most of the IL15Rα activity of the polypeptide with respect to the wild-type human IL15Rα polypeptide (such as natural or artificial variants known in the art); and
(iv) a fusion protein comprising (a) a full-length human IL15Rα wild-type, a functional fragment or a variant thereof, and (b) a heterologous protein.

In some embodiments, the IL15Rα polypeptide is a human IL15Rα polypeptide, wherein the human IL15Rα polypeptide comprises the amino acid sequence set forth in SEQ ID NO: 21, e.g., with or without the signal peptide. In some embodiments, the IL15Rα polypeptide comprises the ectodomain of the full-length human IL15Rα polypeptide. In some embodiments, the ectodomain of the full-length human IL15Rα polypeptide comprises the amino acid sequence set forth in SEQ ID NO: 51. In some embodiments, the ectodomain of the full-length human IL15Rα polypeptide comprises the amino acid sequence encoded by the nucleotide sequence set forth in SEQ ID NO: 52. In other embodiments, the IL15Rα polypeptide comprises the transmembrane domain and/or intracellular domain of the full-length human IL15Rα polypeptide. In some embodiments, the transmembrane domain of the full-length human IL15Rα polypeptide comprises the amino acid sequence set forth in SEQ ID NO: 49. In some embodiments, the intracellular domain of the full-length human IL15Rα polypeptide comprises the amino acid sequence set forth in SEQ ID NO: 50. In other embodiments, the IL15Rα polypeptide comprises the transmembrane region and/or intracellular domain of a heterologous polypeptide.

In some embodiments, the human IL15Rα polypeptide comprises an amino acid sequence having at least about 50%, at least about 60%, at least about 70%, at least about 80%, at least about 85%, at least about 90%, at least about 95%, at least about 98%, at least about 99%, or 100% identity to the amino acid sequence of SEQ ID NO: 124. In some embodiments, the human IL15Rα polypeptide comprises an amino acid sequence having at about least about 90%, at least about 95%, at least about 98%, at least about 99%, or 100% identity to the amino acid sequence of SEQ ID NO: 124. In some embodiments, the human IL15Rα polypeptide comprises an amino acid sequence encoded by a nucleotide sequence having at least about 50%, at least about 60%, at least about 70%, at least about 80%, at least about 85%, at least about 90%, at least about 95%, at least about 98%, at least about 99%, or 100% identity to the nucleotide sequence of SEQ ID NO: 125, wherein the human IL15Rα polypeptide is capable of binding to a human IL15 polypeptide. In some embodiments, the human IL15Rα polypeptide comprises an amino acid sequence encoded by a nucleotide sequence having at least about 90%, at least about 95%, at least about 98%, at least about 99%, or 100% identity to a nucleotide sequence of SEQ ID NO: 125, wherein the human IL15Rα polypeptide is capable of binding to a human IL15 polypeptide.

In certain embodiments, the human IL15Rα polypeptide of the disclosure comprises an amino acid sequence listed in SEQ ID NO: 124, and comprising one or more conservative substitutions, wherein the conservative substitutions do not significantly affect the binding activity of the IL15Rα polypeptide to its ligand, i.e., the IL15Rα polypeptide binds to IL15 after the substitutions.

In some embodiments, the disclosure provides an mRNA comprising an ORF encoding a fusion protein, wherein the ORF comprises a nucleotide sequence having at least about 50%, at least about 60%, at least about 70%, at least about 80%, at least about 85%, at least about 90%, at least about 95%, at least about 98%, at least about 99%, or 100% identity to the nucleic acid sequence of SEQ ID NO: 125. In some embodiments, the ORF comprises a nucleotide sequence having at least about at least about 90%, at least about 95%, at least about 98%, at least about 99%, or 100% identity to the nucleic acid sequence of SEQ ID NO: 125.

Extended sushi domain of IL15Rα

In some embodiments, the disclosure provides IL15 fusion protein comprising an IL15 polypeptide described herein operably linked to an extended sushi domain of an IL15Rα polypeptide. As used herein, an “extended IL15Rα Sushi polypeptide” refers to a contiguous region of the IL15Rα ectodomain that spans the entire length of the IL15Rα Sushi domain and includes (i) at least one amino acid residue (e.g., 1-15 amino acid residues) before the N-terminus of the IL15Rα Sushi domain; (ii) at least one amino acid residue (e.g., 1-15 amino acid residues) after the C-terminus of the IL15Rα Sushi domain; or (iii) both (i) and (ii). In some embodiments, the extended IL15Rα Sushi polypeptide is encoded by exon 2 of the IL15Rα gene and at least one codon (e.g., 1-15 codons) at the 5′end of exon 3 of the IL15Rα gene. In some embodiments, the extended IL15Rα Sushi polypeptide has increased binding affinity for the IL15 polypeptide compared to the IL15Rα Sushi polypeptide. Methods for measuring IL15Rα sushi polypeptide binding affinity to IL15 are described in the art, see, e.g., Bouchaud, et al (2008) J Mol Biol 382:1-12.

In some embodiments, the IL15 fusion protein comprises an IL15 polypeptide described herein operably linked to an extended sushi domain of a human IL15Rα polypeptide. In some embodiments, the extended sushi domain of a human IL15Rα polypeptide comprises a contiguous region of the human IL15Rα ectodomain that begins at or near the N-terminus of human IL15Rα (e.g., begins at residue 31, 32, or 33 of full-length human IL15Rα with signal peptide, optionally wherein the full-length human IL15Rα with signal peptide has the amino acid sequence set forth in SEQ ID NO: 21) and extends to include at least one amino acid residue of the ectodomain that follows the fourth cysteine from the signal peptide (e.g., ends at an amino acid residue of the ectodomain that is at least one amino acid residue down from residue 93 of full-length human IL15Rα with signal peptide, optionally wherein the full-length human IL15Rα with signal peptide has the amino acid sequence set forth in SEQ ID NO: 21).

In some embodiments, the extended sushi domain of a human IL15Rα polypeptide comprises a contiguous region of human IL15Rα that begins at or near the N-terminus of human IL15Rα (i.e., begins at residue 31, 32, or 33 of full-length human IL15Rα with signal peptide, optionally wherein the full-length human IL15Rα with signal peptide has the amino acid sequence set forth in SEQ ID NO: 21) and extends to include 1, 2, 3, 4, 5, 6, 7, 8, 9, 10, 11, 12, 13, 14, or 15 amino acid residues that follow the fourth cysteine from the signal peptide. In some embodiments, the contiguous region of human IL15Rα begins at residue 31, 32, or 33 of full-length human IL15Rα with signal peptide and ends at residue 94, 95, 96, 97, 98, 99, 100, 101, 102, 103, 104, 105, 106, 107, 108, 109, or 110, optionally wherein the full-length human IL15Rα with signal peptide has the amino acid sequence set forth in SEQ ID NO: 21.

In some embodiments, the extended sushi domain of a human IL15Rα polypeptide comprises a contiguous region of human IL15Rα that is at least 62, 63, 64, 65, 66, 67, 68, 69, 70, 71, 72, 73, 74, 75, 76, 77, 78, 79, or 80 amino acid residues in length, wherein the contiguous region begins at or near the N-terminus of human IL15Rα (i.e., begins at residue 31, 32, or 33 of full-length human IL15Rα with signal peptide, optionally wherein the full-length human IL15Rα with signal peptide has the amino acid sequence set forth in SEQ ID NO: 21). In some embodiments, the contiguous region is 62 amino acid residues in length. In some embodiments, the contiguous region is 66 amino acid residues in length. In some embodiments, the contiguous region is 78 amino acid residues in length. In some embodiments, the contiguous region not more than 80 amino acid residues in length.

In some embodiments, the extended sushi domain of a human IL15Rα polypeptide comprises or consists of an amino acid sequence having at least about 70%, about 75%, about 80%, about 85%, about 90%, about 95%, or about 99% sequence identity to the amino acid sequence of SEQ ID NO: 17. In some embodiments, the extended sushi domain of a human IL15Rα polypeptide comprises or consists of an amino acid sequence having at least about 90%, about 95%, about 98%, or about 99% sequence identity to the amino acid sequence of SEQ ID NO: 17. In some embodiments, the extended sushi domain of a human IL15Rα polypeptide comprises or consists of the amino acid sequence of SEQ ID NO: 17. In some embodiments, the extended sushi domain of a human IL15Rα polypeptide comprises or consists of an amino acid sequence encoded by a nucleotide sequence having at least about 70%, about 75%, about 80%, about 85%, about 90%, about 95%, or about 99% sequence identity to a nucleotide sequence selected from SEQ ID NOs: 29-31. In some embodiments, the extended sushi domain of a human IL15Rα polypeptide comprises or consists of an amino acid sequence encoded by a nucleotide sequence having at least about 90%, about 95%, about 98%, or about 99% sequence identity to a nucleotide sequence selected from SEQ ID NOs: 29-31. In some embodiments, the extended sushi domain of a human IL15Rα polypeptide comprises or consists of an amino acid sequence encoded by a nucleotide sequence selected from SEQ ID NOs: 29-31.

In some embodiments, the extended sushi domain of a human IL15Rα polypeptide comprises or consists of an amino acid sequence having at least about 70%, about 75%, about 80%, about 85%, about 90%, about 95%, or about 99% sequence identity to the amino acid sequence of SEQ ID NO: 18. In some embodiments, the extended sushi domain of a human IL15Rα polypeptide comprises or consists of an amino acid sequence having at least about 90%, about 95%, about 98%, or about 99% sequence identity to the amino acid sequence of SEQ ID NO: 18. In some embodiments, the extended sushi domain of a human IL15Rα polypeptide comprises or consists of the amino acid sequence of SEQ ID NO: 18. In some embodiments, the extended sushi domain of a human IL15Rα polypeptide comprises or consists of an amino acid sequence encoded by a nucleotide sequence having at least about 70%, about 75%, about 80%, about 85%, about 90%, about 95%, or about 99% sequence identity to a nucleotide sequence selected from SEQ ID NOs: 32 and 33. In some embodiments, the extended sushi domain of a human IL15Rα polypeptide comprises or consists of an amino acid sequence encoded by a nucleotide sequence having at least about 90%, about 95%, about 98%, or about 99% sequence identity to a nucleotide sequence selected from SEQ ID NOs: 32 and 33. In some embodiments, the extended sushi domain of a human IL15Rα polypeptide comprises or consists of an amino acid sequence encoded by a nucleotide sequence selected from SEQ ID NOs: 32 and 33.

In some embodiments, the disclosure provides an mRNA encoding an IL15 fusion protein described herein, wherein the IL15 fusion protein comprises an extended sushi domain of a human IL15Rα polypeptide. In some embodiments, the mRNA comprises a nucleotide sequence having at least about 70%, about 75%, about 80%, about 85%, about 90%, about 95%, or about 99% sequence identity to a nucleotide sequence selected from SEQ ID NOs: 32 and 33.

In some embodiments, the mRNA comprises a nucleotide sequence having at least about 90%, about 95%, about 98%, or about 99% sequence identity to a nucleotide sequence selected from SEQ ID NOs: 32 and 33. In some embodiments, the mRNA comprises a nucleotide sequence encoding the extended sushi domain of a human IL15Rα polypeptide, wherein the nucleotide sequence is selected from SEQ ID NOs: 32 and 33.

In some embodiments, the mRNA comprises a nucleotide sequence having at least about 70%, about 75%, about 80%, about 85%, about 90%, about 95%, or about 99% sequence identity to a nucleotide sequence selected from SEQ ID NOs: 29-31. In some embodiments, the mRNA comprises a nucleotide sequence having at least about 90%, about 95%, about 98%, or about 99% sequence identity to a nucleotide sequence selected from SEQ ID NOs: 29-31. In some embodiments, the mRNA comprises a nucleotide sequence encoding the extended sushi domain of a human IL15Rα polypeptide, wherein the nucleotide sequence is selected from SEQ ID NOs: 29-31.

### Apolipoprotein A (ApoA) Polypeptide

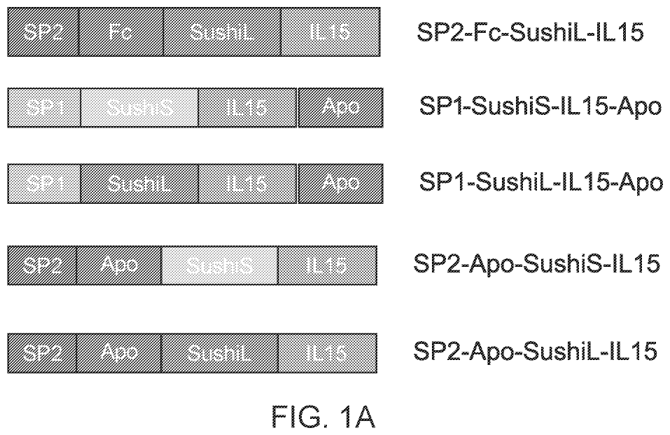

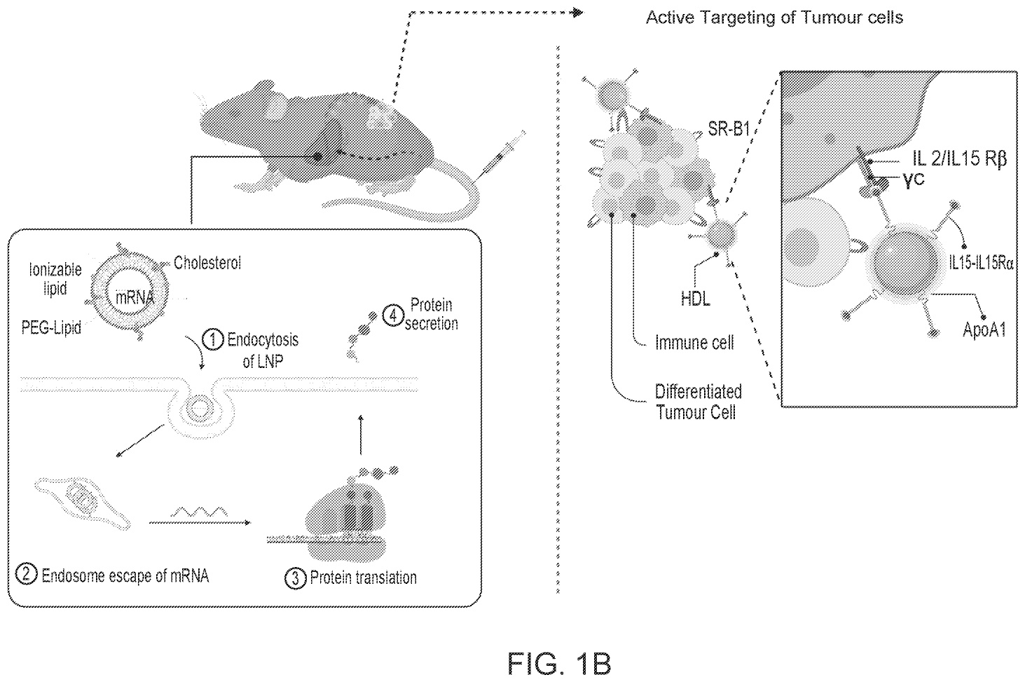

In some embodiments, the disclosure provides an mRNA encoding an IL15 fusion protein comprising an IL15 polypeptide, an IL15Rα polypeptide (e.g., an extended sushi domain of IL15Rα), and an ApoA polypeptide. ApoA is a protein component of high-density lipoproteins (HDL). As shown in FIG. 1B, the disclosure provides exemplary embodiments in which mRNA-encoded IL15/ExtSushi fusion protein containing ApoA (IL15/ExtSushi/ApoA fusion proteins) are expressed in the liver following in vivo administration. HDL circulates collecting cholesterol from tissues and accumulates in the liver (see, e.g., Francis, GA Biochim Biophys Acta (2010) 1801:1286-93). Without being bound by theory, IL15 fusion proteins of the disclosure comprising ApoA expressed in the liver assemble to form HDL particles, which are then transported from the liver to target cell populations and/or target tissues. Several scavenger receptors are known to bind HDL, e.g., scavenger receptor type BI (SR-BI). Moreover, SR-B1 has elevated expression levels in a variety of cancer tissues (see, e.g., Hoekstra, et al (2017) Curr Opin Lipidol 28:255-260). Accordingly, and without being bound by theory, HDL particles comprising an IL15 fusion protein of the disclosure are effectively transported to target cancer tissues and/or target cancer cells expressing HDL scavenger receptors (e.g., SR-BI), where the particles are captured and the IL15 fusion protein functions to stimulate or promote an anti-tumor immune response (e.g., via activation NK cells, NKT cells, CD8 T cells, or a combination thereof).

In some embodiments, the ApoA polypeptide is selected from an ApoA-I polypeptide, an ApoA-II polypeptide, an ApoA-III polypeptide, an ApoA-IV polypeptide, and an ApoA-V polypeptide or functional equivalent variants or fragments thereof (e.g., variants or fragments that assemble to form HDL particles).

In some embodiments, the ApoA polypeptide is an ApoA-1 polypeptide. ApoA-I refers to the mature form of the pre-proApoA-I protein that are a major component of HDL particles. ApoA-I is synthesized as a precursor (pre-proApoA-I) containing a secretion signal sequence that is eliminated to make way for the precursor. In some embodiments, the ApoA-1 polypeptide is a human ApoA-1 polypeptide. In some embodiments, human ApoA-1 has an amino acid sequence according to UniProt accession number P02647. In some embodiments, the human ApoA-1 polypeptide comprises an amino acid sequence encoded by the human ApoA-1 gene. In some embodiments, the human ApoA-1 polypeptide comprises an amino acid sequence that is the mature protein encoded by the human ApoA-1 gene. The ApoA-1 gene encodes a signal peptide of 18 amino acids, a pro-peptide of 6 amino acids in length, and a mature protein that is 243 amino acids (see, e.g., NCBI accession number X02162).

In some embodiments, the ApoA polypeptide is selected from:
(i) the mature human ApoA-1 polypeptide (e.g., having the same or essentially the same length as wild-type human ApoA-1 polypeptide without a signal peptide and pro-peptide);
(ii) a functional fragment of the mature human ApoA-1 polypeptide (e.g., a truncated (e.g., deletion of carboxy, amino terminal, or internal regions) sequence shorter than an ApoA-1 wildtype; but still retaining ApoA-1 activity);
(iii) a functional variant of the mature human ApoA-1 polypeptide (e.g., full-length, mature, or truncated ApoA-1 proteins in which one or more amino acids have been replaced, e.g., variants that retain all or most of the activity with respect to the wild-type ApoA-1 polypeptide); and
(iv) a fusion protein comprising (a) a mature human ApoA-1 wild-type, a functional fragment or a variant thereof, and (b) a heterologous protein.

As used herein, “functional fragment of the mature human ApoA-1 polypeptide” or “a functional variant of the mature human ApoA-1 polypeptide” is understood to mean polypeptides that retain the capacity to assemble to form HDL particles and/or retain their capacity to interact with HDL scavenger receptors (e.g., SR-BI). The capacity to interact with the HDL receptor is determined essentially as described by Monaco et al (EMBO.J., 1987, 6:3253-3260) either through ApoA-I binding studies to the membrane of hepatocytes or through the determination of ApoA-I or its variant's capacity to inhibit HDL bonding to the receptors of hepatocyte membranes. In some embodiments, the dissociation constant of the ApoA-I variant bond to hepatocyte membranes is at least 10⁻⁸ M, 10⁻⁷ M, 10⁻⁶ M, 10⁻⁵ M, or 10⁻⁴ M.

In some embodiments, the ApoA-I polypeptide has a high serum half-life in relation to wild-type ApoA-I mentioned, making it possible to reach serum levels of ApoA-I higher than those observed with ApoA-I. Methods for determining the serum half-life of a protein and, in particular of ApoA-I, are known in the art and include, among others, using methods based on metabolic labelling with marked proteins described by Eisenberg, S. etal (J. Lipid Res., 1973, 14:446-458), by Blumetal. (J. Clin. Invest., 1977, 60:795-807) and by Graversen et al (J Cardiovasc Pharma col., 2008, 51:170-177). An example of said variants showing a higher half-life is, for example, the variant known as Milano (which contains the R173C mutation).

In some embodiments, a human ApoA polypeptide of the disclosure comprises or consists of an amino acid sequence having at least about 50%, at least about 60%, at least about 70%, at least about 80%, at least about 85%, at least about 90%, at least about 95%, at least about 98%, or at least about 99% identity to an amino acid sequence set forth in SEQ ID NO: 14.

In some embodiments, a human ApoA polypeptide of the disclosure comprises or consists of an amino acid sequence having at least about 90%, at least about 95%, at least about 98%, or at least about 99% identity to an amino acid sequence set forth in SEQ ID NO: 14. In some embodiments, a human ApoA polypeptide of the disclosure comprises or consists of an amino acid sequence set forth in SEQ ID NO: 14.

In some embodiments, a human ApoA polypeptide of the disclosure comprises or consists of an amino acid sequence encoded by a nucleotide sequence having at least about 50%, at least about 60%, at least about 70%, at least about 80%, at least about 85%, at least about 90%, at least about 95%, at least about 98%, or at least about 99% identity to a nucleotide sequence selected from SEQ ID NOs: 34-37. In some embodiments, a human ApoA polypeptide of the disclosure comprises or consists of an amino acid sequence encoded by a nucleotide sequence having at least about 90%, at least about 95%, at least about 98%, or at least about 99% identity to a nucleotide sequence selected from SEQ ID NOs: 34-37. In some embodiments, a human ApoA polypeptide of the disclosure comprises or consists of an amino acid sequence encoded by a nucleotide sequence selected from SEQ ID NOs: 34-37.

In some embodiments, the human ApoA polypeptide is capable of assembling to form HDL particles and/or to bind to HDL scavenger receptors (e.g., SR-BI) with a dissociation constant of at least 10⁻⁸ M, 10⁻⁷ M, 10⁻⁶ M, 10⁻⁵ M, or 10⁻⁴ M.

In some embodiments, a human ApoA polypeptide of the disclosure comprises an amino acid sequence listed in SEQ ID NO: 14 with one or more conservative substitutions. In some embodiments, the conservative substitutions do not significantly affect the capacity of the human ApoA polypeptide to assemble to form HDL particles and/or to bind to HDL scavenger receptors (e.g., SR-BI) with a dissociation constant of at least 10⁻⁸ M, 10⁻⁷ M, 10⁻⁶ M, 10⁻⁵ M, or 10⁻⁴ M.

In some embodiments, the disclosure provides an mRNA comprises an ORF encoding a fusion protein, wherein the ORF comprises a nucleotide sequence encoding having at least 50%, at least 60%, at least 70%, at least 80%, at least 85%, at least 90%, at least 95%, at least 98%, at least 99%, or 100% identity to a nucleotide sequence selected from SEQ ID NOs: 34-37. In some embodiments, the ORF comprises a nucleotide sequence having at least about 85% identity to a nucleotide sequence selected from SEQ ID NOs: 34-37. In some embodiments, the ORF comprises a nucleotide sequence having at least about 90% identity to a nucleotide sequence selected from SEQ ID NOs: 34-37. In some embodiments, the ORF comprises a nucleotide sequence having at least about 95% identity to a nucleotide sequence selected from SEQ ID NOs: 34-37. In some embodiments, the ORF comprises a nucleotide sequence selected from SEQ ID NOs: 34-37.

### Signal Peptide

In some embodiments, the disclosure provides an mRNA encoding an IL15 fusion protein described herein, wherein the fusion protein comprises an N-terminal signal peptide. As would be understood by one of ordinary skill in the art, a protein encoded by an mRNA requiring expression in a cell and eventual secretion into the medium needs a signal peptide. The signal peptide is an amino acid sequence capable of promoting access to the cell secretory pathway for proteins having the signal peptide at their N-terminal end. Thus, the mRNA comprises an open reading frame encoding the fusion protein, wherein the ORF comprises 5′ to 3′ a nucleotide sequence encoding the signal peptide and a nucleotide sequence encoding the fusion protein.

Suitable signal peptides for use in the present disclosure are known in the art. Non-limiting examples include the signal peptides of tissue plasminogen activator (tPA), signal peptides of growth hormone, GM-CSF, and immunoglobulins.

In some embodiments, the signal peptide is the signal peptide of ApoA-1. In some embodiments, an mRNA encoding an IL15 fusion protein of the disclosure comprises an ORF comprising from 5′ to 3′ (i) a nucleotide sequence encoding the signal peptide of ApoA-1, and (ii) a nucleotide sequence encoding an IL15 fusion protein described herein.

In some embodiments, the signal peptide is the signal peptide of human IgG heavy chain. In some embodiments, the signal peptide of human IgG heavy chain comprises or consists of the amino acid sequence of SEQ ID NO: 13. In some embodiments, the signal peptide of human IgG heavy chain comprises or consists of an amino acid sequence having at least about 70%, about 75%, about 80%, about 85%, about 90%, about 95%, or about 99% identity to SEQ ID NO: 13.

In some embodiments, an mRNA encoding an IL15 fusion protein of the disclosure comprises an ORF comprising from 5′ to 3′ (i) a nucleotide sequence encoding the signal peptide of human IgG heavy chain, wherein the nucleotide sequence is set forth in SEQ ID NOs: 24-26, and (ii) a nucleotide sequence encoding an IL15 fusion protein described herein. In some embodiments, an mRNA encoding an IL15 fusion protein of the disclosure comprises an ORF comprising from 5′ to 3′ (i) a nucleotide sequence encoding the signal peptide of human IgG heavy chain, wherein the nucleotide sequence has at least about 70%, about 75%, about 80%, about 85%, about 90%, about 95%, or about 99% identity to the nucleotide sequence set forth in SEQ ID NOs: 24-26, and (ii) a nucleotide sequence encoding an IL15 fusion protein described herein.

In some embodiments, the signal peptide is the signal peptide of human IL15Rα. In some embodiments, the signal peptide of human IL15Rα comprises or consists of the amino acid sequence of SEQ ID NO: 12. In some embodiments, the signal peptide is the signal peptide of human IL15Rα. In some embodiments, the signal peptide of human IL15Rα comprises or consists of an amino acid sequence having at least about 70%, about 75%, about 80%, about 85%, about 90%, about 95%, or about 99% sequence identity to SEQ ID NO: 12. In some embodiments, an mRNA encoding an IL15 fusion protein of the disclosure comprises an ORF comprising from 5′ to 3′ (i) a nucleotide sequence encoding the signal peptide of human IL15Rα, wherein the nucleotide sequence is set forth in SEQ ID NOs: 27 or 28, and (ii) a nucleotide sequence encoding an IL15 fusion protein described herein. In some embodiments, an mRNA encoding an IL15 fusion protein of the disclosure comprises an ORF comprising from 5′ to 3′ (i) a nucleotide sequence encoding the signal peptide of human IL15Rα, wherein the nucleotide sequence has at least about 70%, about 75%, about 80%, about 85%, about 90%, about 95%, or about 99% identity to the nucleotide sequence set forth in SEQ ID NOs: 27 or 28 and (ii) a nucleotide sequence encoding an IL15 fusion protein described herein.

### Linkers

In some embodiments, the disclosure provides an IL15 fusion protein, wherein one or more components of the IL15 fusion protein (e.g., signal peptide, IL15 polypeptide, IL15Rα polypeptide, ApoA polypeptide) are operably linked via a linker.

In some embodiments, the linker is a peptide linker, including from one amino acid to about 200 amino acids. In some embodiments, the linker comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, or at least 40 amino acids.

In some embodiments, the linker is a GS (Gly/Ser) linkers, for example, comprising (GnS)m, wherein n is an integer from 1 to 20 and m is an integer from 1 to 20 (SEQ ID NO: 132). In some embodiments, the GS linker can comprise (GGGGS)o (SEQ ID NO: 53), wherein o is an integer from 1 to 5. In some embodiments, the GS linker can comprise GGSGGGGSGG (SEQ ID NO: 54), GGSGGGGG (SEQ ID NO: 55), or GSGSGSGS (SEQ ID NO: 56). In a particular embodiment, the linker is G6S (GGGGGGS) (SEQ ID NO: 57).

In some embodiments, the linker is a Gly-rich linker, for example, comprising (Gly)p, wherein p is an integer from 1 to 40 (SEQ ID NO: 79). In some embodiments, a Gly-rich linker can comprise GGGGG (SEQ ID NO: 58), GGGGGG (SEQ ID NO: 59), GGGGGGG (SEQ ID NO: 60) or GGGGGGGG (SEQ ID NO: 61).

In some embodiments, the linker comprises (EAAAK)q (SEQ ID NO: 62), wherein q is an integer from 1 to 5. In one embodiment, the linker can comprise (EAAAK)₃, i.e., EAAAKEAAAKEAAAK (SEQ ID NO: 63).

Further exemplary linkers include, but not limited to, GGGGSLVPRGSGGGGS (SEQ ID NO: 64), GSGSGS (SEQ ID NO: 65), GGGGSLVPRGSGGGG (SEQ ID NO: 66), GGSGGHMGSGG (SEQ ID NO: 67), GGSGGSGGSGG (SEQ ID NO: 68), GGSGG (SEQ ID NO: 69), GSGSGSGS (SEQ ID NO: 56), GGGSEGGGSEGGGSEGGG (SEQ ID NO: 70), AAGAATAA (SEQ ID NO: 71), GGSSG (SEQ ID NO: 72), GSGGGTGGGSG (SEQ ID NO: 73), GSGSGSGSGGSG (SEQ ID NO: 74), GSGGSGSGGSGGSG (SEQ ID NO: 75), GGGSGGGSGGGS (SEQ ID NO: 76), GGS(GGGS)₃LQ (SEQ ID NO: 77), and GSGGSGGSGGSGGS (SEQ ID NO: 78).

In some embodiments, the linker comprises GGGSGGGSGGGS (SEQ ID NO: 76). In some embodiments, the linker comprises GGS(GGGS)₃LQ (SEQ ID NO: 77).

In some embodiments, the disclosure provides an mRNA encoding an IL15 fusion protein described herein, wherein the mRNA comprises nucleotides encoding the linkers disclosed herein that are constructed to fuse the ORF or ORFs encoding the components of the IL15 fusion protein (e.g., ORF or ORFs encoding a signal peptide, IL15 polypeptide, IL15Rα polypeptide, and ApoA polypeptide).

### IL15 Fusion Proteins

In some embodiments, the disclosure provides an mRNA encoding an IL15 fusion protein comprising an IL15 polypeptide described herein, an IL15Rα polypeptide described herein, and an ApoA polypeptide described herein.

In some embodiments, the IL15 fusion protein comprises from N-terminus to C-terminus: (i) an IL15 polypeptide; (ii) an IL15Rα polypeptide; and (iii) an ApoA polypeptide. In some embodiments, the IL15 polypeptide and the IL15Rα polypeptide are operably linked without a linker. In some embodiments, the IL15 polypeptide and the IL15Rα polypeptide are operably linked via a linker described herein. In some embodiments, the IL15Rα polypeptide and the ApoA polypeptide are operably linked without a linker. In some embodiments, the IL15Rα polypeptide and the ApoA polypeptide are operably linked via a linker described herein.

In some embodiments, the IL15 fusion protein comprises from N-terminus to C-terminus: (i) an IL15Rα polypeptide; (ii) an IL15 polypeptide; and (iii) an ApoA polypeptide. In some embodiments, the IL15Rα polypeptide and the IL15 polypeptide are operably linked without a linker. In some embodiments, the IL15Rα polypeptide and the IL15 polypeptide are operably linked via a linker described herein. In some embodiments, the IL15 polypeptide and the ApoA polypeptide are operably linked without a linker. In some embodiments, the IL15 polypeptide and the ApoA polypeptide are operably linked via a linker described herein.

In some embodiments, the IL15 fusion protein comprises from N-terminus to C-terminus: (i) an ApoA polypeptide; (ii) an IL15 polypeptide; and (iii) an IL15Rα polypeptide. In some embodiments, the ApoA polypeptide and the IL15 polypeptide are operably linked without a linker. In some embodiments, the ApoA polypeptide and the IL15 polypeptide are operably linked via a linker described herein. In some embodiments, the IL15 polypeptide and the IL15Rα polypeptide are operably linked without a linker. In some embodiments, the IL15 polypeptide and the IL15Rα polypeptide are operably linked via a linker described herein.

In some embodiments, the IL15 fusion protein comprises from N-terminus to C-terminus: (i) an ApoA polypeptide; (ii) an IL15Rα polypeptide; and (iii) an IL15 polypeptide. In some embodiments, the ApoA polypeptide and the IL15Rα polypeptide are operably linked without a linker. In some embodiments, the ApoA polypeptide and the IL15Rα polypeptide are operably linked via a linker described herein. In some embodiments, the IL15Rα polypeptide and the IL15 polypeptide are operably linked without a linker. In some embodiments, the IL15Rα polypeptide and the IL15 polypeptide are operably linked via a linker described herein.

In some embodiments, the IL15 fusion protein comprises from N-terminus to C-terminus: (i) a human IL15 polypeptide; (ii) an extended sushi domain of a human IL15Rα polypeptide; and (iii) a human ApoA-1 polypeptide. In some embodiments, the human IL15 polypeptide and the extended sushi domain are operably linked without a linker. In some embodiments, the human IL15 polypeptide and the extended sushi domain are operably linked via a linker described herein. In some embodiments, the extended sushi domain and the human ApoA-1 polypeptide are operably linked without a linker. In some embodiments, the IL15Rα polypeptide and the human ApoA polypeptide are operably linked via a linker described herein.

In some embodiments, the IL15 fusion protein comprises from N-terminus to C-terminus: (i) an extended sushi domain of a human IL15Rα polypeptide; (ii) a human IL15 polypeptide; and (iii) a human ApoA-1 polypeptide. In some embodiments, the extended sushi domain and the human IL15 polypeptide are operably linked without a linker. In some embodiments, the extended sushi domain and the human IL15 polypeptide are operably linked via a linker described herein. In some embodiments, the human IL15 polypeptide and the human ApoA-1 polypeptide are operably linked without a linker. In some embodiments, the human IL15 polypeptide and the human ApoA-1 polypeptide are operably linked via a linker described herein.

In some embodiments, the IL15 fusion protein comprises from N-terminus to C-terminus: (i) a human ApoA-1 polypeptide; (ii) a human IL15 polypeptide; and (iii) an extended sushi domain of a human IL15Rα polypeptide. In some embodiments, the human ApoA-1 polypeptide and the human IL15 polypeptide are operably linked without a linker. In some embodiments, the human ApoA-1 polypeptide and the human IL15 polypeptide are operably linked via a linker described herein. In some embodiments, the human IL15 polypeptide and the extended sushi domain are operably linked without a linker. In some embodiments, the human IL15 polypeptide and the extended sushi domain are operably linked via a linker described herein.

In some embodiments, the IL15 fusion protein comprises from N-terminus to C-terminus: (i) a human ApoA-1 polypeptide; (ii) an extended sushi domain of a human IL15Rα polypeptide; and (iii) a human IL15 polypeptide. In some embodiments, the human ApoA-1 polypeptide and the extended sushi domain are operably linked without a linker. In some embodiments, the human ApoA-1 polypeptide and the extended sushi domain are operably linked via a linker described herein. In some embodiments, the extended sushi domain and the human IL15 polypeptide are operably linked without a linker. In some embodiments, the extended sushi domain and the human IL15 polypeptide are operably linked via a linker described herein.

In some embodiments, the IL15 fusion protein comprises or consists of an amino acid sequence selected from SEQ ID NOs: 2, 4, 9, 11, 22, 23, 121, and 123. In some embodiments, the IL15 fusion protein comprises or consists of an amino acid sequence having at least about 70%, about 75%, about 80%, about 85%, about 90%, about 95%, or about 99% sequence identity to an amino acid sequence selected from SEQ ID NOs: 2, 4, 9, 11, 22, 23, 121, and 123. In some embodiments, the IL15 fusion protein comprises or consists of an amino acid sequence having at least about 90%, about 91%, about 92%, about 93%, about 94%, about 95%, about 96%, about 97%, about 98%, or about 99% sequence identity to an amino acid sequence selected from SEQ ID NOs: 2, 4, 9, 11, 22, 23, 121, and 123. In some embodiments, the IL15 fusion protein comprises or consists of an amino acid sequence having one or more conservative substitutions relative to an amino acid sequence selected from SEQ ID NOs: 2, 4, 9, 11, 22, 23, 121, and 123, wherein the conservative substitutions do not significantly affect the binding activity of the IL15 fusion protein to its receptor.

In some embodiments, the IL15 fusion protein comprises or consists of an amino acid sequence selected from SEQ ID NOs: 2, 4, 11, 23, and 121. In some embodiments, the IL15 fusion protein comprises or consists of an amino acid sequence having at least about 70%, about 75%, about 80%, about 85%, about 90%, about 95%, or about 99% sequence identity to an amino acid sequence selected from SEQ ID NOs: 2, 4, 11, 23, and 121. In some embodiments, the IL15 fusion protein comprises or consists of an amino acid sequence having at least about 90%, about 91%, about 92%, about 93%, about 94%, about 95%, about 96%, about 97%, about 98%, or about 99% sequence identity to an amino acid sequence selected from SEQ ID NOs: 2, 4, 11, 23, and 121. In some embodiments, the IL15 fusion protein comprises or consists of an amino acid sequence having one or more conservative substitutions relative to an amino acid sequence selected from SEQ ID NOs: 2, 4, 11, 23, and 121, wherein the conservative substitutions do not significantly affect the binding activity of the IL15 fusion protein to its receptor.

In some embodiments, the IL15 fusion protein comprises or consists of an amino acid sequence set forth in SEQ ID NO: 2. In some embodiments, the IL15 fusion protein comprises or consists of an amino acid sequence having at least about 70%, about 75%, about 80%, about 85%, about 90%, about 95%, or about 99% sequence identity to SEQ ID NO: 2. In some embodiments, the IL15 fusion protein comprises or consists of an amino acid sequence having at least about 90%, about 91%, about 92%, about 93%, about 94%, about 95%, about 96%, about 97%, about 98%, or about 99% sequence identity to SEQ ID NO: 2. In some embodiments, the IL15 fusion protein comprises or consists of an amino acid sequence encoded by a nucleotide sequence set forth in SEQ ID NO: 1. In some embodiments, the IL15 fusion protein comprises or consists of an amino acid sequence encoded by a nucleotide sequence having at least about 70%, about 75%, about 80%, about 85%, about 90%, about 95%, or about 99% sequence identity to the nucleotide sequence forth in SEQ ID NO: 1. In some embodiments, the IL15 fusion protein comprises or consists of an amino acid sequence encoded by a nucleotide sequence having at least about 90%, about 91%, about 92%, about 93%, about 94%, about 95%, about 96%, about 97%, about 98%, or about 99% sequence identity to the nucleotide sequence forth in SEQ ID NO: 1.

In some embodiments, the IL15 fusion protein comprises or consists of an amino acid sequence set forth in SEQ ID NO: 4. In some embodiments, the IL15 fusion protein comprises or consists of an amino acid sequence having at least about 70%, about 75%, about 80%, about 85%, about 90%, about 95%, or about 99% sequence identity to SEQ ID NO: 4. In some embodiments, the IL15 fusion protein comprises or consists of an amino acid sequence having at least about 90%, about 95%, about 98%, or about 99% sequence identity to SEQ ID NO: 4. In some embodiments, the IL15 fusion protein comprises or consists of an amino acid sequence encoded by a nucleotide sequence set forth in SEQ ID NO: 3. In some embodiments, the IL15 fusion protein comprises or consists of an amino acid sequence encoded by a nucleotide sequence having at least about 70%, about 75%, about 80%, about 85%, about 90%, about 95%, or about 99% sequence identity to the nucleotide sequence forth in SEQ ID NO: 3. In some embodiments, the IL15 fusion protein comprises or consists of an amino acid sequence encoded by a nucleotide sequence having at least about 90%, about 91%, about 92%, about 93%, about 94%, about 95%, about 96%, about 97%, about 98%, or about 99% sequence identity to the nucleotide sequence forth in SEQ ID NO: 3.

In some embodiments, the IL15 fusion protein comprises or consists of an amino acid sequence set forth in SEQ ID NO: 9. In some embodiments, the IL15 fusion protein comprises or consists of an amino acid sequence having at least about 70%, about 75%, about 80%, about 85%, about 90%, about 95%, or about 99% sequence identity to SEQ ID NO: 9. In some embodiments, the IL15 fusion protein comprises or consists of an amino acid sequence having at least about 90%, about 95%, about 98%, or about 99% sequence identity to SEQ ID NO: 9. In some embodiments, the IL15 fusion protein comprises or consists of an amino acid sequence encoded by a nucleotide sequence set forth in SEQ ID NO: 8. In some embodiments, the IL15 fusion protein comprises or consists of an amino acid sequence encoded by a nucleotide sequence having at least about 70%, about 75%, about 80%, about 85%, about 90%, about 95%, or about 99% sequence identity to the nucleotide sequence forth in SEQ ID NO: 8. In some embodiments, the IL15 fusion protein comprises or consists of an amino acid sequence encoded by a nucleotide sequence having at least about 90%, about 91%, about 92%, about 93%, about 94%, about 95%, about 96%, about 97%, about 98%, or about 99% sequence identity to the nucleotide sequence forth in SEQ ID NO: 8.

In some embodiments, the IL15 fusion protein comprises or consists of an amino acid sequence set forth in SEQ ID NO: 11. In some embodiments, the IL15 fusion protein comprises or consists of an amino acid sequence having at least about 70%, about 75%, about 80%, about 85%, about 90%, about 95%, or about 99% sequence identity to SEQ ID NO: 11. In some embodiments, the IL15 fusion protein comprises or consists of an amino acid sequence having at least about 90%, about 91%, about 92%, about 93%, about 94%, about 95%, about 96%, about 97%, about 98%, or about 99% sequence identity to SEQ ID NO: 11. In some embodiments, the IL15 fusion protein comprises or consists of an amino acid sequence encoded by a nucleotide sequence set forth in SEQ ID NO: 10. In some embodiments, the IL15 fusion protein comprises or consists of an amino acid sequence encoded by a nucleotide sequence having at least about 70%, about 75%, about 80%, about 85%, about 90%, about 95%, or about 99% sequence identity to the nucleotide sequence forth in SEQ ID NO: 10. In some embodiments, the IL15 fusion protein comprises or consists of an amino acid sequence encoded by a nucleotide sequence having at least about about 90%, about 91%, about 92%, about 93%, about 94%, about 95%, about 96%, about 97%, about 98%, or about 99% sequence identity to the nucleotide sequence forth in SEQ ID NO: 10.

In some embodiments, the IL15 fusion protein comprises or consists of an amino acid sequence set forth in SEQ ID NO: 22. In some embodiments, the IL15 fusion protein comprises or consists of an amino acid sequence having at least about 70%, about 75%, about 80%, about 85%, about 90%, about 95%, or about 99% sequence identity to SEQ ID NO: 22. In some embodiments, the IL15 fusion protein comprises or consists of an amino acid sequence having at least about 90%, about 91%, about 92%, about 93%, about 94%, about 95%, about 96%, about 97%, about 98%, or about 99% sequence identity to SEQ ID NO: 22.

In some embodiments, the IL15 fusion protein comprises or consists of an amino acid sequence set forth in SEQ ID NO: 23. In some embodiments, the IL15 fusion protein comprises or consists of an amino acid sequence having at least about 70%, about 75%, about 80%, about 85%, about 90%, about 95%, or about 99% sequence identity to SEQ ID NO: 23. In some embodiments, the IL15 fusion protein comprises or consists of an amino acid sequence having at least about 90%, about 91%, about 92%, about 93%, about 94%, about 95%, about 96%, about 97%, about 98%, or about 99% sequence identity to SEQ ID NO: 23.

In some embodiments, the IL15 fusion protein comprises or consists of an amino acid sequence set forth in SEQ ID NO: 123. In some embodiments, the IL15 fusion protein comprises or consists of an amino acid sequence having at least about 70%, about 75%, about 80%, about 85%, about 90%, about 95%, or about 99% sequence identity to SEQ ID NO: 123. In some embodiments, the IL15 fusion protein comprises or consists of an amino acid sequence having at least about 90%, about 95%, about 98%, or about 99% sequence identity to SEQ ID NO: 123. In some embodiments, the IL15 fusion protein comprises or consists of an amino acid sequence encoded by a nucleotide sequence set forth in SEQ ID NO: 122. In some embodiments, the IL15 fusion protein comprises or consists of an amino acid sequence encoded by a nucleotide sequence having at least about 70%, about 75%, about 80%, about 85%, about 90%, about 95%, or about 99% sequence identity to the nucleotide sequence forth in SEQ ID NO: 122. In some embodiments, the IL15 fusion protein comprises or consists of an amino acid sequence encoded by a nucleotide sequence having at least about 90%, about 91%, about 92%, about 93%, about 94%, about 95%, about 96%, about 97%, about 98%, or about 99% sequence identity to the nucleotide sequence forth in SEQ ID NO: 122.

In some embodiments, the IL15 fusion protein comprises or consists of an amino acid sequence set forth in SEQ ID NO: 121. In some embodiments, the IL15 fusion protein comprises or consists of an amino acid sequence having at least about 70%, about 75%, about 80%, about 85%, about 90%, about 95%, or about 99% sequence identity to SEQ ID NO: 121. In some embodiments, the IL15 fusion protein comprises or consists of an amino acid sequence having at least about 90%, about 95%, about 98%, or about 99% sequence identity to SEQ ID NO: 121. In some embodiments, the IL15 fusion protein comprises or consists of an amino acid sequence encoded by a nucleotide sequence set forth in SEQ ID NO: 120. In some embodiments, the IL15 fusion protein comprises or consists of an amino acid sequence encoded by a nucleotide sequence having at least about 70%, about 75%, about 80%, about 85%, about 90%, about 95%, or about 99% sequence identity to the nucleotide sequence forth in SEQ ID NO: 120. In some embodiments, the IL15 fusion protein comprises or consists of an amino acid sequence encoded by a nucleotide sequence having at least about 90%, about 91%, about 92%, about 93%, about 94%, about 95%, about 96%, about 97%, about 98%, or about 99% sequence identity to the nucleotide sequence forth in SEQ ID NO: 120.

In some embodiments, an IL15 fusion protein described herein comprising ApoA polypeptide has increased circulation half-life following in vivo administration as compared to a control IL15 fusion protein (e.g., an IL15 fusion protein comprising an Fc domain, optionally wherein the IL15 fusion protein comprises the amino acid sequence of SEQ ID NO: 127). In some embodiments, an IL15 fusion protein comprising an ApoA polypeptide described herein has increased accumulation in a target tissue and/or target cell population (e.g., tumor tissue) compared to a control IL15 fusion protein (e.g., an IL15 fusion protein comprising an Fc domain, optionally wherein the IL15 fusion protein comprises the amino acid sequence of SEQ ID NO: 127). Methods to measure pharmacokinetics and/or biodistribution of an IL15 fusion protein following in vivo administration are known in the art and are described in the Examples. For example, a suitable method for use in the present disclosure comprises an IL15 ELISA to quantify IL15 fusion protein present in a serum sample or tissue sample (e.g., tumor sample) collected from a subject following in vivo administration.

In some embodiments, an IL15 fusion protein described herein comprising an ApoA polypeptide induces activation and/or proliferation of immune cells (e.g., CD8 T cells, NK cells, NKT cells) following ex vivo or in vivo administration. In some embodiments, the IL15 fusion protein comprising ApoA polypeptide activates T cells, NK cells, NKT cells, or a combination thereof. In some embodiments, the IL15 fusion protein comprising ApoA polypeptide activates T cells, NK cells, NKT cells, or a combination thereof (e.g., following ex vivo or in vivo administration) to a greater extent than a control IL15 fusion protein (e.g., an IL15 fusion protein comprising an Fc domain, optionally wherein the IL15 fusion protein comprises the amino acid sequence of SEQ ID NO: 127). Methods to measure proliferation and/or activation status of immune cell subsets are known in the art and are described in the Examples. For example, to measure proliferation and/or activation following in vivo administration, a tissue sample (e.g., lymphatic tissue, spleen, or liver sample) is obtained and total numbers and activation status of immune cell subsets are quantified by flow cytometry. NK and T cell activation can be measured by analyzing surface expression of an activation marker (e.g., CD25 and CD69) on an NK cell or T cell by flow cytometry. Moreover, Ki67 staining can be used as a marker of proliferation among immune cell subsets evaluated by flow cytometry.

In some embodiments, the disclosure provides an mRNA encoding an IL15 fusion protein described herein. In some embodiments, the mRNA comprises an ORF encoding the IL15 fusion protein. In some embodiments, the ORF comprises from 5′ to 3′ a nucleotide sequence encoding a signal peptide described herein operably linked to a nucleotide sequence encoding the IL15 fusion protein. In some embodiments, the ORF comprises a nucleotide sequence set forth in any one of SEQ ID Nos: 1, 3, 8, 10, 120, or 122. In some embodiments, the ORF comprises a nucleotide sequence having at least about 60%, about 65%, about 70%, about 75%, about 80%, about 85%, about 90%, about 95%, or about 99% sequence identity to a nucleotide sequence set forth in any one of SEQ ID Nos: 1, 3, 8, 10, 120, or 122. In some embodiments, the ORF comprises a nucleotide sequence having at least about 90%, about 91%, about 92%, about 93%, about 94%, about 95%, about 96%, about 97%, about 98%, or about 99% sequence identity to a nucleotide sequence set forth in any one of SEQ ID Nos: 1, 3, 8, 10, 120, or 122. In some embodiments, the ORF comprises a nucleotide sequence encoding an IL15 fusion protein, wherein the nucleotide sequence encoding the IL15 fusion protein has at least about 60%, about 65%, about 70%, about 75%, about 80%, about 85%, about 90%, about 95%, or about 99% sequence identity to any one of SEQ ID NOs: 120 or 122. In some embodiments, the nucleotide sequence encoding the IL15 fusion protein has at least about 90%, about 91%, about 92%, about 93%, about 94%, about 95%, about 96%, about 97%, about 98%, or about 99% sequence identity to any one of SEQ ID NOs: 120 or 122. In some embodiments, the nucleotide sequence encoding the IL15 fusion protein is set forth in any one of SEQ ID NOs: 120 or 122.

In some embodiments, the ORF comprises a nucleotide sequence set forth in any one of SEQ ID Nos: 1, 10, and 120. In some embodiments, the ORF comprises a nucleotide sequence having at least about 60%, about 65%, about 70%, about 75%, about 80%, about 85%, about 90%, about 95%, or about 99% sequence identity to a nucleotide sequence set forth in any one of SEQ ID Nos: 1, 10, and 120. In some embodiments, the ORF comprises a nucleotide sequence having at least about 90%, about 91%, about 92%, about 93%, about 94%, about 95%, about 96%, about 97%, about 98%, or about 99% sequence identity to a nucleotide sequence set forth in any one of SEQ ID Nos: 1, 10, and 120.

In some embodiments, the disclosure provides an mRNA comprising an ORF encoding an IL15 fusion protein, wherein the ORF comprises the nucleotide sequence set forth in SEQ ID NO: 1. In some embodiments, the ORF comprises a nucleotide sequence having at least about 70%, about 75%, about 80%, about 85%, about 90%, about 95%, or about 99% sequence identity to the nucleotide sequence set forth in SEQ ID NO: 1. In some embodiments, the ORF comprises a nucleotide sequence having at least about 90%, about 91%, about 92%, about 93%, about 94%, about 95%, about 96%, about 97%, about 98%, or about 99% sequence identity to the nucleotide sequence set forth in SEQ ID NO: 1.

In some embodiments, the disclosure provides an mRNA comprising an ORF encoding an IL15 fusion protein, wherein the ORF comprises the nucleotide sequence set forth in SEQ ID NO: 3. In some embodiments, the ORF comprises a nucleotide sequence having at least about 70%, about 75%, about 80%, about 85%, about 90%, about 95%, or about 99% sequence identity to the nucleotide sequence set forth in SEQ ID NO: 3. In some embodiments, the ORF comprises a nucleotide sequence having at least about 90%, about 91%, about 92%, about 93%, about 94%, about 95%, about 96%, about 97%, about 98%, or about 99% sequence identity to the nucleotide sequence set forth in SEQ ID NO: 3.

In some embodiments, the disclosure provides an mRNA comprising an ORF encoding an IL15 fusion protein, wherein the ORF comprises the nucleotide sequence set forth in SEQ ID NO: 8. In some embodiments, the ORF comprises a nucleotide sequence having at least about 70%, about 75%, about 80%, about 85%, about 90%, about 95%, or about 99% sequence identity to the nucleotide sequence set forth in SEQ ID NO: 8. In some embodiments, the ORF comprises a nucleotide sequence having at least about 90%, about 91%, about 92%, about 93%, about 94%, about 95%, about 96%, about 97%, about 98%, or about 99% sequence identity to the nucleotide sequence set forth in SEQ ID NO: 8.

In some embodiments, the disclosure provides an mRNA comprising an ORF encoding an IL15 fusion protein, wherein the ORF comprises the nucleotide sequence set forth in SEQ ID NO: 10. In some embodiments, the ORF comprises a nucleotide sequence having at least about 70%, about 75%, about 80%, about 85%, about 90%, about 95%, or about 99% sequence identity to the nucleotide sequence set forth in SEQ ID NO: 10. In some embodiments, the ORF comprises a nucleotide sequence having at least about 90%, about 91%, about 92%, about 93%, about 94%, about 95%, about 96%, about 97%, about 98%, or about 99% sequence identity to the nucleotide sequence set forth in SEQ ID NO: 10.

In some embodiments, the disclosure provides an mRNA comprising an ORF encoding an IL15 fusion protein, wherein the ORF comprises the nucleotide sequence set forth in SEQ ID NO: 120. In some embodiments, the ORF comprises a nucleotide sequence having at least about 70%, about 75%, about 80%, about 85%, about 90%, about 95%, or about 99% sequence identity to the nucleotide sequence set forth in SEQ ID NO: 120. In some embodiments, the ORF comprises a nucleotide sequence having at least about 90%, about 91%, about 92%, about 93%, about 94%, about 95%, about 96%, about 97%, about 98%, or about 99% sequence identity to the nucleotide sequence set forth in SEQ ID NO: 120.

In some embodiments, the disclosure provides an mRNA comprising an ORF encoding an IL15 fusion protein, wherein the ORF comprises the nucleotide sequence set forth in SEQ ID NO: 122. In some embodiments, the ORF comprises a nucleotide sequence having at least about 70%, about 75%, about 80%, about 85%, about 90%, about 95%, or about 99% sequence identity to the nucleotide sequence set forth in SEQ ID NO: 122. In some embodiments, the ORF comprises a nucleotide sequence having at least about 90%, about 91%, about 92%, about 93%, about 94%, about 95%, about 96%, about 97%, about 98%, or about 99% sequence identity to the nucleotide sequence set forth in SEQ ID NO: 122.

In some embodiments, sequence tags or amino acids, are added to the sequences of mRNAs of the disclosure (e.g., at the 5′ or 3′ ends of the ORF), e.g., to facilitate localization. In some embodiments, amino acid residues located at the carboxy, amino terminal, or internal regions of the IL15 fusion protein encoded by an mRNA of the disclosure are optionally deleted.

mRNA Construct Components

An mRNA may be a naturally or non-naturally occurring mRNA. An mRNA may include one or more modified nucleobases, nucleosides, or nucleotides, as described below, in which case it may be referred to as a “modified mRNA” or “mmRNA.” As described herein “nucleoside” is defined as a compound containing a sugar molecule (e.g., a pentose or ribose) or derivative thereof in combination with an organic base (e.g., a purine or pyrimidine) or a derivative thereof (also referred to herein as “nucleobase”). As described herein, “nucleotide” is defined as a nucleoside including a phosphate group.

An mRNA may include a 5′ untranslated region (5′-UTR), a 3′ untranslated region (3′-UTR), and/or a coding region (e.g., an open reading frame). Exemplary 5′ UTRs for use in the constructs are shown in SEQ ID NOs: 80-97 and 107-108. Another exemplary 5′ UTR for use in the constructs is shown in SEQ ID NO: 19. An exemplary 3′ UTR for use in the constructs is shown in SEQ ID NO: 20.

In some embodiments, an mRNA of the disclosure comprises a 5′UTR, wherein the 5′UTR comprises a nucleotide sequence selected from SEQ ID Nos: 80-97 and 107-108. In some embodiments, an mRNA of the disclosure comprises a 5′UTR, wherein the 5′UTR comprises the nucleotide sequence of SEQ ID NO: 19. In some embodiments, the mRNA comprises a 3′UTR, wherein the 3′UTR comprises the nucleotide sequence of SEQ ID NO: 20.

An mRNA may include any suitable number of base pairs, including tens (e.g., 10, 20, 30, 40, 50, 60, 70, 80, 90 or 100), hundreds (e.g., 200, 300, 400, 500, 600, 700, 800, or 900) or thousands (e.g., 1000, 2000, 3000, 4000, 5000, 6000, 7000, 8000, 9000, 10,000) of base pairs.

Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified.

In some embodiments, an mRNA as described herein may include a 5′ cap structure, a chain terminating nucleotide, optionally a Kozak sequence (also known as a Kozak consensus sequence, e.g., SEQ ID NO: 99), a stem loop, a polyA sequence, and/or a polyadenylation signal.

A 5′ cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m7G(5′)ppp(5′)G, commonly written as m7GpppG. A cap species may also be an anti-reverse cap analog. A non-limiting list of possible cap species includes m7GpppG, m7Gpppm7G, m73′dGpppG, m27,03′GpppG, m27,03′GppppG, m27,02′GppppG, m7Gpppm7G, m73′dGpppG, m27,03′GpppG, m27,03′GppppG, and m27,02′GppppG.

An mRNA may instead or additionally include a chain terminating nucleoside. For example, a chain terminating nucleoside may include those nucleosides deoxygenated at the 2′ and/or 3′ positions of their sugar group. Such species may include 3′ deoxyadenosine (cordycepin), 3′ deoxyuridine, 3′ deoxycytosine, 3′ deoxyguanosine, 3′ deoxythymine, and 2′,3′ dideoxynucleosides, such as 2′,3′ dideoxyadenosine, 2′,3′ dideoxyuridine, 2′,3′ dideoxycytosine, 2′,3′ dideoxyguanosine, and 2′,3′ dideoxythymine. In some embodiments, incorporation of a chain terminating nucleotide into an mRNA, for example at the 3′-terminus, may result in stabilization of the mRNA, as described, for example, in International Patent Publication No. WO 2013/103659.

An mRNA may instead or additionally include a stem loop, such as a histone stem loop. A stem loop may include 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail. In some embodiments, a stem loop may affect one or more function(s) of an mRNA, such as initiation of translation, translation efficiency, and/or transcriptional termination.

An mRNA may instead or additionally include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. In some embodiments, a polyA sequence may affect the nuclear export, translation, and/or stability of an mRNA.

An mRNA may instead or additionally include a microRNA binding site.

In some embodiments, an mRNA is a bicistronic mRNA comprising a first coding region and a second coding region with an intervening sequence comprising an internal ribosome entry site (IRES) sequence that allows for internal translation initiation between the first and second coding regions, or with an intervening sequence encoding a self-cleaving peptide, such as a 2A peptide. IRES sequences and 2A peptides are typically used to enhance expression of multiple proteins from the same vector. A variety of IRES sequences are known and available in the art and may be used, including, e.g., the encephalomyocarditis virus IRES.

In one embodiment, the polynucleotides of the present disclosure may include a sequence encoding a self-cleaving peptide. The self-cleaving peptide may be, but is not limited to, a 2A peptide. A variety of 2A peptides are known and available in the art and may be used, including e.g., the foot and mouth disease virus (FMDV) 2A peptide, the equine rhinitis A virus 2A peptide, the Thosea asigna virus 2A peptide, and the porcine teschovirus-1 2A peptide. 2A peptides are used by several viruses to generate two proteins from one transcript by ribosome-skipping, such that a normal peptide bond is impaired at the 2A peptide sequence, resulting in two discontinuous proteins being produced from one translation event. As a non-limiting example, the 2A peptide may have the protein sequence: GSGATNFSLLKQAGDVEENPGP (SEQ ID NO: 100), fragments or variants thereof. In one embodiment, the 2A peptide cleaves between the last glycine and last proline. As another non-limiting example, the polynucleotides of the present disclosure may include a polynucleotide sequence encoding the 2A peptide having the protein sequence GSGATNFSLLKQAGDVEENPGP (SEQ ID NO: 100) fragments or variants thereof. One example of a polynucleotide sequence encoding the 2A peptide is: GGAAGCGGAGCTACTAACTTCAGCCTGCTGAAGCAGGCTGGAGACGTGGAGGAGAA CCCTGGACCT (SEQ ID NO: 101). In one illustrative embodiment, a 2A peptide is encoded by the following sequence: 5′-TCCGGACTCAGATCCGGGGATCTCAAAATTGTCGCTCCTGTCAAACAAACTCTTAAC TTTGATTTACTCAAACTGGCTGGGGATGTAGAAAGCAATCCAGGTCCACTC-3′(SEQ ID NO: 102). The polynucleotide sequence of the 2A peptide may be modified or codon optimized by the methods described herein and/or are known in the art.

In one embodiment, this sequence may be used to separate the coding regions of two or more polypeptides of interest. As a non-limiting example, the sequence encoding the F2A peptide may be between a first coding region A and a second coding region B (A-F2Apep-B). The presence of the F2A peptide results in the cleavage of the one long protein between the glycine and the proline at the end of the F2A peptide sequence (NPGP (SEQ ID NO: 131) is cleaved to result in NPG and P) thus creating separate protein A (with 21 amino acids of the F2A peptide attached, ending with NPG) and separate protein B (with 1 amino acid, P, of the F2A peptide attached). Likewise, for other 2A peptides (P2A, T2A and E2A), the presence of the peptide in a long protein results in cleavage between the glycine and proline at the end of the 2A peptide sequence (NPGP (SEQ ID NO: 131) is cleaved to result in NPG and P). Protein A and protein B may be the same or different peptides or polypeptides of interest. In particular embodiments, protein A is a polypeptide that induces immunogenic cell death and protein B is another polypeptide that stimulates an inflammatory and/or immune response and/or regulates immune responsiveness (as described further below).

Modified mRNAs

In some embodiments, an mRNA of the disclosure comprises one or more modified nucleobases, nucleosides, or nucleotides (termed “modified mRNAs” or “mmRNAs”). In some embodiments, modified mRNAs may have useful properties, including enhanced stability, intracellular retention, enhanced translation, and/or the lack of a substantial induction of the innate immune response of a cell into which the mRNA is introduced, as compared to a reference unmodified mRNA. Therefore, use of modified mRNAs may enhance the efficiency of protein production, intracellular retention of nucleic acids, as well as possess reduced immunogenicity.

In some embodiments, an mRNA includes one or more (e.g., 1, 2, 3 or 4) different modified nucleobases, nucleosides, or nucleotides. In some embodiments, an mRNA includes one or more (e.g., 1, 2, 3, 4, 5, 6, 7, 8, 9, 10, 20, 30, 40, 50, 60, 70, 80, 90, 100, or more) different modified nucleobases, nucleosides, or nucleotides. In some embodiments, the modified mRNA may have reduced degradation in a cell into which the mRNA is introduced, relative to a corresponding unmodified mRNA.

In some embodiments, the modified nucleobase is a modified uracil. Exemplary nucleobases and nucleosides having a modified uracil include pseudouridine (W), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridineor 5-bromo-uridine), 3-methyl-uridine (m3U), 5-methoxy-uridine (mo5U), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (rm5U), 1-taurinomethyl-pseudouridine, 5-taurinomethyl-2-thio-uridine(rm5s2U), 1-taurinomethyl-4-thio-pseudouridine, 5-methyl-uridine (m5U, i.e., having the nucleobase deoxythymine), 1-methyl-pseudouridine (mlW), 5-methyl-2-thio-uridine (m5s2U), 1-methyl-4-thio-pseudouridine (mls4W), 4-thio-1-methyl-pseudouridine, 3-methyl-pseudouridine (m3W), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp3U), 1-methyl-3-(3-amino-3-carboxypropyl)pseudouridine (acp3 W), 5-(isopentenylaminomethyl)uridine (inm5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (Wm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), and 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m3C), N4-acetyl-cytidine (ac4C), 5-formyl-cytidine (f5C), N4-methyl-cytidine (m4C), 5-methyl-cytidine (m5C), 5-halo-cytidine (e.g., 5-iodo-cytidine), 5-hydroxymethyl-cytidine (hm5C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-i-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k2C), α-thio-cytidine, 2′-O-methyl-cytidine (Cm), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-O-trimethyl-cytidine (m42Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include D-thio-adenosine, 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m1A), 2-methyl-adenine (m2A), N6-methyl-adenosine (m6A), 2-methylthio-N6-methyl-adenosine (ms2m6A), N6-isopentenyl-adenosine (i6A), 2-methylthio-N6-isopentenyl-adenosine (ms2i6A), N6-(cis-hydroxyisopentenyl)adenosine (io6A), 2-methylthio-N6-(cis-hydroxyisopentenyl)adenosine (ms2io6A), N6-glycinylcarbamoyl-adenosine (g6A), N6-threonylcarbamoyl-adenosine (t6A), N6-methyl-N6-threonylcarbamoyl-adenosine (m6t6A), 2-methylthio-N6-threonylcarbamoyl-adenosine (ms2g6A), N6,N6-dimethyl-adenosine (m62A), N6-hydroxynorvalylcarbamoyl-adenosine (hn6A), 2-methylthio-N6-hydroxynorvalylcarbamoyl-adenosine (ms2hn6A), N6-acetyl-adenosine (ac6A), 7-methyl-adenine, 2-methylthio-adenine, 2-methoxy-adenine, α-thio-adenosine, 2′-O-methyl-adenosine (Am), N6,2′-O-dimethyl-adenosine (m6Am), N6,N6,2′-O-trimethyl-adenosine (m62Am), 1,2′-O-dimethyl-adenosine (mlAm), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include α-thio-guanosine, inosine (I), 1-methyl-inosine (mlI), wyosine (imG), methylwyosine (mimG), 4-demethyl-wyosine (imG-14), isowyosine (imG2), wybutosine (yW), peroxywybutosine (o2yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQO), 7-aminomethyl-7-deaza-guanosine (preQ1), archaeosine (G+), 7-deaza-8-aza-guanosine, 6-thio-guanosine, 6-thio-7-deaza-guanosine, 6-thio-7-deaza-8-aza-guanosine, 7-methyl-guanosine (m7G), 6-thio-7-methyl-guanosine, 7-methyl-inosine, 6-methoxy-guanosine, 1-methyl-guanosine (m1G), N2-methyl-guanosine (m2G), N2,N2-dimethyl-guanosine (m22G), N2,7-dimethyl-guanosine (m2,7G), N2, N2,7-dimethyl-guanosine (m2,2,7G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m2Gm), N2,N2-dimethyl-2′-O-methyl-guanosine (m22Gm), 1-methyl-2′-O-methyl-guanosine (m1Gm), N2,7-dimethyl-2′-O-methyl-guanosine (m2,7Gm), 2′-O-methyl-inosine (Im), 1,2′-O-dimethyl-inosine (m1Im), 2′-O-ribosylguanosine (phosphate) (Gr(p)), 1-thio-guanosine, 06-methyl-guanosine, 2′-F-ara-guanosine, and 2′-F-guanosine.

In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is pseudouridine (W), N1-methylpseudouridine (mlW), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, or 2′-O-methyl uridine. In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is a modified cytosine. Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), 2-thio-5-methyl-cytidine. In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is a modified adenine. Exemplary nucleobases and nucleosides having a modified adenine include 7-deaza-adenine, 1-methyl-adenosine (m1A), 2-methyl-adenine (m2A), N6-methyl-adenosine (m6A). In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is a modified guanine. Exemplary nucleobases and nucleosides having a modified guanine include inosine (I), 1-methyl-inosine (mlI), wyosine (imG), methylwyosine (mimG), 7-deaza-guanosine, 7-cyano-7-deaza-guanosine (preQO), 7-aminomethyl-7-deaza-guanosine (preQ1), 7-methyl-guanosine (m7G), 1-methyl-guanosine (m1G), 8-oxo-guanosine, 7-methyl-8-oxo-guanosine. In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is 1-methyl-pseudouridine (mlW), 5-methoxy-uridine (mo5U), 5-methyl-cytidine (m5C), pseudouridine (W), α-thio-guanosine, or α-thio-adenosine. In some embodiments, an mRNA of the disclosure includes a combination of one or more of the aforementioned modified nucleobases (e.g., a combination of 2, 3 or 4 of the aforementioned modified nucleobases).

In some embodiments, the mRNA comprises pseudouridine (W). In some embodiments, the mRNA comprises pseudouridine (W) and 5-methyl-cytidine (m5C). In some embodiments, the mRNA comprises 1-methyl-pseudouridine (mlW). In some embodiments, the mRNA comprises 1-methyl-pseudouridine (mlW) and 5-methyl-cytidine (m5C). In some embodiments, the mRNA comprises 2-thiouridine (s2U). In some embodiments, the mRNA comprises 2-thiouridine and 5-methyl-cytidine (m5C). In some embodiments, the mRNA comprises 5-methoxy-uridine (mo5U). In some embodiments, the mRNA comprises 5-methoxy-uridine (mo5U) and 5-methyl-cytidine (m5C). In some embodiments, the mRNA comprises 2′-O-methyl uridine. In some embodiments, the mRNA comprises 2′-O-methyl uridine and 5-methyl-cytidine (m5C). In some embodiments, the mRNA comprises N6-methyl-adenosine (m6A). In some embodiments, the mRNA comprises N6-methyl-adenosine (m6A) and 5-methyl-cytidine (m5C).

In certain embodiments, an mRNA of the disclosure is uniformly modified (i.e., fully modified, modified through-out the entire sequence) for a particular modification. In some embodiments, an mRNA of the disclosure is modified wherein at least 95%, at least 96%, at least 97%, at least 98%, at least 99% or 100% of a specified nucleotide or nucleobase is modified.

For example, an mRNA can be uniformly modified with 5-methyl-cytidine (m5C), meaning that all cytosine residues in the mRNA sequence are replaced with 5-methyl-cytidine (m5C).

Similarly, mRNAs of the disclosure can be uniformly modified for any type of nucleoside residue present in the sequence by replacement with a modified residue such as those set forth above. In some embodiments, an mRNA of the disclosure is uniformly modified with 1-methyl pseudouridine (mlW), meaning that all uridine residues in the mRNA sequence are replaced with 1-methyl pseudouridine (mlW). In some embodiments, at least 95%, at least 96%, at least 97%, at least 98%, at least 99% or 100% of uridines are 1-methyl pseudouridine (m1w).

In some embodiments, an mRNA of the disclosure may be modified in a coding region (e.g., an open reading frame encoding a polypeptide). In other embodiments, an mRNA may be modified in regions besides a coding region. For example, in some embodiments, a 5′-UTR and/or a 3′-UTR are provided, wherein either or both may independently contain one or more different nucleoside modifications. In such embodiments, nucleoside modifications may also be present in the coding region.

Examples of nucleoside modifications and combinations thereof that may be present in mmRNAs of the present disclosure include, but are not limited to, those described in PCT Patent Application Publications: WO2012045075, WO2014081507, WO2014093924, WO2014164253, and WO2014159813.

The mmRNAs of the disclosure can include a combination of modifications to the sugar, the nucleobase, and/or the internucleoside linkage. These combinations can include any one or more modifications described herein.

In certain embodiments, the modified nucleosides may be partially or completely substituted for the natural nucleotides of the mRNAs of the disclosure. As a non-limiting example, the natural nucleotide uridine may be substituted with a modified nucleoside described herein. In another non-limiting example, the natural nucleoside uridine may be partially substituted (e.g., about 0.1%, 1%, 5%, 10%, 15%, 20%, 25%, 30%, 35%, 40%, 45%, 50%, 55%, 60%, 65%, 70%, 75%, 80%, 85%, 90%, 95% or 99.9% of the natural uridines) with at least one of the modified nucleoside disclosed herein.

The mRNAs of the present disclosure, or regions thereof, may be codon optimized. Codon optimization methods are known in the art and may be useful for a variety of purposes: matching codon frequencies in host organisms to ensure proper folding, bias GC content to increase mRNA stability or reduce secondary structures, minimize tandem repeat codons or base runs that may impair gene construction or expression, customize transcriptional and translational control regions, insert or remove proteins trafficking sequences, remove/add post translation modification sites in encoded proteins (e.g., glycosylation sites), add, remove or shuffle protein domains, insert or delete restriction sites, modify ribosome binding sites and mRNA degradation sites, adjust translation rates to allow the various domains of the protein to fold properly, or to reduce or eliminate problem secondary structures within the polynucleotide. Codon optimization tools, algorithms and services are known in the art; non-limiting examples include services from GeneArt (Life Technologies), DNA2.0 (Menlo Park, CA) and/or proprietary methods. In one embodiment, the mRNA sequence is optimized using optimization algorithms, e.g., to optimize expression in mammalian cells or enhance mRNA stability.

In certain embodiments, the present disclosure includes polynucleotides having at least 80%, at least 85%, at least 90%, at least 95%, at least 98%, or at least 99% sequence identity to any of the polynucleotide sequences described herein. mRNAs of the present disclosure may be produced by means available in the art, including but not limited to in vitro transcription (IVT) and synthetic methods. Enzymatic (IVT), solid-phase, liquid-phase, combined synthetic methods, small region synthesis, and ligation methods may be utilized. In one embodiment, mRNAs are made using IVT enzymatic synthesis methods. Methods of making polynucleotides by IVT are known in the art and are described in International Application PCT/US2013/30062, the contents of which are incorporated herein by reference in their entirety. Accordingly, the present disclosure also includes polynucleotides, e.g., DNA, constructs and vectors that may be used to in vitro transcribe an mRNA described herein.

Non-natural modified nucleobases may be introduced into polynucleotides, e.g., mRNA, during synthesis or post-synthesis. In certain embodiments, modifications may be on internucleoside linkages, purine or pyrimidine bases, or sugar. In particular embodiments, the modification may be introduced at the terminal of a polynucleotide chain or anywhere else in the polynucleotide chain; with chemical synthesis or with a polymerase enzyme. Examples of modified nucleic acids and their synthesis are disclosed in PCT application No. PCT/US2012/058519. Synthesis of modified polynucleotides is also described in Verma and Eckstein, Annual Review of Biochemistry, vol. 76, 99-134 (1998).

Either enzymatic or chemical ligation methods may be used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1(3), 165-187 (1990).

### Untranslated Regions (UTRs)

Translation of a polynucleotide comprising an open reading frame encoding a polypeptide can be controlled and regulated by a variety of mechanisms that are provided by various cis-acting nucleic acid structures. For example, naturally-occurring, cis-acting RNA elements that form hairpins or other higher-order (e.g., pseudoknot) intramolecular mRNA secondary structures can provide a translational regulatory activity to a polynucleotide, wherein the RNA element influences or modulates the initiation of polynucleotide translation, particularly when the RNA element is positioned in the 5′ UTR close to the 5′-cap structure (Pelletier and Sonenberg (1985) Cell 40(3):515-526; Kozak (1986) Proc Natl Acad Sci 83:2850-2854).

Untranslated regions (UTRs) are nucleic acid sections of a polynucleotide before a start codon (5′ UTR) and after a stop codon (3′ UTR) that are not translated. In some embodiments, a polynucleotide (e.g., a ribonucleic acid (RNA), e.g., a messenger RNA (mRNA)) of the invention comprising an open reading frame (ORF) encoding a polypeptide further comprises UTR (e.g., a 5′ UTR or functional fragment thereof, a 3′ UTR or functional fragment thereof, or a combination thereof).

Cis-acting RNA elements can also affect translation elongation, being involved in numerous frameshifting events (Namy et al., (2004) Mol Cell 13(2):157-168). Internal ribosome entry sequences (IRES) represent another type of cis-acting RNA element that are typically located in 5′ UTRs, but have also been reported to be found within the coding region of naturally-occurring mRNAs (Holcik et al. (2000) Trends Genet 16(10):469-473). In cellular mRNAs, IRES often coexist with the 5′-cap structure and provide mRNAs with the functional capacity to be translated under conditions in which cap-dependent translation is compromised (Gebauer et al., (2012) Cold Spring Harb Perspect Biol 4(7):a012245). Another type of naturally-occurring cis-acting RNA element comprises upstream open reading frames (uORFs). Naturally-occurring uORFs occur singularly or multiply within the 5′ UTRs of numerous mRNAs and influence the translation of the downstream major ORF, usually negatively (with the notable exception of GCN4 mRNA in yeast and ATF4 mRNA in mammals, where uORFs serve to promote the translation of the downstream major ORF under conditions of increased eIF2 phosphorylation (Hinnebusch (2005) Annu Rev Microbiol 59:407-450)). Additional exemplary translational regulatory activities provided by components, structures, elements, motifs, and/or specific sequences comprising polynucleotides (e.g., mRNA) include, but are not limited to, mRNA stabilization or destabilization (Baker & Parker (2004) Curr Opin Cell Biol 16(3):293-299), translational activation (Villalba et al., (2011) Curr Opin Genet Dev 21(4):452-457), and translational repression (Blumer et al., (2002) Mech Dev 110(1-2):97-112). Studies have shown that naturally-occurring, cis-acting RNA elements can confer their respective functions when used to modify, by incorporation into, heterologous polynucleotides (Goldberg-Cohen et al., (2002) J Biol Chem 277(16):13635-13640).

### Functional RNA Elements

In some embodiments, the disclosure provides polynucleotides comprising a modification (e.g., an RNA element), wherein the modification provides a desired translational regulatory activity. Such modifications are described in PCT Application No. PCT/US2018/033519, herein incorporated by reference in its entirety.

In some embodiments, the disclosure provides a polynucleotide comprising a 5′ untranslated region (UTR), an initiation codon, a full open reading frame encoding a polypeptide, a 3′ UTR, and at least one modification, wherein the at least one modification provides a desired translational regulatory activity, for example, a modification that promotes and/or enhances the translational fidelity of mRNA translation. In some embodiments, the desired translational regulatory activity is a cis-acting regulatory activity. In some embodiments, the desired translational regulatory activity is an increase in the residence time of the 43S pre-initiation complex (PIC) or ribosome at, or proximal to, the initiation codon. In some embodiments, the desired translational regulatory activity is an increase in the initiation of polypeptide synthesis at or from the initiation codon. In some embodiments, the desired translational regulatory activity is an increase in the amount of polypeptide translated from the full open reading frame. In some embodiments, the desired translational regulatory activity is an increase in the fidelity of initiation codon decoding by the PIC or ribosome. In some embodiments, the desired translational regulatory activity is inhibition or reduction of leaky scanning by the PIC or ribosome. In some embodiments, the desired translational regulatory activity is a decrease in the rate of decoding the initiation codon by the PIC or ribosome. In some embodiments, the desired translational regulatory activity is inhibition or reduction in the initiation of polypeptide synthesis at any codon within the mRNA other than the initiation codon. In some embodiments, the desired translational regulatory activity is inhibition or reduction of the amount of polypeptide translated from any open reading frame within the mRNA other than the full open reading frame. In some embodiments, the desired translational regulatory activity is inhibition or reduction in the production of aberrant translation products. In some embodiments, the desired translational regulatory activity is a combination of one or more of the foregoing translational regulatory activities.

Accordingly, the present disclosure provides a polynucleotide, e.g., an mRNA, comprising an RNA element that comprises a sequence and/or an RNA secondary structure(s) that provides a desired translational regulatory activity as described herein. In some aspects, the mRNA comprises an RNA element that comprises a sequence and/or an RNA secondary structure(s) that promotes and/or enhances the translational fidelity of mRNA translation. In some aspects, the mRNA comprises an RNA element that comprises a sequence and/or an RNA secondary structure(s) that provides a desired translational regulatory activity, such as inhibiting and/or reducing leaky scanning. In some aspects, the disclosure provides an mRNA that comprises an RNA element that comprises a sequence and/or an RNA secondary structure(s) that inhibits and/or reduces leaky scanning thereby promoting the translational fidelity of the mRNA.

In some embodiments, the RNA element comprises natural and/or modified nucleotides. In some embodiments, the RNA element comprises of a sequence of linked nucleotides, or derivatives or analogs thereof, that provides a desired translational regulatory activity as described herein. In some embodiments, the RNA element comprises a sequence of linked nucleotides, or derivatives or analogs thereof, that forms or folds into a stable RNA secondary structure, wherein the RNA secondary structure provides a desired translational regulatory activity as described herein. RNA elements can be identified and/or characterized based on the primary sequence of the element (e.g., GC-rich element), by RNA secondary structure formed by the element (e.g. stem-loop), by the location of the element within the RNA molecule (e.g., located within the 5′ UTR of an mRNA), by the biological function and/or activity of the element (e.g., “translational enhancer element”), and any combination thereof.

In some embodiments, the disclosure provides an mRNA having one or more structural modifications that inhibits leaky scanning and/or promotes the translational fidelity of mRNA translation, wherein at least one of the structural modifications is a GC-rich RNA element. In some embodiments, the disclosure provides an mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA. In one embodiment, the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA. In another embodiment, the GC-rich RNA element is located 15-30, 15-20, 15-25, 10-15, or 5-10 nucleotides upstream of a Kozak consensus sequence. In another embodiment, the GC-rich RNA element is located immediately adjacent to a Kozak consensus sequence in the 5′ UTR of the mRNA.

In some embodiments, the disclosure provides a GC-rich RNA element which comprises a sequence of 3-30, 5-25, 10-20, 15-20, about 20, about 15, about 12, about 10, about 7, about 6 or about 3 nucleotides, derivatives or analogs thereof, linked in any order, wherein the sequence composition is 70-80% cytosine, 60-70% cytosine, 50%-60% cytosine, 40-50% cytosine, 30-40% cytosine bases. In some embodiments, the disclosure provides a GC-rich RNA element which comprises a sequence of 3-30, 5-25, 10-20, 15-20, about 20, about 15, about 12, about 10, about 7, about 6 or about 3 nucleotides, derivatives or analogs thereof, linked in any order, wherein the sequence composition is about 80% cytosine, about 70% cytosine, about 60% cytosine, about 50% cytosine, about 40% cytosine, or about 30% cytosine.

In some embodiments, the disclosure provides a GC-rich RNA element which comprises a sequence of 20, 19, 18, 17, 16, 15, 14, 13, 12, 11, 10, 9, 8, 7, 6, 5, 4, or 3 nucleotides, or derivatives or analogs thereof, linked in any order, wherein the sequence composition is 70-80% cytosine, 60-70% cytosine, 50%-60% cytosine, 40-50% cytosine, or 30-40% cytosine. In some embodiments, the disclosure provides a GC-rich RNA element which comprises a sequence of 20, 19, 18, 17, 16, 15, 14, 13, 12, 11, 10, 9, 8, 7, 6, 5, 4, or 3 nucleotides, or derivatives or analogs thereof, linked in any order, wherein the sequence composition is about 80% cytosine, about 70% cytosine, about 60% cytosine, about 50% cytosine, about 40% cytosine, or about 30% cytosine.

In some embodiments, the disclosure provides an mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA, wherein the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA, and wherein the GC-rich RNA element comprises a sequence of 3, 4, 5, 6, 7, 8, 9, 10, 11, 12, 13, 14, 15, 16, 17, 18, 19, or 20 nucleotides, or derivatives or analogs thereof, linked in any order, wherein the sequence composition is >50% cytosine. In some embodiments, the sequence composition is >55% cytosine, >60% cytosine, >65% cytosine, >70% cytosine, >75% cytosine, >80% cytosine, >85% cytosine, or >90% cytosine.

In some embodiments, the disclosure provides an mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA, wherein the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA, and wherein the GC-rich RNA element comprises a sequence of about 3-30, 5-25, 10-20, 15-20 or about 20, about 15, about 12, about 10, about 6 or about 3 nucleotides, or derivatives or analogues thereof, wherein the sequence comprises a repeating GC-motif, wherein the repeating GC-motif is [CCG]n, wherein n=1 to 10 (SEQ ID NO: 103), e.g., n=2 to 8, n=3 to 6, or n=4 to 5. In some embodiments, the sequence comprises a repeating GC-motif [CCG]n (SEQ ID NO: 128), wherein n=1, 2, 3, 4 or 5. In some embodiments, the sequence comprises a repeating GC-motif [CCG]n, wherein n=1, 2, or 3. In some embodiments, the sequence comprises a repeating GC-motif [CCG]n, wherein n=1. In some embodiments, the sequence comprises a repeating GC-motif [CCG]n, wherein n=2. In some embodiments, the sequence comprises a repeating GC-motif [CCG]n, wherein n=3. In some embodiments, the sequence comprises a repeating GC-motif [CCG]n (SEQ ID NO: 129), wherein n=4. In some embodiments, the sequence comprises a repeating GC-motif [CCG]n (SEQ ID NO: 130), wherein n=5.

In some embodiments, the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA. In another embodiment, the GC-rich RNA element is located about 15-30, 15-20, 15-25, 10-15, or 5-10 nucleotides upstream of a Kozak consensus sequence. In another embodiment, the GC-rich RNA element is located immediately adjacent to a Kozak consensus sequence in the 5′ UTR of the mRNA.

In some embodiments, the disclosure provides a modified mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA, wherein the GC-rich RNA element comprises any one of the sequences provided herein. In some embodiments, the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA. In some embodiments, the GC-rich RNA element is located about 15-30, 15-20, 15-25, 10-15, or 5-10 nucleotides upstream of a Kozak consensus sequence. In some embodiments, the GC-rich RNA element is located immediately adjacent to a Kozak consensus sequence in the 5′ UTR of the mRNA.

In some embodiments, the disclosure provides an mRNA comprising at least one modification, wherein at least one modification is a GC-rich RNA element comprising the sequence set forth in SEQ ID NO: 104, or derivatives or analogs thereof, preceding a Kozak consensus sequence in the 5′ UTR of the mRNA. In some embodiments, the GC-rich element comprises the sequence as set forth in SEQ ID NO: 104 located immediately adjacent to and upstream of the Kozak consensus sequence in the 5′ UTR of the mRNA. In some embodiments, the GC-rich element comprises the sequence as set forth in SEQ ID NO: 104 located 1, 2, 3, 4, 5, 6, 7, 8, 9 or 10 bases upstream of the Kozak consensus sequence in the 5′ UTR of the mRNA. In other embodiments, the GC-rich element comprises the sequence as set forth in SEQ ID NO: 104 located 1-3, 3-5, 5-7, 7-9, 9-12, or 12-15 bases upstream of the Kozak consensus sequence in the 5′ UTR of the mRNA.

In some embodiments, the disclosure provides an mRNA comprising at least one modification, wherein at least one modification is a GC-rich RNA element comprising the sequence as set forth SEQ ID NO: 105, or derivatives or analogs thereof, preceding a Kozak consensus sequence in the 5′ UTR of the mRNA. In some embodiments, the GC-rich element comprises the sequence as set forth SEQ ID NO: 105 located immediately adjacent to and upstream of the Kozak consensus sequence in the 5′ UTR of the mRNA. In some embodiments, the GC-rich element comprises the sequence as set forth SEQ ID NO: 105 located 1, 2, 3, 4, 5, 6, 7, 8, 9 or 10 bases upstream of the Kozak consensus sequence in the 5′ UTR of the mRNA. In other embodiments, the GC-rich element comprises the sequence as set forth SEQ ID NO: 105 located 1-3, 3-5, 5-7, 7-9, 9-12, or 12-15 bases upstream of the Kozak consensus sequence in the 5′ UTR of the mRNA.

In some embodiments, the disclosure provides an mRNA comprising at least one modification, wherein at least one modification is a GC-rich RNA element comprising the sequence as set forth in SEQ ID NO: 106, or derivatives or analogs thereof, preceding a Kozak consensus sequence in the 5′ UTR of the mRNA. In some embodiments, the GC-rich element comprises the sequence as set forth in SEQ ID NO: 106 located immediately adjacent to and upstream of the Kozak consensus sequence in the 5′ UTR of the mRNA. In some embodiments, the GC-rich element comprises the sequence as set forth in SEQ ID NO: 106 located 1, 2, 3, 4, 5, 6, 7, 8, 9 or 10 bases upstream of the Kozak consensus sequence in the 5′ UTR of the mRNA. In other embodiments, the GC-rich element comprises the sequence as set forth in SEQ ID NO: 106 located 1-3, 3-5, 5-7, 7-9, 9-12, or 12-15 bases upstream of the Kozak consensus sequence in the 5′ UTR of the mRNA.

In some embodiments, the disclosure provides an mRNA comprising at least one modification, wherein at least one modification is a GC-rich RNA element comprising the sequence set forth in SEQ ID NO: 104, or derivatives or analogs thereof, preceding a Kozak consensus sequence in the 5′ UTR of the mRNA, wherein the 5′ UTR comprises the sequence set forth in SEQ ID NO: 97.

In some embodiments, the GC-rich element comprises the sequence set forth in SEQ ID NO: 104 located immediately adjacent to and upstream of the Kozak consensus sequence in a 5′ UTR sequence described herein. In some embodiments, the GC-rich element comprises the sequence set forth in SEQ ID NO: 104 located 1, 2, 3, 4, 5, 6, 7, 8, 9 or 10 bases upstream of the Kozak consensus sequence in the 5′ UTR of the mRNA, wherein the 5′ UTR comprises the sequence shown in SEQ ID NO: 97.

In other embodiments, the GC-rich element comprises the sequence set forth in SEQ ID NO: 104 located 1-3, 3-5, 5-7, 7-9, 9-12, or 12-15 bases upstream of the Kozak consensus sequence in the 5′ UTR of the mRNA, wherein the 5′ UTR comprises the sequence set forth in SEQ ID NO: 97.

In some embodiments, the 5′ UTR comprises the sequence set forth in SEQ ID NO: 94.

In some embodiments, the 5′ UTR comprises the sequence set forth in SEQ ID NO: 95.

In some embodiments, the 5′ UTR comprises the sequence set forth in SEQ ID NO: 19.

In some embodiments, the disclosure provides an mRNA comprising at least one modification, wherein at least one modification is a GC-rich RNA element comprising a stable RNA secondary structure comprising a sequence of nucleotides, or derivatives or analogs thereof, linked in an order which forms a hairpin or a stem-loop. In one embodiment, the stable RNA secondary structure is upstream of the Kozak consensus sequence. In another embodiment, the stable RNA secondary structure is located about 30, about 25, about 20, about 15, about 10, or about 5 nucleotides upstream of the Kozak consensus sequence. In another embodiment, the stable RNA secondary structure is located about 20, about 15, about 10 or about 5 nucleotides upstream of the Kozak consensus sequence. In another embodiment, the stable RNA secondary structure is located about 5, about 4, about 3, about 2, about 1 nucleotides upstream of the Kozak consensus sequence. In another embodiment, the stable RNA secondary structure is located about 15-30, about 15-20, about 15-25, about 10⁻¹⁵, or about 5-10 nucleotides upstream of the Kozak consensus sequence. In another embodiment, the stable RNA secondary structure is located 12-15 nucleotides upstream of the Kozak consensus sequence. In another embodiment, the stable RNA secondary structure has a deltaG of about −30 kcal/mol, about −20 to −30 kcal/mol, about −20 kcal/mol, about −10 to −20 kcal/mol, about −10 kcal/mol, about −5 to −10 kcal/mol.

In another embodiment, the modification is operably linked to an open reading frame encoding a polypeptide and wherein the modification and the open reading frame are heterologous.

In another embodiment, the sequence of the GC-rich RNA element is comprised exclusively of guanine (G) and cytosine (C) nucleobases.

RNA elements that provide a desired translational regulatory activity as described herein can be identified and characterized using known techniques, such as ribosome profiling.

Ribosome profiling is a technique that allows the determination of the positions of PICs and/or ribosomes bound to mRNAs (see e.g., Ingolia et al., (2009) Science 324(5924):218-23, incorporated herein by reference). The technique is based on protecting a region or segment of mRNA, by the PIC and/or ribosome, from nuclease digestion. Protection results in the generation of a 30-bp fragment of RNA termed a ‘footprint’. The sequence and frequency of RNA footprints can be analyzed by methods known in the art (e.g., RNA-seq). The footprint is roughly centered on the A-site of the ribosome. If the PIC or ribosome dwells at a particular position or location along an mRNA, footprints generated at these positions would be relatively common. Studies have shown that more footprints are generated at positions where the PIC and/or ribosome exhibits decreased processivity and fewer footprints where the PIC and/or ribosome exhibits increased processivity (Gardin et al., (2014) eLife 3:e03735). In some embodiments, residence time or the time of occupancy of the PIC or ribosome at a discrete position or location along a polynucleotide comprising any one or more of the RNA elements described herein is determined by ribosome profiling.

A UTR can be homologous or heterologous to the coding region in a polynucleotide. In some embodiments, the UTR is homologous to the ORF encoding the polypeptide. In some embodiments, the UTR is heterologous to the ORF encoding the polypeptide. In some embodiments, the polynucleotide comprises two or more 5′ UTRs or functional fragments thereof, each of which has the same or different nucleotide sequences. In some embodiments, the polynucleotide comprises two or more 3′ UTRs or functional fragments thereof, each of which has the same or different nucleotide sequences.

In some embodiments, the 5′ UTR or functional fragment thereof, 3′ UTR or functional fragment thereof, or any combination thereof is sequence optimized.

In some embodiments, the 5′UTR or functional fragment thereof, 3′ UTR or functional fragment thereof, or any combination thereof comprises at least one chemically modified nucleobase, e.g., N1 methylpseudouracil or 5-methoxyuracil.

UTRs can have features that provide a regulatory role, e.g., increased or decreased stability, localization and/or translation efficiency. A polynucleotide comprising a UTR can be administered to a cell, tissue, or organism, and one or more regulatory features can be measured using routine methods. In some embodiments, a functional fragment of a 5′ UTR or 3′ UTR comprises one or more regulatory features of a full length 5′ or 3′ UTR, respectively.

Natural 5′UTRs bear features that play roles in translation initiation. They harbor signatures like Kozak sequences that are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′ UTRs also have been known to form secondary structures that are involved in elongation factor binding.

By engineering the features typically found in abundantly expressed genes of specific target organs, one can enhance the stability and protein production of a polynucleotide. For example, introduction of 5′ UTR of liver-expressed mRNA, such as albumin, serum amyloid A, Apolipoprotein A/B/E, transferrin, alpha fetoprotein, erythropoietin, or Factor VIII, can enhance expression of polynucleotides in hepatic cell lines or liver. Likewise, use of 5′UTR from other tissue-specific mRNA to improve expression in that tissue is possible for muscle (e.g., MyoD, Myosin, Myoglobin, Myogenin, Herculin), for endothelial cells (e.g., Tie-1, CD36), for myeloid cells (e.g., C/EBP, AML1, G-CSF, GM-CSF, CD11b, MSR, Fr-1, i-NOS), for leukocytes (e.g., CD45, CD18), for adipose tissue (e.g., CD36, GLUT4, ACRP30, adiponectin) and for lung epithelial cells (e.g., SP-A/B/C/D).

In some embodiments, UTRs are selected from a family of transcripts whose proteins share a common function, structure, feature or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new polynucleotide.

In some embodiments, the 5′ UTR and the 3′ UTR can be heterologous. In some embodiments, the 5′ UTR can be derived from a different species than the 3′ UTR. In some embodiments, the 3′ UTR can be derived from a different species than the 5′ UTR.

Co-owned International Patent Application No. PCT/US2014/021522 (Publ. No. WO/2014/164253, incorporated herein by reference in its entirety) provides a listing of exemplary UTRs that can be utilized in the polynucleotide of the present invention as flanking regions to an ORF.

Exemplary UTRs of the application include, but are not limited to, one or more 5′UTR and/or 3′UTR derived from the nucleic acid sequence of: a globin, such as an α- or P-globin (e.g., a Xenopus, mouse, rabbit, or human globin); a strong Kozak translational initiation signal; a CYBA (e.g., human cytochrome b-245 a polypeptide); an albumin (e.g., human albumin7); a HSD17B4 (hydroxysteroid (17-0) dehydrogenase); a virus (e.g., a tobacco etch virus (TEV), a Venezuelan equine encephalitis virus (VEEV), a Dengue virus, a cytomegalovirus (CMV) (e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., elF4G); a glucose transporter (e.g., hGLUT1 (human glucose transporter 1)); an actin (e.g., human a or R actin); a GAPDH; a tubulin; a histone; a citric acid cycle enzyme; a topoisomerase (e.g., a 5′UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the R subunit of mitochondrial H+-ATP synthase); a growth hormone e (e.g., bovine (bGH) or human (hGH)); an elongation factor (e.g., elongation factor 1 al (EEF1A1)); a manganese superoxide dismutase (MnSOD); a myocyte enhancer factor 2A (MEF2A); a 0-F1-ATPase, a creatine kinase, a myoglobin, a granulocyte-colony stimulating factor (G-CSF); a collagen (e.g., collagen type I, alpha 2 (CollA2), collagen type I, alpha 1 (CollA1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nntl); calreticulin (Calr); a procollagen-lysine, 2-oxoglutarate 5-dioxygenase 1 (Plodl); and a nucleobindin (e.g., Nucbl).

In some embodiments, the 5′ UTR is selected from the group consisting of a 0-,globin 5′ UTR; a 5′UTR containing a strong Kozak translational initiation signal; a cytochrome b-245 a polypeptide (CYBA) 5′ UTR; a hydroxysteroid (17-0) dehydrogenase (HSD17B4) 5′ UTR; a Tobacco etch virus (TEV) 5′ UTR; a Venezuelan equine encephalitis virus (TEEV) 5′ UTR; a 5′ proximal open reading frame of rubella virus (RV) RNA encoding nonstructural proteins; a Dengue virus (DEN) 5′ UTR; a heat shock protein 70 (Hsp70) 5′ UTR; a eIF4G 5′ UTR; a GLUT1 5′ UTR; functional fragments thereof and any combination thereof.

In some embodiments, the 3′ UTR is selected from the group consisting of a 0-,globin 3′ UTR; a CYBA 3′ UTR; an albumin 3′ UTR; a growth hormone (GH) 3′ UTR; a VEEV 3′ UTR; a hepatitis B virus (HBV) 3′ UTR; α-globin 3′UTR; a DEN 3′ UTR; a PAV barley yellow dwarf virus (BYDV-PAV) 3′ UTR; an elongation factor 1 al (EEF1A1) 3′ UTR; a manganese superoxide dismutase (MnSOD) 3′ UTR; a R subunit of mitochondrial H(+)-ATP synthase (0-mRNA) 3′ UTR; a GLUT1 3′ UTR; a MEF2A 3′ UTR; a 0-F1-ATPase 3′ UTR; functional fragments thereof and combinations thereof.

Wild-type UTRs derived from any gene or mRNA can be incorporated into the polynucleotides of the invention. In some embodiments, a UTR can be altered relative to a wild type or native UTR to produce a variant UTR, e.g., by changing the orientation or location of the UTR relative to the ORF; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR.

Additionally, one or more synthetic UTRs can be used in combination with one or more non-synthetic UTRs. See, e.g., Mandal and Rossi, Nat. Protoc. 2013 8(3):568-82, the contents of which are incorporated herein by reference in their entirety.

UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs.

In some embodiments, the polynucleotide comprises multiple UTRs, e.g., a double, a triple or a quadruple 5′ UTR or 3′ UTR. For example, a double UTR comprises two copies of the same UTR either in series or substantially in series. For example, a double beta-globin 3′UTR can be used (see US2010/0129877, the contents of which are incorporated herein by reference in its entirety).

In certain embodiments, the disclosure provides an mRNA comprising a 5′ UTR and/or a 3′ UTR selected from any of the UTRs disclosed herein. In some embodiments, the 5′ UTR comprises:

5′ UTR-023 (Upstream UTR),
(SEQ ID NO: 107)
(GGGAAAUAAGAGAGAAAAGAAGAGUAAGAAGAAAUAUAAGAGCCACC);
5′ UTR-002 (Upstream UTR)
(SEQ ID NO: 81
(GGGAGAUCAGAGAGAAAAGAAGAGUAAGAAGAAAUAUAAGAGCCACC);
5′ UTR-003 (Upstream UTR) (See WO2016/100812);
5′ UTR-004 (Upstream UTR)
(SEQ ID NO: 82)
(GGGAGACAAGCUUGGCAUUCCGGUACUGUUGGUAAAGCCACC);
5′ UTR-006 (Upstream UTR) (See WO2016/100812);
5′ UTR-008 (Upstream UTR)
(SEQ ID NO: 83)
(GGGAAUUAACAGAGAAAAGAAGAGUAAGAAGAAAUAUAAGAGCCACC);
5′ UTR-009 (Upstream UTR)
(SEQ ID NO: 84)
(GGGAAAUUAGACAGAAAAGAAGAGUAAGAAGAAAUAUAAGAGCCACC);
5′ UTR-010, Upstream
(SEQ ID NO: 85)
(GGGAAAUAAGAGAGUAAAGAACAGUAAGAAGAAAUAUAAGAGCCACC);
5′ UTR-011 (Upstream UTR)
(SEQ ID NO: 86)
(GGGAAAAAAGAGAGAAAAGAAGACUAAGAAGAAAUAUAAGAGCCACC);
5′ UTR-012 (Upstream UTR)
(SEQ ID NO: 87)
(GGGAAAUAAGAGAGAAAAGAAGAGUAAGAAGAUAUAUAAGAGCCACC);
5′ UTR-013 (Upstream UTR)
(SEQ ID NO: 88)
(GGGAAAUAAGAGACAAAACAAGAGUAAGAAGAAAUAUAAGAGCCACC);
5′ UTR-014 (Upstream UTR)
(SEQ ID NO: 89)
(GGGAAAUUAGAGAGUAAAGAACAGUAAGUAGAAUUAAAAGAGCCACC);
5′ UTR-015 (Upstream UTR)
(SEQ ID NO: 90)
(GGGAAAUAAGAGAGAAUAGAAGAGUAAGAAGAAAUAUAAGAGCCACC);
5′ UTR-016 (Upstream UTR)
(SEQ ID NO: 91)
(GGGAAAUAAGAGAGAAAAGAAGAGUAAGAAGAAAAUUAAGAGCCACC);
5′ UTR-017 (Upstream UTR);
(SEQ ID NO: 92)
(GGGAAAUAAGAGAGAAAAGAAGAGUAAGAAGAAAUUUAAGAGCCACC);
or
5′ UTR-024 (Upstream UTR) 5′ UTR
(SEQ ID NO: 108)
(UCAAGCUUUUGGACCCUCGUACAGAAGCUAAUACGACUCACUAUAGGGA
AAUAAGAGAGAAAAGAAGAGUAAGAAGAAAUAUAAGAGCCACC).

In some embodiments, an mRNA of the disclosure comprises (i) a 5′ UTR comprising a nucleotide sequence having at least about 60%, at least about 70%, at least about 80%, at least about 90%, at least about 95%, at least about 96%, at least about 97%, at least about 98%, at least about 99%, or about 100% identity to a nucleotide sequence selected from SEQ ID NOs: 19, 80-97, and 107-108; and/or (ii) a Y UTR sequences comprising a nucleotide sequence having at least about 60%, at least about 70%, at least about 80%, at least about 90%, at least about 95%, at least about 96%, at least about 97%, at least about 98%, at least about 99%, or about 100% identity to the nucleotide sequence of SEQ ID NO: 20. In some embodiments, an mRNA of the disclosure comprises (i) a 5′ UTR comprising a nucleotide sequence having at least about 90%, at least about 95%, at least about 96%, at least about 97%, at least about 98%, at least about 99%, or about 100% identity to a nucleotide sequence selected from SEQ ID NOs: 19, 80-97, and 107-108; and/or (ii) a Y UTR sequences comprising a nucleotide sequence having at least about 90%, at least about 95%, at least about 96%, at least about 97%, at least about 98%, at least about 99%, or about 100% identity to the nucleotide sequence of SEQ ID NO: 20.

In certain embodiments, an mRNA of the disclosure comprises (i) a 5′ UTR comprising a nucleotide sequence having at least about 60%, at least about 70%, at least about 80%, at least about 90%, at least about 95%, at least about 96%, at least about 97%, at least about 98%, at least about 99%, or about 100% identity to a nucleotide sequence selected from SEQ ID NOs: 19, 80-97, or 107-108; and (i) a 3′ UTR sequences comprising a nucleotide sequence having at least about 60%, at least about 70%, at least about 80%, at least about 90%, at least about 95%, at least about 96%, at least about 97%, at least about 98%, at least about 99%, or about 100% identity to the nucleotide sequence of SEQ ID NO: 20. In certain embodiments, an mRNA of the disclosure comprises (i) a 5′ UTR comprising a nucleotide sequence having at least about 90%, at least about 95%, at least about 96%, at least about 97%, at least about 98%, at least about 99%, or about 100% identity to a nucleotide sequence selected from SEQ ID NOs: 19, 80-97, or 107-108; and (i) a 3′ UTR sequences comprising a nucleotide sequence having at least about 90%, at least about 95%, at least about 96%, at least about 97%, at least about 98%, at least about 99%, or about 100% identity to the nucleotide sequence of SEQ ID NO: 20.

In some embodiments, the 5′ UTR comprises a nucleotide sequence set forth in SEQ ID NO: 19, 80-97, or 107-108. In some embodiments, the 3′ UTR comprises a nucleotide sequence set forth in SEQ ID NO: 20. In some embodiments, the 5′ UTR comprises a nucleotide sequence set forth in SEQ ID NO: 19, 80-97, or 107-108 and the 3′ UTR comprises nucleotide sequence set forth in SEQ ID NO: 20.

In some embodiments, an mRNA of the disclosure comprises a combinations of features. For example, the ORF can be flanked by a 5′UTR that comprises a strong Kozak translational initiation signal and/or a 3′UTR comprising an oligo(dT) sequence for templated addition of a poly-A tail. A 5′UTR can comprise a first polynucleotide fragment and a second polynucleotide fragment from the same and/or different UTRs (see, e.g., US2010/0293625, herein incorporated by reference in its entirety).

Other non-UTR sequences can be used as regions or subregions within an mRNA of the disclosure. For example, introns or portions of intron sequences can be incorporated into an mRNA of the disclosure. Incorporation of intronic sequences can increase protein production as well as mRNA expression levels. In some embodiments, an mRNA of the disclosure comprises an internal ribosome entry site (IRES) instead of or in addition to a UTR (see, e.g., Yakubov et al., Biochem. Biophys. Res. Commun. 2010 394(1):189-193, the contents of which are incorporated herein by reference in their entirety). In some embodiments, an mRNA comprises an IRES instead of a 5′ UTR sequence. In some embodiments, the mRNA comprises an ORF and a viral capsid sequence. In some embodiments, the mRNA comprises a synthetic 5′ UTR in combination with a non-synthetic 3′ UTR.

In some embodiments, the UTR can also include at least one translation enhancer polynucleotide, translation enhancer element, or translational enhancer elements (collectively, “TEE,” which refers to nucleic acid sequences that increase the amount of polypeptide or protein produced from a polynucleotide. As a non-limiting example, the TEE can be located between the transcription promoter and the start codon. In some embodiments, the 5′ UTR comprises a TEE. In one aspect, a TEE is a conserved element in a UTR that can promote translational activity of a nucleic acid such as, but not limited to, cap-dependent or cap-independent translation.

MicroRNA (miRNA) Binding Sites

In some embodiments, an mRNA of the disclosure comprises one or more regulatory elements, for example, microRNA (miRNA) binding sites, transcription factor binding sites, structured mRNA sequences and/or motifs, artificial binding sites engineered to act as pseudo-receptors for endogenous nucleic acid binding molecules, and combinations thereof. In some embodiments, mRNAs including such regulatory elements are referred to as including “sensor sequences.” Non-limiting examples of sensor sequences are described in U.S. Publication 2014/0200261, the contents of which are incorporated herein by reference in their entirety.

In some embodiments, an mRNA of the disclosure comprises an open reading frame (ORF) encoding a polypeptide of interest and further comprises one or more miRNA binding site(s). Inclusion or incorporation of miRNA binding site(s) provides for regulation of polynucleotides of the disclosure, and in turn, of the polypeptides encoded therefrom, based on tissue-specific and/or cell-type specific expression of naturally-occurring miRNAs.

A miRNA, e.g., a natural-occurring miRNA, is a 19-25 nucleotide long noncoding RNA that binds to an mRNA and down-regulates gene expression either by reducing stability or by inhibiting translation of the polynucleotide. A miRNA sequence comprises a “seed” region, i.e., a sequence in the region of positions 2-8 of the mature miRNA. A miRNA seed can comprise positions 2-8 or 2-7 of the mature miRNA. In some embodiments, a miRNA seed can comprise 7 nucleotides (e.g., nucleotides 2-8 of the mature miRNA), wherein the seed-complementary site in the corresponding miRNA binding site is flanked by an adenosine (A) opposed to miRNA position 1. In some embodiments, a miRNA seed can comprise 6 nucleotides (e.g., nucleotides 2-7 of the mature miRNA), wherein the seed-complementary site in the corresponding miRNA binding site is flanked by an adenosine (A) opposed to miRNA position 1. See, for example, Grimson A, Farh K K, Johnston W K, Garrett-Engele P, Lim LP, Bartel DP; Mol Cell. 2007 Jul. 6; 27(1):91-105. miRNA profiling of the target cells or tissues can be conducted to determine the presence or absence of miRNA in the cells or tissues. In some embodiments, an mRNA of the disclosure comprises one or more microRNA binding sites, microRNA target sequences, microRNA complementary sequences, or microRNA seed complementary sequences. Such sequences can correspond to, e.g., have complementarity to, any known microRNA such as those taught in US Publication US2005/0261218 and US Publication US2005/0059005, the contents of each of which are incorporated herein by reference in their entirety.

As used herein, the term “microRNA (miRNA or miR) binding site” refers to a sequence within an mRNA including in the 5′UTR and/or 3′UTR, that has sufficient complementarity to all or a region of a miRNA to interact with, associate with or bind to the miRNA. In some embodiments, an mRNA of the disclosure comprising an ORF encoding a polypeptide of interest and further comprises one or more miRNA binding site(s). In exemplary embodiments, a 5′UTR and/or 3′UTR of the mRNA comprises the one or more miRNA binding site(s).

A miRNA binding site having sufficient complementarity to a miRNA refers to a degree of complementarity sufficient to facilitate miRNA-mediated regulation of an mRNA, e.g., miRNA-mediated translational repression or degradation of the mRNA. In exemplary aspects of the disclosure, a miRNA binding site having sufficient complementarity to the miRNA refers to a degree of complementarity sufficient to facilitate miRNA-mediated degradation of the mRNA, e.g., miRNA-guided RNA-induced silencing complex (RISC)-mediated cleavage of mRNA. The miRNA binding site can have complementarity to, for example, a 19-25 nucleotide miRNA sequence, to a 19-23 nucleotide miRNA sequence, or to a 22 nucleotide miRNA sequence. A miRNA binding site can be complementary to only a portion of a miRNA, e.g., to a portion less than 1, 2, 3, or 4 nucleotides of the full length of a naturally-occurring miRNA sequence. Full or complete complementarity (e.g., full complementarity or complete complementarity over all or a significant portion of the length of a naturally-occurring miRNA) is preferred when the desired regulation is mRNA degradation.

In some embodiments, a miRNA binding site includes a sequence that has complementarity (e.g., partial or complete complementarity) with a miRNA seed sequence. In some embodiments, the miRNA binding site includes a sequence that has complete complementarity with a miRNA seed sequence. In some embodiments, a miRNA binding site includes a sequence that has complementarity (e.g., partial or complete complementarity) with a miRNA sequence. In some embodiments, the miRNA binding site includes a sequence that has complete complementarity with a miRNA sequence. In some embodiments, a miRNA binding site has complete complementarity with a miRNA sequence but for 1, 2, or 3 nucleotide substitutions, terminal additions, and/or truncations.

In some embodiments, the miRNA binding site is the same length as the corresponding miRNA. In other embodiments, the miRNA binding site is one, two, three, four, five, six, seven, eight, nine, ten, eleven or twelve nucleotide(s) shorter than the corresponding miRNA at the 5′ terminus, the 3′ terminus, or both. In still other embodiments, the microRNA binding site is two nucleotides shorter than the corresponding microRNA at the 5′ terminus, the 3′ terminus, or both.

The miRNA binding sites that are shorter than the corresponding miRNAs are still capable of degrading the mRNA incorporating one or more of the miRNA binding sites or preventing the mRNA from translation.

In some embodiments, the miRNA binding site binds the corresponding mature miRNA that is part of an active RISC containing Dicer. In another embodiment, binding of the miRNA binding site to the corresponding miRNA in RISC degrades the mRNA containing the miRNA binding site or prevents the mRNA from being translated. In some embodiments, the miRNA binding site has sufficient complementarity to miRNA so that a RISC complex comprising the miRNA cleaves the polynucleotide comprising the miRNA binding site. In other embodiments, the miRNA binding site has imperfect complementarity so that a RISC complex comprising the miRNA induces instability in the polynucleotide comprising the miRNA binding site. In another embodiment, the miRNA binding site has imperfect complementarity so that a RISC complex comprising the miRNA represses transcription of the polynucleotide comprising the miRNA binding site.

In some embodiments, the miRNA binding site has one, two, three, four, five, six, seven, eight, nine, ten, eleven or twelve mismatch(es) from the corresponding miRNA.

In some embodiments, the miRNA binding site has at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one contiguous nucleotides complementary to at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one, respectively, contiguous nucleotides of the corresponding miRNA.

By engineering one or more miRNA binding sites into an mRNA of the disclosure, the mRNA can be targeted for degradation or reduced translation, provided the miRNA in question is available. This can reduce off-target effects upon delivery of the mRNA. For example, if an mRNA of the disclosure is not intended to be delivered to a tissue or cell but ends up is said tissue or cell, then a miRNA abundant in the tissue or cell can inhibit the expression of the gene of interest if one or multiple binding sites of the miRNA are engineered into the 5′UTR and/or 3′UTR of the mRNA.

Conversely, miRNA binding sites can be removed from mRNA sequences in which they naturally occur in order to increase protein expression in specific tissues. For example, a binding site for a specific miRNA can be removed from an mRNA to improve protein expression in tissues or cells containing the miRNA.

In one embodiment, an mRNA of the disclosure can include at least one miRNA-binding site in the 5′UTR and/or 3′UTR in order to regulate cytotoxic or cytoprotective mRNA therapeutics to specific cells such as, but not limited to, normal and/or cancerous cells. In another embodiment, a polynucleotide of the disclosure can include two, three, four, five, six, seven, eight, nine, ten, or more miRNA-binding sites in the 5′-UTR and/or 3′-UTR in order to regulate cytotoxic or cytoprotective mRNA therapeutics to specific cells such as, but not limited to, normal and/or cancerous cells.

Regulation of expression in multiple tissues can be accomplished through introduction or removal of one or more miRNA binding sites, e.g., one or more distinct miRNA binding sites. The decision whether to remove or insert a miRNA binding site can be made based on miRNA expression patterns and/or their profilings in tissues and/or cells in development and/or disease. Identification of miRNAs, miRNA binding sites, and their expression patterns and role in biology have been reported (e.g., Bonauer et al., Curr Drug Targets 2010 11:943-949; Anand and Cheresh Curr Opin Hematol 2011 18:171-176; Contreras and Rao Leukemia 2012 26:404-413 (2011 Dec 20. doi: 10.1038/leu.2011.356); Bartel Cell 2009 136:215-233; Landgraf et al, Cell, 2007 129:1401-1414; Gentner and Naldini, Tissue Antigens. 2012 80:393-403 and all references therein; each of which is incorporated herein by reference in its entirety).

miRNAs and miRNA binding sites can correspond to any known sequence, including non-limiting examples described in U.S. Publication Nos. 2014/0200261, 2005/0261218, and 2005/0059005, each of which are incorporated herein by reference in their entirety.

Examples of tissues where miRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-ld, miR-149), kidney (miR-192, miR-194, miR-204), and lung epithelial cells (let-7, miR-133, miR-126).

Specifically, miRNAs are known to be differentially expressed in immune cells (also called hematopoietic cells), such as antigen presenting cells (APCs) (e.g., dendritic cells and macrophages), macrophages, monocytes, B lymphocytes, T lymphocytes, granulocytes, natural killer cells, etc. Immune cell specific miRNAs are involved in immunogenicity, autoimmunity, the immune response to infection, inflammation, as well as unwanted immune response after gene therapy and tissue/organ transplantation. Immune cell specific miRNAs also regulate many aspects of development, proliferation, differentiation and apoptosis of hematopoietic cells (immune cells). For example, miR-142 and miR-146 are exclusively expressed in immune cells, particularly abundant in myeloid dendritic cells. It has been demonstrated that the immune response to a polynucleotide can be shut-off by adding miR-142 binding sites to the 3′-UTR of the polynucleotide, enabling more stable gene transfer in tissues and cells. miR-142 efficiently degrades exogenous polynucleotides in antigen presenting cells and suppresses cytotoxic elimination of transduced cells (e.g., Annoni A et al., blood, 2009, 114, 5152-5161; Brown B D, et al., Nat med. 2006, 12(5), 585-591; Brown B D, et al., blood, 2007, 110(13): 4144-4152, each of which is incorporated herein by reference in its entirety).

An antigen-mediated immune response can refer to an immune response triggered by foreign antigens, which, when entering an organism, are processed by the antigen presenting cells and displayed on the surface of the antigen presenting cells. T cells can recognize the presented antigen and induce a cytotoxic elimination of cells that express the antigen.

Introducing a miR-142 binding site into the 5′UTR and/or 3′UTR of an mRNA of the disclosure can selectively repress gene expression in antigen presenting cells through miR-142 mediated degradation, limiting antigen presentation in antigen presenting cells (e.g., dendritic cells) and thereby preventing antigen-mediated immune response after the delivery of the mRNA. The mRNA is then stably expressed in target tissues or cells without triggering cytotoxic elimination.

In one embodiment, binding sites for miRNAs that are known to be expressed in immune cells, in particular, antigen presenting cells, can be engineered into an mRNA of the disclosure to suppress the expression of the polynucleotide in antigen presenting cells through miRNA mediated RNA degradation, subduing the antigen-mediated immune response. Expression of the mRNA is maintained in non-immune cells where the immune cell specific miRNAs are not expressed. For example, in some embodiments, to prevent an immunogenic reaction against a liver specific protein, any miR-122 binding site can be removed and a miR-142 (and/or mirR-146) binding site can be engineered into the 5′UTR and/or 3′UTR of an mRNA of the disclosure.

To further drive the selective degradation and suppression in APCs and macrophage, an mRNA of the disclosure can include a further negative regulatory element in the 5′UTR and/or 3′UTR, either alone or in combination with miR-142 and/or miR-146 binding sites. As a non-limiting example, the further negative regulatory element is a Constitutive Decay Element (CDE).

Immune cell specific miRNAs include, but are not limited to, hsa-let-7α-2-3p, hsa-let-7α-3p, hsa-7α-5p, hsa-let-7c, hsa-let-7e-3p, hsa-let-7e-5p, hsa-let-7g-3p, hsa-let-7g-5p, hsa-let-7i-3p, hsa-let-7i-5p, miR-10α-3p, miR-lOa-5p, miR-1184, hsa-let-7f-1--3p, hsa-let-7f-2--5p, hsa-let-7f-5p, miR-125b-1-3p, miR-125b-2-3p, miR-125b-5p, miR-1279, miR-130α-3p, miR-130α-5p, miR-132-3p, miR-132-5p, miR-142-3p, miR-142-5p, miR-143-3p, miR-143-5p, miR-146α-3p, miR-146α-5p, miR-146b-3p, miR-146b-5p, miR-147a, miR-147b, miR-148α-5p, miR-148α-3p, miR-150-3p, miR-150-5p, miR-151b, miR-155-3p, miR-155-5p, miR-15α-3p, miR-15α-5p, miR-15b-5p, miR-15b-3p, miR-16-1-3p, miR-16-2-3p, miR-16-5p, miR-17-5p, miR-181α-3p, miR-181α-5p, miR-181α-2-3p, miR-182-3p, miR-182-5p, miR-197-3p, miR-197-5p, miR-21-5p, miR-21-3p, miR-214-3p, miR-214-5p, miR-223-3p, miR-223-5p, miR-221-3p, miR-221-5p, miR-23b-3p, miR-23b-5p, miR-24-1-5p,miR-24-2-5p, miR-24-3p, miR-26α-1-3p, miR-26α-2-3p, miR-26α-5p, miR-26b-3p, miR-26b-5p, miR-27α-3p, miR-27α-5p, miR-27b-3p,miR-27b-5p, miR-28-3p, miR-28-5p, miR-2909, miR-29α-3p, miR-29α-5p, miR-29b-1-5p, miR-29b-2-5p, miR-29c-3p, miR-29c-5p,, miR-30e-3p, miR-30e-5p, miR-331-5p, miR-339-3p, miR-339-5p, miR-345-3p, miR-345-5p, miR-346, miR-34α-3p, miR-34α-5p, miR-363-3p, miR-363-5p, miR-372, miR-377-3p, miR-377-5p, miR-493-3p, miR-493-5p, miR-542, miR-548b-5p, miR548c-5p, miR-548i, miR-548j, miR-548n, miR-574-3p, miR-598, miR-718, miR-935, miR-99α-3p, miR-99α-5p, miR-99b-3p, and miR-99b-5p. Furthermore, novel miRNAs can be identified in immune cell through micro-array hybridization and microtome analysis (e.g., Jima D D et al, Blood, 2010, 116:e118-e127; Vaz C et al., BMC Genomics, 2010, 11,288, the content of each of which is incorporated herein by reference in its entirety.) In some embodiments, an mRNA of the disclosure comprises a miRNA binding site, wherein the miRNA binding site comprises one or more nucleotide sequences selected from SEQ ID Nos: 113 or 115, including one or more copies of any one or more of the miRNA binding site sequences. In some embodiments, an mRNA of the disclosure further comprises at least one, two, three, four, five, six, seven, eight, nine, ten, or more of the same or different miRNA binding sites selected from SEQ ID NOs: 113 or 115, including any combination thereof.

Some embodiments, an mRNA of the disclosure comprises at least one miR-122 binding site, at least two miR-122 binding sites, at least three miR-122 binding sites, at least four miR-122 binding sites, or at least five miR-122 binding sites. In one aspect, the miRNA binding site binds miR-122 or is complementary to miR-122. In another aspect, the miRNA binding site binds to miR-122-3p or miR-122-5p. In a particular aspect, the miRNA binding site comprises a nucleotide sequence at least 80%, at least 85%, at least 90%, at least 95%, or 100% identical to SEQ ID NO: 113, wherein the miRNA binding site binds to miR-122. In another particular aspect, the miRNA binding site comprises a nucleotide sequence at least 80%, at least 85%, at least 90%, at least 95%, or 100% identical to SEQ ID NO: 113, wherein the miRNA binding site binds to miR-122.

In some embodiments, a miRNA binding site is inserted in the mRNA of the disclosure in any position of the polynucleotide (e.g., the 5′UTR and/or 3′UTR). In some embodiments, the 5′UTR comprises a miRNA binding site. In some embodiments, the 3′UTR comprises a miRNA binding site. In some embodiments, the 5′UTR and the 3′UTR comprise a miRNA binding site. The insertion site in the mRNA can be anywhere in the mRNA as long as the insertion of the miRNA binding site in the mRNA does not interfere with the translation of a functional polypeptide in the absence of the corresponding miRNA; and in the presence of the miRNA, the insertion of the miRNA binding site in the mRNA and the binding of the miRNA binding site to the corresponding miRNA are capable of degrading the mRNA or preventing the translation of the mRNA.

In some embodiments, a miRNA binding site is inserted in at least about 30 nucleotides downstream from the stop codon of an ORF in an mRNA of the disclosure comprising the ORF. In some embodiments, a miRNA binding site is inserted in at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, or at least about 100 nucleotides downstream from the stop codon of an ORF in an mRNA of the disclosure. In some embodiments, a miRNA binding site is inserted in about 10 nucleotides to about 100 nucleotides, about 20 nucleotides to about 90 nucleotides, about 30 nucleotides to about 80 nucleotides, about 40 nucleotides to about 70 nucleotides, about 50 nucleotides to about 60 nucleotides, about 45 nucleotides to about 65 nucleotides downstream from the stop codon of an ORF in an mRNA of the disclosure.

miRNA gene regulation can be influenced by the sequence surrounding the miRNA such as, but not limited to, the species of the surrounding sequence, the type of sequence (e.g., heterologous, homologous, exogenous, endogenous, or artificial), regulatory elements in the surrounding sequence and/or structural elements in the surrounding sequence. The miRNA can be influenced by the 5′UTR and/or 3′UTR. As a non-limiting example, a non-human 3′UTR can increase the regulatory effect of the miRNA sequence on the expression of a polypeptide of interest compared to a human 3′UTR of the same sequence type.

In one embodiment, other regulatory elements and/or structural elements of the 5′UTR can influence miRNA mediated gene regulation. One example of a regulatory element and/or structural element is a structured IRES (Internal Ribosome Entry Site) in the 5′UTR, which is necessary for the binding of translational elongation factors to initiate protein translation. EIF4A2 binding to this secondarily structured element in the 5′-UTR is necessary for miRNA mediated gene expression (Meijer H A et al., Science, 2013, 340, 82-85, herein incorporated by reference in its entirety). The mRNAs of the disclosure can further include this structured 5′UTR in order to enhance microRNA mediated gene regulation.

At least one miRNA binding site can be engineered into the 3′UTR of an mRNA of the disclosure. In this context, at least two, at least three, at least four, at least five, at least six, at least seven, at least eight, at least nine, at least ten, or more miRNA binding sites can be engineered into a 3′UTR of an mRNA of the disclosure. For example, 1 to 10, 1 to 9, 1 to 8, 1 to 7, 1 to 6, 1 to 5, 1 to 4, 1 to 3, 2, or 1 miRNA binding sites can be engineered into the 3′UTR of an mRNA of the disclosure. In one embodiment, miRNA binding sites incorporated into an mRNA of the disclosure can be the same or can be different miRNA sites. A combination of different miRNA binding sites incorporated into an mRNA of the disclosure can include combinations in which more than one copy of any of the different miRNA sites are incorporated. In another embodiment, miRNA binding sites incorporated into an mRNA of the disclosure can target the same or different tissues in the body. As a non-limiting example, through the introduction of tissue-, cell-type-, or disease-specific miRNA binding sites in the 3′-UTR of an mRNA of the disclosure, the degree of expression in specific cell types (e.g., hepatocytes, myeloid cells, endothelial cells, cancer cells, etc.) can be reduced.

In one embodiment, a miRNA binding site can be engineered near the 5′ terminus of the 3′UTR, about halfway between the 5′ terminus and 3′ terminus of the 3′UTR and/or near the 3′ terminus of the 3′UTR in an mRNA of the disclosure. As a non-limiting example, a miRNA binding site can be engineered near the 5′ terminus of the 3′UTR and about halfway between the 5′ terminus and 3′ terminus of the 3′UTR. As another non-limiting example, a miRNA binding site can be engineered near the 3′ terminus of the 3′UTR and about halfway between the 5′ terminus and 3′ terminus of the 3′UTR. As yet another non-limiting example, a miRNA binding site can be engineered near the 5′ terminus of the 3′UTR and near the 3′ terminus of the 3′UTR.

In another embodiment, a 3′UTR can comprise 1, 2, 3, 4, 5, 6, 7, 8, 9, or 10 miRNA binding sites. The miRNA binding sites can be complementary to a miRNA, miRNA seed sequence, and/or miRNA sequences flanking the seed sequence.

An mRNA of the disclosure can be engineered for more targeted expression in specific tissues, cell types, or biological conditions based on the expression patterns of miRNAs in the different tissues, cell types, or biological conditions. Through introduction of tissue-specific miRNA binding sites, an mRNA of the disclosure can be designed for optimal protein expression in a tissue or cell, or in the context of a biological condition.

In some embodiments, an mRNA of the disclosure can include at least one miRNA in order to dampen expression of the encoded polypeptide in a tissue or cell of interest. As a non-limiting example, an mRNA of the disclosure can include at least one miR-122 binding site in order to dampen expression of an encoded polypeptide of interest in the liver. As another non-limiting example an mRNA of the disclosure can include at least one miR-142-3p binding site, miR-142-3p seed sequence, miR-142-3p binding site without the seed, miR-142-5p binding site, miR-142-5p seed sequence, miR-142-5p binding site without the seed, miR-146 binding site, miR-146 seed sequence and/or miR-146 binding site without the seed sequence.

In some embodiments, an mRNA of the disclosure can comprise at least one miRNA binding site in the 3′UTR in order to selectively degrade mRNA therapeutics in the immune cells to subdue unwanted immunogenic reactions caused by therapeutic delivery. As a non-limiting example, the miRNA binding site can make an mRNA of the disclosure more unstable in antigen presenting cells. Non-limiting examples of these miRNAs include mir-142-5p, mir-142-3p, mir-146α-5p, and mir-146-3p.

In one embodiment, an mRNA of the disclosure comprises at least one miRNA sequence in a region of the mRNA that can interact with an RNA binding protein.

In some embodiments, the mRNA of the disclosure (e.g., a RNA, e.g., an mRNA) comprising (i) a sequence-optimized nucleotide sequence (e.g., an ORF) and (ii) a miRNA binding site (e.g., a miRNA binding site that binds to miR-142).

In some embodiments, the mRNA of the disclosure comprises a uracil-modified sequence encoding a polypeptide disclosed herein and a miRNA binding site disclosed herein, e.g., a miRNA binding site that binds to miR-142. In some embodiments, the uracil-modified sequence encoding a polypeptide comprises at least one chemically modified nucleobase, e.g., 5-methoxyuracil. In some embodiments, at least 95% of a type of nucleobase (e.g., uracil) in a uracil-modified sequence encoding a polypeptide of the disclosure are modified nucleobases. In some embodiments, at least 95% of uracil in a uracil-modified sequence encoding a polypeptide is 5-methoxyuridine. In some embodiments, the mRNA comprising a nucleotide sequence encoding a polypeptide disclosed herein and a miRNA binding site is formulated with a delivery agent, e.g., a compound having the Formula (I), e.g., Compound 1.

### Formulations

### Lipid Compositions

The present disclosure provides pharmaceutical compositions with advantageous properties. The lipid compositions described herein may be advantageously used in lipid nanoparticle compositions for the delivery of therapeutic and/or prophylactic agents, e.g., mRNAs, to mammalian cells or organs. For example, the lipids described herein have little or no immunogenicity. For example, the lipid compounds disclosed herein have a lower immunogenicity as compared to a reference lipid (e.g., MC3, KC2, or DLinDMA). For example, a formulation comprising a lipid disclosed herein and a therapeutic or prophylactic agent, e.g., mRNA, has an increased therapeutic index as compared to a corresponding formulation which comprises a reference lipid (e.g., MC3, KC2, or DLinDMA) and the same therapeutic or prophylactic agent.

In some embodiments, the present disclosure provides pharmaceutical compositions comprising:
(a) an mRNA comprising a nucleotide sequence encoding an IL15 fusion protein described herein; and
(b) a delivery agent.

### Lipid Nanoparticle Compositions

In some embodiments, nucleic acids of the disclosure (e.g., mRNA encoding IL15 fusion protein described herein) are formulated as lipid nanoparticle (LNP) compositions. Lipid nanoparticles typically comprise amino lipid, phospholipid, structural lipid and PEG lipid components along with the nucleic acid cargo of interest. The lipid nanoparticles of the invention can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/052117; PCT/US2012/069610; PCT/US2017/027492; PCT/US2016/059575; PCT/US2016/069491; PCT/US2016/069493; and PCT/US2014/066242, all of which are incorporated by reference herein in their entirety.

In some embodiments, the lipid nanoparticle comprises components described in U.S. Pat. No. 9,868,692B2; U.S. Pat. No. 10,195,156B2; U.S. Pat. No. 10,022,435B2; US2020/0069599A1; US2018/0243230A1; U.S. Pat. No. 10,556,018B2; US2018/0000953A1; US2020/0315967A1; US2019/0142971A1; U.S. Pat. No. 9,925,277B2; US2019/0054112A1; U.S. Pat. No. 8,680,069B2; US2019/0167811A1; US2020/0121809A1; US2019/0022247A1; U.S. Pat. No. 9,834,510B2; U.S. Pat. No. 9,593,077B2, each of which are incorporated by reference herein in their entirety. In some embodiments, the lipid nanoparticle is prepared according to methods described in any one of the foregoing references.

In some embodiments, the lipid nanoparticle comprises at least one ionizable cationic lipid, at least one non-cationic lipid, at least one sterol, and/or at least one polyethylene glycol (PEG)-modified lipid.

In some embodiments, the lipid nanoparticle comprises a molar ratio of 20-60% amino lipid relative to the other lipid components. For example, the lipid nanoparticle may comprise a molar ratio of 20-50%, 20-40%, 20-30%, 30-60%, 30-50%, 30-40%, 40-60%, 40-50%, or 50-60% amino lipid. In some embodiments, the lipid nanoparticle comprises a molar ratio of 20%, 30%, 40%, 50, or 60% amino lipid.

In some embodiments, the lipid nanoparticle comprises a molar ratio of 5-25% phospholipid relative to the other lipid components. For example, the lipid nanoparticle may comprise a molar ratio of 5-30%, 5-15%, 5-10%, 10⁻²⁵%, 10⁻²⁰%, 10⁻²⁵%, 15-25%, 15-20%, 20-25%, or 25-30% phospholipid. In some embodiments, the lipid nanoparticle comprises a molar ratio of 5%, 10%, 15%, 20%, 25%, or 30% non-cationic lipid.

In some embodiments, the lipid nanoparticle comprises a molar ratio of 25-55% structural lipid relative to the other lipid components. For example, the lipid nanoparticle may comprise a molar ratio of 10⁻⁵⁵%, 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% structural lipid. In some embodiments, the lipid nanoparticle comprises a molar ratio of 10%, 15%, 20%, 25%, 30%, 35%, 40%, 45%, 50%, or 55% structural lipid.

In some embodiments, the lipid nanoparticle comprises a molar ratio of 0.5-15% PEG lipid relative to the other lipid components. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10⁻¹⁵% PEG lipid. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-lipid.

In some embodiments, the lipid nanoparticle comprises a molar ratio of 20-60% amino lipid, 5-25% phospholipid, 25-55% structural lipid, and 0.5-15% PEG lipid.

In some embodiments, the lipid nanoparticle comprises a molar ratio of 20-60% amino lipid, 5-30% phospholipid, 10⁻⁵⁵% structural lipid, and 0.5-15% PEG lipid. In some embodiments, the lipid nanoparticle comprises an agent for enhanced delivery to target cells, e.g., liver cells and/or splenic cells. In some embodiments, the lipid nanoparticle comprises components and/or formulations described in US2022/0296517A1, which is herein incorporated by reference. In some embodiments, the lipid nanoparticle is prepared according to a method described in US2022/0296517A1.

In some embodiments, the lipid nanoparticle comprises an agent for enhanced delivery to an immune cell. In some embodiments, the lipid nanoparticle comprises components and/or formulations described in US 2019/0314291A1, which is herein incorporated by reference. In some embodiments, the lipid nanoparticle is prepared according to a method described in US 2019/0314291A1.

Ionizable Amino lipid

In some aspects, the disclosure relates to a compound of Formula (I):

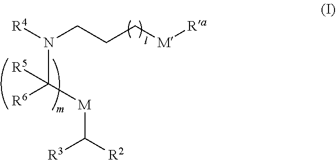

or its N-oxide, or a salt or isomer thereof, wherein R′^{a} is R′^{branched}; wherein
R′^{branched} is:

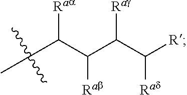

wherein

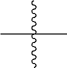

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH, wherein n is selected from the group consisting of 1, 2, 3, 4, and 5, and

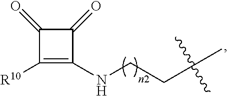

wherein

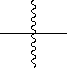

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R^{s} is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
1 is selected from the group consisting of 1, 2, 3, 4, and 5; and
m is selected from the group consisting of 5, 6, 7, 8, 9, 10, 11, 12, and 13.

In some embodiments of the compounds of Formula (I), R′^{a} is R′^{branched}; R′^{branched} is

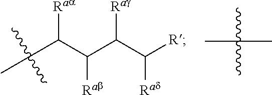

denotes a point of attachment; R^{aα}, R^{aβ}, R^{aγ}, and R^{aδ} are each H; R² and R3 are each C₁₋₁₄ alkyl; R⁴ is —(CH₂)ₙOH; n is 2; each R^{s} is H; each R⁶ is H; M and M′ are each —C(O)O—; R′ is a C₁₋₁₂ alkyl; 1 is 5; and m is 7.

In some embodiments of the compounds of Formula (I), R′^{a} is R′^{branched}; R′^{branched} is

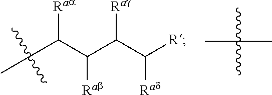

denotes a point of attachment; R^{aα}, R^{aβ}, R^{aγ}, and R^{aδ} are each H; R² and R3 are each C₁₋₁₄ alkyl; R⁴ is —(CH₂)ₙOH; n is 2; each R^{s} is H; each R⁶ is H; M and M′ are each —C(O)O—; R′ is a C₁₋₁₂ alkyl; 1 is 3; and m is 7.

In some embodiments of the compounds of Formula (I), R′^{a} is R′^{branched}; R′^{branched} is

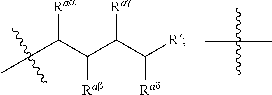

denotes a point of attachment; R^{aα} is C₂₋₁₂ alkyl; R^{aβ}, R^{aγ}, and R^{aδ} are each H; R² and R³ are each C₁₋₁₄ alkyl; R⁴ is

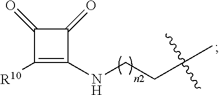

R¹⁰ NH(C₁₋₆ alkyl); n2 is 2; R⁵ is H; each R⁶ is H; M and M′ are each —C(O)O—; R′ is a C₁₋₁₂ alkyl; 1 is 5; and m is 7.

In some embodiments of the compounds of Formula (I), R′^{a} is R′^{branched}; R′^{branched} is

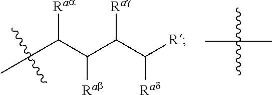

denotes a point of attachment; R^{aα}, R^{aβ}, and R^{aδ} are each H; R^{aγ} is C₂₋₁₂ alkyl; R² and R³ are each C₁₋₁₄ alkyl; R⁴ is —(CH₂)ₙOH; n is 2; each R^{s} is H; each R⁶ is H; M and M′ are each —C(O)O—; R′ is a C₁₋₁₂ alkyl; 1 is 5; and m is 7.

In some embodiments, the compound of Formula (I) is selected from:

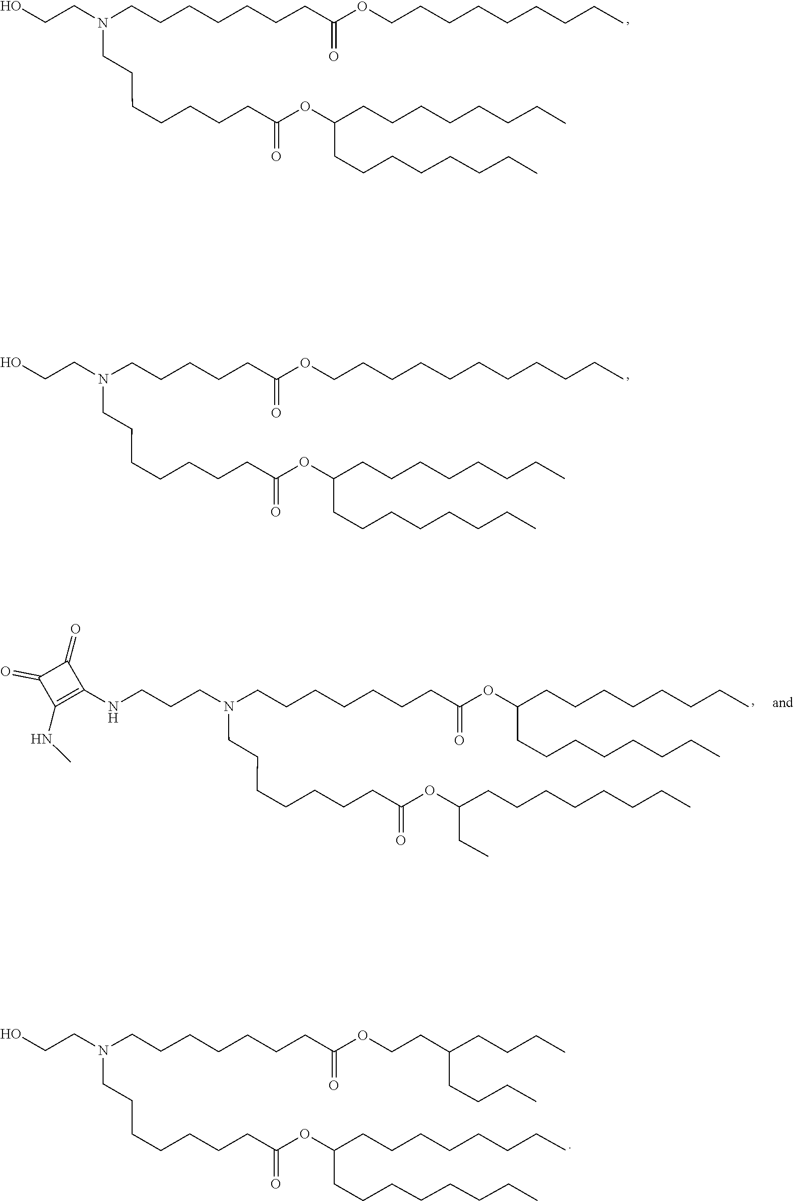 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCC(CC)OC(=O)CCCCCCCN(CCCCCCCC(=O)OC(CCCCCCCC)CCCCCCCC)CCCNc1c(NC)c(=O)c1=O ; CCCCCCCCC(CCCCCCCC)OC(=O)CCCCCCCN(CCO)CCCCCCCC(=O)OCCC(CCCC)CCCC]

In some embodiments, the compound of Formula (I) is Compound 1

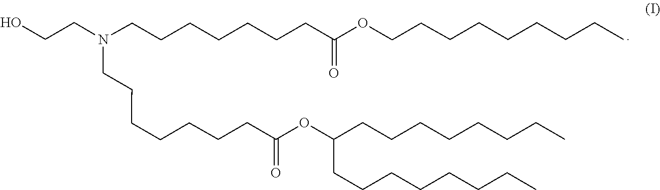 [SMILES: CCCCCCCCCOC(=O)CCCCCCCN(CCO)CCCCCCCC(=O)OC(CCCCCCCC)CCCCCCCC]

In some embodiments, the compound of Formula (I) is:

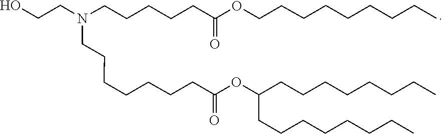 [SMILES: CCCCCCCCCOC(=O)CCCCCN(CCO)CCCCCCCC(=O)OC(CCCCCCCC)CCCCCCCC]

In some embodiments, the compound of Formula (I) is:

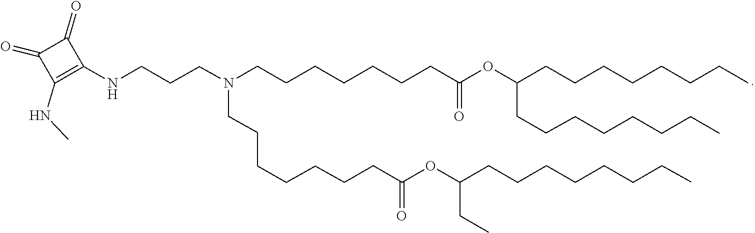 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

In some embodiments, the compound of Formula (I) is:

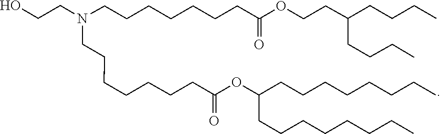 [SMILES: CCCCCCCCC(CCCCCCCC)OC(=O)CCCCCCCN(CCO)CCCCCCCC(=O)OCCC(CCCC)CCCC]

In some aspects, the disclosure relates to a compound of Formula (Ia):

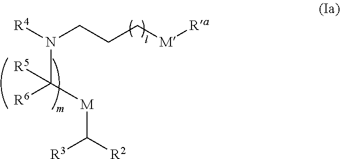

or its N-oxide, or a salt or isomer thereof, wherein R′^{a} is R′^{branched}; wherein
R′^{branched} is:

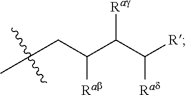

wherein

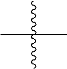

denotes a point of attachment;
wherein R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

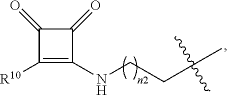

wherein

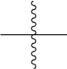

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R^{s} is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
1 is selected from the group consisting of 1, 2, 3, 4, and 5; and
m is selected from the group consisting of 5, 6, 7, 8, 9, 10, 11, 12, and 13.

In some aspects, the disclosure relates to a compound of Formula (Tb):

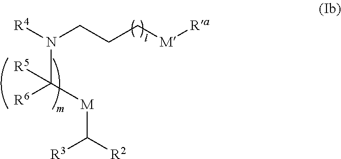

or its N-oxide, or a salt or isomer thereof, wherein R′^{a} is R′^{branched}; wherein
R′^{branched} is:

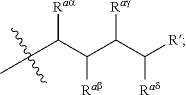

wherein

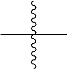

denotes a point of attachment;
wherein R^{aα}, R^{aβ}, R^{aγ}, and Ras are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is —(CH₂)ₙOH, wherein n is selected from the group consisting of 1, 2, 3, 4, and 5;
each R^{s} is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
1 is selected from the group consisting of 1, 2, 3, 4, and 5; and
m is selected from the group consisting of 5, 6, 7, 8, 9, 10, 11, 12, and 13.

In some embodiments of Formula (I) or (Ib), R′^{a} is R′^{branched}; R′^{branched}is

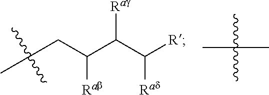

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments of Formula (I) or (Ib), R′^{a} is R′^{branched}; R′^{branched} is

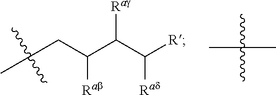

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 3; and m is 7.

In some embodiments of Formula (I) or (Ib), R′^{a} is R′^{branched}; R′^{branched} is

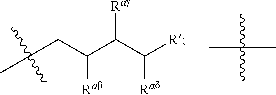

denotes a point of attachment; R^{aβ} and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—;R′ is a C₁₋₁₂ alkyl; 1 is 5; and m is 7.

In some aspects, the disclosure relates to a compound of Formula (Ic):

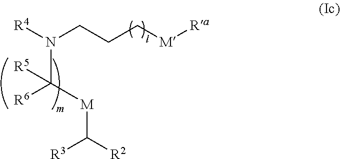

or its N-oxide, or a salt or isomer thereof, wherein R′^{a} is R′^{branched}; wherein
R′^{branched} is:

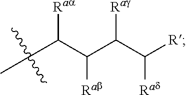

wherein

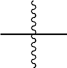

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is

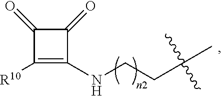

wherein

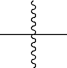

denotes a point of attachment; wherein
R¹⁰ is N(R)₂; each R is independently selected from the group consisting of C₁₋₆ alkyl, C₂₋₃ alkenyl, and H; n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
1 is selected from the group consisting of 1, 2, 3, 4, and 5; and
m is selected from the group consisting of 5, 6, 7, 8, 9, 10, 11, 12, and 13.

In some embodiments, R′^{a} is R′^{branched}; R′^{branched} is

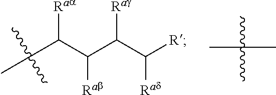

denotes a point of attachment; R^{aβ}, R^{aγ}, and R^{aδ} are each H; R^{aα} is C₂₋₁₂ alkyl; R² and R³ are each C₁₋₁₄ alkyl; R⁴ is

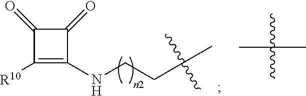

denotes a point of attachment; R¹⁰ is NH(C₁₋₆ alkyl); n2 is 2; each R⁵ is H; each R⁶ is H; M and M′ are each —C(O)O—; R′ is a C₁₋₁₂ alkyl; 1 is 5; and m is 7.

In some embodiments, the compound of Formula (Ic) is:

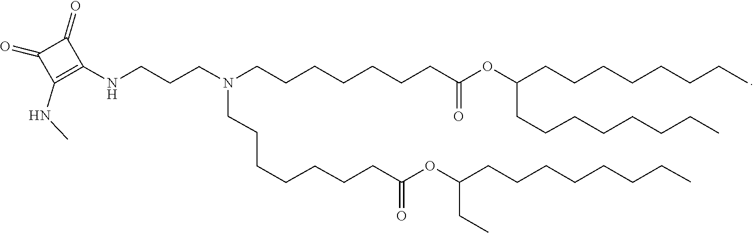 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

In some aspects, the disclosure relates to a compound of Formula (II):

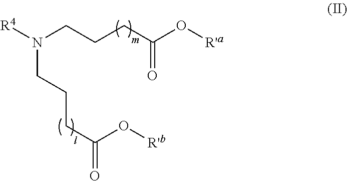

or its N-oxide, or a salt or isomer thereof,
wherein R^{′a} is R′^{branched} or R′^{cyclic}; wherein
R′^{branched} is:

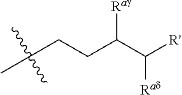

### R and R′^{cyclic} is:

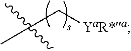

and
R′^{b} is:

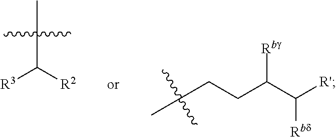

wherein

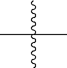

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

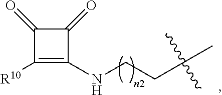

wherein

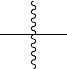

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
Y^{a} is a C₃₋₆ carbocycle;
R*″^{a} is selected from the group consisting of C₁₋₁₅ alkyl and C₂₋₁₅ alkenyl; and
s is 2 or 3;
m is selected from 1, 2, 3, 4, 5, 6, 7, 8, and 9;
1 is selected from 1, 2, 3, 4, 5, 6, 7, 8, and 9.

In some aspects, the disclosure relates to a compound of Formula (II-a):

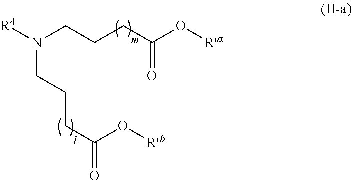

or its N-oxide, or a salt or isomer thereof, wherein R^{′a} is R′^{branched} or R′^{cyclic}; wherein
R′^{branched} is:

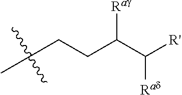

### and R′^{b} is:

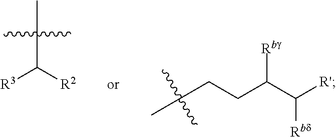

wherein

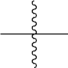

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂).OH wherein n is selected from the group consisting of 1, 2, 3, 4, and 5, and

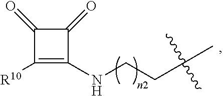

wherein

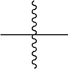

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
1 is selected from 1, 2, 3, 4, 5, 6, 7, 8, and 9.

In some aspects, the disclosure relates to a compound of Formula (II-b):

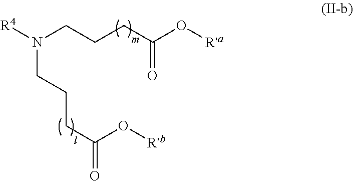

or its N-oxide, or a salt or isomer thereof,
wherein R^{′a} is R′^{branched} or R′^{cyclic}; wherein
R′^{branched} is:

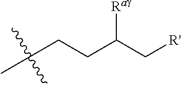

### and R′^{b} is:

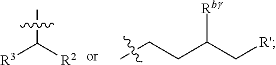

wherein

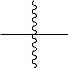

denotes a point of attachment;
R^{aγ} and R^{b}v are each independently selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

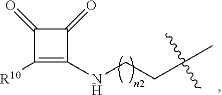

wherein

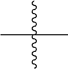

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
1 is selected from 1, 2, 3, 4, 5, 6, 7, 8, and 9.

In some aspects, the disclosure relates to a compound of Formula (II-c):

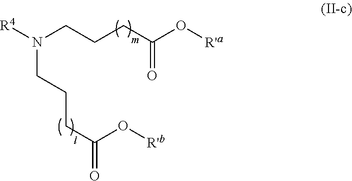

or its N-oxide, or a salt or isomer thereof, wherein R^{′a} is R′^{branched} or R′^{cyclic}; wherein
R′^{branched} is:

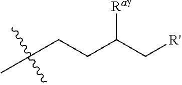

### and R′^{b} is:

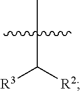

wherein

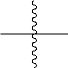

denotes a point of attachment;
wherein R^{aγ} is selected from the group consisting of C₁₋₁₂ alkyl and C₂₋₁₂ alkenyl; R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

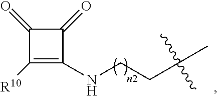

wherein

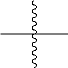

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
R′ is a C₁₋₁₂ alkyl or C₂₋₁₂ alkenyl;
m is selected from 1, 2, 3, 4, 5, 6, 7, 8, and 9;
1 is selected from 1, 2, 3, 4, 5, 6, 7, 8, and 9.

In some aspects, the disclosure relates to a compound of Formula (II-d):

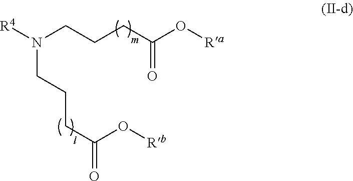

or its N-oxide, or a salt or isomer thereof, wherein R^{′a} is R branched or R′^{cyclic}; wherein
R′^{branched} is:

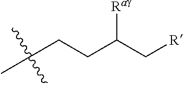

### and R′^{b} is:

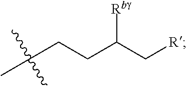

wherein

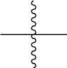

denotes a point of attachment;
wherein R^{aγ} and R^{bγ} are each independently selected from the group consisting of C₁₋₁₂ alkyl and C₂₋₁₂ alkenyl;
R⁴ is selected from the group consisting of —(CH₂)ₙOH wherein n is selected from the group consisting of 1, 2, 3, 4, and 5, and

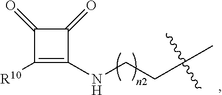

wherein

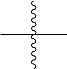

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
1 is selected from 1, 2, 3, 4, 5, 6, 7, 8, and 9.

In some aspects, the disclosure relates to a compound of Formula (II-e):

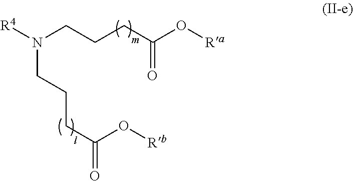

or its N-oxide, or a salt or isomer thereof, wherein R′^{a} is R′^{branched} or R′^{cyclic}; wherein
R′^{branched} is:

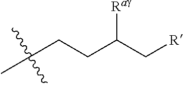

### and R′^{b} is:

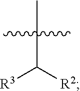

wherein

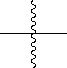

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is —(CH₂)ₙOH wherein n is selected from the group consisting of 1, 2, 3, 4, and 5;
R′ is a C₁₋₁₂ alkyl or C₂₋₁₂ alkenyl;
m is selected from 1, 2, 3, 4, 5, 6, 7, 8, and 9;
1 is selected from 1, 2, 3, 4, 5, 6, 7, 8, and 9.

In some embodiments of the compound of Formula (II), (II-a), (II-b), (II-c), (II-d), or (II-e), m and 1 are each independently selected from 4, 5, and 6. In some embodiments of the compound of Formula (II), (II-a), (II-b), (II-c), (II-d), or (II-e), m and 1 are each 5.

In some embodiments of the compound of Formula (II), (II-a), (II-b), (II-c), (II-d), or (II-e), each R′ independently is a C₁₋₁₂ alkyl. In some embodiments of the compound of Formula (II), (II-a), (II-b), (II-c), (II-d), or (II-e), each R′ independently is a C₂₋₅ alkyl.

In some embodiments of the compound of Formula (II), (II-a), (II-b), (II-c), (II-d), or (II-e), R′^{b} is:

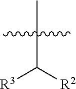

and R² and R³ are each independently a C₁₋₁₄ alkyl. In some embodiments of the compound of Formula (II), (II-a), (II-b), (II-c), (II-d), or (II-e), R′^{b} is:

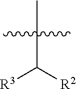

and R² and R³ are each independently a C₆₋₁₀ alkyl. In some embodiments of the compound of Formula (II), (II-a), (II-b), (II-c), (II-d), or (II-e), R′^{b} is

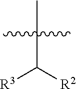

and R² and R³ are each a C₈ alkyl.

In some embodiments of the compound of Formula (II), (II-a), (II-b), (II-c), (II-d), or (II-e), R′^{branched} is:

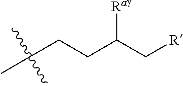

### and R′^{b} is:

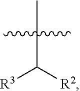

R^{aγ} is a C₁₋₁₂ alkyl and R² and R³ are each independently a C₆₋₁₀ alkyl. In some embodiments of the compound of Formula (II), (II-a), (II-b), (II-c), (II-d), or (II-e), R′^{branched} is:

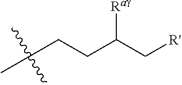

### and R′^{b} is:

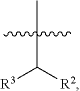

R^{aγ} is a C₂₋₆ alkyl and R² and R³ are each independently a C₆₋₁₀ alkyl. In some embodiments of the compound of Formula (II), (II-a), (II-b), (II-c), (II-d), or (II-e), R′^{branched} is:

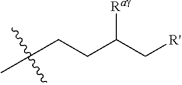

### and R′^{b} is:

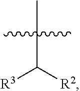

R^{aγ} is a C₂₋₆ alkyl, and R² and R³ are each a C₈ alkyl.

In some embodiments of the compound of Formula (II), (II-a), (II-b), (II-c), (II-d), or (II-e), R′^{branched} is:

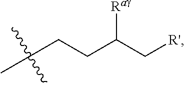

### R′^{b} is:

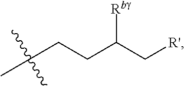

and R^{aγ} and R^{by} are each a C₁₋₁₂ alkyl. In some embodiments of the compound of Formula (II), (II-a), (II-b), (II-c), (II-d), or (II-e), R′^{branched} is:

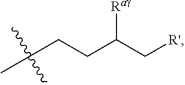

### R′^{b} is:

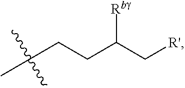

and R^{aγ} and R^{bγ} are each a C₂₋₆ alkyl.

In some embodiments of the compound of Formula (II), (II-a), (II-b), (II-c), (II-d), or (II-e), m and 1 are each independently selected from 4, 5, and 6 and each R′ independently is a C₁₋₁₂ alkyl. In some embodiments of the compound of Formula (II), (II-a), (II-b), (II-c), (II-d), or (II-e), m and 1 are each 5 and each R′ independently is a C₂₋₅ alkyl.

In some embodiments of the compound of Formula (II), (II-a), (II-b), (II-c), (II-d), or (II-e), R′^{branched} is:

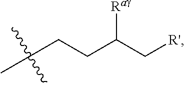

### R′^{b} is:

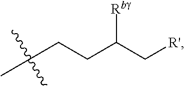

m and 1 are each independently selected from 4, 5, and 6, each R′ independently is a C₁₋₁₂ alkyl, and R^{aγ} and R^{bγ} are each a C₁₋₁₂ alkyl. In some embodiments of the compound of Formula (II), (II-a), (II-b), (II-c), (II-d), or (II-e), R′^{branched} is:

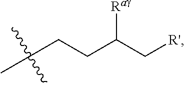

### R′^{b} is:

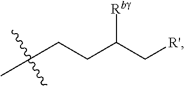

m and 1 are each 5, each R′ independently is a C₂₋₅ alkyl, and R^{aγ} and R^{bγ} are each a C₂₋₆ alkyl.

In some embodiments of the compound of Formula (II), (II-a), (II-b), (II-c), (II-d), or (II-e), R^{branched} is;

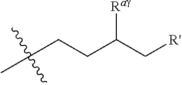

### and R′^{b} is:

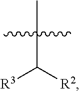

m and 1 are each independently selected from 4, 5, and 6, R′ is a C₁₋₁₂ alkyl, R^{aγ} is a C₁₋₁₂ alkyl and R² and R³ are each independently a C₆₋₁₀ alkyl. In some embodiments of the compound of Formula (II), (II-a), (II-b), (II-c), (II-d), or (II-e), R′^{branched} is:

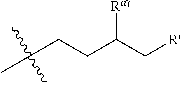

### and R′^{b} is:

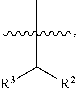

m and 1 are each 5, R′ is a C₂₋₅ alkyl, R^{aγ} is a C₂₋₆ alkyl, and R² and R³ are each a C₈ alkyl.

In some embodiments of the compound of Formula (II), (II-a), (II-b), (II-c), (II-d), or (II-e), R⁴ is

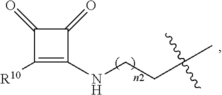

wherein R¹⁰ is NH(C₁₋₆ alkyl) and n2 is 2. In some embodiments of the compound of Formula (II), (II-a), (II-b), (II-c), (II-d), or (II-e), R⁴ is

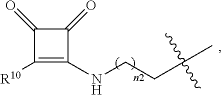

wherein R¹⁰ is NH(CH₃) and n2 is 2.

In some embodiments of the compound of Formula (II), (II-a), (II-b), (II-c), (II-d), or (II-e), R′^{branched} is:

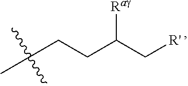

### R′^{b} is:

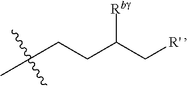

m and 1 are each independently selected from 4 5, and 6, each R′ independently is a C₁₋₁₂ alkyl, R^{aγ} and R^{bγ} are each a C₁₋₁₂ alkyl, and R⁴ is

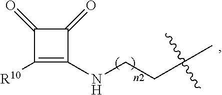

wherein R¹⁰ is NH(C₁₋₆ alkyl), and n2 is 2. In some embodiments of the compound of Formula (II), (II-a), (II-b), (II-c), (II-d), or (II-e), R′^{branched} is:

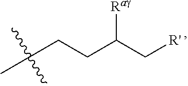

### R′^{b} is:

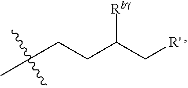

m and 1 are each 5, each R′ independently is a C₂₋₅ alkyl, R^{aγ} and R^{bγ} are each a C₂₋₆ alkyl, and R⁴ is

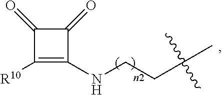

wherein R¹⁰ is NH(CH₃) and n2 is 2.

In some embodiments of the compound of Formula (II), (II-a), (II-b), (II-c), (II-d), or (II-e), R′^{branched} is:

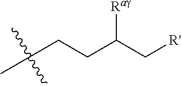

### and R′^{b} is:

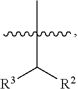

m and 1 are each independently selected from 4, 5, and 6, R′ is a C₁₋₁₂ alkyl, R² and R³ are each independently a C₆₋₁0 alkyl, R^{aγ} is a C₁₋₁₂ alkyl, and R⁴ is

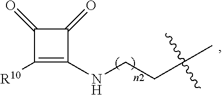

wherein R¹⁰ is NH(C₁₋₆ alkyl) and n2 is 2. In some embodiments of the compound of Formula (II), (II-a), (II-b), (II-c), (II-d), or (II-e), R′^{branched} is:

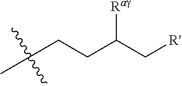

### and R′^{b} is:

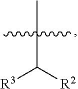

m and 1 are each 5, R′ is a C₂₋₅ alkyl, R^{aγ} is a C₂₋₆ alkyl, R² and R³ are each a C₈ alkyl, and R⁴ is:

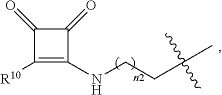

wherein R¹⁰ is NH(CH₃) and n2 is 2.

In some embodiments of the compound of Formula (II), (II-a), (II-b), (II-c), (II-d), or (II-e), R⁴ is —(CH₂)ₙOH and n is 2, 3, or 4. In some embodiments of the compound of Formula (II), (II-a), (II-b), (II-c), (II-d), or (II-e), R⁴ is —(CH₂)ₙOH and n is 2.

In some embodiments of the compound of Formula (II), (II-a), (II-b), (II-c), (II-d), or (II-e), R′^{branched} is:

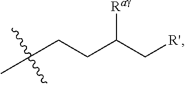

### R′^{b} is:

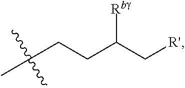

m and 1 are each independently selected from 4, 5, and 6, each R′ independently is a C₁₋₁₂ alkyl, R^{aγ} and R^{bγ} are each a C₁₋₁₂ alkyl, R⁴ is —(CH₂)ₙOH, and n is 2, 3, or 4. In some embodiments of the compound of Formula (II), (II-a), (II-b), (II-c), (II-d), or (II-e), R′^{branched} is:

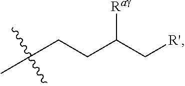

### R′^{b} is:

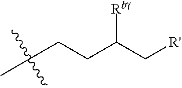

m and 1 are each 5, each R′ independently is a C₂₋₅ alkyl, R^{aγ} and R^{bγ} are each a C₂₋₆ alkyl, R⁴ is —(CH₂)ₙOH, and n is 2.

In some aspects, the disclosure relates to a compound of Formula (II-f):

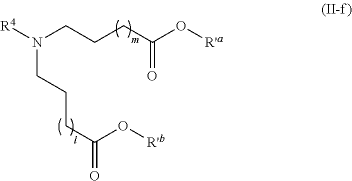

or its N-oxide, or a salt or isomer thereof, wherein R′^{a} is R′^{branched} or R′^{cyclic}; wherein
R′^{branched} is:

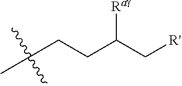

### and R′^{b} is:

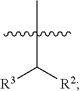

wherein

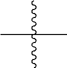

denotes a point of attachment;
R^{aγ} is a C₁₋₁₂ alkyl;
R² and R³ are each independently a C₁₋₁₄ alkyl;
R⁴ is —(CH₂)ₙOH wherein n is selected from the group consisting of 1, 2, 3, 4, and 5;
R′ is a C₁₋₁₂ alkyl;
m is selected from 4, 5, and 6; and
1 is selected from 4, 5, and 6.

In some embodiments of the compound of Formula (II-f), m and 1 are each 5, and n is 2, 3, or 4.

In some embodiments of the compound of Formula (II-f) R′ is a C₂₋₅ alkyl, R^{aγ} is a C₂₋₆ alkyl, and R² and R³ are each a C₆₋₁₀ alkyl.

In some embodiments of the compound of Formula (II-f), m and 1 are each 5, n is 2, 3, or 4, R′ is a C₂₋₅ alkyl, R^{aγ} is a C₂₋₆ alkyl, and R² and R³ are each a C₆₋₁0 alkyl.

In some aspects, the disclosure relates to a compound of Formula (II-g):

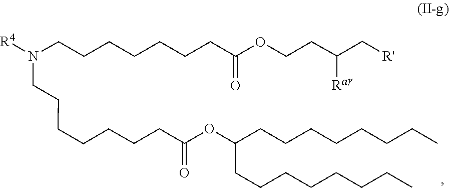

R^{aγ} is a C₂₋₆ alkyl;
R′ is a C₂₋₅ alkyl; and
R⁴ is selected from the group consisting of —(CH₂).OH wherein n is selected from the group consisting of 3, 4, and 5, and

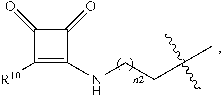

wherein

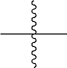

denotes a point of attachment, R¹⁰ is NH(C₁₋₆ alkyl), and n2 is selected from the group consisting of 1, 2, and 3.

In some aspects, the disclosure relates to a compound of Formula (II-h):

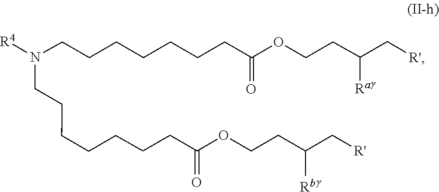

R^{aγ} and R^{bγ} are each independently a C₂₋₆ alkyl;
each R′ independently is a C₂₋₅ alkyl; and
R⁴ is selected from the group consisting of —(CH₂).OH wherein n is selected from the group consisting of 3, 4, and 5, and

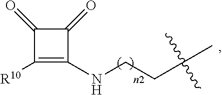

wherein

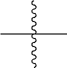

denotes a point of attachment, R¹⁰ is NH(C₁₋₆ alkyl), and n2 is selected from the group consisting of 1, 2, and 3.

In some embodiments of the compound of Formula (II-g) or (II-h), R⁴ is

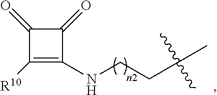

wherein
R¹⁰ is NH(CH₃) and n2 is 2.

In some embodiments of the compound of Formula (II-g) or (II-h), R⁴ is —(CH₂)₂OH.

In some aspects, the disclosure relates to a compound having the Formula (III):

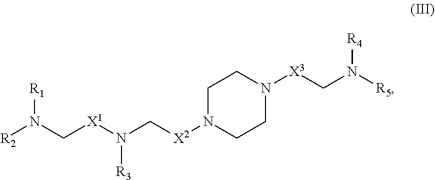

or a salt or isomer thereof, wherein
R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;
each M is independently selected from the group consisting of —C(O)O—, —OC(O)—, —OC(O)O—, —C(O)N(R′)-, —N(R′)C(O)-, —C(O)—, —C(S)—, —C(S)S—, —SC(S)—, —CH(OH)—, —P(O)(OR′)O—, —S(O)₂—, an aryl group, and a heteroaryl group;
X¹, X², and X³ are independently selected from the group consisting of a bond, —CH₂—, —(CH₂)₂—, —CHR—, —CHY—, —C(O)—, —C(O)O—, —OC(O)—, —C(O)—CH₂—, —CH₂—C(O)—, —C(O)O—CH₂—, —OC(O)—CH₂—, —CH₂—C(O)O—, —CH₂—OC(O)—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H; and
each R″ is independently selected from the group consisting of C₃₋₁₂ alkyl and C₃₋₁₂ alkenyl, and wherein:
   i) at least one of X¹, X², and X³ is not —CH₂—; and/or
   ii) at least one of R₁, R₂, R₃, R₄, and Rₛ is —R″MR′.

In some embodiments, R₁, R₂, R₃, R₄, and R₅ are each C₅₋₂₀ alkyl; X¹ is —CH₂—; and X² and X3 are each —C(O)—.

In some embodiments, the compound of Formula (III) is:

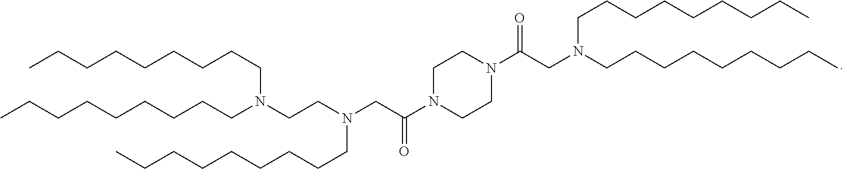 [SMILES: CCCCCCCCCN(CCCCCCCCC)CCN(CCCCCCCCC)CC(=O)N1CCN(C(=O)CN(CCCCCCCCC)CCCCCCCCC)CC1]

positive or partial positive charge at physiological pH.

### Phospholipids

The lipid composition of the lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid of the invention comprises 1,2-distearoyl-sn-glycero-3-phosphocholine (DSPC), 1,2-Distearoyl-sn-glycero-3-phosphoethanolamine (DSPE), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In certain embodiments, a phospholipid useful or potentially useful in the present invention is an analog or variant of DSPC. In certain embodiments, a phospholipid useful or potentially useful in the present invention is a compound of Formula (IV):

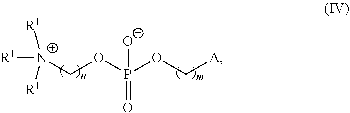

or a salt thereof, wherein:
each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;
nis 1,2,3,4,5,6,7, 8,9,or 10;
m is 0, 1, 2, 3, 4, 5, 6, 7, 8, 9, or 10;

A is of the formula:

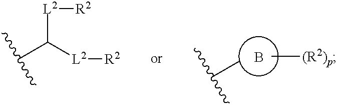

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O) OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;
each instance of R^{N} is independently hydrogen, optionally substituted alkyl, or a nitrogen protecting group; Ring B is optionally substituted carbocyclyl, optionally substituted heterocyclyl, optionally substituted aryl, or optionally substituted heteroaryl; and
p is 1 or 2;
provided that the compound is not of the formula:

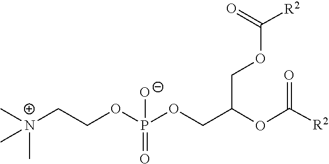

wherein each instance of R² is independently unsubstituted alkyl, unsubstituted alkenyl, or unsubstituted alkynyl.

In some embodiments, the phospholipids may be one or more of the phospholipids described in U.S. Application No. 62/520,530.

### Structural Lipids

The lipid composition of a pharmaceutical composition disclosed herein can comprise one or more structural lipids. As used herein, the term “structural lipid” refers to sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. application Ser. No. 16/493,814.

### Polyethylene Glycol (PEG)-Lipids

The lipid composition of a pharmaceutical composition disclosed herein can comprise one or more polyethylene glycol (PEG) lipids.

As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, the lipid moiety of the PEG-lipids includes those having lengths of from about C₁₄ to about C₂₂, preferably from about C₁₄ to about C₁₆. In some embodiments, a PEG moiety, for example a mPEG-NH₂, has a size of about 1000, 2000, 5000, 10,000, 15,000 or 20,000 daltons. In one embodiment, the PEG-lipid is PEG2k-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

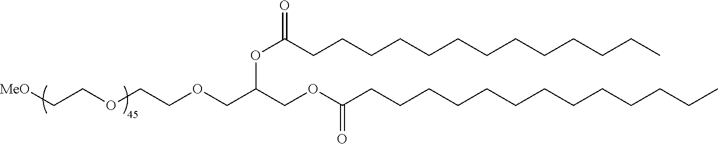

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In certain embodiments, a PEG lipid useful in the present invention is a compound of Formula (V). Provided herein are compounds of Formula (V):

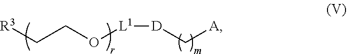

or salts thereof, wherein:
R³ is —OR^{O};
R^{O} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});
D is a moiety obtained by click chemistry or a moiety cleavable under physiological conditions;
m is 0, 1, 2, 3, 4, 5, 6, 7, 8, 9, or 10;

A is of the formula:

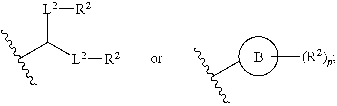

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O)OS(O) S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound of Formula (V) is a PEG-OH lipid (i.e., R³ is —OR^{O}, and R^{O} is hydrogen). In certain embodiments, the compound of Formula (V) is of Formula (V-OH):

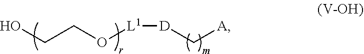

or a salt thereof.

In certain embodiments, a PEG lipid useful in the present invention is a PEGylated fatty acid. In certain embodiments, a PEG lipid useful in the present invention is a compound of Formula (VI). Provided herein are compounds of Formula (VI):

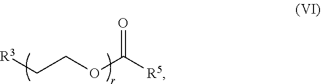

or a salts thereof, wherein:
R³ is —OR^{O};
R^{O} is hydrogen, optionally substituted alkyl or an oxygen protecting group;
r is an integer between 1 and 100, inclusive;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), C(O)N(R^{N}), —NR^{N}C(O), NR^{N}C(O)N(R^{N}), C(O)O, OC(O), OC(O)O, OC(O)N(R^{N}), NR^{N}C(O)O, C(O)S, SC(O), C(═NRN), C(═NRN)N(R^{N}), NR^{N}C(═NRN), NR^{N}C(═NRN)N(R^{N}), C(S), C(S)N(R^{N}), NR^{N}C(S), —NR^{N}C(S)N(R^{N}), S(O), OS(O), S(O)O, OS(O)O, OS(O)₂, S(O)₂O, OS(O)₂O, N(R^{N})S(O), —S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), —N(R^{N})S(O)₂N(R^{N}), OS(O)₂N(R^{N}), or N(R^{N})S(O)₂O; and each instance of R^{N} is independently hydrogen, optionally substituted alkyl, or a nitrogen protecting group.

In certain embodiments, the compound of Formula (VI) is of Formula (VI-OH):

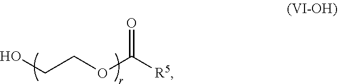

or a salt thereof. In some embodiments, r is 40-50.

In yet other embodiments the compound of Formula (VI) is:

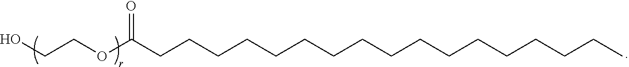

or a salt thereof.

In one embodiment, the compound of Formula (VI) is Compound 2

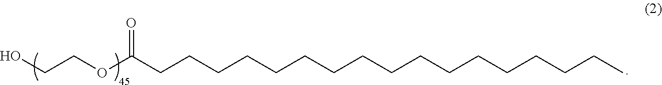

In some aspects, the lipid composition of the pharmaceutical compositions disclosed herein does not comprise a PEG-lipid.

In some embodiments, the PEG-lipids may be one or more of the PEG lipids described in U.S. Application No. U.S. Ser. No. 15/674,872.

In some embodiments, a LNP of the invention comprises an amino lipid of any of Formula I, II or III, a phospholipid comprising DSPC, a structural lipid, and a PEG lipid comprising PEG-DMG.

In some embodiments, a LNP of the invention comprises an amino lipid of any of Formula I, II or III, a phospholipid comprising DSPC, a structural lipid, and a PEG lipid comprising a compound having Formula VI.

In some embodiments, a LNP of the invention comprises an amino lipid of Formula I, II or III, a phospholipid comprising a compound having Formula IV, a structural lipid, and the PEG lipid comprising a compound having Formula V or VI.

In some embodiments, a LNP of the invention comprises an amino lipid of Formula I, II or III, a phospholipid comprising a compound having Formula IV, a structural lipid, and the PEG lipid comprising a compound having Formula V or VI.

In some embodiments, a LNP of the invention comprises an amino lipid of Formula I, II or III, a phospholipid having Formula IV, a structural lipid, and a PEG lipid comprising a compound having Formula VI.

In some embodiments, a LNP of the invention comprises an N:P ratio of from about 2:1 to about 30:1.

In some embodiments, a LNP of the invention comprises an N:P ratio of about 6:1.

In some embodiments, a LNP of the invention comprises an N:P ratio of about 3:1, 4:1, or 5:1.

In some embodiments, a LNP of the invention comprises a wt/wt ratio of the amino lipid component to the RNA of from about 10:1 to about 100:1.

In some embodiments, a LNP of the invention comprises a wt/wt ratio of the amino lipid component to the RNA of about 20:1.

In some embodiments, a LNP of the invention comprises a wt/wt ratio of the amino lipid component to the RNA of about 10:1.

In some embodiments, a LNP of the invention has a mean diameter from about 30 nm to about 150 nm.

In some embodiments, a LNP of the invention has a mean diameter from about 60 nm to about 120 nm.

In some embodiments, a LNP of the disclosure comprises the mRNA therapeutic agent described herein in a concentration from about 0.1 mg/ml to 2 mg/ml such as, but not limited to, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml or greater than 2.0 mg/ml. In some embodiments, a LNP of the disclosure comprises the mRNA therapeutic agent described herein in a concentration of about 2.0 mg/ml.

### Exemplary Lipid Nanoparticle Compositions

In some embodiments, the mRNA of the disclosure is formulated as LNPs. Accordingly, the present disclosure also provides nanoparticle compositions comprising (i) an LNP comprising an ionizable amino lipid (e.g., Compound 1), a phospholipid, a structural lipid, and a PEG-lipid (e.g., PEG-DMG, Compound 2), and (ii) an mRNA comprising an ORF encoding an IL15 fusion protein described herein. In some embodiments, the nanoparticle compositions disclosed herein comprise an LNP or a plurality of LNPs that encapsulate the mRNA of the disclosure.

In some embodiments, a nanoparticle composition of the present disclosure comprises at least one compound according to formula (I) described herein. For example, in some embodiments, the nanoparticle composition comprises Compound 1. In some embodiments, a nanoparticle composition of the present disclosure comprises at least one compound according to formula (I-a), (I-b), or (I-c) described herein. In some embodiments, a nanoparticle composition of the present disclosure comprises at least one compound according to formula (II) described herein. In some embodiments, a nanoparticle composition of the present disclosure comprises at least one compound according to formula (II-a), II-a), (II-b), (II-c), (II-d), (II-e), (11-), (II-g), or (II-h) described herein. In some embodiments, the nanoparticle compositions comprise other components. For example, in some embodiments, the nanoparticle composition comprises one or more other lipids in addition to a lipid according to formula (I), (I-a)-(I-c), (II), or (II-a)-(II-h), such as (i) at least one phospholipid described herein, (ii) at least one structural lipid described herein, (iii) at least one PEG-modified lipid described herein, or (v) any combination thereof. In some embodiments, the PEG-modified lipid comprises a compound according to Formula (V) described herein. In some embodiments, the PEG-modified lipid comprises a compound according to Formula (V-OH) described herein. In some embodiments, the PEG-modified lipid comprises a compound according to Formula (VI) described herein (e.g., Compound 2). In some embodiments, the PEG-modified lipid comprises a compound according to Formula (VI-OH) described herein. In some embodiments, the PEG-modified lipid comprises Compound 2.

In some embodiments, the nanoparticle composition comprises a compound of formula (I) (e.g., Compound 1). In some embodiments, the nanoparticle composition comprises a compound of formula (I) (e.g., Compound 1) and a phospholipid (e.g., DSPC or MSPC). In some embodiments, the nanoparticle composition comprises a compound of formula (I) described herein (e.g., Compound 1), a phospholipid described herein (e.g., DSPC or MSPC), and a sterol described herein (e.g., cholesterol). In some embodiments, the nanoparticle composition comprises a compound of formula (I) described herein (e.g., Compound 1), a phospholipid described herein (e.g., DSPC or MSPC), a sterol described herein (e.g., cholesterol), and a PEG-modified lipid described herein (e.g., PEG-DMG). In some embodiments, the nanoparticle composition comprises a compound of formula (I) described herein (e.g., Compound 1), a phospholipid described herein (e.g., DSPC or MSPC), a sterol described herein (e.g., cholesterol), and a PEG-modified lipid comprising a compound according to Formula (VI) (e.g., Compound 2).

In some embodiments, the nanoparticle composition comprises a lipid composition consisting or consisting essentially of compound of formula (I) described herein (e.g., Compound 1). In some embodiments, the nanoparticle composition comprises a lipid composition consisting or consisting essentially of a compound of formula (I) described herein (e.g., Compound 1) and a phospholipid (e.g., DSPC or MSPC). In some embodiments, the nanoparticle composition comprises a lipid composition consisting or consisting essentially of a compound of formula (I) described herein (e.g., Compound 1), a phospholipid (e.g., DSPC or MSPC), and a sterol (e.g., cholesterol). In some embodiments, the nanoparticle composition comprises a lipid composition consisting or consisting essentially of a compound of formula (I) described herein (e.g., Compound 1), a phospholipid (e.g., DSPC or MSPC), a sterol (e.g., cholesterol), and PEG-modified lipid (e.g., PEG-DMG). In some embodiments, the nanoparticle composition comprises a lipid composition consisting or consisting essentially of a compound of formula (I) described herein (e.g., Compound 1), a phospholipid (e.g., DSPC or MSPC), a sterol (e.g., cholesterol), and PEG-modified lipid comprising a compound according to Formula (VI) (e.g., Compound 2).

In one embodiment, the disclosure provides a nanoparticle composition comprising (1) a lipid composition comprising about 40-60 mole % of a compound of formula (I) described herein (e.g., Compound 1); about 8-16 mole % of phospholipid described herein (e.g., DSPC or MSPC); about 30-45% sterol described herein (e.g., cholesterol); about 1-5% PEG-modified lipid described herein (e.g., PEG-DMG); and (2) an mRNA encoding an IL15 fusion protein described herein.

In one embodiment, the disclosure provides a nanoparticle composition comprising (1) a lipid composition comprising about 45-65 mole % of a compound of formula (I) described herein (e.g., Compound 1); about 5-10 mole % of phospholipid described herein; about 25-40% sterol described herein; about 0.5-5% PEG-modified lipid described herein (e.g., PEG-DMG); and (2) an mRNA encoding an IL15 fusion protein described herein.

In one embodiment, the disclosure provides a nanoparticle composition comprising (1) a lipid composition comprising about 40-60 mole % of a compound of formula (I) described herein (e.g., Compound 1); about 8-16 mole % of phospholipid described herein (e.g., DSPC or MSPC); about 30-45% sterol described herein (e.g., cholesterol); about 1-5% PEG-modified lipid comprising a compound according to Formula VI (e.g., Compound 2); and (2) an mRNA encoding an IL15 fusion protein described herein.

In one embodiment, the disclosure provides a nanoparticle composition comprising (1) a lipid composition comprising about 45-65 mole % of a compound of formula (I) described herein (e.g., Compound 1); about 5-10 mole % of phospholipid described herein; about 25-40% sterol described herein; about 0.5-5% comprising a compound according to Formula VI (e.g., Compound 2); and (2) an mRNA encoding an IL15 fusion protein described herein.

In one embodiment, the disclosure provides a nanoparticle composition comprising (1) a lipid composition comprising about 40-60 mole % of Compound 1; about 8-16 mole % of DSPC; about 30-45% cholesterol; about 1-5% PEG-DMG; and (2) an mRNA encoding an IL15 fusion protein described herein.

In one embodiment, the disclosure provides a nanoparticle composition comprising (1) a lipid composition comprising about 40-60 mole % of Compound 1; about 8-16 mole % of DSPC; about 30-45% cholesterol; about 1-5% Compound 2; and (2) an mRNA encoding an IL15 fusion protein described herein.

In one embodiment, the disclosure provides a nanoparticle composition comprising (1) a lipid composition comprising about 45-65 mole % of Compound 1; about 5-10 mole % of DSPC; about 25-40% cholesterol; about 0.5-5% PEG-DMG; and (2) an mRNA encoding an IL15 fusion protein described herein.

In one embodiment, the disclosure provides a nanoparticle composition comprising (1) a lipid composition comprising about 45-65 mole % of Compound 1; about 5-10 mole % of DSPC; about 25-40% cholesterol; about 0.5-5% Compound 2; and (2) an mRNA encoding an IL15 fusion protein described herein.

Exemplary mRNA Formulations of the Disclosure

In some embodiments, the disclosure provides an mRNA comprising an ORF encoding a fusion protein, wherein the fusion protein comprises from N-terminus to C-terminus a signal peptide described herein, an ApoA polypeptide described herein, an extended sushi domain of an IL15Rα polypeptide described herein, and an IL15 polypeptide described herein, wherein the signal peptide, ApoA polypeptide, extended sushi domain, and IL15 polypeptide are operably linked, optionally via a linker.

In some embodiments, the fusion protein comprises from N-terminus to C-terminus a signal peptide comprising an amino acid sequence having at least about 80%, about 85%, about 90%, about 95%, about 98%, or about 99% identity to SEQ ID NO: 13, an ApoA polypeptide comprising an amino acid sequence having at least about 80%, about 85%, about 90%, about 95%, about 98%, or about 99% identity to SEQ ID NO: 14, an extended sushi domain of an IL15Rα polypeptide comprising an amino acid sequence having at least about 80%, about 85%, about 90%, about 95%, about 98%, or about 99% identity to SEQ ID NO: 17, and an IL15 polypeptide comprising an amino acid sequence having at least about 80%, about 85%, about 90%, about 95%, about 98%, or about 99% identity to SEQ ID NO: 16, wherein the signal peptide, ApoA polypeptide, extended sushi domain, and IL15 polypeptide are operably linked, optionally via a linker.

In some embodiments, the fusion protein comprises from N-terminus to C-terminus a signal peptide comprising an amino acid sequence set forth in SEQ ID NO: 13, an ApoA polypeptide comprising an amino acid sequence set forth in SEQ ID NO: 14, an extended sushi domain of an IL15Rα polypeptide comprising an amino acid sequence set forth in SEQ ID NO: 17, and an IL15 polypeptide comprising an amino acid sequence set forth in SEQ ID NO: 16, wherein the signal peptide, ApoA polypeptide, extended sushi domain, and IL15 polypeptide are operably linked, optionally via a linker.

In some embodiments, the fusion protein comprises from N-terminus to C-terminus a signal peptide comprising an amino acid sequence having at least about 80%, about 85%, about 90%, about 95%, about 98%, or about 99% identity to SEQ ID NO: 13, an ApoA polypeptide comprising an amino acid sequence having at least about 80%, about 85%, about 90%, about 95%, about 98%, or about 99% identity to SEQ ID NO: 14, an extended sushi domain of an IL15Rα polypeptide comprising an amino acid sequence having at least about 80%, about 85%, about 90%, about 95%, about 98%, or about 99% identity to SEQ ID NO: 18, and an IL15 polypeptide comprising an amino acid sequence having at least about 80%, about 85%, about 90%, about 95%, about 98%, or about 99% identity to SEQ ID NO: 16, wherein the signal peptide, ApoA polypeptide, extended sushi domain, and IL15 polypeptide are operably linked, optionally via a linker.

In some embodiments, the fusion protein comprises from N-terminus to C-terminus a signal peptide comprising an amino acid sequence set forth in SEQ ID NO: 13, an ApoA polypeptide comprising an amino acid sequence set forth in SEQ ID NO: 14, an extended sushi domain of an IL15Rα polypeptide comprising an amino acid sequence set forth in SEQ ID NO: 18, and an IL15 polypeptide comprising an amino acid sequence set forth in SEQ ID NO: 16, wherein the signal peptide, ApoA polypeptide, extended sushi domain, and IL15 polypeptide are operably linked, optionally via a linker.

In some embodiments, the disclosure provides an mRNA comprising an ORF encoding a fusion protein, wherein the ORF comprises from 5′ to 3′: (i) a nucleotide sequence encoding a signal peptide described herein; (ii) a nucleotide sequence encoding an ApoA polypeptide described herein; (iii) a nucleotide sequence encoding an extended sushi domain of an IL15Rα polypeptide described herein; (iv) a nucleotide sequence encoding an IL15 polypeptide described herein, wherein (i)-(iv) are operably linked, optionally via a nucleotide sequence encoding a linker described herein.

In some embodiments, the ORF comprises from 5′ to 3′: (i) a nucleotide sequence encoding a signal peptide having at least about 70%, about 75%, about 80%, about 85%, about 90%, about 95%, about 98%, or about 99% to any one of SEQ ID NOs: 24-26; (ii) a nucleotide sequence encoding an ApoA polypeptide having at least about 70%, about 75%, about 80%, about 85%, about 90%, about 95%, about 98%, or about 99% to any one of SEQ ID NOs: 34-37; (iii) a nucleotide sequence encoding an extended sushi domain of an IL15Rα polypeptide having at least about 70%, about 75%, about 80%, about 85%, about 90%, about 95%, about 98%, or about 99% to any one of SEQ ID NOs: 29-31; (iv) a nucleotide sequence encoding an IL15 polypeptide having at least about 70%, about 75%, about 80%, about 85%, about 90%, about 95%, about 98%, or about 99% to any one of SEQ ID NOs: 38-42, wherein (i)-(iv) are operably linked, optionally via a nucleotide sequence encoding a linker described herein.

In some embodiments, the ORF comprises from 5′ to 3′: (i) a nucleotide sequence encoding a signal peptide set forth in any one of SEQ ID NOs: 24-26; (ii) a nucleotide sequence encoding an ApoA polypeptide set forth in any one of SEQ ID NOs: 34-37; (iii) a nucleotide sequence encoding an extended sushi domain of an IL15Rα polypeptide set forth in any one of SEQ ID NOs: 29-31; (iv) a nucleotide sequence encoding an IL15 polypeptide set forth in any one of SEQ ID NOs: 38-42, wherein (i)-(iv) are operably linked, optionally via a nucleotide sequence encoding a linker described herein.

In some embodiments, the ORF comprises from 5′ to 3′: (i) a nucleotide sequence encoding a signal peptide having at least about 70%, about 75%, about 80%, about 85%, about 90%, about 95%, about 98%, or about 99% to any one of SEQ ID NOs: 24-26; (ii) a nucleotide sequence encoding an ApoA polypeptide having at least about 70%, about 75%, about 80%, about 85%, about 90%, about 95%, about 98%, or about 99% to any one of SEQ ID NOs: 34-37; (iii) a nucleotide sequence encoding an extended sushi domain of an IL15Rα polypeptide having at least about 70%, about 75%, about 80%, about 85%, about 90%, about 95%, about 98%, or about 99% to any one of SEQ ID NOs: 32-33; (iv) a nucleotide sequence encoding an IL15 polypeptide having at least about 70%, about 75%, about 80%, about 85%, about 90%, about 95%, about 98%, or about 99% to any one of SEQ ID NOs: 38-42, wherein (i)-(iv) are operably linked, optionally via a nucleotide sequence encoding a linker described herein.

In some embodiments, the ORF comprises from 5′ to 3′: (i) a nucleotide sequence encoding a signal peptide set forth in any one of SEQ ID NOs: 24-26; (ii) a nucleotide sequence encoding an ApoA polypeptide set forth in any one of SEQ ID NOs: 34-37; (iii) a nucleotide sequence encoding an extended sushi domain of an IL15Rα polypeptide set forth in any one of SEQ ID NOs: 32-33; (iv) a nucleotide sequence encoding an IL15 polypeptide set forth in any one of SEQ ID NOs: 38-42, wherein (i)-(iv) are operably linked, optionally via a nucleotide sequence encoding a linker described herein.

In some embodiments, the mRNA comprising an ORF encoding a fusion protein comprises a 5′cap described herein, a 5′ untranslated region (5′UTR) described herein, a 3′UTR described herein, and a polyA tail. In some embodiments, the 3′UTR comprises one or more miR-122 binding sites (e.g., a miR-122 binding site comprising the nucleotide sequence set forth in SEQ ID NOs: 113 or 115).

In some embodiments, the mRNA comprising an ORF encoding a fusion protein comprises a 5′cap, a 5′UTR, a 3′UTR, and a polyA tail, wherein (i) the 5′UTR comprises a nucleotide sequence having at least about 70%, about 75%, about 80%, about 85%, about 90%, about 95%, about 98%, or about 99% to SEQ ID NO: 19; (ii) the 3′UTR comprises a nucleotide sequence having at least about 70%, about 75%, about 80%, about 85%, about 90%, about 95%, about 98%, or about 99% to SEQ ID NO: 20; or (iii) both (i) and (ii).

In some embodiments, the mRNA comprising an ORF encoding a fusion protein comprises a 5′cap, a 5′UTR, a 3′UTR, and a polyA tail, wherein (i) the 5′UTR comprises SEQ ID NO: 19; (ii) the 3′UTR comprises SEQ ID NO: 20; or (iii) both (i) and (ii).

In some embodiments, the mRNA comprising an ORF encoding a fusion protein comprises a 5′cap, a 5′UTR, a 3′UTR, and a polyA tail, wherein (i) the 5′UTR comprises SEQ ID NO: 19; (ii) the 3′UTR comprises SEQ ID NO: 20 and one or more miR-122 binding sites (e.g., a miR-122 binding site comprising the nucleotide sequence set forth in SEQ ID NOs: 113 or 115), wherein the one or more miR-122 binding sites are inserted into the nucleotide sequence of SEQ ID NO: 20; or (iii) both (i) and (ii).

In some embodiments, the mRNA comprising an ORF encoding a fusion protein comprises a 5′cap, a 5′UTR, a 3′UTR, and a polyA tail, wherein (i) the 5′UTR comprises a nucleotide sequence having at least about 70%, about 75%, about 80%, about 85%, about 90%, about 95%, about 98%, or about 99% to SEQ ID NO: 19; (ii) the 3′UTR comprises a nucleotide sequence having at least about 70%, about 75%, about 80%, about 85%, about 90%, about 95%, about 98%, or about 99% to any one of SEQ ID NOs: 98 and 109; or (iii) both (i) and (ii).

In some embodiments, the mRNA comprising an ORF encoding a fusion protein comprises a 5′cap, a 5′UTR, a 3′UTR, and a polyA tail, wherein (i) the 5′UTR comprises SEQ ID NO: 19; (ii) the 3′UTR comprises SEQ ID NOs: 98 or 109; or (iii) both (i) and (ii).

In some embodiments, the mRNA comprising an ORF encoding a fusion protein is chemically modified. In some embodiments, the mRNA comprises at least one chemical modification described herein. In some embodiments, the at least one chemical modification is selected from a modified sugar moiety, a modified internucleoside linkage, a modified nucleobase, and a combination thereof. In some embodiments, the mRNA is fully modified with chemically-modified uridines described herein. In some embodiments, the mRNA is fully modified with N1-methylpseudouridine.

In some embodiments, the disclosure provides an mRNA comprising a 5′cap described herein, a 5′UTR described herein, an ORF encoding a fusion protein described herein, a 3′UTR described herein, and a polyA tail, wherein the mRNA comprises at least one chemical modification described herein, optionally wherein the mRNA is fully modified with chemically-modified uridines described herein (e.g., N1-methylpseudouridine).

In some embodiments, the mRNA comprises (i) a 5′cap, (ii) a 5′UTR comprising a nucleotide sequence having at least about 70%, about 75%, about 80%, about 85%, about 90%, about 95%, about 98%, or about 99% to SEQ ID NO: 19, (iii) an ORF comprising a nucleotide sequence encoding an amino acid sequence having at least about 80%, about 85%, about 90%, about 95%, about 98%, or about 99% identity to any one of SEQ ID NOs: 2, 4, 121, and 123; (iv) a 3′UTR comprising a nucleotide sequence having at least about 70%, about 75%, about 80%, about 85%, about 90%, about 95%, about 98%, or about 99% to SEQ ID NO: 20, optionally further comprising one or more miR-122 binding sites (e.g., a miR-122 binding site comprising the nucleotide sequence set forth in SEQ ID NOs: 113 or 115) inserted into the nucleotide sequence of SEQ ID NO: 20; and (v) a polyA tail, wherein the mRNA comprises at least one chemical modification described herein, optionally wherein the mRNA is fully modified with chemically-modified uridines described herein (e.g., N1-methylpseudouridine).

In some embodiments, the mRNA comprises (i) a 5′cap, (ii) a 5′UTR comprising SEQ ID NO: 19, (iii) an ORF comprising a nucleotide sequence encoding any one of SEQ ID NOs: 2, 4, 121, and 123; (iv) a 3′UTR comprising SEQ ID NO: 20, optionally further comprising one or more miR-122 binding sites (e.g., a miR-122 binding site comprising the nucleotide sequence set forth in SEQ ID NOs: 113 or 115) inserted into the nucleotide sequence of SEQ ID NO: 20; and (v) a polyA tail, wherein the mRNA comprises at least one chemical modification described herein, optionally wherein the mRNA is fully modified with chemically-modified uridines described herein (e.g., N1-methylpseudouridine).

In some embodiments, the mRNA comprises (i) a 5′cap, (ii) a 5′UTR comprising a nucleotide sequence having at least about 70%, about 75%, about 80%, about 85%, about 90%, about 95%, about 98%, or about 99% to SEQ ID NO: 19, (iii) an ORF comprising a nucleotide sequence encoding an amino acid sequence having at least about 80%, about 85%, about 90%, about 95%, about 98%, or about 99% identity to any one of SEQ ID NOs: 2 and 121; (iv) a 3′UTR comprising a nucleotide sequence having at least about 70%, about 75%, about 80%, about 85%, about 90%, about 95%, about 98%, or about 99% to SEQ ID NO: 20, optionally further comprising one or more miR-122 binding sites (e.g., a miR-122 binding site comprising the nucleotide sequence set forth in SEQ ID NOs: 113 or 115) inserted into the nucleotide sequence of SEQ ID NO: 20; and (v) a polyA tail, wherein the mRNA comprises at least one chemical modification described herein, optionally wherein the mRNA is fully modified with chemically-modified uridines described herein (e.g., N1-methylpseudouridine).

In some embodiments, the mRNA comprises (i) a 5′cap, (ii) a 5′UTR comprising SEQ ID NO: 19, (iii) an ORF comprising a nucleotide sequence encoding any one of SEQ ID NOs: 2 and 121; (iv) a 3′UTR comprising SEQ ID NO: 20, optionally further comprising one or more miR-122 binding sites (e.g., a miR-122 binding site comprising the nucleotide sequence set forth in SEQ ID NOs: 113 or 115) inserted into the nucleotide sequence of SEQ ID NO: 20; and (v) a polyA tail, wherein the mRNA comprises at least one chemical modification described herein, optionally wherein the mRNA is fully modified with chemically-modified uridines described herein (e.g., N1-methylpseudouridine).

In some embodiments, the mRNA comprises (i) a 5′cap, (ii) a 5′UTR comprising a nucleotide sequence having at least about 70%, about 75%, about 80%, about 85%, about 90%, about 95%, about 98%, or about 99% to SEQ ID NO: 19, (iii) an ORF comprising a nucleotide sequence having at least about 70%, about 75%, about 80%, about 85%, about 90%, about 95%, about 98%, or about 99% identity to any one of SEQ ID NOs: 1, 3, 120, and 122; (iv) a 3′UTR comprising a nucleotide sequence having at least about 70%, about 75%, about 80%, about 85%, about 90%, about 95%, about 98%, or about 99% to SEQ ID NO: 20, optionally further comprising one or more miR-122 binding sites (e.g., a miR-122 binding site comprising the nucleotide sequence set forth in SEQ ID NOs: 113 or 115) inserted into the nucleotide sequence of SEQ ID NO: 20; and (v) a polyA tail, wherein the mRNA comprises at least one chemical modification described herein, optionally wherein the mRNA is fully modified with chemically-modified uridines described herein (e.g., N1-methylpseudouridine).

In some embodiments, the mRNA comprises (i) a 5′cap, (ii) a 5′UTR comprising SEQ ID NO: 19, (iii) an ORF comprising a nucleotide sequence set forth in any one of SEQ ID NOs: 1, 3, 120, and 122; (iv) a 3′UTR comprising SEQ ID NO: 20, optionally further comprising one or more miR-122 binding sites (e.g., a miR-122 binding site comprising the nucleotide sequence set forth in SEQ ID NOs: 113 or 115) inserted into the nucleotide sequence of SEQ ID NO: 20; and (v) a polyA tail, wherein the mRNA comprises at least one chemical modification described herein, optionally wherein the mRNA is fully modified with chemically-modified uridines described herein (e.g., N1-methylpseudouridine).

In some embodiments, the mRNA comprises (i) a 5′cap, (ii) a 5′UTR comprising a nucleotide sequence having at least about 70%, about 75%, about 80%, about 85%, about 90%, about 95%, about 98%, or about 99% to SEQ ID NO: 19, (iii) an ORF comprising a nucleotide sequence having at least about 70%, about 75%, about 80%, about 85%, about 90%, about 95%, about 98%, or about 99% identity to any one of SEQ ID NOs: 1 and 120; (iv) a 3′UTR comprising a nucleotide sequence having at least about 70%, about 75%, about 80%, about 85%, about 90%, about 95%, about 98%, or about 99% to SEQ ID NO: 20, optionally further comprising one or more miR-122 binding sites (e.g., a miR-122 binding site comprising the nucleotide sequence set forth in SEQ ID NOs: 113 or 115) inserted into the nucleotide sequence of SEQ ID NO: 20; and (v) a polyA tail, wherein the mRNA comprises at least one chemical modification described herein, optionally wherein the mRNA is fully modified with chemically-modified uridines described herein (e.g., N1-methylpseudouridine).

In some embodiments, the mRNA comprises (i) a 5′cap, (ii) a 5′UTR comprising SEQ ID NO: 19, (iii) an ORF comprising a nucleotide sequence set forth in any one of SEQ ID NOs: 1 and 120; (iv) a 3′UTR comprising SEQ ID NO: 20, optionally further comprising one or more miR-122 binding sites (e.g., a miR-122 binding site comprising the nucleotide sequence set forth in SEQ ID NOs: 113 or 115) inserted into the nucleotide sequence of SEQ ID NO: 20; and (v) a polyA tail, wherein the mRNA comprises at least one chemical modification described herein, optionally wherein the mRNA is fully modified with chemically-modified uridines described herein (e.g., N1-methylpseudouridine).

In some embodiments, the disclosure provides a lipid nanoparticle (LNP) comprising the mRNA comprising an ORF encoding the fusion protein, wherein the LNP comprises an ionizable amino lipid, a phospholipid, a structural lipid, and a PEG-modified lipid. In some embodiments, the LNP comprises a molar ratio of 40-60% ionizable amino lipid, 8-16% phospholipid, 30-45% sterol, and 1-5% PEG modified lipid. In some embodiments, the LNP comprises a molar ratio of 45-65% ionizable amino lipid, 5-10% phospholipid, 25-40% sterol, and 0.5-5% PEG-modified lipid.

In some embodiments, the LNP comprises an ionizable amino lipid described herein, a phospholipid described herein, cholesterol, and PEG-DMG. In some embodiments, the LNP comprises a molar ratio of 40-60% ionizable amino lipid, 8-16% phospholipid, 30-45% cholesterol, and 1-5% PEG-DMG. In some embodiments, the LNP comprises a molar ratio of 45-65% ionizable amino lipid, 5-10% phospholipid, 25-40% cholesterol, and 0.5-5% PEG-DMG.

In some embodiments, the LNP comprises an ionizable amino lipid described herein, a phospholipid described herein, cholesterol, and a PEG-modified lipid, wherein the PEG-modified lipid is Compound 2. In some embodiments, the LNP comprises a molar ratio of 40-60% ionizable amino lipid, 8-16% phospholipid, 30-45% cholesterol, and 1-5% Compound 2. In some embodiments, the LNP comprises a molar ratio of 45-65% ionizable amino lipid, 5-10% phospholipid, 25-40% cholesterol, and 0.5-5% Compound 2.

In some embodiments, the LNP comprises an ionizable amino lipid, wherein the ionizable amino lipid is Compound 1, a phospholipid described herein, cholesterol, and a PEG-modified lipid described herein. In some embodiments, the LNP comprises a molar ratio of 40-60% Compound 1, 8-16% phospholipid, 30-45% cholesterol, and 1-5% PEG-modified lipid. In some embodiments, the LNP comprises a molar ratio of 45-65% Compound 1, 5-10% phospholipid, 25-40% cholesterol, and 0.5-5% PEG-modified lipid.

In some embodiments, the LNP comprises an ionizable amino lipid, wherein the ionizable amino lipid is Compound 1, a phospholipid described herein, cholesterol, and PEG-DMG. In some embodiments, the LNP comprises a molar ratio of 40-60% Compound 1, 8-16% phospholipid, 30-45% cholesterol, and 1-5% PEG-DMG. In some embodiments, the LNP comprises a molar ratio of 45-65% Compound 1, 5-10% phospholipid, 25-40% cholesterol, and 0.5-5% PEG-DMG.

In some embodiments, the LNP comprises an ionizable amino lipid, wherein the ionizable amino lipid is Compound 1, a phospholipid described herein, cholesterol, and Compound 2. In some embodiments, the LNP comprises a molar ratio of 40-60% Compound 1, 8-16% phospholipid, 30-45% cholesterol, and 1-5% Compound 2. In some embodiments, the LNP comprises a molar ratio of 45-65% Compound 1, 5-10% phospholipid, 25-40% cholesterol, and 0.5-5% Compound 2.

### Methods of Use

In some embodiments, the disclosure provides a method for treating a cancer in a subject in need thereof, e.g., a human subject. In some embodiments, the disclosure provides a method for enhancing an immune response to a cancer. In some embodiments, the disclosure provides a method for enhancing an immune response to a leukemic cell (e.g., an AML cell). In some embodiments, the disclosure provides a method for enhancing an immune response to a solid tumor. In some embodiments, enhancing an immune response comprises stimulating cytokine production. In another embodiment, enhancing an immune response comprises enhancing cellular immunity (T cell responses), such activating T cells. In some embodiments, enhancing an immune response comprises activating NK cells. Enhancement of an immune response in a subject can be evaluated by a variety of methods established in the art for assessing immune response, including but not limited to determining the level of T cell activation and NK cell activation by intracellular staining of activation markers.

### Disseminated Cancers

In some embodiments, the disclosure provides a method for treating a disseminated cancer in a subject in need thereof, e.g., a human subject. In some embodiments, treatment of a disseminated cancer comprises enhancing an immune response to the disseminated cancer. Disseminated cancers include metastatic cancers and cancers located within the circulation, e.g., the blood, of a subject which do not ordinarily form solid tumors. Disseminated cancers that do not ordinarily form solid tumors include, but are not limited to, cancers having significant myeloid populations, as well as multiple myeloma and B cell leukemias.

In some embodiments, the disseminated cancer is a hematological cancer. As used herein, the term “hematological cancer” includes a lymphoma, leukemia, myeloma or a lymphoid malignancy, as well as a cancer of the spleen and lymph nodes. Exemplary lymphomas include both B cell lymphomas (a B-cell hematological cancer) and T cell lymphomas. B-cell lymphomas include both Hodgkin's lymphomas and most non-Hodgkin's lymphomas. Non- limiting examples of B cell lymphomas include diffuse large B-cell lymphoma, follicular lymphoma, mucosa-associated lymphatic tissue lymphoma, small cell lymphocytic lymphoma (overlaps with chronic lymphocytic leukemia), mantle cell lymphoma (MCL), Burkitt's lymphoma, mediastinal large B cell lymphoma, Waldenstrom macroglobulinemia, nodal marginal zone B cell lymphoma, splenic marginal zone lymphoma, intravascular large B-cell lymphoma, primary effusion lymphoma, lymphomatoid granulomatosis. Non-limiting examples of T cell lymphomas include extranodal T cell lymphoma, cutaneous T cell lymphomas, anaplastic large cell lymphoma, and angioimmunoblastic T cell lymphoma. Hematological malignancies also include leukemia, such as, but not limited to, secondary leukemia, chronic lymphocytic leukemia, acute myelogenous leukemia, chronic myelogenous leukemia, and acute lymphoblastic leukemia. Hematological malignancies further include myelomas, such as, but not limited to, multiple myeloma and smoldering multiple myeloma. Other hematological and/or B cell- or T-cell-associated cancers are encompassed by the term hematological malignancy.

In some embodiments, the disseminated cancer is a myeloid malignancy. Myeloid malignancies include myelodysplastic syndrome (MDS), myeloproliferative disorders or neoplasms (MPD) and acute myeloid leukemia (AML).

In some embodiments, the disseminated cancer is a metastases of a primary tumor. In some embodiments, the disseminated cancer is a metastases of a previous metastases of a primary tumor. In some embodiments, disseminated cancer cells are detached from a primary tumor or metastases and enter the circulation. Such disseminated cancer cells can form tumors in locations distal from the primary tumor or metastases from which the cells are derived.

### Solid Tumors

In some embodiments, the disclosure provides a method for treating a solid tumor in a subject in need thereof, e.g., a human subject. In some embodiments, treatment of a solid tumor comprises enhancing an immune response to the solid tumor.

In some embodiments, the method comprises intratumoral administration of the compositions and/or mRNAs disclosed herein. In some embodiments, intratumoral administration promotes an immune response systemically. In some embodiments, intratumoral administration results in the shrinking or delaying of untreated tumors by promotion of an immune response systemically.

A “solid tumor” includes, but is not limited to, sarcoma, melanoma, carcinoma, or other solid tumor cancer. “Sarcoma” refers to a tumor which is made up of a substance like the embryonic connective tissue and is generally composed of closely packed cells embedded in a fibrillar or homogeneous substance. Sarcomas include, but are not limited to, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma. The term “melanoma” refers to a tumor arising from the melanocytic system of the skin and other organs. Melanomas include, for example, acra-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, metastatic melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas include, e.g., acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidernoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma viflosum.

Cancers and/or tumors amenable to treatment in accordance with the methods of the instant invention include those accessible via direct intratumoral and/or regional administration, i.e., administration in the region of a target tumor. For example, tumors accessible to administration with a simple syringe injection are readily amenable to treatment. Also amenable to treatment are tumors in which injection requires some imaging and/or guided administration, and/or those in which injection is possible via image-guided percutaneous injection, or catheter/cannula directly into site, or endoscopy.

In some embodiments, the solid tumor comprises a tumor microenvironment that is immunogenic. In some embodiments, immunogenic tumor microenvironments are characterized by greater T-cell infiltration and Th1 cytokine expression. In some embodiments, the solid tumors comprise a tumor microenvironment that is immunologically barren. In some embodiments, immunologically barren tumor microenvironments are characterized by sparse T-cell infiltrate. In some embodiments, the solid tumor is resistant and/or unresponsive to immune checkpoint therapy. Mosley et al. describe these various tumor microenvironments (Mosley et al.

Rational Selection of Syngenic Preclinical Tumor Models for Immunotherapeutic Drug Discovery, Cancer Immunology Research, doi: 10.1158/2326-6066.CIR-16-0114 (2016), incorporated herein by this reference).

In certain embodiments, the mRNAs described herein can be used to modulate tumor microenvironments and/or can be selected for treatment based on the tumor microenvironment in the subject to be treated. In some embodiments, the mRNAs are used to treat a tumor that has an inflamed tumor microenvironment. In some embodiments, the mRNAs are used to treat a tumor that has an immunosuppressive tumor microenvironment. In some embodiments, the mRNAs are used to treat a tumor that has an immunologically barren tumor microenvironment.

In some embodiments, any of the methods described herein comprise administering to the subject a composition of the disclosure (or lipid nanoparticle thereof, or pharmaceutical composition thereof) comprising: an mRNA encoding an IL15 fusion protein described herein.

Compositions of the disclosure are administered to the subject at an effective amount. In general, an effective amount of the composition will allow for efficient production of the encoded polypeptide in the cell. Metrics for efficiency may include polypeptide translation (indicated by polypeptide expression), level of mRNA degradation, and immune response indicators.

The methods of the disclosure for treating a cancer (e.g., solid tumor or disseminated cancer such as a myeloid malignancy) can be used in a variety of clinical or therapeutic applications. For example, the methods can be used to stimulate anti-cancer immunity in a subject with a cancer (e.g., anti-malignancy immunity in a subject with a myeloid malignancy).

In certain embodiments, a subject is administered at least one mRNA composition described herein. In related embodiments, the subject is provided with or administered a nanoparticle (e.g., a lipid nanoparticle) comprising the mRNA. In further related embodiments, the subject is provided with or administered a pharmaceutical composition of the disclosure to the subject. In particular embodiments, the pharmaceutical composition comprises an mRNA as described herein, or it comprises a nanoparticle comprising the mRNA. In particular embodiments, the mRNA is present in a nanoparticle, e.g., a lipid nanoparticle. In particular embodiments, the mRNA or nanoparticle is present in a pharmaceutical composition.

In some embodiments, the mRNA, nanoparticle, or pharmaceutical composition is administered to the patient parenterally. In particular embodiments, the subject is a mammal, e.g., a human. In various embodiments, the subject is provided with an effective amount of the mRNA.

The methods of treating cancer can further include treatment of the subject with additional agents that enhance an anti-tumor response in the subject and/or that are cytotoxic to the tumor (e.g., chemotherapeutic agents). Suitable therapeutic agents for use in combination therapy include small molecule chemotherapeutic agents, including protein tyrosine kinase inhibitors, as well as biological anti-cancer agents, such as anti-cancer antibodies, including but not limited to those discussed further below. Combination therapy can include administering to the subject an immune checkpoint inhibitor to enhance anti-tumor immunity, such as PD-1 inhibitors, PD-L1 inhibitors and CTLA-4 inhibitors, and combinations thereof (e.g., a PD-1 inhibitor+a CTLA-4 inhibitor, a PD-L1 inhibitor+a CTLA-4 inhibitor or a PD-1 inhibitor+a PD-L1 inhibitor). In one embodiment, an agent that modulates an immune checkpoint is an antibody. In another embodiment, an agent that modulates an immune checkpoint is a protein or small molecule modulator. In another embodiment, the agent (such as an mRNA) encodes an antibody modulator of an immune checkpoint. Non-limiting examples of immune checkpoint inhibitors that can be used in combination therapy include pembrolizumab, alemtuzumab, nivolumab, pidilizumab, ofatumumab, MEDIO680 and PDR001, AMP-224, PF-06801591, BGB-A317, REGN2810, SHR-1210, TSR-042, affimer, avelumab (MSB0010718C), atezolizumab (MPDL3280A), durvalumab (MEDI4736), BMS936559, ipilimumab, tremelimumab, AGEN1884, MEDI6469 and MOXR0916.

In one embodiment, a single dose of the mRNA of the disclosure (e.g., an mRNA encoding an IL15 fusion protein described herein) is used in combination with treatment with at least one immune checkpoint inhibitor (e.g., anti-CTLA-4, anti-PD-L1, anti-PD-1 or combinations thereof). In another embodiment, multiple doses (e.g., Q7Dx3) of the mRNA of the disclosure (e.g., an mRNA encoding an IL15 fusion protein described herein) are used in combination with treatment with at least one immune checkpoint inhibitor (e.g., anti-CTLA-4, anti-PD-L1, anti-PD-1 or combinations thereof). Treatment with the immune checkpoint inhibitor(s) can comprise administration of a single dose of the checkpoint inhibitor(s) or, more typically, administration of multiple doses of the checkpoint inhibitors(s).

A pharmaceutical composition including one or more mRNAs of the disclosure may be administered to a subject by any suitable route. In some embodiments, compositions of the disclosure are administered by one or more of a variety of routes, including parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g. by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. In some embodiments, a composition may be administered intravenously, intramuscularly, intradermally, intra-arterially, intratumorally, subcutaneously, or by inhalation. In some embodiments, a composition is administered intramuscularly. However, the present disclosure encompasses the delivery of compositions of the disclosure by any appropriate route taking into consideration likely advances in the sciences of drug delivery. In general, the most appropriate route of administration will depend upon a variety of factors including the nature of the pharmaceutical composition including one or more mRNAs (e.g., its stability in various bodily environments such as the bloodstream and gastrointestinal tract), and the condition of the patient (e.g., whether the patient is able to tolerate particular routes of administration).

In certain embodiments, compositions of the disclosure may be administered at dosage levels sufficient to deliver from about 0.0001 mg/kg to about 10 mg/kg, from about 0.001 mg/kg to about 10 mg/kg, from about 0.005 mg/kg to about 10 mg/kg, from about 0.01 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.1 mg/kg to about 10 mg/kg, from about 1 mg/kg to about 5 mg/kg, from about 2 mg/kg to about 5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, or from about 0.1 mg/kg to about 1 mg/kg in a given dose, where a dose of 1 mg/kg provides 1 mg of mRNA or nanoparticle per 1 kg of subject body weight. In particular embodiments, a dose of about 0.005 mg/kg to about 5 mg/kg of mRNA or nanoparticle of the disclosure may be administrated.

A dose may be administered one or more times per day, in the same or a different amount, to obtain a desired level of mRNA expression and/or effect (e.g., a therapeutic effect). The desired dosage may be delivered, for example, three times a day, two times a day, once a day, every other day, every third day, every week, every two weeks, every three weeks, or every four weeks. In certain embodiments, the desired dosage is delivered using multiple administrations of a single dose (e.g., two, three, four, five, six, seven, eight, nine, ten, eleven, twelve, thirteen, fourteen, or more administrations), referred to as “fractionated dosing”. For example, a desired dosage of 2 mg/kg per week can be administered to a subject over the course of the week by administering 0.67 mg/kg three times a week instead of a single bolus dose of 2 mg/kg. In some embodiments, the fractionated dosing regimen results in enhanced anti-cancer efficacy relative to a single bolus of the same total dose. In some embodiments, the fractionated dosing regimen results in less toxicity relative to a single bolus of the same total dose. In some embodiments, a fractionated dosing regimen is better tolerated by a subject relative to a single bolus dose. In some embodiments, the enhanced efficacy of fractionated dosing is due to greater or enhanced exposure to the mRNA encoded polypeptides. Methods for measuring exposure include, but are not limited to, determining the concentration of the mRNA encoded polypeptides in a sample, determining the half-life of the mRNA encoded polypeptides, and/or determining the area under the curve (AUC) of drug concentration in a sample (e.g., blood plasma) versus time.

In some embodiments, a single dose may be administered, for example, prior to or after a surgical procedure or in the instance of an acute disease, disorder, or condition. The specific therapeutically effective, prophylactically effective, or otherwise appropriate dose level for any particular patient will depend upon a variety of factors including the severity and identify of a disorder being treated, if any; the one or more mRNAs employed; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well known in the medical arts.

In some embodiments, a pharmaceutical composition of the disclosure may be administered in combination with another agent, for example, another therapeutic agent, a prophylactic agent, and/or a diagnostic agent. By “in combination with,” it is not intended to imply that the agents must be administered at the same time and/or formulated for delivery together, although these methods of delivery are within the scope of the present disclosure. For example, one or more compositions including one or more different mRNAs may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions of the disclosure, or imaging, diagnostic, or prophylactic compositions thereof in combination with agents that improve their bioavailability, reduce and/or modify their metabolism, inhibit their excretion, and/or modify their distribution within the body.

Exemplary therapeutic agents that may be administered in combination with the compositions of the disclosure include, but are not limited to, cytotoxic, chemotherapeutic, hypomethylating agents, pro-apoptotic agents, small molecules/kinase inhibitors, and other therapeutic agents including therapeutics approved for cancer, such as AML or MDS, now or at a later date. Cytotoxic agents may include, for example, taxol, cytochalasin B, gramicidin D, ethidium bromide, emetine, mitomycin, etoposide, teniposide, vincristine, vinblastine, colchicine, doxorubicin, daunorubicin, dihydroxyanthracinedione, mitoxantrone, mithramycin, actinomycin D, 1-dehydrotestosterone, glucocorticoids, procaine, tetracaine, lidocaine, propranolol, puromycin, maytansinoids, rachelmycin, and analogs thereof. Radioactive ions may also be used as therapeutic agents and may include, for example, radioactive iodine, strontium, phosphorous, palladium, cesium, iridium, cobalt, yttrium, samarium, and praseodymium. Other therapeutic agents may include, for example, antimetabolites (e.g., methotrexate, 6 mercaptopurine, 6 thioguanine, cytarabine, and 5 fluorouracil, and decarbazine), alkylating agents (e.g., mechlorethamine, thiotepa, chlorambucil, rachelmycin, melphalan, carmustine, lomustine, cyclophosphamide, busulfan, dibromomannitol, streptozotocin, mitomycin C, and cis-dichlorodiamine platinum (II) (DDP), and cisplatin), anthracyclines (e.g., daunorubicin and doxorubicin), antibiotics (e.g., dactinomycin, bleomycin, mithramycin, and anthramycin), and anti-mitotic agents (e.g., vincristine, vinblastine, taxol, and maytansinoids).

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects).

### Kits

In some embodiments, the disclosure provides a kit comprising the mRNAs described herein. For example, in some embodiments the kit comprises an mRNA encoding an IL15 fusion protein described herein. In some embodiments, the disclosure provides a kit comprising a container comprising an mRNA described herein. In some embodiments, the kit comprises the mRNA formulated in a lipid nanoparticle. Accordingly, in some embodiments, a kit comprises a container comprising a lipid nanoparticle encapsulating the mRNAs described herein, and an optional pharmaceutically acceptable carrier, or a pharmaceutical composition, and a package insert comprising instructions for administration of the lipid nanoparticle or pharmaceutical composition. In some embodiments, a kit comprises a container comprising a lipid nanoparticle encapsulating the mRNAs described herein, and an optional pharmaceutically acceptable carrier, or a pharmaceutical composition, and a package insert comprising instructions for administration of the lipid nanoparticle or pharmaceutical composition for treating or delaying progression of a cancer (e.g., solid tumor or disseminated cancer such as a myeloid malignancy) in an individual. In some aspects, the package insert further comprises instructions for administration of the lipid nanoparticle or pharmaceutical composition in combination with a composition comprising a checkpoint inhibitor polypeptide and an optional pharmaceutically acceptable carrier for treating or delaying progression of a cancer (e.g., solid tumor or disseminated cancer such as a myeloid malignancy) in an individual.

In some embodiments, a kit comprises a medicament comprising a lipid nanoparticle encapsulating the mRNAs described herein, and an optional pharmaceutically acceptable carrier, or a pharmaceutical composition, and a package insert comprising instructions for administration of the medicament alone or in combination with a composition comprising a checkpoint inhibitor polypeptide and an optional pharmaceutically acceptable carrier. In some embodiments, a kit comprises a medicament comprising a lipid nanoparticle encapsulating the mRNAs described herein, and an optional pharmaceutically acceptable carrier, or a pharmaceutical composition, and a package insert comprising instructions for administration of the medicament alone or in combination with a composition comprising a checkpoint inhibitor polypeptide and an optional pharmaceutically acceptable carrier for treating or delaying progression of a cancer (e.g., solid tumor or disseminated cancer such as a myeloid malignancy) in an individual. In some aspects, the kit further comprises a package insert comprising instructions for administration of the first medicament prior to, current with, or subsequent to administration of the second medicament for treating or delaying progression of a cancer (e.g., solid tumor or disseminated cancer such as a myeloid malignancy) in an individual.

In some embodiments, the disclosure provides a kit comprising a container comprising an mRNA encoding an IL15 fusion protein described herein, or a composition (e.g., lipid nanoparticle) thereof described herein, and a package insert comprising instructions for administration of the mRNA or composition thereof for treating or delaying progression of a cancer (e.g., solid tumor or disseminated cancer such as a myeloid malignancy) in an individual.

In some embodiments, the disclosure provides a kit comprising a container comprising an lipid nanoparticle encapsulating an mRNA encoding an IL15 fusion protein described herein, and an optionally pharmaceutically acceptable carrier or pharmaceutical composition thereof, and a package insert comprising instructions for administration of the lipid nanoparticle or pharmaceutical composition thereof for treating or delaying progression of a cancer (e.g., solid tumor or disseminated cancer such as a myeloid malignancy) in an individual.

In some embodiments, the disclosure provides a kit comprising a container comprising an mRNA encoding an IL15 fusion protein described herein, or a composition (e.g., lipid nanoparticle) thereof described herein, and a package insert comprising instructions for administration of the mRNA or composition thereof for reducing or inhibiting tumor growth in an individual.

In some embodiments, the disclosure provides a kit comprising a container comprising an lipid nanoparticle encapsulating an mRNA encoding an IL15 fusion protein described herein, and an optionally pharmaceutically acceptable carrier or pharmaceutical composition thereof, and a package insert comprising instructions for administration of the lipid nanoparticle or pharmaceutical composition thereof for reducing or inhibiting tumor growth in an individual.

In some embodiments, the disclosure provides a kit comprising a container comprising an mRNA encoding an IL15 fusion protein described herein, or a composition (e.g., lipid nanoparticle) thereof described herein, and a package insert comprising instructions for administration of the mRNA or composition thereof for inducing an anti-tumor immune response in an individual.

In some embodiments, the disclosure provides a kit comprising a container comprising an lipid nanoparticle encapsulating an mRNA encoding an IL15 fusion protein described herein, and an optionally pharmaceutically acceptable carrier or pharmaceutical composition thereof, and a package insert comprising instructions for administration of the lipid nanoparticle or pharmaceutical composition thereof for inducing an anti-tumor immune response in an individual.

### Definitions

Administering: As used herein, “administering” refers to a method of delivering a composition to a subject or patient. A method of administration may be selected to target delivery (e.g., to specifically deliver) to a specific region or system of a body. For example, an administration may be parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g. by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter.

Approximately, about: As used herein, the terms “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Cancer: As used herein, “cancer” is a condition involving abnormal and/or unregulated cell growth. The term cancer encompasses benign and malignant cancers. Exemplary non-limiting cancers include adrenal cortical cancer, advanced cancer, anal cancer, aplastic anemia, bileduct cancer, bladder cancer, bone cancer, bone metastasis, brain tumors, brain cancer, breast cancer, childhood cancer, cancer of unknown primary origin, Castleman disease, cervical cancer, colorectal cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, myelodysplastic syndrome (including refractory anemias and refractory cytopenias), myeloproliferative neoplasms or diseases (including polycythemia vera, essential thrombocytosis and primary myelofibrosis), liver cancer (e.g., hepatocellular carcinoma), non-small cell lung cancer, small cell lung cancer, lung carcinoid tumor, lymphoma of the skin, malignant mesothelioma, multiple myeloma, myelodysplasia syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor and secondary cancers caused by cancer treatment. In some embodiments, the cancer is liver cancer (e.g., hepatocellular carcinoma), ovarian cancer or colorectal cancer. In other embodiments, the cancer is a blood-based cancer or a hematopoietic cancer. In some embodiments, the cancer is a myeloid malignancy, such as AML.

Cleavable Linker: As used herein, the term “cleavable linker” refers to a linker, typically a peptide linker (e.g., about 5-30 amino acids in length, typically about 10-20 amino acids in length) that can be incorporated into multicistronic mRNA constructs such that equimolar levels of multiple genes can be produced from the same mRNA. Non-limiting examples of cleavable linkers include the 2A family of peptides, including F2A, P2A, T2A and E2A, first discovered in picornaviruses, that when incorporated into an mRNA construct (e.g., between two polypeptide domains) function by making the ribosome skip the synthesis of a peptide bond at C-terminus of the 2A element, thereby leading to separation between the end of the 2A sequence and the next peptide downstream.

Conjugated: As used herein, the term “conjugated,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. In some embodiments, two or more moieties may be conjugated by direct covalent chemical bonding. In other embodiments, two or more moieties may be conjugated by ionic bonding or hydrogen bonding.

Contacting: As used herein, the term “contacting” means establishing a physical connection between two or more entities. For example, contacting a cell with an mRNA or a lipid nanoparticle composition means that the cell and mRNA or lipid nanoparticle are made to share a physical connection. Methods of contacting cells with external entities both in vivo, in vitro, and ex vivo are well known in the biological arts. In exemplary embodiments of the disclosure, the step of contacting a mammalian cell with a composition (e.g., an isolated mRNA, nanoparticle, or pharmaceutical composition of the disclosure) is performed in vivo. For example, contacting a lipid nanoparticle composition and a cell (for example, a mammalian cell) which may be disposed within an organism (e.g., a mammal) may be performed by any suitable administration route (e.g., parenteral administration to the organism, including intravenous, intramuscular, intradermal, and subcutaneous administration). For a cell present in vitro, a composition (e.g., a lipid nanoparticle or an isolated mRNA) and a cell may be contacted, for example, by adding the composition to the culture medium of the cell and may involve or result in transfection. Moreover, more than one cell may be contacted by a nanoparticle composition.

Disseminated cancer: As used herein the term “disseminated cancer” refers to circulating cancer cells within a subject. In some embodiments, disseminated cancer cells have detached from a primary tumor or metastases. In some embodiments, disseminated cancers include those that do not ordinarily form solid tumors and are found throughout the circulation of a subject, e.g., in the blood of a subject. In some embodiments, disseminated cancer cells are those derived from the hematopoietic lineage. In some embodiments, disseminated cancers include those having significant myeloid populations such as myeloid malignancies, along with lymphomas, leukemias etc.

Encapsulate: As used herein, the term “encapsulate” means to enclose, surround, or encase. In some embodiments, a compound, an mRNA, or other composition may be fully encapsulated, partially encapsulated, or substantially encapsulated. For example, in some embodiments, an mRNA of the disclosure may be encapsulated in a lipid nanoparticle, e.g., a liposome.

Effective amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent. In some embodiments, a therapeutically effective amount is an amount of an agent to be delivered (e.g., nucleic acid, drug, therapeutic agent, diagnostic agent or prophylactic agent) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Fragment: A “fragment,” as used herein, refers to a portion. For example, fragments of proteins may include polypeptides obtained by digesting full-length protein isolated from cultured cells or obtained through recombinant DNA techniques.

Heterologous: As used herein, “heterologous” indicates that a sequence (e.g., an amino acid sequence or the nucleic acid that encodes an amino acid sequence) is not normally present in a given polypeptide or nucleic acid. For example, an amino acid sequence that corresponds to a domain or motif of one protein may be heterologous to a second protein.

Hydrophobic amino acid: As used herein, a “hydrophobic amino acid” is an amino acid having an uncharged, nonpolar side chain. Examples of naturally occurring hydrophobic amino acids are alanine (Ala), valine (Val), leucine (Leu), isoleucine (Ile), proline (Pro), phenylalanine (Phe), methionine (Met), and tryptophan (Trp).

Identity: As used herein, the term “identity” refers to the overall relatedness between polymeric molecules, e.g., between nucleic acid molecules (e.g., DNA molecules and/or RNA molecules) and/or between polypeptide molecules. Calculation of the percent identity of two mRNA sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux et al., Nucleic Acids Research, 12(1): 387,1984, BLASTP, BLASTN, and FASTA, Altschul, S. F. et al., J. Molec. Biol., 215, 403, 1990.

Immune checkpoint inhibitor: An “immune checkpoint inhibitor” or simply “checkpoint inhibitor” refers to a molecule that prevents immune cells from being turned off by cancer cells. As used herein, the term checkpoint inhibitor refers to polypeptides (e.g., antibodies) or polynucleotides encoding such polypeptides (e.g., mRNAs) that neutralize or inhibit inhibitory checkpoint molecules such as cytotoxic T-lymphocyte-associated protein 4 (CTLA-4), programmed death 1 receptor (PD-1), or PD-1 ligand 1 (PD-L1).

Immune response: The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues. In some cases, the administration of a nanoparticle comprising a lipid component and an encapsulated therapeutic agent can trigger an immune response, which can be caused by (i) the encapsulated therapeutic agent (e.g., an mRNA), (ii) the expression product of such encapsulated therapeutic agent (e.g., a polypeptide encoded by the mRNA), (iii) the lipid component of the nanoparticle, or (iv) a combination thereof.

Insertion: As used herein, an “insertion” or an “addition” refers to a change in an amino acid or nucleotide sequence resulting in the addition of one or more amino acid residues or nucleotides, respectively, to a molecule as compared to a reference sequence, for example, the sequence found in a naturally-occurring molecule.

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Liposome: As used herein, by “liposome” is meant a structure including a lipid-containing membrane enclosing an aqueous interior. Liposomes may have one or more lipid membranes. Liposomes include single-layered liposomes (also known in the art as unilamellar liposomes) and multi-layered liposomes (also known in the art as multilamellar liposomes).

Linker: As used herein, a “linker” (including a membrane linker, a subunit linker, and a heterologous polypeptide linker as referred to herein) refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end, and to a payload, e.g., a detectable or therapeutic agent, at a second end. The linker can be of sufficient length as to not interfere with incorporation into a nucleic acid sequence. The linker can be used for any useful purpose, such as to form polynucleotide multimers (e.g., through linkage of two or more chimeric polynucleotides molecules or IVT polynucleotides) or polynucleotides conjugates, as well as to administer a payload, as described herein. Examples of chemical groups that can be incorporated into the linker include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers and derivatives thereof., Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bond include an amido bond can be cleaved for example by the use of tris(2-carboxyethyl)phosphine (TCEP), or other reducing agents, and/or photolysis, as well as an ester bond can be cleaved for example by acidic or basic hydrolysis.

Metastasis: As used herein, the term “metastasis” means the process by which cancer spreads from the place at which it first arose as a primary tumor to distant locations in the body. A secondary tumor that arose as a result of this process may be referred to as “a metastasis.” mRNA: As used herein, an “mRNA” refers to a messenger ribonucleic acid. An mRNA may be naturally or non-naturally occurring. For example, an mRNA may include modified and/or non-naturally occurring components such as one or more nucleobases, nucleosides, nucleotides, or linkers. An mRNA may include a cap structure, a chain terminating nucleoside, a stem loop, a polyA sequence, and/or a polyadenylation signal. An mRNA may have a nucleotide sequence encoding a polypeptide. Translation of an mRNA, for example, in vivo translation of an mRNA inside a mammalian cell, may produce a polypeptide. Traditionally, the basic components of an mRNA molecule include at least a coding region, a 5′-untranslated region (5′-UTR), a 3′UTR, a 5′ cap and a polyA sequence. microRNA (miRNA): As used herein, a “microRNA (miRNA)” is a small non-coding RNA molecule which may function in post-transcriptional regulation of gene expression (e.g., by RNA silencing, such as by cleavage of the mRNA, destabilization of the mRNA by shortening its polyA tail, and/or by interfering with the efficiency of translation of the mRNA into a polypeptide by a ribosome). A mature miRNA is typically about 22 nucleotides long. microRNA-122 (miR-122): As used herein, “microRNA-122 (miR-122)” refers to any native miR-122 from any vertebrate source, including, for example, humans, unless otherwise indicated. miR-122 is typically highly expressed in the liver, where it may regulate fatty-acid metabolism. miR-122 levels are reduced in liver cancer, for example, hepatocellular carcinoma. miR-122 is one of the most highly-expressed miRNAs in the liver, where it regulates targets including but not limited to CAT-1, CD320, AldoA, Hjv, Hfe, ADAM10, IGFR1, CCNG1, and ADAM17. Mature human miR-122 may have a sequence of AACGCCAUUAUCACACUAAAUA (SEQ ID NO: 112, corresponding to hsa-miR-122-3p) or UGGAGUGUGACAAUGGUGUUUG (SEQ ID NO: 114, corresponding to hsa-miR-122-5p). microRNA-21 (miR-21): As used herein, “microRNA-21 (miR-21)” refers to any native miR-21 from any vertebrate source, including, for example, humans, unless otherwise indicated. miR-21 levels are increased in liver cancer, for example, hepatocellular carcinoma, as compared to normal liver. Mature human miR-21 may have a sequence of UAGCUUAUCAGACUGAUGUUGA (SEQ ID NO: 116, corresponding to hsa-miR-21-5p) or 5′- CAACACCAGUCGAUGGGCUGU - 3′ (SEQ ID NO: 117, corresponding to hsa-miR-21-3p). microRNA-142 (miR-142): As used herein, “microRNA-142 (miR-142)” refers to any native miR-142 from any vertebrate source, including, for example, humans, unless otherwise indicated. miR-142 is typically highly expressed in myeloid cells. Mature human miR-142 may have a sequence of UGUAGUGUUUCCUACUUUAUGGA (SEQ ID NO: 118, corresponding to hsa-miR-142-3p) or CAUAAAGUAGAAAGCACUACU (SEQ ID NO: 119, corresponding to hsa-miR-142-5p).

microRNA (miRNA) binding site: As used herein, a “microRNA (miRNA) binding site” refers to a miRNA target site or a miRNA recognition site, or any nucleotide sequence to which a miRNA binds or associates. In some embodiments, a miRNA binding site represents a nucleotide location or region of an mRNA to which at least the “seed” region of a miRNA binds. It should be understood that “binding” may follow traditional Watson-Crick hybridization rules or may reflect any stable association of the miRNA with the target sequence at or adjacent to the microRNA site.

miRNA seed: As used herein, a “seed” region of a miRNA refers to a sequence in the region of positions 2-8 of a mature miRNA, which typically has perfect Watson-Crick complementarity to the miRNA binding site. A miRNA seed may include positions 2-8 or 2-7 of a mature miRNA. In some embodiments, a miRNA seed may comprise 7 nucleotides (e.g., nucleotides 2-8 of a mature miRNA), wherein the seed-complementary site in the corresponding miRNA binding site is flanked by an adenine (A) opposed to miRNA position 1. In some embodiments, a miRNA seed may comprise 6 nucleotides (e.g., nucleotides 2-7 of a mature miRNA), wherein the seed-complementary site in the corresponding miRNA binding site is flanked by an adenine (A) opposed to miRNA position 1. When referring to a miRNA binding site, an miRNA seed sequence is to be understood as having complementarity (e.g., partial, substantial, or complete complementarity) with the seed sequence of the miRNA that binds to the miRNA binding site.

Modified: As used herein “modified” refers to a changed state or structure of a molecule of the disclosure. Molecules may be modified in many ways including chemically, structurally, and functionally. In one embodiment, the mRNA molecules of the present disclosure are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

Myeloid Malignancy: As used herein “myeloid malignancy” refers to both chronic and acute clonal disorders that are characterized by acquired somatic mutation(s) in hematopoietic progenitor cells, such as myelodysplastic disorders (MDS) and myeloproliferative neoplasms (MPN). Exemplary myeloid malignancies include, but are not limited to, acute myeloid leukemia (AML) and chronic meylomonocytic leukemia (CMML). Further, MPNs comprise a variety of disorders, such as chronic myeloid leukemia (CML) and non-CML MPNs such as polycythemia vera (PV), essential thrombocythemia (ET) and primary myelofibrosis (PMF).

Nanoparticle: As used herein, “nanoparticle” refers to a particle having any one structural feature on a scale of less than about 1000 nm that exhibits novel properties as compared to a bulk sample of the same material. Routinely, nanoparticles have any one structural feature on a scale of less than about 500 nm, less than about 200 nm, or about 100 nm. Also routinely, nanoparticles have any one structural feature on a scale of from about 50 nm to about 500 nm, from about 50 nm to about 200 nm or from about 70 to about 120 nm. In exemplary embodiments, a nanoparticle is a particle having one or more dimensions of the order of about 1-1000 nm. In other exemplary embodiments, a nanoparticle is a particle having one or more dimensions of the order of about 10-500 nm. In other exemplary embodiments, a nanoparticle is a particle having one or more dimensions of the order of about 50-200 nm. A spherical nanoparticle would have a diameter, for example, of between about 50-100 or 70-120 nanometers. A nanoparticle most often behaves as a unit in terms of its transport and properties. It is noted that novel properties that differentiate nanoparticles from the corresponding bulk material typically develop at a size scale of under 1000 nm, or at a size of about 100 nm, but nanoparticles can be of a larger size, for example, for particles that are oblong, tubular, and the like. Although the size of most molecules would fit into the above outline, individual molecules are usually not referred to as nanoparticles.

Nucleic acid: As used herein, the term “nucleic acid” is used in its broadest sense and encompasses any compound and/or substance that includes a polymer of nucleotides. These polymers are often referred to as polynucleotides. Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof. Operably linked: As used herein, the phrase “operably linked” refers to a functional connection between two or more molecules, constructs, transcripts, entities, moieties or the like.

Patient: As used herein, “patient” refers to a subject who may seek or be in need of treatment, requires treatment, is receiving treatment, will receive treatment, or a subject who is under care by a trained professional for a particular disease or condition. In particular embodiments, a patient is a human patient. In some embodiments, a patient is a patient suffering from cancer (e.g., liver cancer or colorectal cancer).

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio

Pharmaceutically acceptable excipient: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P.H. Stahl and C.G. Wermuth (eds.), Wiley-VCH, 2008, and Berge et al., Journal of Pharmaceutical Science, 66, 1-19 (1977), each of which is incorporated herein by reference in its entirety.

Polypeptide: As used herein, the term “polypeptide” or “polypeptide of interest” refers to a polymer of amino acid residues typically joined by peptide bonds that can be produced naturally (e.g., isolated or purified) or synthetically.

Subject: As used herein, the term “subject” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, a subject may be a patient.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Targeting moiety: As used herein, a “targeting moiety” is a compound or agent that may target a nanoparticle to a particular cell, tissue, and/or organ type.

Therapeutic Agent: The term “therapeutic agent” refers to any agent that, when administered to a subject, has a therapeutic, diagnostic, and/or prophylactic effect and/or elicits a desired biological and/or pharmacological effect.

Transfection: As used herein, the term “transfection” refers to methods to introduce a species (e.g., a polynucleotide, such as an mRNA) into a cell.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Tumor Microenvironment”: As used herein, “tumor microenvironment” refers to the cellular compositions within a tumor with respect to the presence or absence of infiltrating immune and/or inflammatory cells, as well as the type(s) of such cells within the tumor. In some embodiments, a tumor microenvironment is an “inflamed tumor microenvironment”, which refers to the presence of immune and/or inflammatory cells infiltrated into the tumor, with the predominant cell type being granulocytes. In some embodiments, a tumor microenvironment is an “immunosuppressive tumor microenvironment”, which refers to the presence of immune and/or inflammatory cells infiltrated into the tumor, with the predominant cell types being monocytic cells and macrophages. In some embodiments, a tumor microenvironment is an “immunologically barren tumor microenvironment”, which refers to an absence of significant infiltration into the tumor of immune and/or inflammatory cells.

Preventing: As used herein, the term “preventing” refers to partially or completely inhibiting the onset of one or more symptoms or features of cancer, including preventing a relapse or recurrence after successful treatment.

Tumor: As used herein, a “tumor” is an abnormal growth of tissue, whether benign or malignant.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

### Equivalents and Scope

Those skilled in the art will recognize, or be able to ascertain using no more than routine experimentation, many equivalents to the specific embodiments in accordance with the disclosure described herein. The scope of the present disclosure is not intended to be limited to the Description below, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

### EXAMPLES

### Example 1: In Vitro Expression of mRNA-Encoded Chimeric IL15 Fusion Proteins

Expression of mRNAs encoding fusion proteins containing human IL15 (hIL15) were evaluated in vitro. The fusion proteins contained the following components:
(i) a mature form of hIL15 having an amino acid sequence set forth by SEQ ID NO: 16;
(ii) a 66 amino acid residue region of the hIL15Rα extracellular domain spanning the Sushi domain having the amino acid sequence set forth by SEQ ID NO: 18 (referred to as “SushiS” throughout the Examples and Figures) or a 78 amino acid residue region of the hIL15Rα extracellular domain spanning the Sushi domain having the amino acid sequence set forth by SEQ ID NO: 17 (referred to as “SushiL” throughout the Examples and Figures);
(iii) human apolipoprotein A-1 (referred to in the Examples and Figures as “ApoA”), as set forth by SEQ ID NO: 14; and
(iv) a signal peptide from the human IgG heavy chain having the amino acid sequence of SEQ ID NO: 13 (referred to as “SP2” throughout the Examples and Figures) or a signal peptide from human IL15Rα having the amino acid sequence of SEQ ID NO: 12 (referred to as “SP1” throughout the Examples and Figures).

Each fusion protein encoded an N-terminal signal peptide (SP1 or SP2) followed by components (i)-(iii) in different arrangements. The components were either directly fused or joined by a linker (e.g., a Gly-Ser linker). The fusion proteins that were evaluated are indicated in Table 1 and shown schematically by FIG. 1A.

IL15 fusion proteins containing hIL15, a Sushi domain of the hIL15Rα, and an IgG-Fc fragment are undergoing clinical development (see, e.g., Chen, et al (2015) J. Immunother Cancer 3(Suppl2):P347); Furuya, et al (2019) J Transl Med 17:29). To evaluate mRNA encoding IL15 fusion protein variants containing Apo-A, a reference mRNA encoding an IL15 fusion protein containing an Fc domain was used. The reference mRNA encoded a fusion protein containing N-terminus to C-terminus: an SP2 signal peptide set forth by SEQ ID NO: 13, Fc domain set forth by SEQ ID NO: 15, SushiL set forth by SEQ ID NO: 17, and mature hIL15 polypeptide set forth by SEQ ID NO: 16. The reference fusion protein is also indicated in Table 1 and shown schematically by FIG. 1A.

TABLE 1
Amino acid and nucleotide sequences
of chimeric IL15 fusion proteins

| | SEQ ID NO | SEQ ID NO |
|---|---|---|
| Fusion Protein | (amino acid) | (DNA) |
| SP2-Fc-SushiL-IL15 | 6 | 5 |
| (reference IL15 fusion protein) | | |
| SP2-ApoA-SushiS-IL15 | 4 | 3 |
| SP2-ApoA-SushiL-IL15 | 2 | 1 |
| SP1-SushiS-IL15-ApoA | 9 | 8 |
| SP1-SushiL-IL15-ApoA | 11 | 10 |

The mRNAs were constructed with an ORF sequence encoding the fusion protein. The mRNA sequences included the 5′UTR and 3′UTR having the sequences set forth in Table 2 below.

TABLE 2
5′UTR and 3′UTR sequences of the IL15 Fusion Protein-encoding mRNAs

| | | SEQ | |
|---|---|---|---|
| | | ID | |
| Identifier | Name | NO | Nucleotide sequence |
| 5′UTR | 5′UTR v1.1 | 19 | GGGAAAUAAGAGAGAAAAGAAGAGUAAGAAGAAAUAUAA |
| | | | GACCCCGGCGCCGCCACC |
| 3′UTR | 3′UTR v1.1 | 20 | UGAUAAUAGGCUGGAGCCUCGGUGGCCUAGCUUCUUGCC |
| | | | CCUUGGGCCUCCCCCCAGCCCCUCCUCCCCUUCCUGCAC |
| | | | CCGUACCCCCGUGGUCUUUGAAUAAAGUCUGAGUGGGCG |
| | | | GC |

The mRNA sequences were prepared by in vitro-transcription and fully modified with N1-methyl pseudouridine (m^{l}ψ) in place of uracil. Furthermore, the mRNAs were synthesized to have a polyA-tail, and a Cap 1 structure.

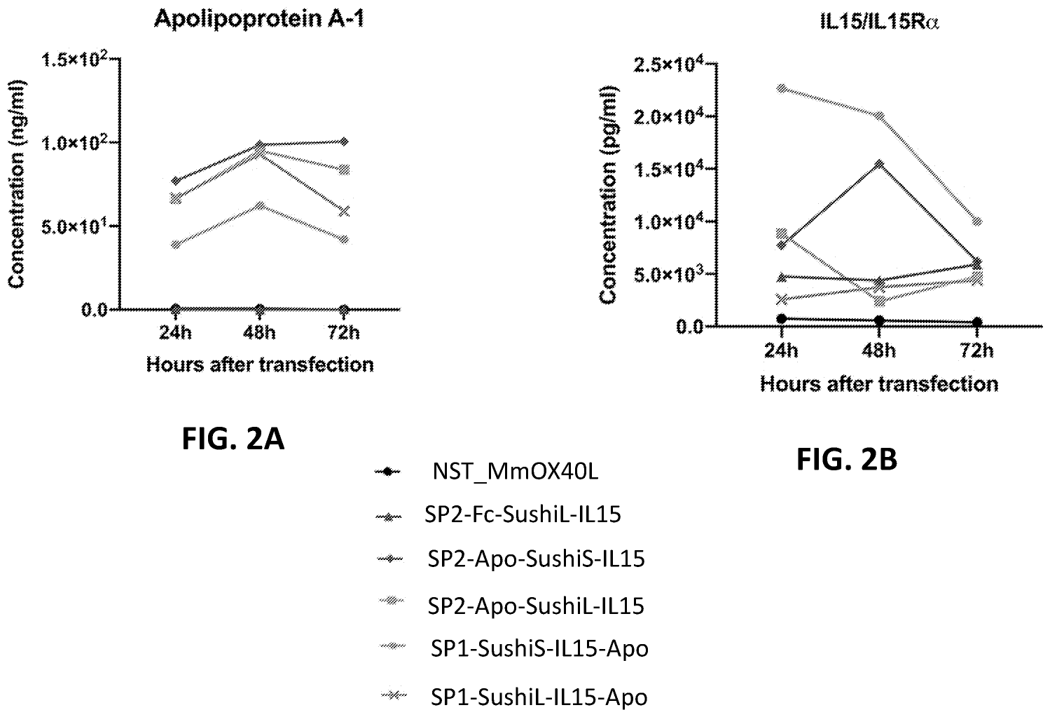

The expression of the mRNA-encoded fusions proteins was evaluated. An mRNA encoding non-translatable (NST) murine OX40 ligand was used as a negative control. Briefly, 1 g of mRNA was complexed with TransIT and transfected into HEK293T cells. Cell supernatant was collected at 24 hours, 48 hours, and 72 hours following transfection. Levels of human ApoA and IL15/IL15Rα were quantified by an ELISA. The IL15/IL15Rα ELISA is specific to the IL15/IL15Rα complex. As shown in FIG. 2A, expression of human ApoA was detected for cells transfected with mRNA encoding fusion proteins containing ApoA at each of the time points tested. As shown in FIG. 2B, expression of IL15/IL15Rα was detected in cells transfected with mRNA encoding chimeric IL15 fusions proteins, but not for cells transfected with the negative control mRNA.

### Example 2: In Vitro Cellular Proliferation Induced by mRNA Encoding Chimeric IL15 Fusion Proteins

A cellular proliferation assay was used to determine whether the mRNA described in Example 1 generated bioactive IL15 following expression. HEK-293T cells were transfected with the mRNA encoding the different IL-15 variants complexed in TransIt, and 24 hours later supernatants were stored at −80° C. Proliferation was measured in CTLL2 cells and Mo7e cells. CTLL2 cells are a murine cytotoxic T cell line that have positive expression of both the IL15Rα chain and the IL15βγ complex. Mo7e cells are human megakaryocytic leukemic cells that only express the IL15βγ complex. Proliferation of both cell lines can be induced by the presence of IL15 fused to an IL15Rα Sushi domain. Proliferation of Mo7e cells indicates the activity of the IL15Rα domain.

Briefly, 1×10⁴ cells (CTLL-2 or Mo7e) were treated with supernatants from HEK-293T cells transfected with mRNA complexed with TransIT mRNA. Negative control cells were transfected with mRNA encoding NST murine OX40 ligand. At 48 hours following treatment, the cells were pulsed with 0.5 μCi of tritiated thymidine ([³H]TdR). At 8 hours thereafter, the cells were harvested and ³HTdR was quantified using a scintillation counter in order to quantify proliferation. As the signal peptide is cleaved during secretion, the fusion protein present in the supernatant corresponds to the mature fusion protein (i.e., the mRNA encoded protein minus the signal peptide) and the assay measured cell proliferation induced by the mature protein. For example, transfection with an mRNA having an ORF encoding the SP2-Fc-SushiL-IL15 reference protein secrete a fusion protein that is Fc-SushiL-IL15 and the cellular proliferation assay was used to measure activity of the mature protein.

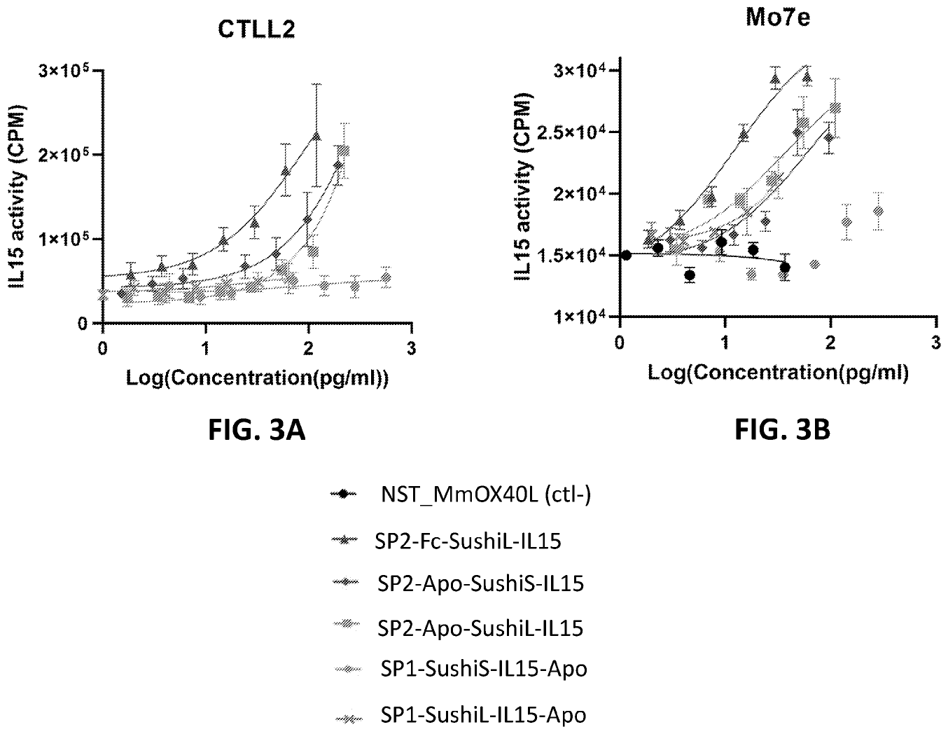

As shown in FIGS. 3A-3B, the highest levels of cellular proliferation were observed for the reference mRNA (EC50 91.57 nM in CTLL2 and 12.37 nM in Mo7e). Activity of mRNA-encoding IL15 fusion proteins having an N-terminal ApoA domain (SP2-Fc-SushiL-IL15; SP2-Apo-SushiS-IL15; SP2-Apo-SushiL-IL15) was also detected in both cell lines. However, cellular proliferation was comparable to the negative control for mRNA-encoded IL15 fusion proteins having a C-terminal ApoA domain (SP1-SushiS-IL15-Apo; SP1-SushiL-IL15-Apo). These data indicate that the arrangement of components in the IL15 fusion protein contributes to IL15 bioactivity.

### Example 3: Biodistribution Analysis of TransIT-Complexed mRNA Encoding Luciferase

The kinetics and biodistribution of expression of mRNA complexed with TransIT was evaluated following in vivo administration by intravenous (tail vein) injection. mRNA encoding luciferase was initially used to establish mRNA expression distributed across various tissues. Briefly, mRNA with an ORF encoding luciferase was complexed with TransIT. Negative control mRNA encoded a non-bioluminescent protein. The TransIT-complexed mRNA was administered to C57BL/6 mice by tail vein injection at a dose of 10 μg mRNA per mouse. At various time points following injection, whole-body luciferase activity was measured by in vivo bioluminescence imaging using a D-luciferin/IVIS protocol. At 24 hours following injection, mice euthanized and tissues were harvested for ex vivo quantification of luciferase expression on a per-tissue basis.

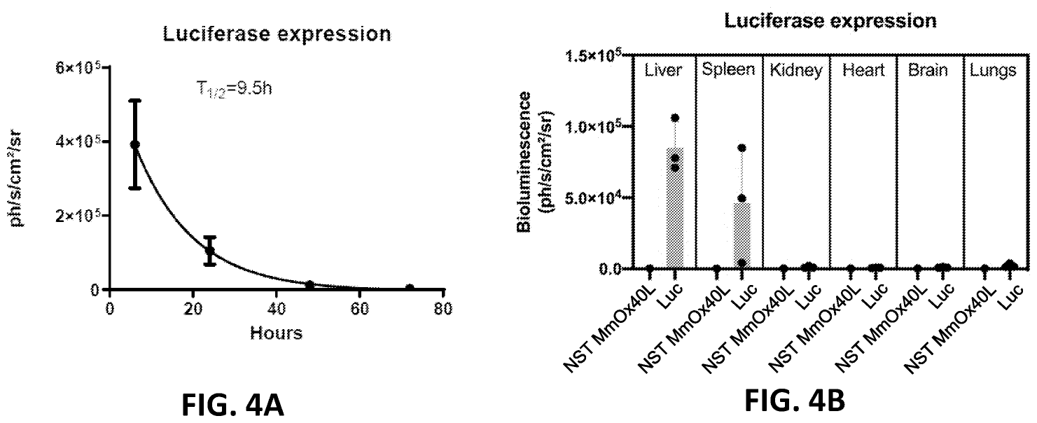

As shown in FIG. 4A, based on measuring luciferase activity over time following administration of the TransIT-complexed mRNA, the half-life of luciferase expression was determined to be approximately 9.5 hours. Expression was undetectable at 72 hours. As shown in FIG. 4B, the liver and spleen from mice administered the luciferase-mRNA had the highest luciferase expression compared to the background luminescence of tissues harvested from control mice. Luciferase expression was minimal in kidney, heart, brain, and lung. These data demonstrate that TransIT-complexed mRNA administered by intravenous injection results in detectable expression of encoded luciferase, predominantly in the liver and spleen.

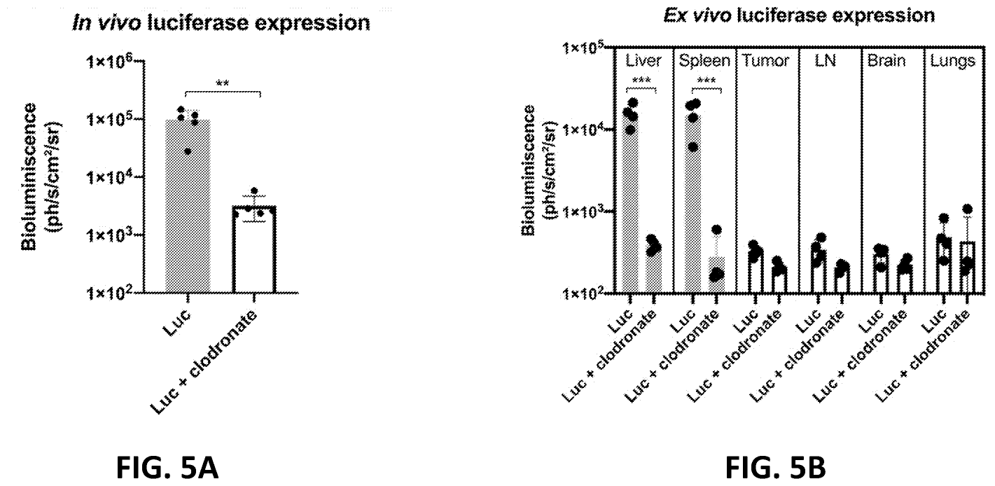

Expression of mRNA was further evaluated in the absence of macrophages (e.g., spleen macrophages, Kupffer cells). To deplete macrophages, mice were administered clodronate liposomes by tail vein injection (200 μL/20 μg per mouse). At 24 hours following clodronate administration, the mice received mRNA encoding luciferase complexed with TransIT (10 g mRNA/mouse). Control mice received TransIT-formulated luciferase-mRNA only. At 24 hours following luciferase-mRNA administration, whole-body luciferase activity was quantified by IVIS using a D-luciferin protocol and tissues were harvested to quantify luciferase activity on a per-tissue basis. As shown in FIG. 5A, while luciferase activity was detectable in mice that received luciferase-mRNA only, luciferase activity was significantly abrogated in mice that received the clodronate liposomes followed by luciferase-mRNA. As shown in FIG. 5B, the mice that received luciferase-mRNA only had detectable luciferase activity in the liver and spleen. However, mice that first received the clodronate liposomes had significantly reduced luciferase activity in both liver and spleen. These results indicate macrophages (e.g., spleen macrophages, Kupffer cells) are necessary for uptake and expression of the mRNA complexed in TransIT following in vivo administration.

### Example 4: In Vivo Expression of mRNA Encoding Chimeric IL15 Fusion Proteins

Having established an administration protocol that yields mRNA expression in vivo, the expression of mRNA encoding chimeric IL15 fusion proteins as described in Example 1 was evaluated. The mRNAs were complexed to TransIT prior to administration.

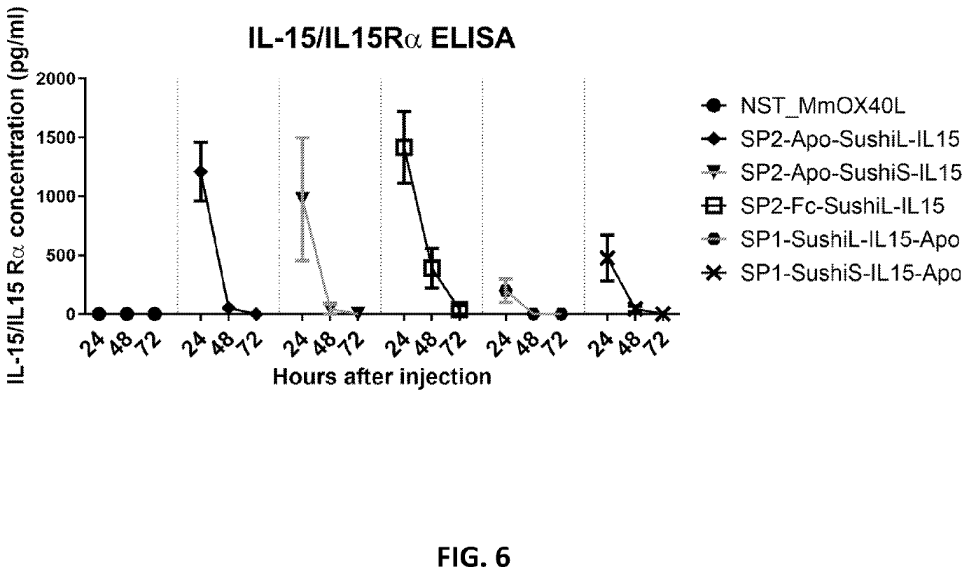

The TransIT-complexed mRNA was administered to C57BL/6 mice by intravenous (tail vein) injection. Control mice received NST murine OX40L mRNA formulated in TransIT. Each mouse was administered a dose of 5 μg mRNA. Serum was collected at 24 hours, 48 hours, and 72 hours following administration and evaluated for level of IL15/IL15Rα by ELISA. As shown in FIG. 6, elevated levels were detected for mice administered reference mRNA (SP2-Fc-SushiL-IL15) and mRNA encoding chimeric IL15 fusion protein having an N-terminal ApoA domain (SP2-Apo-SushiL-Il15; SP2-Apo-SushiS-IL15).

### Example 5: Immune Profile Following In Vivo Administration of mRNA Encoding Chimeric IL15 Fusion Proteins

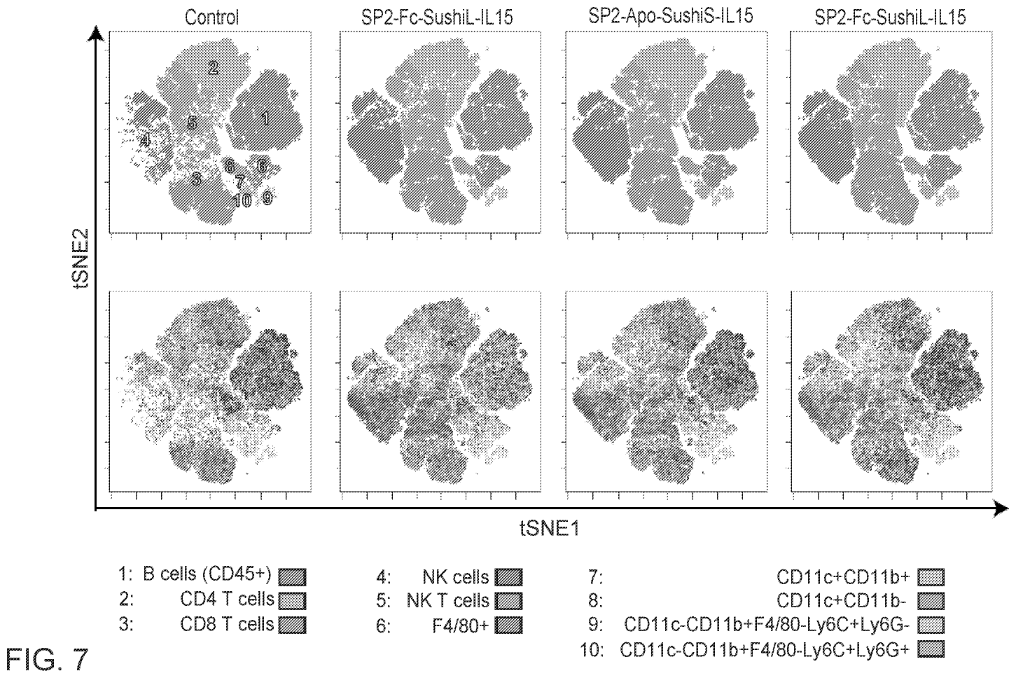

The immune profile of mice administered mRNA encoding chimeric IL15 fusion proteins was evaluated. The mRNA encoded SP2-Apo-SushiL-IL15, SP2-Apo-SushiS-IL15, or SP2-Fc-SushiL-IL15 as described in Example 1. Control mice received mRNA encoding non-translatable (NST) murine OX40 ligand. The IL15 mRNAs and control mRNA were formulated using the TransIT preparation. The TransIT-complexed mRNA was administered to C57BL/6 mice by intravenous (tail vein) injection. Each mouse received a dose of 10 μg mRNA. Five days later, livers were harvested from the mice and flow cytometry was performed to identify immune cell populations that were B cells, CD4 T cells, CD8 T cells, NK cells, NKT cells, macrophages (F4/80+), dendritic cells (CD1lc+CD1lb+ and CD1lc+CD1ib-), and myeloid-derived suppressor cells (CD1ic-CD1lb+F4/80-Ly6C+Ly6G- and CD1ic-CD1lb+F4/80-Ly6C+Ly6G+). t-SNE clustering of the flow cytometry data was performed to identify immune cell subpopulations is shown in FIG. 7. Staining for Ki67 was evaluated as a measure of proliferation for each immune cell subpopulation. Mice administered the chimeric IL15 fusion proteins exhibited increased levels of expansion and proliferation among NK cells, NK T cells, and CD8 T cells. Moreover, the level of proliferation among these cell subsets was similar in mice that received mRNA encoding the Apo-containing chimeric IL15 fusion proteins (SP2-Apo-SushiL-IL15 or SP2-Apo-SushiS-IL15) to mice that received reference mRNA encoding the Fc-containing chimeric IL15 fusion protein.

Further evaluation of immune cell subpopulations was performed by flow cytometry. Briefly, mice were administered the TransIT-complexed mRNA described above by intravenous (tail vein) injection. Each mouse received a dose of 10 μg mRNA. Control mice received TransIT-complexed mRNA encoding NST murine OX40 ligand. Livers were harvested from the mice immediately following injection, at 2 days following injection, or at 5 days following injection, and labeled for flow cytometry analysis. The markers indicated in the below Table 3 were used to identify NK cell, T cell, and macrophage populations. Ki67 was also measured as a marker of cell proliferation and intracellular cytokine staining was performed to measure expression of IFNγ.

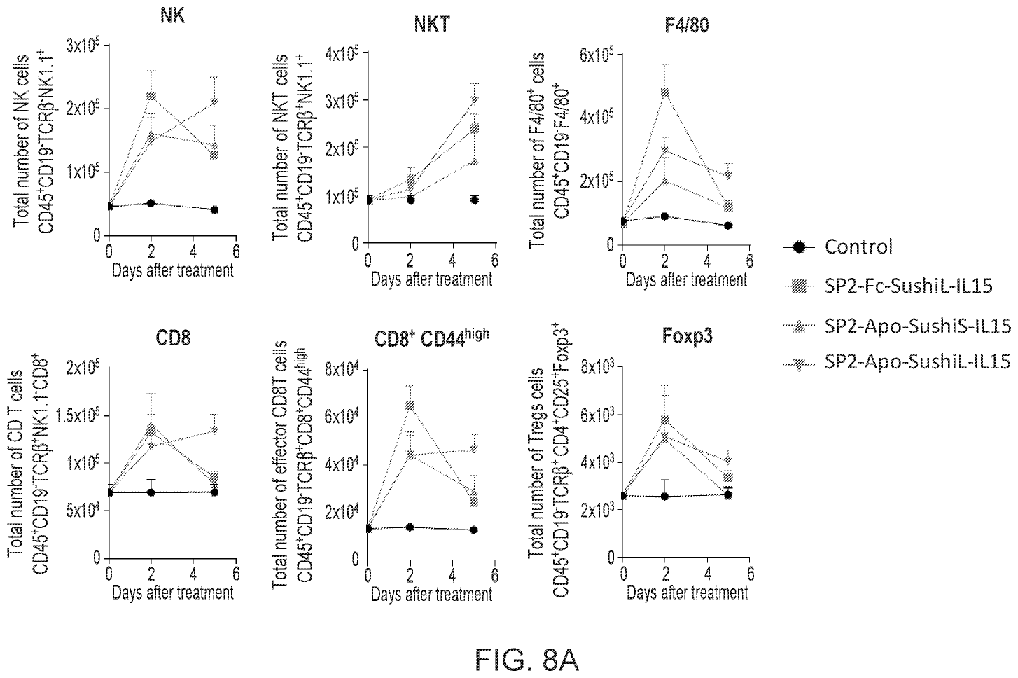

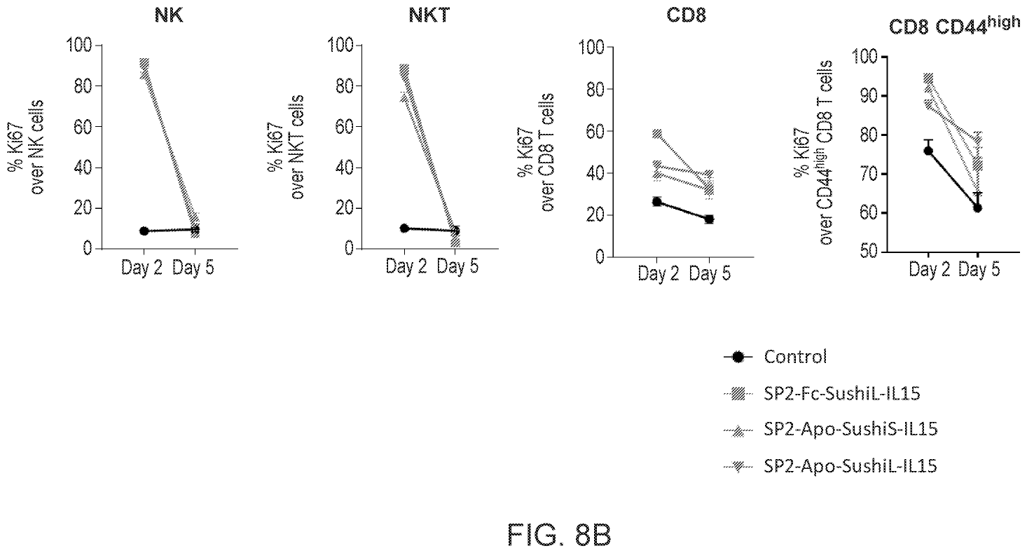

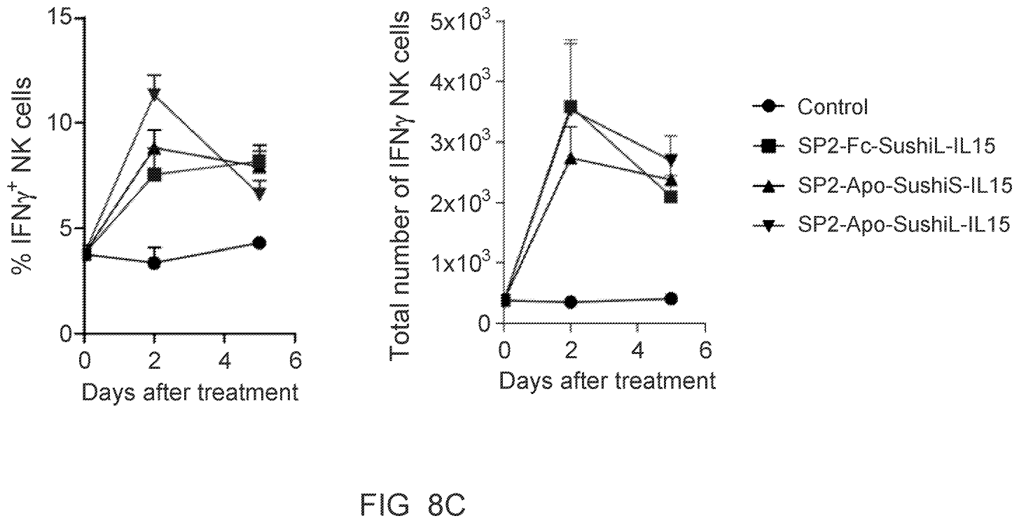

As shown in FIG. 8A, mice that received mRNA encoding the chimeric IL15 fusion proteins had elevated levels of the indicated immune cell populations compared to control mice.

In particular, administration of mRNA encoding SP2-Apo-SushiL-IL15 was found to maintain expansion of NK and CD8 T cell populations for the duration of the study (up to 5 days following injection).

As shown in FIG. 8B, mice that received mRNA encoding chimeric IL15 fusion protein had elevated Ki67 expression in NK cells, NKT cells, CD8 T cells, and effector CD8 T cells at 2 days following administration as compared to control mice.

As shown in FIG. 8C, mice that received mRNA encoding chimeric IL15 fusion protein had elevated IFN7 expression in NK cells. Shown is the percentage of NK cells expressing IFN7 (left panel) and the total number of IFN7-expressing NK cells.

TABLE 3
Immune Cell Profiling by Flow Cytometry

| Immune Cell | |
|---|---|
| Population | Identifying Markers |
| NK cells | CD45+ CD19neg TCRβneg NK1.1+ |
| NKT cells | CD45+ CD19neg TCRβ+ NK1.1+ |
| F4/80+ cells | CD45+ CD19neg F4/80+ |
| CD8 T cells | CD45+ CD19neg TCRβ+ NK1.1neg CD8+ |
| Effector | CD45+ CD19neg TCRβ+ NK1.1neg CD8+ CD44hi |
| CD8 T cells | |
| Tregs | CD45+ CD19neg TCRβ+ CD4+ CD25+ Foxp3+ |

### Example 6: mRNA Encoding Chimeric IL15 Fusion Proteins Reduce Tumor Burden in Mouse Models

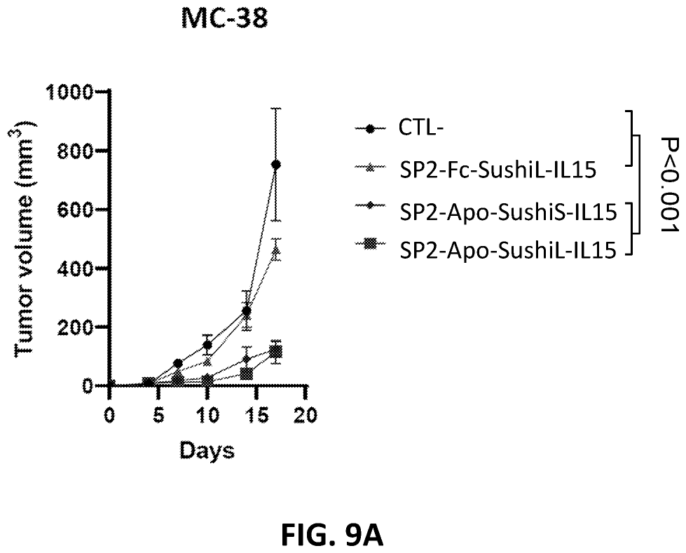

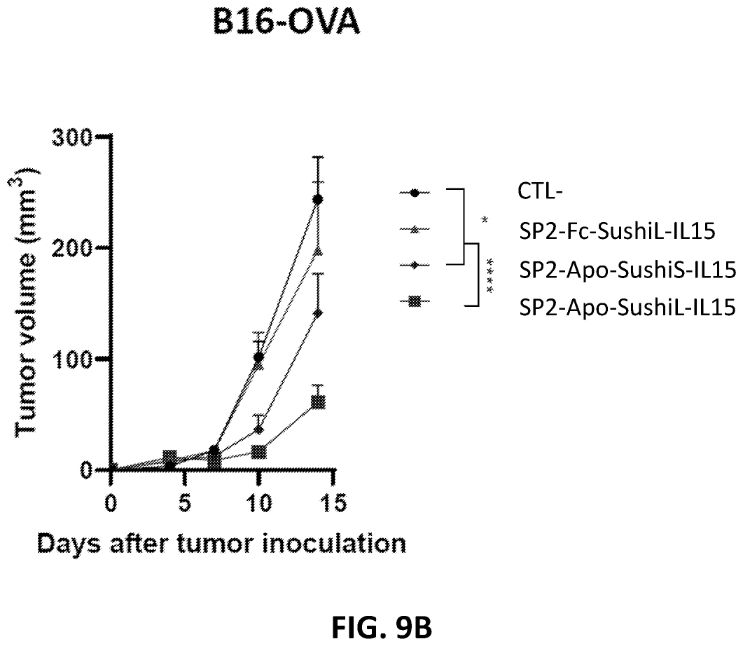

Antitumor effects of mRNA encoding chimeric IL15 fusion proteins were evaluated in tumor-bearing mice. The mRNAs were evaluated in a mouse flank tumor models established using MC38 colon tumor cells or B16-Ova melanoma cells. For the MC38 tumor model, tumor cells were inoculated and on days 5 and 10 following tumor cell implantation, mice received an intravenous injection of the TransIT-complexed mRNA encoding chimeric IL15 fusion proteins. The mRNA encoded SP2-Apo-SushiL-IL15, SP2-Apo-SushiS-IL15, or SP2-Fc-SushiL-IL15 fusion proteins as described in Example 1. Each mRNA was formulated using the TransIT. The mice received a dose of 10 μg mRNA per mouse. Control mice received TransIT-complexed mRNA encoding NST murine OX40 ligand. Tumor growth (mean diameter) was measured over time. As shown in FIG. 9A, tumor growth was controlled in mice administered mRNA encoding SP2-Apo-SushiS-IL15 or SP2-Apo-SushiL-IL15. In contrast, mice administered reference mRNA encoding SP2-Fc-SushiL-IL15 had comparable tumor growth to control mice.

For the B16Ova tumor model, mice were inoculated with B16Ova cells and on days 5 and 10 following tumor cell injection, the mice received a TransIT-complexed mRNA described above. Control mice received TransIT-complexed mRNA encoding NST murine OX40 ligand. Tumor growth (mean diameter) was measured over time. As shown in FIG. 9B, tumor growth was controlled in mice administered mRNA encoding Apo-containing fusion proteins, particularly for mRNA encoding SP2-Apo-SushiL-IL15. In contrast, mice administered reference mRNA encoding SP2-Fc-SushiL-IL15 had comparable tumor growth to control mice. The antitumor effect of SP2-Apo-SushiL-IL15 was higher than the effect of SP2-Fc-SushiL-IL15.

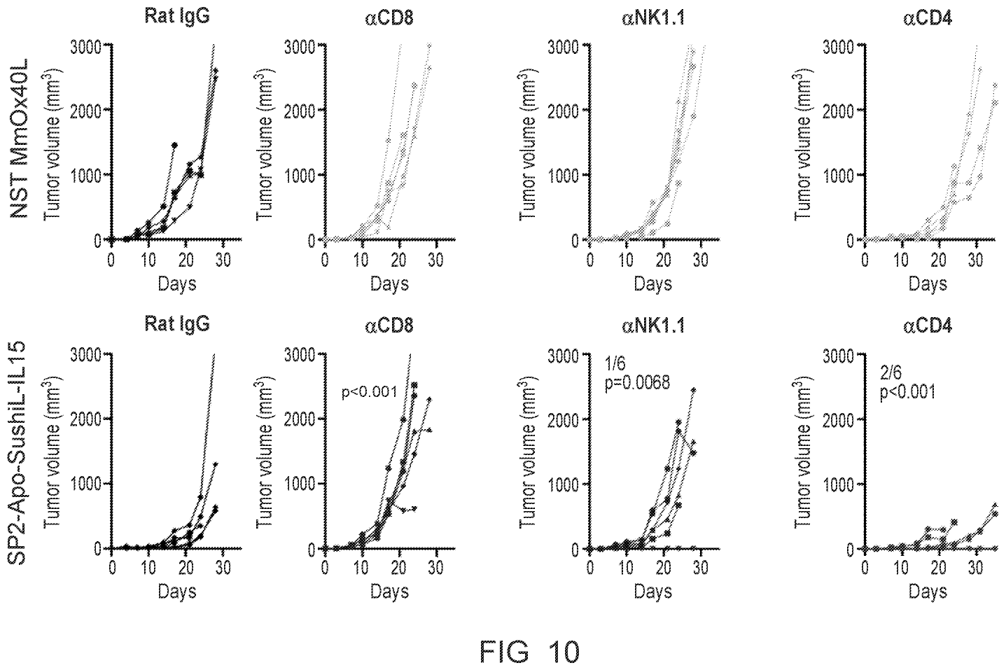

The effector immune cell populations involved in the antitumor effects induced by SP2-Apo-SushiL-IL15 were evaluated. MC38 tumor cells were inoculated in C57BL/6 mice. At day 5 and 10 post tumor cell inoculation, mice were treated intravenously with mRNA encoding SP2-Fc-SushiL-IL15 complexed in TransIT (10 pg mRNA/mice). Anti-CD8 mAb, anti-CD4 mAb or anti-NK1.1 (200 μg/mice) were administered intra-peritoneally on days 4, 7, 11, 13, and 18. Tumor growth (mean diameter) was measured over time. Control mice received TransIT-complexed mRNA encoding NST murine OX40 ligand. As shown in FIG. 10, CD8+T cell depletion abrogated the antitumor effect of the IL15 fusion protein, while NK cell depletion had a minor effect. In contrast, CD4+T lymphocyte depletion synergized with IL15 and markedly enhanced the antitumor effect.

### Example 7: Evaluation of IL15 Fusion Protein Accumulation in Tumors

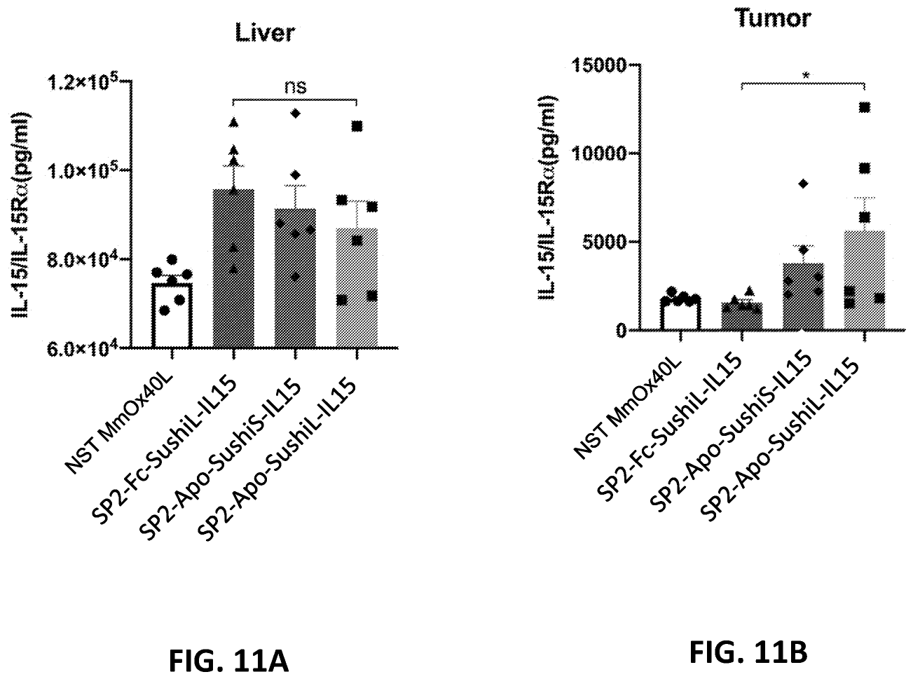

The expression of IL15/IL15Rα was evaluated in tumor bearing mice following administration of mRNAs encoding IL15 fusion proteins. C57BL/6 mice were inoculated with MC38 tumors. At day 5 post tumor inoculation, the mice were injected intravenously with 10 μg mRNA encoding SP2-Apo-SushiS-IL15 or SP2-Apo-SushiL-IL15 complexed with TransIT or g reference mRNA (encoding SP2-Fc-SushiL-IL15) complexed with TransIT. Control mice received TransIT-complexed mRNA encoding NST murine OX40 ligand. At 24 hours following mRNA administration, tumors and livers were harvested and IL15/IL15Rα was quantified by ELISA. As shown in FIG. 11A, IL15 was similarly induced in the liver by the reference mRNA and mRNA encoding SP2-Apo-SushiS-IL15 or SP2-Apo-SushiL-IL15. However, higher amounts of IL15 were detected in the tumor when the IL15 mRNA encoding SP2-Apo-SushiL-IL15 was administered (FIG. 11B). These data indicate that accumulation of SP2-Apo-SushiL-IL15 in tumors provides for an enhanced anti-tumor immune response.

The livers harvested from mice at 24 hours following administration of the mRNA were also evaluated by histology. The livers of mice that received IL15 fusion protein had comparable hematoxylin and eosin staining to livers of control mice (data not shown). This indicates that administration of mRNA encoding IL15 fusion protein does not induce significant liver toxicity.

### Materials and Methods

Cell lines and culture media

HEK-293T cells were maintained at 37° C. in 5% CO2 and were grown in Dulbecco's Modified Eagle's Medium (DMEM) with Glutamax (Gibco, Invitrogen, Carlsbad, CA, USA) containing 10% heat-inactivated FBS (Sigma-Aldrich, Dorset, UK), 100 IU/mL penicillin, 100 g/mL streptomycin (Biowhittaker, Walkersville, MD, USA) and 50 mmol/L 2-mercaptoethanol (Gibco). CTLL-2 is a stable subclone of cytotoxic T-lymphocytes originally isolated from a C57BL/6 mouse, and 200U/mL of IL-2 (Preprotech, Germany) was added in its culture medium. CTLL-2 cells were maintained at 37° C. in 5% CO2 and were grown in RPMI-1640 medium (Gibco-Invitrogen, Carlsbad, CA, USA) supplemented with 20% fetal bovine serum, 100 IU/mL penicillin, and 100 g/mL streptomycin. M07E cell is a cell line derived from human megakaryocytic leukemic. M07E cells were maintained in Iscove's Modified Dulbecco's Medium (IMDM) containing 20% heat-inactivated FBS (Sigma-Aldrich), 100 IU/mL penicillin and 100 g/mL streptomycin (Biowhittaker), and 50 mmol/L 2-mercaptoethanol (Gibco). The medium was supplemented with 100 ng/ml of human Granulocyte-macrophage colony-stimulating factor (GM-CSF). MC-38 and B16OVA cell lines were maintained at 37° C. in 5% CO2 and were grown in RPMI medium (RPMI 1640) with Glutamax (Gibco, Invitrogen, Carlsbad, CA, USA) containing 10% heat-inactivated FBS (Sigma-Aldrich, Dorset, UK), 100 IU/mL penicillin, 100 g/mL streptomycin (Biowhittaker, Walkersville, MD, USA) and 50 mmol/L 2-mercaptoethanol (Gibco). B16OVA cells were maintained with 400 μg/ml of Geneticin. After 7-9 days in culture, cells were tested for mycoplasma contamination, and 5×10⁵ cells per mice were injected subcutaneously.

Animal handling

Female C57BL/6 mice (8-10 weeks old) were purchased from Harlan Laboratories (Barcelona, Spain) and were housed at least 7 days on a 12 h light/dark cycle before injection. Mice had free access to food and water throughout the course of the experiments and were maintained under pathogen-free conditions in the animal facility of Cima Universidad de Navarra.

For antitumor evaluation, mice were randomized at the beginning of each experiment. Tumor growth was monitored twice a week with an electronic caliper, and mice were sacrificed when the tumor reached 15 mm.

For the study of the immune cells involved in the antitumor effect, mice receive 200 μg of anti-CD4 (clone GK1.5, BioXCell, L'Aigle, France), anti-CD80 (clone H35-17.2, in house), anti-PD-1 (clone RMP1-14, BioXCell), or anti-NK1.1 (clone PK136, BioXCell). InvivoMab rat IgG2b (clone LTF-2, BioXCell) was used as control Antibody-mediated cellular depletion was initiated one day prior to treatment with mRNA.

Polymer/lipid-based formulation of mRNA

For in vitro transfection of HEK-293T, mRNA was formulated with the TranslT-mRNA Transfection Kit (Mirus Bio Corporation, Madison, WI, USA). One microgram of mRNA in 250 μl was mixed with 5 1 of TranslT-mRNA reagent and 5 1 of TransIT Boost reagent per well. mRNA complex was then vortexed, incubated at room temperature for 2 minutes, and added to each well within 5 minutes.

For mRNA delivery, mice were injected in the tail vein with 10 micrograms of mRNA in 190 μl of cold DMEM was mixed with 5.6 1 of TransIT-mRNA reagent and 3.6 1 of TransIT Boost reagent per mouse. mRNA complex was then vortexed, incubated at room temperature for 2 minutes, and injected into mice within 5 minutes. The total volume of mRNA complex was 200 1.

Pharmacokinetic evaluation in vitro

3×10⁵ HEK-293T cells were cultured per well. 24h later, supernatants were removed, and 2.5 mL of DMEM free (without FBS) were added to each well in order to transfect the cells with the mRNA variants. 6 h, 24h, 48h, and 72h after transfection supernatant were collected and stored at −80° C. until analysis.

To determine the concentration of IL15/IL15Rα, the Human IL15/IL15R alpha Complex DuoSet ELISA (R&D Systems, Inc., Minneapolis, USA) was used following the manufacturer's instructions.

In order to determine the concentration of Apolipoprotein A-I, the Human apoAI ELISA PRO kit (Mabtech, Nacka, Sweden) was used following the manufacturer's instructions.

Proliferation assay

The bioactivity of IL15 reference and IL15 variants was tested in CTLL-2 and Mo7e cells.

CTLL-2 and Mo7e cells were washed three times with PBS and 10⁴ cells/well in 50 μl of RPMI medium or IMDM medium respectively, were plated in a 96-well plate. WHO Reference Reagent Interleukin-15 (NIBSC code: 95/554) was used as standard. Standard and samples were serially diluted. The plates were then incubated for 48h, and subsequently, the microcultures were pulsed with 0.5 Ci of tritiated thymidine ([3H]TdR) 8h before being harvested. Cells were harvested using a Micro Beta Filter Mate-96 harvester (Perkin Elmer), and [3H]TdR incorporation to the nuclei adsorbed onto the filters was measured using an automated Topcount liquid scintillation counter (Packard).

Pharmacokinetic evaluation in vivo

C57BL/6 mice were intravenously injected with 10 μg of TransIT-formulated IL15 mRNA variants as explained above. 24 h, 48h, and 72h after mRNA inoculation, blood samples were collected. The blood samples were immediately centrifuged at 3000 rpm for 15 minutes to isolate the serum, and IL15/IL15Rα was performed. Serum were aliquoted and stored at −80° C. until analysis.

Pharmacodynamic and safety evaluation in vivo

Mice were injected i.v. with 10 μg of TransIT-formulated IL15 mRNA variants. Two and five days after mRNA injection, spleens, tumors, livers, and lymph nodes were surgically harvested. The organs were disrupted mechanically, and tumors were incubated in collagenase and DNase (Roche) for 30 min at 37° C. Erythrocytes were lysed with ACK buffer.

Dissociated cells from livers and tumors were centrifuged with Percoll© (GE Healthcare, Chalfont St Giles, UK) at 40% (500 g, 10 min, 20° C.), making a gradient in order to eliminate parenchymal cells.

Cells isolated from livers, tumors, and spleens were used to study the activating, functional, and proliferative status of T cells, NK cells, and dendritic subsets. To reduce nonspecific staining, samples were pretreated with Fc-Block (anti-CD16/32, eBioscience, San Diego, CA, USA). CytoFlex LX cytometer (Beckman coulter, High Wycombe, UK) was used for cell acquisition, and data analysis was performed using FlowJo 10 (Tree Star Inc., Ashland, OR, USA).

Biodistribution analysis

5×10⁵ MC-38 cells were inoculated subcutaneously on the right flank of C57BL/6 mice. On day five, 10 μg of mRNA encoding luciferase complexed with TransIT were injected intravenously per mouse. 6 h, 24h, 48h, and 72h later, in vivo imaging was performed upon intraperitoneal injection of D-luciferin (1.5 mg/mouse) as substrate and visualized using PhotonIMAGERTM (Biospace Lab, Paris, France).

For biodistribution analysis, mice received the TransIT- formulated luciferase mRNA. 24h later, mice were sacrificed, and ex vivo luciferase expression was quantified in spleen, lungs, kidney, brain, heart, liver, and tumor.

To evaluate the luciferase expression in the absence of Kupffer cells, clodronate liposomes (200 μl/20g) (Liposoma BV, Amsterdam, The Netherlands) were administered to mice. 24h hours later, TransIT-formulated luciferase mRNA was administered to mice with or without pretreatment with clodronate liposomes. The luciferase expression was analyzed 24 hours after by bioluminescence. Then, mice were sacrificed, and ex vivo luciferase expression was quantified.

The threshold of bioluminescence signals was automatically determined using M3 Vision software (Biospace Lab). Bioluminescence signals were accordingly filtered against background noise. Regions of interest were defined as regions above a threshold and automatically gated by predefined program tools. Photon emission intensity (ph/s/cm2/sr) was calculated from data of emitted photons from the respective regions of interest using the M3 Vision software.

Determination of IL15/IL15Rα in the tumor microenvironment

To determine IL15/IL15Rα protein levels in the tumor microenvironment, the tumor was first homogenized by mechanic disruption with a pestle in PBS buffer with Complete Protease Inhibitors. After centrifugation, the supernatant was collected and stored at −80° C. for further use. 1:2 dilutions of tumor supernatant were used to evaluate the protein level of IL15/IL15Rα using the Human IL15/IL15R alpha Complex DuoSet ELISA (R&D Systems), following the manufacturer's instructions. The colorimetric reaction was measured in a spectrophotometer (=450 nm).

Statistical analysis

GraphPad Prism version 8.2.1 software (GraphPad Software, Inc., San Diego, CA) was used for statistical analysis. Data were analyzed by t-test for comparisons between two groups and one-way ANOVA followed by Sidak's multiple comparisons test for three or more groups. Longitudinal data were fitted to the indicated equations and compared with an extra sum-of-squares F test. P values <0.05 were considered to be statistically significant.

SEQUENCE LISTING

| Name / | | SEQ ID |
|---|---|---|
| Identifier | Sequence | NO |
| SP2-ApoA-SushiL- | AUGGAGUUCGGCUUGAGCUGGGUGUUCCUAGUGGCCAUCUUGAAG | 1 |
| IL15 (RNA) | GGCGUGCAGUGCGACGAGCCUCCUCAGAGCCCUUGGGACAGAGUG | |
| | AAGGACUUGGCCACCGUGUACGUGGACGUGCUUAAGGACAGCGGC | |
| | AGAGACUACGUGAGCCAGUUCGAGGGCAGCGCCCUGGGCAAGCAG | |
| | CUGAACCUGAAGCUGCUGGACAACUGGGACAGCGUGACCAGCACC | |
| | UUCAGCAAGCUGAGAGAGCAGCUGGGCCCUGUGACCCAGGAGUUC | |
| | UGGGACAACCUGGAGAAGGAGACAGAGGGCCUGAGACAGGAGAUG | |
| | AGCAAGGACUUAGAGGAGGUGAAGGCCAAGGUGCAGCCUUACCUG | |
| | GACGACUUCCAGAAGAAGUGGCAGGAGGAGAUGGAGCUGUACAGA | |
| | CAGAAGGUGGAGCCUCUGAGAGCCGAGCUGCAGGAGGGCGCACGG | |
| | CAGAAGCUGCACGAACUGCAGGAGAAGCUGAGCCCUCUGGGCGAG | |
| | GAAAUGAGAGACAGAGCCAGAGCCCACGUGGAUGCCCUGAGAACC | |
| | CACCUGGCCCCUUACUCGGACGAGCUGCGACAGAGACUGGCCGCC | |
| | AGACUGGAGGCCCUGAAGGAGAACGGCGGCGCAAGACUCGCAGAG | |
| | UACCACGCCAAGGCCACCGAGCACCUGAGCACCCUGAGCGAGAAG | |
| | GCUAAGCCUGCUCUGGAGGACCUGCGGCAGGGCCUGCUGCCUGUG | |
| | CUGGAGAGCUUCAAGGUGAGCUUCCUGUCCGCACUGGAAGAAUAC | |
| | ACCAAGAAGCUGAACACCCAGGGCGGCGGAGGAUCGGGUGGAGGU | |
| | GGUAGUGGUGGUGGCGGUUCCAUCACCUGCCCUCCUCCUAUGAGC | |
| | GUGGAGCACGCCGACAUCUGGGUAAAGUCUUACAGCCUGUACAGC | |
| | AGAGAGAGAUACAUCUGCAACAGCGGCUUCAAGAGAAAGGCCGGC | |
| | ACCAGCAGCCUGACCGAGUGUGUCCUGAACAAGGCAACCAACGUG | |
| | GCCCACUGGACCACCCCUAGCCUGAAGUGCAUCAGAGAUCCAGCC | |
| | CUUGUGCACCAAAGACCUGCCCCUCCAUCAGGAGGUUCCGGCGGU | |
| | GGAGGAUCAGGCGGCGGCAGCGGAGGUGGCGGAUCCCUGCAGAAC | |
| | UGGGUAAACGUGAUCAGCGACCUGAAGAAGAUCGAAGAUCUGAUC | |
| | CAGAGCAUGCACAUCGACGCCACCCUGUACACCGAGAGCGACGUG | |
| | CACCCUAGCUGCAAGGUGACCGCCAUGAAGUGCUUCCUGCUGGAA | |
| | CUCCAGGUUAUCUCCCUGGAGAGUGGCGACGCCAGCAUCCACGAC | |
| | ACCGUGGAGAACCUGAUCAUCCUGGCCAACAACAGCCUGAGCAGC | |
| | AACGGCAACGUGACUGAAAGCGGCUGUAAGGAGUGCGAGGAGCUC | |
| | GAGGAGAAGAACAUCAAGGAGUUCCUGCAGAGCUUCGUCCACAUC | |
| | GUGCAGAUGUUCAUCAACACCAGC | |
| | Signal peptide ″SP2″ (bold); ApoA (underline); | |
| | SushiL (bold italics); mature IL15 (bold | |
| | underline) | |
| SP2-ApoA-SushiL- | MEFGLSWVFLVAILKGVQCDEPPQSPWDRVKDLATVYVDVLKDSG | 2 |
| IL15 (aa) | RDYVSQFEGSALGKQLNLKLLDNWDSVTSTFSKLREQLGPVTQEF | |
| | WDNLEKETEGLRQEMSKDLEEVKAKVQPYLDDFQKKWQEEMELYR | |
| | QKVEPLRAELQEGARQKLHELQEKLSPLGEEMRDRARAHVDALRT | |
| | HLAPYSDELRQRLAARLEALKENGGARLAEYHAKATEHLSTLSEK | |
| | AKPALEDLRQGLLPVLESFKVSFLSALEEYTKKLNTQGGGGSGGG | |
| | GSGGGGSITCPPPMSVEHADIWVKSYSLYSRERYICNSGFKRKAG | |
| | TSSLTECVLNKATNVAHWTTPSLKCIRDPALVHQRPAPPSGGSGG | |
| | GGSGGGSGGGGSLQNWVNVISDLKKIEDLIQSMHIDATLYTESDV | |
| | HPSCKVTAMKCFLLELQVISLESGDASIHDTVENLIILANNSLSS | |
| | NGNVTESGCKECEELEEKNIKEFLQSFVHIVQMFINTS | |
| | Signal peptide ″SP2″ (bold); ApoA (underline); | |
| | SushiL (bold italics); mature IL15 (bold | |
| | underline) | |
| SP2-ApoA-SushiS- | AUGGAGUUCGGCUUAAGCUGGGUGUUCCUCGUGGCCAUCUUAAAG | 3 |
| IL15 (RNA) | GGCGUGCAGUGCGACGAGCCUCCUCAGAGCCCUUGGGACAGAGUG | |
| | AAGGACCUCGCCACCGUGUACGUGGACGUGCUCAAGGACAGCGGC | |
| | AGAGACUACGUGAGCCAGUUCGAGGGCAGCGCCCUGGGCAAGCAG | |
| | CUGAACCUGAAGCUGCUGGACAACUGGGACAGCGUGACCAGCACC | |
| | UUCAGCAAGCUGAGAGAGCAGCUGGGCCCUGUGACCCAGGAGUUC | |
| | UGGGACAACCUGGAGAAGGAGACUGAGGGCCUGAGACAGGAGAUG | |
| | AGCAAGGAUCUGGAGGAGGUGAAGGCCAAGGUGCAGCCUUACCUG | |
| | GACGACUUCCAGAAGAAGUGGCAGGAGGAGAUGGAGCUGUACAGA | |
| | CAGAAGGUGGAGCCUCUGAGAGCCGAGCUGCAGGAGGGCGCACGU | |
| | CAGAAGCUGCACGAACUUCAGGAGAAGCUGAGCCCUCUGGGCGAA | |
| | GAAAUGAGAGACAGAGCCAGAGCCCAUGUGGACGCCCUGAGAACC | |
| | CACCUGGCCCCUUACAGCGACGAACUGAGACAAAGACUGGCCGCC | |
| | AGACUGGAGGCCCUGAAGGAGAACGGCGGUGCGAGAUUAGCUGAG | |
| | UACCACGCCAAGGCCACCGAGCACCUGAGCACCCUGAGCGAGAAG | |
| | GCUAAGCCUGCCCUCGAGGACCUGCGACAAGGCCUGCUGCCUGUG | |
| | CUGGAGAGCUUCAAGGUGAGCUUCCUGUCAGCACUUGAAGAGUAC | |
| | ACCAAGAAGCUGAACACCCAGGGCGGCGGCGGAAGCGGCGGUGGC | |
| | GGCAGUGGAGGCGGUGGAAGCAUCACCUGCCCGCCUCCUAUGAGC | |
| | GUGGAGCACGCCGACAUCUGGGUAAAGUCCUACAGCCUGUACAGC | |
| | AGAGAGAGAUACAUCUGCAACAGCGGCUUCAAGAGAAAGGCCGGC | |
| | ACCAGCAGCCUGACCGAGUGCGUGCUUAAUAAGGCCACUAACGUG | |
| | GCCCACUGGACCACCCCUAGCCUGAAGUGCAUCAGAGACGGCGGU | |
| | UCAGGAGGCGGAGGUUCCGGUGGAGGCUCAGGUGGAGGUGGUAGC | |
| | CUGCAGAACUGGGUAAACGUGAUCAGCGACCUGAAGAAGAUCGAA | |
| | GAUCUCAUCCAGAGCAUGCACAUCGACGCCACCCUGUACACCGAG | |
| | AGCGACGYGCACCCUAGCYGCAAGGUGACCGCCAUGAAGYGCUUC | |
| | CUGCUGGAACUGCAGGUGAUAAGCCUGGAGUCUGGCGACGCCAGC | |
| | AUCCACGACACCGUGGAGAACCUGAUCAUCCUGGCCAACAACAGC | |
| | CUGAGCAGCAACGGCAACGUAACCGAGUCC GCUGUAAGGAGUGC | |
| | GAGGAGUUGGAGGAGAAGAACAUCAAGGAGUUCCUGCAGAGCUUC | |
| | GUUCACAUCGUGCAGAUGUUCAUCAACACCAGC | |
| | Signal peptide ″SP2″ (bold); ApoA (underline); | |
| | SushiS (bold italics); mature IL15 (bold | |
| | underline) | |
| SP2-ApoA-SushiS- | MEFGLSWVFLVAILKGVQCDEPPQSPWDRVKDLATVYVDVLKDSG | 4 |
| IL15 (aa) | RDYVSQFEGSALGKQLNLKLLDNWDSVTSTFSKLREQLGPVTQEF | |
| | WDNLEKETEGLRQEMSKDLEEVKAKVQPYLDDFQKKWQEEMELYR | |
| | QKVEPLRAELQEGARQKLHELQEKLSPLGEEMRDRARAHVDALRT | |
| | HLAPYSDELRQRLAARLEALKENGGARLAEYHAKATEHLSTLSEK | |
| | AKPALEDLRQGLLPVLESFKVSFLSALEEYTKKLNTQGGGGSGGG | |
| | GSGGGGSITCPPPMSVEHADIWVKSYSLYSRERYICNSGFKRKAG | |
| | TSSLTECVLNKATNVAHWTTPSLKCIRDGGSGGGGSGGGSGGGGS | |
| | LQNWVNVISDLKKIEDLIQSMHIDATLYTESDVHPSCKVTAMKCE | |
| | LLELQVISLESGDASIHDTVENLIILANNSLSSNGNVTESGCKEC | |
| | EELEEKNIKEFLQSFVHIVQMFINTS | |
| | Signal peptide ″SP2″ (bold; ApoA | |
| | (underline); SushiS (bold italics); mature | |
| | IL15 (bold underline) | |
| SP2-Fc-SushiL- | AUGGAGUUCGGCUUGAGCUGGGUGUUCUUAGUGGCCAUCUUAAAG | 5 |
| IL15 (RNA) | GGCGUGCAGUGCGAGCCUAAGAGCUGCGACAAGACCCACACCUGC | |
| | CCUCCUUGCCCGGCCCCAGAGUUGCUUGGCGGCCCUAGCGUCUUC | |
| | CUCUUCCCUCCUAAGCCUAAGGACACCCUGAUGAUCAGCAGAACC | |
| | CCUGAGGUGACCUGCGUGGUGGUGGCCGUGAGCCACGAGGACCCU | |
| | GAAGUAAAGUUCAACUGGUACGUGGACGGCGUGGAGGUGCACAAC | |
| | GCCAAGACCAAGCCUAGAGAGGAGCAGUACAACAGCACCUACAGA | |
| | GUGGUGAGCGUGCUGACCGUGCUGCACCAGGACUGGCUGAACGGC | |
| | AAGGAGUACAAGUGCAAGGUGAGCAACAAGGCCCUGCCUGCACCU | |
| | AUCGAGAAGACCAUCAGCAAGGCCAAGGGCCAACCUCGAGAGCCU | |
| | CAGGUGUACACCCUGCCUCCUAGCAGAGACGAGCUGACCAAGAAC | |
| | CAGGUGAGCCUGACCUGCCUGGUGAAGGGCUUCUACCCUAGCGAC | |
| | AUCGCCGUGGAGUGGGAGAGCAACGGCCAACCAGAGAACAACUAC | |
| | AAGACCACCCCUCCUGUGCUGGACAGCGACGGCAGCUUCUUCCUC | |
| | UACAGCAAGCUGACUGUUGACAAGAGCAGAUGGCAGCAGGGCAAC | |
| | GUGUUCAGCUGCAGCGUGAUGCACGAGGCGCUGCACAACCACUAC | |
| | ACCCAGAAGAGUCUUUCCCUGAGCCCUGGCAAGAUAACGUGUCCG | |
| | CCUCCUAUGAGCGUGGAGCACGCCGACAUCUGGGUGAAGAGUUAC | |
| | AGCCUGUAUUCCAGAGAGAGAUACAUCUGCAACAGCGGCUUCAAG | |
| | AGAAAGGCCGGCACCAGCAGCUUAACCGAGUGCGUCCUUAACAAG | |
| | GCUACCAACGUGGCCCACUGGACAACGCCGAGCCUGAAGUGCAUC | |
| | CGCGACCCUGCCCUGGUGCACCAACGUCCAGCACCUCCGAGUGGC | |
| | GGCAGUGGUGGCGGAGGCUCAGGCGGUGGUAGUGGAGGAGGCGGA | |
| | AGUCUGCAGAAUUGGGUGAACGUGAUCAGCGACCUGAAGAAGAUC | |
| | GAGGACCUGAUCCAGAGCAUGCACAUCGACGCCACCCUGUACACC | |
| | GAGAGCGACGUGCACCCUAGCUGUAAGGUAACCGCCAUGAAGUGC | |
| | UUCCUGCUGGAGCUGCAGGUGAUUAGCCUGGAGAGCGGCGACGCC | |
| | AGCAUCCACGACACCGUGGAGAACCUGAUCAUCCUGGCCAACAAU | |
| | UCUCUCAGUUCUAACGGUAACGUGACCGAAUCGGGUUGUAAGGAG | |
| | UGCGAGGAGCUGGAGGAGAAGAACAUCAAGGAGUUCCUGCAGAGC | |
| | UUCGUGCACAUUGUGCAGAUGUUCAUCAACACCAGC | |
| | Signal peptide ″SP2″ (bold); Fc domain | |
| | (underline); SushiL (bold italics); mature | |
| | IL15 (bold underline) | |
| SP2-Fc-SushiL- | MEFGLSWVFLVAILKGVQCEPKSCDKTHTCPPCPAPELLGGPSVF | 6 |
| IL15 (aa) | LFPPKPKDTLMISRTPEVTCVVVAVSHEDPEVKFNWYVDGVEVHN | |
| | AKTKPREEQYNSTYRVVSVLTVLHQDWLNGKEYKCKVSNKALPAP | |
| | IEKTISKAKGQPREPQVYTLPPSRDELTKNQVSLTCLVKGFYPSD | |
| | IAVEWESNGQPENNYKTTPPVLDSDGSFFLYSKLTVDKSRWQQGN | |
| | VFSCSVMHEALHNHYTQKSLSLSPGKITCPPPMSVEHADIWVKSY | |
| | SLYSRERYICNSGFKRKAGTSSLTECVLNKATNVAHWTTPSLKCI | |
| | RDPALVHQRPAPPSGGSGGGGSGGGSGGGGSLQNWVNVISDLKKI | |
| | EDLIQSMHIDATLYTESDVHPSCKVTAMKCFLLELQVISLESGDA | |
| | SIHDTVENLIILANNSLSSNGNVTESGCKECEELEEKNIKEFLQS | |
| | FVHIVQMFINTS | |
| | Signal peptide ″SP2″ (bold); Fc domain | |
| | (underline); SushiL (bold italics); mature | |
| | IL15 (bold underline) | |
| NST-MmOX40L | AAGGAAGGGGAAGGGGUUCAACCCCUGGAAGAGAAUCUGGAAAAC | 7 |
| (RNA) | GGAUCAAGGCCAAGAUUCAAGAGGAAGAAGACGCUAAGGCUGGAG | |
| | GUCUCUGGGAUCAAGGGAGCAGGGAAGCUUCUGAGCUUCAUCUAA | |
| | GUCUGCCUGCAACUCUCUUCCUCUCCGGCAAAGGACCCUCCAAUC | |
| | CAAAGACUCAGAGGAGCAGUUACCAGAAGAGAGGAAGGGCAACUA | |
| | UUCAUCAGCUCAUACAAGAAAGAGUAUCAAACUAAGGAGGAGCAG | |
| | AACAAUUCGGUUGUCAUCAAGAGCGAAGGGCUUUAUAUCAUCUAC | |
| | CUGAAGGGCUCCUUUUUCCAGGAGGUCAAGAUUGACCUUCAUUUC | |
| | CGGGAGGAUCAUAAUCCCAUCUCUAUUCCAAAGCUGAACGAAGGU | |
| | CGAAGGAUUGUCUUCACUGAGGAGGCCUCUUUGGCUUUCAAAGAU | |
| | AAAGUUUACCUGACUGUAAAAGCUCCUGAUACUCUCUGCGAACAC | |
| | CUCCAGAUAAAAGAAGGGGAGCUGAUUGUUGUCCAGCUAACGCCU | |
| | GGAUACUGAGCUCCUGAAGGAUCUUACCACAGCACUGAGAACCAA | |
| | GUACCACUG | |
| SP1-SushiS-IL15- | AUGGCUCCUCGACGCGCUCGGGGAUGCCGGACCCUCGGUUUGCCG | 8 |
| ApoA (RNA) | GCGUUGCUACUCCUUCUCCUACUCCGCCCACCUGCCACUAGAGGC | |
| | AUCACGUGCCCUCCUCCCAUGUCCGUGGAACACGCAGACAUCUGG | |
| | GUCAAGAGCUACAGCUUGUACUCCAGGGAGCGGUACAUUUGUAAC | |
| | UCUGGUUUCAAGCGUAAAGCCGGCACGUCCAGCCUGACGGAGUGC | |
| | GUGUUGAACAAGGCCACGAAUGUCGCCCACUGGACAACACCUAGU | |
| | CUCAAAUGCAUUAGAGACGGCGGCUCUGGAGGUGGAGGAUCCGGC | |
| | GGAGGAUCUGGAGGAGGAGGCAGCCUGCAGAACUGGGUGAAUGUA | |
| | AUAAGUGAUUUGAAGAAGAUUGAAGAUCUUAUUCAAUCUAUGCAU | |
| | AUUGAUGCUACUUUAUAUACGGAAAGUGAUGUUCACCCCAGUUGC | |
| | AAAGUAACAGCAAUGAAGUGCUUUCUCUUGGAGUUACAAGUUAUU | |
| | UCACUUGAGUCCGGAGAUGCAAGUAUUCAUGAUACAGUAGAGAAU | |
| | CUGAUCAUCCUAGCAAACAACAGUUUGUCUUCUAAUGGGAAUGUA | |
| | ACAGAAUCUGGAUGCAAAGAAUGUGAGGAACUGGAGGAGAAGAAC | |
| | AUUAAAGAAUUCUUGCAGAGUUUUGUACAUAUUGUCCAAAUGUUC | |
| | AUCAACACUUCUGGCGCGCCCGAUGAACCACCCCAGAGCCCCUGG | |
| | GAUCGAGUGAAGGACCUGGCCACUGUGUACGUGGAUGUGCUCAAA | |
| | GACAGCGGCAGAGACUAUGUGUCCCAGUUUGAAGGCUCCGCCUUG | |
| | GGCAAGCAGCUAAACCUAAAGCUCCUUGACAACUGGGACAGCGUG | |
| | ACCUCCACCUUCAGCAAGCUGCGCGAACAGCUCGGCCCUGUGACC | |
| | CAGGAGUUCUGGGAUAACCUGGAGAAGGAGACAGAGGGCCUGAGG | |
| | CAGGAGAUGAGCAAGGACCUAGAGGAGGUGAAGGCCAAGGUGCAG | |
| | CCCUACCUGGACGACUUCCAGAAGAAGUGGCAGGAGGAGAUGGAG | |
| | CUCUACCGCCAGAAGGUGGAGCCGCUGCGCGCAGAGCUCCAAGAA | |
| | GGUGCCAGACAGAAGCUGCACGAGCUGCAAGAGAAGCUGUCUCCA | |
| | CUGGGCGAAGAGAUGCGCGACAGAGCUCGCGCGCAUGUGGACGCG | |
| | CUGCGCACGCAUCUGGCACCUUACAGCGAUGAGCUCCGCCAGCGC | |
| | UUGGCCGCGCGCCUUGAGGCUCUCAAGGAGAACGGCGGCGCCAGA | |
| | CUGGCCGAGUACCACGCUAAGGCGACCGAGCAUCUGAGCACGCUC | |
| | AGCGAGAAGGCAAAGCCCGCGCUCGAGGACCUCCGACAAGGACUG | |
| | CUGCCCGUGCUGGAGAGCUUCAAGGUCAGCUUCCUGAGCGCUCUC | |
| | GAGGAGUACACUAAGAAGCUCAACACCCAG | |
| | Signal peptide ″SP1″ (bold); SushiS (bold | |
| | italics); mature IL15 (bold underline); ApoA | |
| | (underline) | |
| SP1-SushiS-IL15- | MAPRRARGCRTLGLPALLLLLLLRPPATRGITCPPPMSVEHADIW | 9 |
| ApoA (aa) | VKSYSLYSRERYICNSGFKRKAGTSSLTECVLNKATNVAHWTTPS | |
| | LKCIRDGGSGGGGSGGGSGGGGSLQNWVNVISDLKKIEDLIQSMH | |
| | IDATLYTESDVHPSCKVTAMKCFLLELQVISLESGDASIHDTVEN | |
| | LIILANNSLSSNGNVTESGCKECEELEEKNIKEFLQSFVHIVQMF | |
| | INTSGAPDEPPQSPWDRVKDLATVYVDVLKDSGRDYVSQFEGSAL | |
| | GKQLNLKLLDNWDSVTSTFSKLREQLGPVTQEFWDNLEKETEGLR | |
| | QEMSKDLEEVKAKVQPYLDDFQKKWQEEMELYRQKVEPLRAELQE | |
| | GARQKLHELQEKLSPLGEEMRDRARAHVDALRTHLAPYSDELRQR | |
| | LAARLEALKENGGARLAEYHAKATEHLSTLSEKAKPALEDLRQGL | |
| | LPVLESFKVSFLSALEEYTKKLNTQ | |
| | Signal peptide ″SP1″ (bold); SushiS (bold | |
| | italics); mature IL15 (bold underline); ApoA | |
| | (underline) | |
| SP1-SushiL-IL15- | AUGGCCCCUCGGCGGGCCAGAGGCUGCAGAACCUUAGGCCUCCCA | 10 |
| ApoA (RNA) | GCCCUUCUAUUGCUCUUGCUACUCAGACCUCCAGCCACCAGAGGC | |
| | AUCACCUGCCCUCCUCCUAUGAGCGUGGAGCACGCCGACAUCUGG | |
| | GUAAAGUCCUACAGCCUGUACAGCAGAGAGAGAUACAUCUGCAAC | |
| | AGCGGCUUCAAGAGAAAGGCCGGCACCAGCAGCCUGACCGAGUGC | |
| | GUGCUGAACAAGGCCACCAACGUGGCCCACUGGACCACCCCUAGC | |
| | CUGAAGUGCAUCAGGGACCCAGCUCUAGUGCACCAGCGGCCUGCC | |
| | CCUCCUAGCGGCGGCUCGGGUGGCGGUGGAUCGGGUGGUGGUUCC | |
| | GGCGGAGGAGGCUCUCUCCAGAACUGGGUUAACGUGAUCAGCGAC | |
| | CUGAAGAAGAUCGAGGACCUGAUCCAGAGCAUGCACAUCGACGCC | |
| | ACCCUGUACACCGAGAGCGACGUGCACCCUAGCUGCAAGGUGACC | |
| | GCCAUGAAGUGCUUCCUGCUGGAGCUGCAGGUAAUUUCUCUGGAG | |
| | AGCGGCGACGCCAGCAUCCACGACACCGYGGAGAACCYGAYCAYC | |
| | CUGGCCAACAACAGCCUGAGCAGCAACGGCAACGUGACUGAGUCC | |
| | GGAUGCAAGGAGUGCGAGGAGCUGGAGGAGAAGAACAUCAAGGAG | |
| | UUCCUGCAGAGCUUCGUGCAUAUCGUGCAGAUGUUCAUCAACACC | |
| | AGCGGCGCCCCUGACGAGCCUCCUCAGAGCCCUUGGGACAGAGUG | |
| | AAGGACCUGGCCACCGUGUACGUGGACGUGUUAAAGGACAGCGGC | |
| | AGAGACUACGUGAGCCAGUUCGAGGGCAGCGCCCUGGGCAAGCAG | |
| | CUGAACCUGAAGCUGCUGGACAACUGGGACAGCGUGACCAGCACC | |
| | UUCAGCAAGCUGAGAGAGCAGCUGGGCCCUGUGACCCAGGAGUUC | |
| | UGGGACAACCUGGAGAAGGAAACUGAGGGCCUGAGACAGGAGAUG | |
| | AGCAAGGACCUUGAAGAAGUGAAGGCCAAGGUGCAGCCUUACCUG | |
| | GACGACUUCCAGAAGAAGUGGCAGGAGGAGAUGGAGCUGUACAGA | |
| | CAGAAGGUGGAGCCUCUGAGAGCCGAACUUCAGGAGGGCGCUCGC | |
| | CAGAAGCUGCACGAGCUACAAGAGAAGCUGAGCCCUCUGGGCGAA | |
| | GAGAUGCGCGACCGGGCUAGGGCCCAUGUUGACGCCCUGAGAACC | |
| | CACCUGGCCCCUUACAGCGAUGAACUGCGCCAGCGCCUGGCCGCC | |
| | AGACUGGAGGCCCUGAAGGAGAACGGCGGCGCUCGACUAGCCGAG | |
| | UACCACGCCAAGGCUACUGAGCACCUGAGCACUCUGAGCGAGAAG | |
| | GCGAAGCCGGCCCUGGAGGAUCUCAGGCAAGGCCUGCUGCCUGUG | |
| | CUGGAGUCAUUCAAGGUGAGCUUCCUGUCCGCGCUCGAAGAGUAC | |
| | ACCAAGAAGUUGAACACCCAG | |
| | Signal peptide ″SP1″ (bold); SushiL (bold | |
| | italics); mature IL15 (bold underline); ApoA | |
| | (underline | |
| SP1-SushiL-IL15- | MAPRRARGCRTLGLPALLLLLLLRPPATRGITCPPPMSVEHADIW | 11 |
| ApoA (aa) | VKSYSLYSRERYICNSGFKRKAGTSSLTECVLNKATNVAHWTTPS | |
| | LKCIRDPALVHQRPAPPSGGSGGGGSGGGSGGGGSLQNWVNVISD | |
| | LKKIEDLIQSMHIDATLYTESDVHPSCKVTAMKCFLLELQVISLE | |
| | SGDASIHDTVENLIILANNSLSSNGNVTESGCKECEELEEKNIKE | |
| | FLQSFVHIVQMFINTSGAPDEPPQSPWDRVKDLATVYVDVLKDSG | |
| | RDYVSQFEGSALGKQLNLKLLDNWDSVTSTFSKLREQLGPVTQEF | |
| | WDNLEKETEGLRQEMSKDLEEVKAKVQPYLDDFQKKWQEEMELYR | |
| | QKVEPLRAELQEGARQKLHELQEKLSPLGEEMRDRARAHVDALRT | |
| | HLAPYSDELRQRLAARLEALKENGGARLAEYHAKATEHLSTLSEK | |
| | AKPALEDLRQGLLPVLESFKVSFLSALEEYTKKLNTQ | |
| | Signal peptide ″SP1″ (bold); SushiL (bold | |
| | italics); mature IL15 (bold underline); ApoA | |
| | (underline); | |
| Human IL15Rα | MAPRRARGCRTLGLPALLLLLLLRPPATRG | 12 |
| signal peptide | | |
| (SP1) (aa) | | |
| Human IgG heavy | MEFGLSWVFLVAILKGVQC | 13 |
| chain signal | | |
| peptide | | |
| (SP2) (aa) | | |
| Human ApoA-1 | DEPPQSPWDRVKDLATVYVDVLKDSGRDYVSQFEGSALGKQLNLK | 14 |
| (aa) | LLDNWDSVTSTFSKLREQLGPVTQEFWDNLEKETEGLRQEMSKDL | |
| | EEVKAKVQPYLDDFQKKWQEEMELYRQKVEPLRAELQEGARQKLH | |
| | ELQEKLSPLGEEMRDRARAHVDALRTHLAPYSDELRQRLAARLEA | |
| | LKENGGARLAEYHAKATEHLSTLSEKAKPALEDLRQGLLPVLESF | |
| | KVSFLSALEEYTKKLNTQ | |
| Fc domain (aa) | EPKSCDKTHTCPPCPAPELLGGPSVFLFPPKPKDTLMISRTPEVT | 15 |
| | CVVVAVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSRDELTKNQVSLTCLVKGFYPSDIAVEWESNGQPENNYKTTP | |
| | PVLDSDGSFFLYSKLTVDKSRWQQGNVFSCSVMHEALHNHYTQKS | |
| | LSLSPGK | |
| Mature human | NWVNVISDLKKIEDLIQSMHIDATLYTESDVHPSCKVTAMKCFLL | 16 |
| IL15 (aa) | ELQVISLESGDASIHDTVENLIILANNSLSSNGNVTESGCKECEE | |
| | LEEKNIKEFLQSFVHIVQMFINTS | |
| SushiL (aa) | ITCPPPMSVEHADIWVKSYSLYSRERYICNSGFKRKAGTSSLTEC | 17 |
| | VLNKATNVAHWTTPSLKCIRDPALVHQRPAPPS | |
| SushiS (aa) | ITCPPPMSVEHADIWVKSYSLYSRERYICNSGFKRKAGTSSLTEC | 18 |
| | VLNKATNVAHWTTPSLKCIRD | |
| 5v1.1 5′ UTR | GGGAAAUAAGAGAGAAAAGAAGAGUAAGAAGAAAUAUAAGACCCC | 19 |
| (RNA) | GGCGCCGCCACC | |
| 3v1.1 3′ UTR | UGAUAAUAGGCUGGAGCCUCGGUGGCCUAGCUUCUUGCCCCUUGG | 20 |
| (RNA) | GCCUCCCCCCAGCCCCUCCUCCCCUUCCUGCACCCGUACCCCCGU | |
| | GGUCUUUGAAUAAAGUCUGAGUGGGCGGC | |
| Wild-type Full- | MAPRRARGCRTLGLPALLLLLLLRPPATRGITCPPPMSVEHADIW | 21 |
| length human | VKSYSLYSRERYICNSGFKRKAGTSSLTECVLNKATNVAHWTTPS | |
| IL15Rα (aa) | LKCIRDPALVHQRPAPPSTVTTAGVTPQPESLSPSGKEPAASSPS | |
| | SNNTAATTAAIVPGSQLMPSKSPSTGTTEISSHESSHGTPSQTTA | |
| | KNWELTASASHQPPGVYPQGHSDTTVAISTSTVLLCGLSAVSLLA | |
| | CYLKSRQTPPLASVEMEAMEALPVTWGTSSRDEDLENCSHHL | |
| | Signal peptide (italicized), sushi domain of | |
| | the wild-type IL15Rα (double underlined; | |
| | transmembrane domain (bold); intracellular | |
| | domain (underline) | |
| SushiS-IL15-ApoA | ITCPPPMSVEHADIWVKSYSLYSRERYICNSGFKRKAGTSSLTEC | 22 |
| (aa) | VLNKATNVAHWTTPSLKCIRDGGSGGGGSGGGSGGGGSLQNWVNV | |
| | ISDLKKIEDLIQSMHIDATLYTESDVHPSCKVTAMKCFLLELQVI | |
| | SLESGDASIHDTVENLIILANNSLSSNGNVTESGCKECEELEEKN | |
| | IKEFLQSFVHIVQMFINTSGAPDEPPQSPWDRVKDLATVYVDVLK | |
| | DSGRDYVSQFEGSALGKQLNLKLLDNWDSVTSTFSKLREQLGPVT | |
| | QEFWDNLEKETEGLRQEMSKDLEEVKAKVQPYLDDFQKKWQEEME | |
| | LYRQKVEPLRAELQEGARQKLHELQEKLSPLGEEMRDRARAHVDA | |
| | LRTHLAPYSDELRQRLAARLEALKENGGARLAEYHAKATEHLSTL | |
| | SEKAKPALEDLRQGLLPVLESFKVSFLSALEEYTKKLNTQ | |
| | SushiS (bold italics); mature IL15 (bold | |
| | underline); ApoA (underline) | |
| SushiL-IL15-ApoA | ITCPPPMSVEHADIWVKSYSLYSRERYICNSGFKRKAGTSSLTEC | 23 |
| (aa) | VLNKATNVAHWTTPSLKCIRDPALVHQRPAPPSGGSGGGGSGGGS | |
| | GGGGSLQNWVNVISDLKKIEDLIQSMHIDATLYTESDVHPSCKVT | |
| | AMKCFLLELQVISLESGDASIHDTVENLIILANNSLSSNGNVTES | |
| | GCKECEELEEKNIKEFLQSFVHIVQMFINTSGAPDEPPQSPWDRV | |
| | KDLATVYVDVLKDSGRDYVSQFEGSALGKQLNLKLLDNWDSVTST | |
| | FSKLREQLGPVTQEFWDNLEKETEGLRQEMSKDLEEVKAKVQPYL | |
| | DDFQKKWQEEMELYRQKVEPLRAELQEGARQKLHELQEKLSPLGE | |
| | EMRDRARAHVDALRTHLAPYSDELRQRLAARLEALKENGGARLAE | |
| | YHAKATEHLSTLSEKAKPALEDLRQGLLPVLESFKVSFLSALEEY | |
| | TKKLNTQ | |
| | SushiL (bold italics); mature IL15 (bold | |
| | underline); ApoA (underline) | |
| SP2 (RNA-1) | AUGGAGUUCGGCUUGAGCUGGGUGUUCCUAGUGGCCAUCUUGAAG | 24 |
| | GGCGUGCAGUGC | |
| SP2 (RNA-2) | AUGGAGUUCGGCUUAAGCUGGGUGUUCCUCGUGGCCAUCUUAAAG | 25 |
| | GGCGUGCAGUGC | |
| SP2 (RNA-3) | AUGGAGUUCGGCUUGAGCUGGGUGUUCUUAGUGGCCAUCUUAAAG | 26 |
| | GGCGUGCAGUGC | |
| SP1 (RNA-1) | AUGGCUCCUCGACGCGCUCGGGGAUGCCGGACCCUCGGUUUGCCG | 27 |
| | GCGUUGCUACUCCUUCUCCUACUCCGCCCACCUGCCACUAGAGGC | |
| SP1 (RNA-2) | AUGGCCCCUCGGCGGGCCAGAGGCUGCAGAACCUUAGGCCUCCCA | 28 |
| | GCCCUUCUAUUGCUCUUGCUACUCAGACCUCCAGCCACCAGAGGC | |
| SushiL (RNA-1) | AUCACCUGCCCUCCUCCUAUGAGCGUGGAGCACGCCGACAUCUGG | 29 |
| | GUAAAGUCUUACAGCCUGUACAGCAGAGAGAGAUACAUCUGCAAC | |
| | AGCGGCUUCAAGAGAAAGGCCGGCACCAGCAGCCUGACCGAGUGU | |
| | GUCCUGAACAAGGCAACCAACGUGGCCCACUGGACCACCCCUAGC | |
| | CUGAAGUGCAUCAGAGAUCCAGCCCUUGUGCACCAAAGACCUGCC | |
| | CCUCCAUCA | |
| SushiL (RNA-2) | AUAACGUGUCCGCCUCCUAUGAGCGUGGAGCACGCCGACAUCUGG | 30 |
| | GUGAAGAGUUACAGCCUGUAUUCCAGAGAGAGAUACAUCUGCAAC | |
| | AGCGGCUUCAAGAGAAAGGCCGGCACCAGCAGCUUAACCGAGUGC | |
| | GUCCUUAACAAGGCUACCAACGUGGCCCACUGGACAACGCCGAGC | |
| | CUGAAGUGCAUCCGCGACCCUGCCCUGGUGCACCAACGUCCAGCA | |
| | CCUCCGAGU | |
| SushiL (RNA-3) | AUCACCUGCCCUCCUCCUAUGAGCGUGGAGCACGCCGACAUCUGG | 31 |
| | GUAAAGUCCUACAGCCUGUACAGCAGAGAGAGAUACAUCUGCAAC | |
| | AGCGGCUUCAAGAGAAAGGCCGGCACCAGCAGCCUGACCGAGUGC | |
| | GUGCUGAACAAGGCCACCAACGUGGCCCACUGGACCACCCCUAGC | |
| | CUGAAGUGCAUCAGGGACCCAGCUCUAGUGCACCAGCGGCCUGCC | |
| | CCUCCUAGC | |
| SushiS (RNA-1) | AUCACCUGCCCGCCUCCUAUGAGCGUGGAGCACGCCGACAUCUGG | 32 |
| | GUAAAGUCCUACAGCCUGUACAGCAGAGAGAGAUACAUCUGCAAC | |
| | AGCGGCUUCAAGAGAAAGGCCGGCACCAGCAGCCUGACCGAGUGC | |
| | GUGCUUAAUAAGGCCACUAACGUGGCCCACUGGACCACCCCUAGC | |
| | CUGAAGUGCAUCAGAGAC | |
| SushiS (RNA-2) | AUCACGUGCCCUCCUCCCAUGUCCGUGGAACACGCAGACAUCUGG | 33 |
| | GUCAAGAGCUACAGCUUGUACUCCAGGGAGCGGUACAUUUGUAAC | |
| | UCUGGUUUCAAGCGUAAAGCCGGCACGUCCAGCCUGACGGAGUGC | |
| | GUGUUGAACAAGGCCACGAAUGUCGCCCACUGGACAACACCUAGU | |
| | CUCAAAUGCAUUAGAGAC | |
| ApoA (RNA-1) | GACGAGCCUCCUCAGAGCCCUUGGGACAGAGUGAAGGACUUGGCC | 34 |
| | ACCGUGUACGUGGACGUGCUUAAGGACAGCGGCAGAGACUACGUG | |
| | AGCCAGUUCGAGGGCAGCGCCCUGGGCAAGCAGCUGAACCUGAAG | |
| | CUGCUGGACAACUGGGACAGCGUGACCAGCACCUUCAGCAAGCUG | |
| | AGAGAGCAGCUGGGCCCUGUGACCCAGGAGUUCUGGGACAACCUG | |
| | GAGAAGGAGACAGAGGGCCUGAGACAGGAGAUGAGCAAGGACUUA | |
| | GAGGAGGUGAAGGCCAAGGUGCAGCCUUACCUGGACGACUUCCAG | |
| | AAGAAGUGGCAGGAGGAGAUGGAGCUGUACAGACAGAAGGUGGAG | |
| | CCUCUGAGAGCCGAGCUGCAGGAGGGCGCACGGCAGAAGCUGCAC | |
| | GAACUGCAGGAGAAGCUGAGCCCUCUGGGCGAGGAAAUGAGAGAC | |
| | AGAGCCAGAGCCCACGUGGAUGCCCUGAGAACCCACCUGGCCCCU | |
| | UACUCGGACGAGCUGCGACAGAGACUGGCCGCCAGACUGGAGGCC | |
| | CUGAAGGAGAACGGCGGCGCAAGACUCGCAGAGUACCACGCCAAG | |
| | GCCACCGAGCACCUGAGCACCCUGAGCGAGAAGGCUAAGCCUGCU | |
| | CUGGAGGACCUGCGGCAGGGCCUGCUGCCUGUGCUGGAGAGCUUC | |
| | AAGGUGAGCUUCCUGUCCGCACUGGAAGAAUACACCAAGAAGCUG | |
| | AACACCCAG | |
| ApoA (RNA-2) | GACGAGCCUCCUCAGAGCCCUUGGGACAGAGUGAAGGACCUCGCC | 35 |
| | ACCGUGUACGUGGACGUGCUCAAGGACAGCGGCAGAGACUACGUG | |
| | AGCCAGUUCGAGGGCAGCGCCCUGGGCAAGCAGCUGAACCUGAAG | |
| | CUGCUGGACAACUGGGACAGCGUGACCAGCACCUUCAGCAAGCUG | |
| | AGAGAGCAGCUGGGCCCUGUGACCCAGGAGUUCUGGGACAACCUG | |
| | GAGAAGGAGACUGAGGGCCUGAGACAGGAGAUGAGCAAGGAUCUG | |
| | GAGGAGGUGAAGGCCAAGGUGCAGCCUUACCUGGACGACUUCCAG | |
| | AAGAAGUGGCAGGAGGAGAUGGAGCUGUACAGACAGAAGGUGGAG | |
| | CCUCUGAGAGCCGAGCUGCAGGAGGGCGCACGUCAGAAGCUGCAC | |
| | GAACUUCAGGAGAAGCUGAGCCCUCUGGGCGAAGAAAUGAGAGAC | |
| | AGAGCCAGAGCCCAUGUGGACGCCCUGAGAACCCACCUGGCCCCU | |
| | UACAGCGACGAACUGAGACAAAGACUGGCCGCCAGACUGGAGGCC | |
| | CUGAAGGAGAACGGCGGUGCGAGAUUAGCUGAGUACCACGCCAAG | |
| | GCCACCGAGCACCUGAGCACCCUGAGCGAGAAGGCUAAGCCUGCC | |
| | CUCGAGGACCUGCGACAAGGCCUGCUGCCUGUGCUGGAGAGCUUC | |
| | AAGGUGAGCUUCCUGUCAGCACUUGAAGAGUACACCAAGAAGCUG | |
| | AACACCCAG | |
| ApoA (RNA-3) | GAUGAACCACCCCAGAGCCCCUGGGAUCGAGUGAAGGACCUGGCC | 36 |
| | ACUGUGUACGUGGAUGUGCUCAAAGACAGCGGCAGAGACUAUGUG | |
| | UCCCAGUUUGAAGGCUCCGCCUUGGGCAAGCAGCUAAACCUAAAG | |
| | CUCCUUGACAACUGGGACAGCGUGACCUCCACCUUCAGCAAGCUG | |
| | CGCGAACAGCUCGGCCCUGUGACCCAGGAGUUCUGGGAUAACCUG | |
| | GAGAAGGAGACAGAGGGCCUGAGGCAGGAGAUGAGCAAGGACCUA | |
| | GAGGAGGUGAAGGCCAAGGUGCAGCCCUACCUGGACGACUUCCAG | |
| | AAGAAGUGGCAGGAGGAGAUGGAGCUCUACCGCCAGAAGGUGGAG | |
| | CCGCUGCGCGCAGAGCUCCAAGAAGGUGCCAGACAGAAGCUGCAC | |
| | GAGCUGCAAGAGAAGCUGUCUCCACUGGGCGAAGAGAUGCGCGAC | |
| | AGAGCUCGCGCGCAUGUGGACGCGCUGCGCACGCAUCUGGCACCU | |
| | UACAGCGAUGAGCUCCGCCAGCGCUUGGCCGCGCGCCUUGAGGCU | |
| | CUCAAGGAGAACGGCGGCGCCAGACUGGCCGAGUACCACGCUAAG | |
| | GCGACCGAGCAUCUGAGCACGCUCAGCGAGAAGGCAAAGCCCGCG | |
| | CUCGAGGACCUCCGACAAGGACUGCUGCCCGUGCUGGAGAGCUUC | |
| | AAGGUCAGCUUCCUGAGCGCUCUCGAGGAGUACACUAAGAAGCUC | |
| | AACACCCAG | |
| ApoA (RNA-4) | GACGAGCCUCCUCAGAGCCCUUGGGACAGAGUGAAGGACCUGGCC | 37 |
| | ACCGUGUACGUGGACGUGUUAAAGGACAGCGGCAGAGACUACGUG | |
| | AGCCAGUUCGAGGGCAGCGCCCUGGGCAAGCAGCUGAACCUGAAG | |
| | CUGCUGGACAACUGGGACAGCGUGACCAGCACCUUCAGCAAGCUG | |
| | AGAGAGCAGCUGGGCCCUGUGACCCAGGAGUUCUGGGACAACCUG | |
| | GAGAAGGAAACUGAGGGCCUGAGACAGGAGAUGAGCAAGGACCUU | |
| | GAAGAAGUGAAGGCCAAGGUGCAGCCUUACCUGGACGACUUCCAG | |
| | AAGAAGUGGCAGGAGGAGAUGGAGCUGUACAGACAGAAGGUGGAG | |
| | CCUCUGAGAGCCGAACUUCAGGAGGGCGCUCGCCAGAAGCUGCAC | |
| | GAGCUACAAGAGAAGCUGAGCCCUCUGGGCGAAGAGAUGCGCGAC | |
| | CGGGCUAGGGCCCAUGUUGACGCCCUGAGAACCCACCUGGCCCCU | |
| | UACAGCGAUGAACUGCGCCAGCGCCUGGCCGCCAGACUGGAGGCC | |
| | CUGAAGGAGAACGGCGGCGCUCGACUAGCCGAGUACCACGCCAAG | |
| | GCUACUGAGCACCUGAGCACUCUGAGCGAGAAGGCGAAGCCGGCC | |
| | CUGGAGGAUCUCAGGCAAGGCCUGCUGCCUGUGCUGGAGUCAUUC | |
| | AAGGUGAGCUUCCUGUCCGCGCUCGAAGAGUACACCAAGAAGUUG | |
| | AACACCCAG | |
| Mature IL15 | AACUGGGUAAACGUGAUCAGCGACCUGAAGAAGAUCGAAGAUCUG | 38 |
| (RNA-1) | AUCCAGAGCAUGCACAUCGACGCCACCCUGUACACCGAGAGCGAC | |
| | GUGCACCCUAGCUGCAAGGUGACCGCCAUGAAGUGCUUCCUGCUG | |
| | GAACUCCAGGUUAUCUCCCUGGAGAGUGGCGACGCCAGCAUCCAC | |
| | GACACCGUGGAGAACCUGAUCAUCCUGGCCAACAACAGCCUGAGC | |
| | AGCAACGGCAACGUGACUGAAAGCGGCUGUAAGGAGUGCGAGGAG | |
| | CUCGAGGAGAAGAACAUCAAGGAGUUCCUGCAGAGCUUCGUCCAC | |
| | AUCGUGCAGAUGUUCAUCAACACCAGC | |
| Mature IL15 | AACUGGGUAAACGUGAUCAGCGACCUGAAGAAGAUCGAAGAUCUC | 39 |
| (RNA-2) | AUCCAGAGCAUGCACAUCGACGCCACCCUGUACACCGAGAGCGAC | |
| | GUGCACCCUAGCUGCAAGGUGACCGCCAUGAAGUGCUUCCUGCUG | |
| | GAACUGCAGGUGAUAAGCCUGGAGUCUGGCGACGCCAGCAUCCAC | |
| | GACACCGUGGAGAACCUGAUCAUCCUGGCCAACAACAGCCUGAGC | |
| | AGCAACGGCAACGUAACCGAGUCCGGCUGUAAGGAGUGCGAGGAG | |
| | UUGGAGGAGAAGAACAUCAAGGAGUUCCUGCAGAGCUUCGUUCAC | |
| | AUCGUGCAGAUGUUCAUCAACACCAGC | |
| Mature IL15 | AAUUGGGUGAACGUGAUCAGCGACCUGAAGAAGAUCGAGGACCUG | 40 |
| (RNA-3) | AUCCAGAGCAUGCACAUCGACGCCACCCUGUACACCGAGAGCGAC | |
| | GUGCACCCUAGCUGUAAGGUAACCGCCAUGAAGUGCUUCCUGCUG | |
| | GAGCUGCAGGUGAUUAGCCUGGAGAGCGGCGACGCCAGCAUCCAC | |
| | GACACCGUGGAGAACCUGAUCAUCCUGGCCAACAAUUCUCUCAGU | |
| | UCUAACGGUAACGUGACCGAAUCGGGUUGUAAGGAGUGCGAGGAG | |
| | CUGGAGGAGAAGAACAUCAAGGAGUUCCUGCAGAGCUUCGUGCAC | |
| | AUUGUGCAGAUGUUCAUCAACACCAGC | |
| Mature IL15 | AACUGGGUGAAUGUAAUAAGUGAUUUGAAGAAGAUUGAAGAUCUU | 41 |
| (RNA-4) | AUUCAAUCUAUGCAUAUUGAUGCUACUUUAUAUACGGAAAGUGAU | |
| | GUUCACCCCAGUUGCAAAGUAACAGCAAUGAAGUGCUUUCUCUUG | |
| | GAGUUACAAGUUAUUUCACUUGAGUCCGGAGAUGCAAGUAUUCAU | |
| | GAUACAGUAGAGAAUCUGAUCAUCCUAGCAAACAACAGUUUGUCU | |
| | UCUAAUGGGAAUGUAACAGAAUCUGGAUGCAAAGAAUGUGAGGAA | |
| | CUGGAGGAGAAGAACAUUAAAGAAUUCUUGCAGAGUUUUGUACAU | |
| | AUUGUCCAAAUGUUCAUCAACACUUCU | |
| Mature IL15 | AACUGGGUUAACGUGAUCAGCGACCUGAAGAAGAUCGAGGACCUG | 42 |
| (RNA-5) | AUCCAGAGCAUGCACAUCGACGCCACCCUGUACACCGAGAGCGAC | |
| | GUGCACCCUAGCUGCAAGGUGACCGCCAUGAAGUGCUUCCUGCUG | |
| | GAGCUGCAGGUAAUUUCUCUGGAGAGCGGCGACGCCAGCAUCCAC | |
| | GACACCGUGGAGAACCUGAUCAUCCUGGCCAACAACAGCCUGAGC | |
| | AGCAACGGCAACGUGACUGAGUCCGGAUGCAAGGAGUGCGAGGAG | |
| | CUGGAGGAGAAGAACAUCAAGGAGUUCCUGCAGAGCUUCGUGCAU | |
| | AUCGUGCAGAUGUUCAUCAACACCAGC | |
| Fc domain (RNA- | GAGCCUAAGAGCUGCGACAAGACCCACACCUGCCCUCCUUGCCCG | 43 |
| 1) | GCCCCAGAGUUGCUUGGCGGCCCUAGCGUCUUCCUCUUCCCUCCU | |
| | AAGCCUAAGGACACCCUGAUGAUCAGCAGAACCCCUGAGGUGACC | |
| | UGCGUGGUGGUGGCCGUGAGCCACGAGGACCCUGAAGUAAAGUUC | |
| | AACUGGUACGUGGACGGCGUGGAGGUGCACAACGCCAAGACCAAG | |
| | CCUAGAGAGGAGCAGUACAACAGCACCUACAGAGUGGUGAGCGUG | |
| | CUGACCGUGCUGCACCAGGACUGGCUGAACGGCAAGGAGUACAAG | |
| | UGCAAGGUGAGCAACAAGGCCCUGCCUGCACCUAUCGAGAAGACC | |
| | AUCAGCAAGGCCAAGGGCCAACCUCGAGAGCCUCAGGUGUACACC | |
| | CUGCCUCCUAGCAGAGACGAGCUGACCAAGAACCAGGUGAGCCUG | |
| | ACCUGCCUGGUGAAGGGCUUCUACCCUAGCGACAUCGCCGUGGAG | |
| | UGGGAGAGCAACGGCCAACCAGAGAACAACUACAAGACCACCCCU | |
| | CCUGUGCUGGACAGCGACGGCAGCUUCUUCCUCUACAGCAAGCUG | |
| | ACUGUUGACAAGAGCAGAUGGCAGCAGGGCAACGUGUUCAGCUGC | |
| | AGCGUGAUGCACGAGGCGCUGCACAACCACUACACCCAGAAGAGU | |
| | CUUUCCCUGAGCCCUGGCAAG | |
| Wild Type human | MRISKPHLRSISIQCYLCLLLNSHFLTEAGIHVFILGCFSAGLPK | 44 |
| full-Length IL15 | 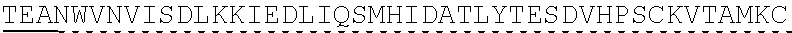 | |
| (aa) | 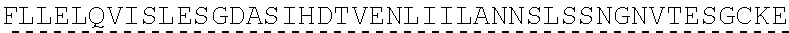 | |
| | 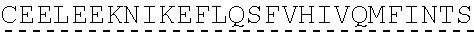 | |
| | signal peptide (italized); propetide (solid | |
| | underlined); mature IL15 (dot underlined) | |
| Wild Type Full- | AUGCGCAUCAGCAAACCUCAUUUACGGAGUAUCAGCAUCCAGUGC | 45 |
| Length human | UAUCUCUGCCUGCUUCUGAACAGUCAUUUUCUGACUGAGGCGGGC | |
| IL15 (RNA) | AUUCAUGUCUUUAUUUUAGGCUGCUUUUCCGCAGGUCUGCCCAAA | |
| | 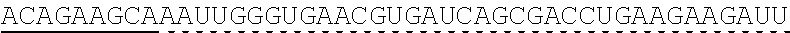 | |
| | 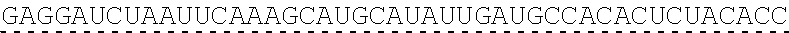 | |
| | 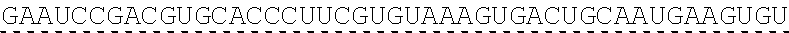 | |
| | 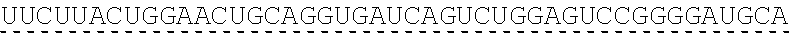 | |
| | 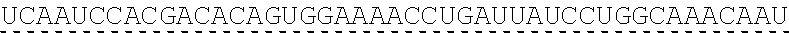 | |
| | 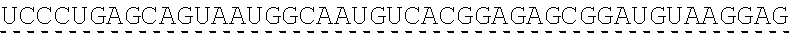 | |
| | 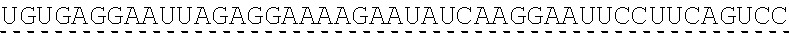 | |
| | 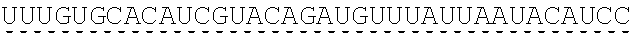 | |
| | signal peptide (italized); propetide (solid | |
| | underlined); mature IL15 (dot underlined) | |
| Wild-type Full- | AUGGCUCCCCGCCGCGCGCGAGGCUGUCGCACCCUCGGACUUCCU | 46 |
| Length human | GCACUCUUGCUUUUGCUCCUCCUUAGACCCCCUGCAACCAGAGGG | |
| IL15Rα (RNA) | AUAACCUGUCCACCUCCAAUGAGCGUCGAGCACGCAGACAUUUGG | |
| | GUGAAAUCAUACAGUCUGUACAGUAGAGAGCGGUACAUCUGCAAC | |
| | AGUGGGUUUAAAAGAAAAGCAGGCACUUCAUCUCUGACAGAGUGC | |
| | GUGCUGAACAAAGCAACUAAUGUAGCUCAUUGGACAACCCCAUCA | |
| | CUGAAAUGCAUUAGAGAUCCAGCUCUGGUGCAUCAAAGACCAGCA | |
| | CCACCAAGUACCGUAACAACCGCAGGGGUGACCCCUCAGCCUGAG | |
| | UCCCUAUCUCCCUCCGGCAAGGAGCCAGCAGCAUCUUCACCUAGC | |
| | UCCAAUAACACCGCAGCUACCACUGCCGCCAUAGUCCCCGGAAGC | |
| | CAGCUGAUGCCUAGUAAAUCUCCCUCAACAGGUACCACCGAAAUU | |
| | UCUAGCCAUGAGUCCUCGCACGGCACCCCGUCACAGACUACAGCU | |
| | AAAAACUGGGAGCUAACGGCUUCGGCAUCCCACCAACCUCCAGGC | |
| | GUUUAUCCCCAAGGUCACUCCGACACUACUGUGGCGAUUAGCACA | |
| | AGUACCGUCCUUCUGUGUGGACUGAGUGCAGUUUCAUUGCUGGCC | |
| | UGUUAUCUGAAAUCUCGCCAGACCCCUCCCCUCGCCAGUGUUGAG | |
| | AUGGAAGCCAUGGAAGCACUUCCUGUGACUUGGGGAACAUCCUCG | |
| | AGGGACGAGGACCUCGAGAACUGCUCUCACCACCUG | |
| | Signal peptide (italicized); sushi domain of | |
| | the wild-type IL15Rα (double underlined); | |
| | transmembrane domain (bold); intracellular | |
| | domain (underline) | |
| sushi domain of | CPPPMSVEHADIWVKSYSLYSRERYICNSGFKRKAGTSSLTECVL | 47 |
| the wild-type | NKATNVAHWTTPSLKC | |
| IL15Rα (aa) | | |
| sushi domain of | UGUCCACCUCCAAUGAGCGUCGAGCACGCAGACAUUUGGGUGAAA | 48 |
| the wild-type | UCAUACAGUCUGUACAGUAGAGAGCGGUACAUCUGCAACAGUGGG | |
| IL15Rα (RNA) | UUUAAAAGAAAAGCAGGCACUUCAUCUCUGACAGAGUGCGUGCUG | |
| | AACAAAGCAACUAAUGUAGCUCAUUGGACAACCCCAUCACUGAAA | |
| | UGC | |
| Human IL15Rα | VAISTSTVLLCGLSAVSLLACYL | 49 |
| transmembrane | | |
| domain (aa) | | |
| Human IL15Rα | KSRQTPPLASVEMEAMEALPVTWGTSSRDEDLENCSHHL | 50 |
| intracellular | | |
| domain (aa) | | |
| Wild-type human | MAPRRARGCRTLGLPALLLLLLLRPPATRGITCPPPMSVEHADIW | 51 |
| IL15Rα | VKSYSLYSRERYICNSGFKRKAGTSSLTECVLNKATNVAHWTTPS | |
| ectodomain (aa) | LKCIRDPALVHQRPAPPSTVTTAGVTPQPESLSPSGKEPAASSPS | |
| | SNNTAATTAAIVPGSQLMPSKSPSTGTTEISSHESSHGTPSQTTA | |
| | KNWELTASASHQPPGVYPQGHSDTT | |
| | Signal peptide (italicized), sushi domain of | |
| | the wild-type IL15Rα (double underlined) | |
| Wild-type human | AUGGCUCCCCGCCGCGCGCGAGGCUGUCGCACCCUCGGACUUCCU | 52 |
| IL15Rα | GCACUCUUGCUUUUGCUCCUCCUUAGACCCCCUGCAACCAGAGGG | |
| ectodomain (RNA) | AUAACCUGUCCACCUCCAAUGAGCGUCGAGCACGCAGACAUUUGG | |
| | GUGAAAUCAUACAGUCUGUACAGUAGAGAGCGGUACAUCUGCAAC | |
| | AGUGGGUUUAAAAGAAAAGCAGGCACUUCAUCUCUGACAGAGUGC | |
| | GUGCUGAACAAAGCAACUAAUGUAGCUCAUUGGACAACCCCAUCA | |
| | CUGAAAUGCAUUAGAGAUCCAGCUCUGGUGCAUCAAAGACCAGCA | |
| | CCACCAAGUACCGUAACAACCGCAGGGGUGACCCCUCAGCCUGAG | |
| | UCCCUAUCUCCCUCCGGCAAGGAGCCAGCAGCAUCUUCACCUAGC | |
| | UCCAAUAACACCGCAGCUACCACUGCCGCCAUAGUCCCCGGAAGC | |
| | CAGCUGAUGCCUAGUAAAUCUCCCUCAACAGGUACCACCGAAAUU | |
| | UCUAGCCAUGAGUCCUCGCACGGCACCCCGUCACAGACUACAGCU | |
| | AAAAACUGGGAGCUAACGGCUUCGGCAUCCCACCAACCUCCAGGC | |
| | GUUUAUCCCCAAGGUCACUCCGACACUACU | |
| | Signal peptide (italicized) ; sushi domain of | |
| | the wild-type IL15Rα (double underlined) | |
| Linker | (GGGGS) o; o = 1-5 | 53 |
| Linker | GGSGGGGSGG | 54 |
| Linker | GGSGGGGG | 55 |
| Linker | GSGSGSGS | 56 |
| Linker | GGGGGGS | 57 |
| Linker | GGGGG | 58 |
| Linker | GGGGGG | 59 |
| Linker | GGGGGGG | 60 |
| Linker | GGGGGGGG | 61 |
| Linker | (EAAAK)_{q} q = 1-5 | 62 |
| Linker | EAAAKEAAAKEAAAK | 63 |
| Linker | GGGGSLVPRGSGGGGS | 64 |
| Linker | GSGSGS | 65 |
| Linker | GGGGSLVPRGSGGGG | 66 |
| Linker | GGSGGHMGSGG | 67 |
| Linker | GGSGGSGGSGG | 68 |
| Linker | GGSGG | 69 |
| Linker | GGGSEGGGSEGGGSEGGG | 70 |
| Linker | AAGAATAA | 71 |
| Linker | GGSSG | 72 |
| Linker | GSGGGTGGGSG | 73 |
| Linker | GSGSGSGSGGSG | 74 |
| Linker | GSGGSGSGGSGGSG | 75 |
| Linker | GGGSGGGSGGGS | 76 |
| Linker | GGS (GGGS) 3LQ | 77 |
| Linker | GSGGSGGSGGSGGS | 78 |
| Linker | (Gly)p; p = 1-40 | 79 |
| 5′ UTR-001 | GGGAAAUAAGAGAGAAAAGAAGAGUAAGAAGAAAUAUAAGACCCC | 80 |
| | GGCGCCACC | |
| 5′ UTR-002 | GGGAGAUCAGAGAGAAAAGAAGAGUAAGAAGAAAUAUAAGAGCCA | 81 |
| | CC | |
| 5′ UTR-004 | GGGAGACAAGCUUGGCAUUCCGGUACUGUUGGUAAAGCCACC | 82 |
| 5′ UTR-008 | GGGAAUUAACAGAGAAAAGAAGAGUAAGAAGAAAUAUAAGAGCCA | 83 |
| | CC | |
| 5′ UTR-009 | GGGAAAUUAGACAGAAAAGAAGAGUAAGAAGAAAUAUAAGAGCCA | 84 |
| | CC | |
| 5′ UTR-010 | GGGAAAUAAGAGAGUAAAGAACAGUAAGAAGAAAUAUAAGAGCCA | 85 |
| | CC | |
| 5′ UTR-011 | GGGAAAAAAGAGAGAAAAGAAGACUAAGAAGAAAUAUAAGAGCCA | 86 |
| | CC | |
| 5′ UTR-012 | GGGAAAUAAGAGAGAAAAGAAGAGUAAGAAGAUAUAUAAGAGCCA | 87 |
| | CC | |
| 5′ UTR-013 | GGGAAAUAAGAGACAAAACAAGAGUAAGAAGAAAUAUAAGAGCCA | 88 |
| | CC | |
| 5′ UTR-014 | GGGAAAUUAGAGAGUAAAGAACAGUAAGUAGAAUUAAAAGAGCCA | 89 |
| | CC | |
| 5′ UTR-015 | GGGAAAUAAGAGAGAAUAGAAGAGUAAGAAGAAAUAUAAGAGCCA | 90 |
| | CC | |
| 5′ UTR-016 | GGGAAAUAAGAGAGAAAAGAAGAGUAAGAAGAAAAUUAAGAGCCA | 91 |
| | CC | |
| 5′ UTR-017 | GGGAAAUAAGAGAGAAAAGAAGAGUAAGAAGAAAUUUAAGAGCCA | 92 |
| | CC | |
| 5′ UTR-018 | GGGAAAUAAGAGAGAAAAGAAGAGUAAGAAGAAAUAUAAGA | 93 |
| 5′ UTR-019 | GGGAAATAAGAGAGAAAAGAAGAGTAAGAAGAAATATAAGACCCC | 94 |
| | GGCGCCGCCACC | |
| 5′ UTR-020 | GGGAAATAAGAGAGAAAAGAAGAGTAAGAAGAAATATAAGACCCC | 95 |
| | GGCGCCACC | |
| 5′ UTR-021 | GGGAAATAAGAGAGAAAAGAAGAGTAAGAAGAAATATAAGAGCCA | 96 |
| | CC | |
| 5′ UTR-022 | GGGAAATAAGAGAGAAAAGAAGAGTAAGAAGAAATATAAGA | 97 |
| 3′ UTR with miR- | UGAUAAUAGGCUGGAGCCUCGGUGGCCUAGCUUCUUGCCCCUUGG | 98 |
| 122 | GCCUCCCCCCAGCCCCUCCUCCCCUUCCUGCACCCGUACCCCCCA | |
| | AACACCAUUGUCACACUCCAGUGGUCUUUGAAUAAAGUCUGAGUG | |
| | GGCGGC | |
| Kozak consensus | CCR (A/G) CCAUGG | |
| sequence | | |
| 2A peptide | GSGATNFSLLKQAGDVEENPGP | 100 |
| 2A peptide (DNA) | GGAAGCGGAGCTACTAACTTCAGCCTGCTGAAGCAGGCTGGAGAC | 101 |
| | GTGGAGGAGAACCCTGGACCT | |
| 2A peptide (DNA) | TCCGGACTCAGATCCGGGGATCTCAAAATTGTCGCTCCTGTCAAA | 102 |
| | CAAACTCTTAACTTTGATTTACTCAAACTGGCTGGGGATGTAGAA | |
| | AGCAATCCAGGTCCACTC | |
| GC-rich motif | [CCG]n; n = 1-10 | 103 |
| V1 GC-rich RNA | CCCCGGCGCC | 104 |
| element | | |
| V2 GC-rich RNA | CCCCGGC | |
| element | | |
| EK GC-rich RNA | GCCGCC | |
| element | | |
| 5′ UTR-023 | GGGAAAUAAGAGAGAAAAGAAGAGUAAGAAGAAAUAUAAGAGCCA | 107 |
| | CC | |
| 5′ UTR-024 | UCAAGCUUUUGGACCCUCGUACAGAAGCUAAUACGACUCACUAUA | 108 |
| | GGGAAAUAAGAGAGAAAAGAAGAGUAAGAAGAAAUAUAAGAGCCA | |
| | CC | |
| 3′ UTR | UGAUAAUAGGCUGGAGCCUCGGUGGCCAUGCUUCUUGCCCCUUGG | 109 |
| | GCCUCCCCCCAGCCCCUCCUCCCCUUCCUGCACCCGUACCCCCCA | |
| | AACACCAUUGUCACACUCCAGUGGUCUUUGAAUAAAGUCUGAGUG | |
| | GGCGGC | |
| 3′ UTR with mi- | UGAUAAUAGGCUGGAGCCUCGGUGGCCAUGCUUCUUGCCCCUUGG | 110 |
| 122 and mi- | GCCCAAACACCAUUGUCACACUCCAUCCCCCCAGCCCCUCCUCCC | |
| 142.3p sites | CUUCCUCCAUAAAGUAGGAAACACUACAUGCACCCGUACCCCCGU | |
| | GGUCUUUGAAUAAAGUCUGAGUGGGCGGC | |
| miR-122 | CCUUAGCAGAGCUGUGGAGUGUGACAAUGGUGUUUGUGUCUAAAC | 111 |
| | UAUCAAACGCCAUUAUCACACUAAAUAGCUACUGCUAGGC | |
| miR-122-3p | AACGCCAUUAUCACACUAAAUA | 112 |
| miR-122-3p | UAUUUAGUGUGAUAAUGGCGUU | 113 |
| binding site | | |
| miR-122-5p | UGGAGUGUGACAAUGGUGUUUG | 114 |
| miR-122-5p | CAAACACCAUUGUCACACUCCA | 115 |
| binding site | | |
| hsa-miR-21-5p | UAGCUUAUCAGACUGAUGUUGA | 116 |
| hsa-miR-21-3p | CAACACCAGUCGAUGGGCUGU | 117 |
| hsa-miR-142-3p | UGUAGUGUUUCCUACUUUAUGGA | 118 |
| hsa-miR-142-5p | CAUAAAGUAGAAAGCACUACU | 119 |
| ApoA-SushiL-IL15 | GACGAGCCUCCUCAGAGCCCUUGGGACAGAGUGAAGGACUUGGCC | 120 |
| (RNA) | ACCGUGUACGUGGACGUGCUUAAGGACAGCGGCAGAGACUACGUG | |
| | AGCCAGUUCGAGGGCAGCGCCCUGGGCAAGCAGCUGAACCUGAAG | |
| | CUGCUGGACAACUGGGACAGCGUGACCAGCACCUUCAGCAAGCUG | |
| | AGAGAGCAGCUGGGCCCUGUGACCCAGGAGUUCUGGGACAACCUG | |
| | GAGAAGGAGACAGAGGGCCUGAGACAGGAGAUGAGCAAGGACUUA | |
| | GAGGAGGUGAAGGCCAAGGUGCAGCCUUACCUGGACGACUUCCAG | |
| | AAGAAGUGGCAGGAGGAGAUGGAGCUGUACAGACAGAAGGUGGAG | |
| | CCUCUGAGAGCCGAGCUGCAGGAGGGCGCACGGCAGAAGCUGCAC | |
| | GAACUGCAGGAGAAGCUGAGCCCUCUGGGCGAGGAAAUGAGAGAC | |
| | AGAGCCAGAGCCCACGUGGAUGCCCUGAGAACCCACCUGGCCCCU | |
| | UACUCGGACGAGCUGCGACAGAGACUGGCCGCCAGACUGGAGGCC | |
| | CUGAAGGAGAACGGCGGCGCAAGACUCGCAGAGUACCACGCCAAG | |
| | GCCACCGAGCACCUGAGCACCCUGAGCGAGAAGGCUAAGCCUGCU | |
| | CUGGAGGACCUGCGGCAGGGCCUGCUGCCUGUGCUGGAGAGCUUC | |
| | AAGGUGAGCUUCCUGUCCGCACUGGAAGAAUACACCAAGAAGCUG | |
| | AACACCCAGGGCGGCGGAGGAUCGGGUGGAGGUGGUAGUGGUGGU | |
| | GGCGGUUCCAUCACCUGCCCUCCUCCUAUGAGCGUGGAGCACGCC | |
| | GACAUCUGGGUAAAGUCUUACAGCCUGUACAGCAGAGAGAGAUAC | |
| | AUCUGCAACAGCGGCUUCAAGAGAAAGGCCGGCACCAGCAGCCUG | |
| | ACCGAGUGUGUCCUGAACAAGGCAACCAACGUGGCCCACUGGACC | |
| | ACCCCUAGCCUGAAGUGCAUCAGAGAUCCAGCCCUUGUGCACCAA | |
| | AGACCUGCCCCUCCAUCAGGAGGUUCCGGCGGUGGAGGAUCAGGC | |
| | GGCGGCAGCGGAGGUGGCGGAUCCCUGCAGAACUGGGUAAACGUG | |
| | AUCAGCGACCUGAAGAAGAUCGAAGAUCUGAUCCAGAGCAUGCAC | |
| | AUCGACGCCACCCUGUACACCGAGAGCGACGUGCACCCUAGCUGC | |
| | AAGGUGACCGCCAUGAAGUGCUUCCUGCUGGAACUCCAGGUUAUC | |
| | UCCCUGGAGAGUGGCGACGCCAGCAUCCACGACACCGUGGAGAAC | |
| | CUGAUCAUCCUGGCCAACAACAGCCUGAGCAGCAACGGCAACGUG | |
| | ACUGAAAGCGGCUGUAAGGAGUGCGAGGAGCUCGAGGAGAAGAAC | |
| | AUCAAGGAGUUCCUGCAGAGCUUCGUCCACAUCGUGCAGAUGUUC | |
| | AUCAACACCAGC | |
| | ApoA (underline); SushiL (bold italics); | |
| | mature IL15 (bold underline) | |
| ApoA-SushiL-IL15 | DEPPQSPWDRVKDLATVYVDVLKDSGRDYVSQFEGSALGKQLNLK | 121 |
| (aa) | LLDNWDSVTSTFSKLREQLGPVTQEFWDNLEKETEGLRQEMSKDL | |
| | EEVKAKVQPYLDDFQKKWQEEMELYRQKVEPLRAELQEGARQKLH | |
| | ELQEKLSPLGEEMRDRARAHVDALRTHLAPYSDELRQRLAARLEA | |
| | LKENGGARLAEYHAKATEHLSTLSEKAKPALEDLRQGLLPVLESE | |
| | KVSFLSALEEYTKKLNTQGGGGSGGGGSGGGGSITCPPPMSVEHA | |
| | DIWVKSYSLYSRERYICNSGFKRKAGTSSLTECVLNKATNVAHWT | |
| | TPSLKCIRDPALVHQRPAPPSGGSGGGGSGGGSGGGGSLQNWVNV | |
| | ISDLKKIEDLIQSMHIDATLYTESDVHPSCKVTAMKCFLLELQVI | |
| | SLESGDASIHDTVENLIILANNSLSSNGNVTESGCKECEELEEKN | |
| | IKEFLQSFVHIVQMFINTS | |
| | ApoA (underline); SushiL (bold italics); | |
| | mature IL15 (bold underline) | |
| ApoA-SushiS-IL15 | GACGAGCCUCCUCAGAGCCCUUGGGACAGAGUGAAGGACCUCGCC | 122 |
| (RNA) | ACCGUGUACGUGGACGUGCUCAAGGACAGCGGCAGAGACUACGUG | |
| | AGCCAGUUCGAGGGCAGCGCCCUGGGCAAGCAGCUGAACCUGAAG | |
| | CUGCUGGACAACUGGGACAGCGUGACCAGCACCUUCAGCAAGCUG | |
| | AGAGAGCAGCUGGGCCCUGUGACCCAGGAGUUCUGGGACAACCUG | |
| | GAGAAGGAGACUGAGGGCCUGAGACAGGAGAUGAGCAAGGAUCUG | |
| | GAGGAGGUGAAGGCCAAGGUGCAGCCUUACCUGGACGACUUCCAG | |
| | AAGAAGUGGCAGGAGGAGAUGGAGCUGUACAGACAGAAGGUGGAG | |
| | CCUCUGAGAGCCGAGCUGCAGGAGGGCGCACGUCAGAAGCUGCAC | |
| | GAACUUCAGGAGAAGCUGAGCCCUCUGGGCGAAGAAAUGAGAGAC | |
| | AGAGCCAGAGCCCAUGUGGACGCCCUGAGAACCCACCUGGCCCCU | |
| | UACAGCGACGAACUGAGACAAAGACUGGCCGCCAGACUGGAGGCC | |
| | CUGAAGGAGAACGGCGGUGCGAGAUUAGCUGAGUACCACGCCAAG | |
| | GCCACCGAGCACCUGAGCACCCUGAGCGAGAAGGCUAAGCCUGCC | |
| | CUCGAGGACCUGCGACAAGGCCUGCUGCCUGUGCUGGAGAGCUUC | |
| | AAGGUGAGCUUCCUGUCAGCACUUGAAGAGUACACCAAGAAGCUG | |
| | AACACCCAGGGCGGCGGCGGAAGCGGCGGUGGCGGCAGUGGAGGC | |
| | GGUGGAAGCAUCACCUGCCCGCCUCCUAUGAGCGUGGAGCACGCC | |
| | GACAUCUGGGUAAAGUCCUACAGCCUGUACAGCAGAGAGAGAUAC | |
| | AUCUGCAACAGCGGCUUCAAGAGAAAGGCCGGCACCAGCAGCCUG | |
| | ACCGAGUGCGUGCUUAAUAAGGCCACUAACGUGGCCCACUGGACC | |
| | ACCCCUAGCCUGAAGUGCAUCAGAGACGGCGGUUCAGGAGGCGGA | |
| | GGUUCCGGUGGAGGCUCAGGUGGAGGUGGUAGCCUGCAGAACUGG | |
| | GUAAACGUGAUCAGCGACCUGAAGAAGAUCGAAGAUCUCAUCCAG | |
| | AGCAUGCACAUCGACGCCACCCUGUACACCGAGAGCGACGUGCAC | |
| | CCUAGCUGCAAGGUGACCGCCAUGAAGUGCUUCCUGCUGGAACUG | |
| | CAGGUGAUAAGCCUGGAGUCUGGCGACGCCAGCAUCCACGACACC | |
| | GUGGAGAACCUGAUCAUCCUGGCCAACAACAGCCUGAGCAGCAAC | |
| | GGCAACGUAACCGAGUCCGGCUGUAAGGAGUGCGAGGAGUUGGAG | |
| | GAGAAGAACAUCAAGGAGUUCCUGCAGAGCUUCGUUCACAUCGUG | |
| | CAGAUGUUCAUCAACACCAGC | |
| | ApoA (underline); SushiS (bold italics); | |
| | mature IL15 (bold underline) | |
| ApoA-SushiS-IL15 | DEPPQSPWDRVKDLATVYVDVLKDSGRDYVSQFEGSALGKQLNLK | 123 |
| (aa) | LLDNWDSVTSTFSKLREQLGPVTQEFWDNLEKETEGLRQEMSKDL | |
| | EEVKAKVQPYLDDFQKKWQEEMELYRQKVEPLRAELQEGARQKLH | |
| | ELQEKLSPLGEEMRDRARAHVDALRTHLAPYSDELRQRLAARLEA | |
| | LKENGGARLAEYHAKATEHLSTLSEKAKPALEDLRQGLLPVLESF | |
| | KVSFLSALEEYTKKLNTQGGGGSGGGGSGGGGSITCPPPMSVEHA | |
| | DIWVKSYSLYSRERYICNSGFKRKAGTSSLTECVLNKATNVAHWT | |
| | TPSLKCIRDGGSGGGGSGGGSGGGGSLQNWVNVISDLKKIEDLIQ | |
| | SMHIDATLYTESDVHPSCKVTAMKCFLLELQVISLESGDASIHDT | |
| | VENLIILANNSLSSNGNVTESGCKECEELEEKNIKEFLQSFVHIV | |
| | QMFINTS | |
| | ApoA (underline); SushiS (bold italics); | |
| | mature IL15 (bold underline) | |
| Wild-type human | ITCPPPMSVEHADIWVKSYSLYSRERYICNSGFKRKAGTSSLTEC | 124 |
| IL15Rα | VLNKATNVAHWTTPSLKCIRDPALVHQRPAPPSTVTTAGVTPQPE | |
| ectodomain | SLSPSGKEPAASSPSSNNTAATTAAIVPGSQLMPSKSPSTGTTEI | |
| (without signal | SSHESSHGTPSQTTAKNWELTASASHQPPGVYPQGHSDTT | |
| peptide) (aa) | | |
| | sushi domain of the wild-type IL15Rα (double | |
| | underlined) | |
| Wild-type human | AUAACCUGUCCACCUCCAAUGAGCGUCGAGCACGCAGACAUUUGG | 125 |
| IL15Rcx | GUGAAAUCAUACAGUCUGUACAGUAGAGAGCGGUACAUCUGCAAC | |
| ectodomain | AGUGGGUUUAAAAGAAAAGCAGGCACUUCAUCUCUGACAGAGUGC | |
| (without signal | GUGCUGAACAAAGCAACUAAUGUAGCUCAUUGGACAACCCCAUCA | |
| peptide) (RNA) | CUGAAAUGCAUUAGAGAUCCAGCUCUGGUGCAUCAAAGACCAGCA | |
| | CCACCAAGUACCGUAACAACCGCAGGGGUGACCCCUCAGCCUGAG | |
| | UCCCUAUCUCCCUCCGGCAAGGAGCCAGCAGCAUCUUCACCUAGC | |
| | UCCAAUAACACCGCAGCUACCACUGCCGCCAUAGUCCCCGGAAGC | |
| | CAGCUGAUGCCUAGUAAAUCUCCCUCAACAGGUACCACCGAAAUU | |
| | UCUAGCCAUGAGUCCUCGCACGGCACCCCGUCACAGACUACAGCU | |
| | AAAAACUGGGAGCUAACGGCUUCGGCAUCCCACCAACCUCCAGGC | |
| | GUUUAUCCCCAAGGUCACUCCGACACUACU | |
| | sushi domain of the wild-type IL15Rα (double | |
| | underlined) | |
| Fc-SushiL-IL15 | GAGCCUAAGAGCUGCGACAAGACCCACACCUGCCCUCCUUGCCCG | 126 |
| (RNA) | GCCCCAGAGUUGCUUGGCGGCCCUAGCGUCUUCCUCUUCCCUCCU | |
| | AAGCCUAAGGACACCCUGAUGAUCAGCAGAACCCCUGAGGUGACC | |
| | UGCGUGGUGGUGGCCGUGAGCCACGAGGACCCUGAAGUAAAGUUC | |
| | AACUGGUACGUGGACGGCGUGGAGGUGCACAACGCCAAGACCAAG | |
| | CCUAGAGAGGAGCAGUACAACAGCACCUACAGAGUGGUGAGCGUG | |
| | CUGACCGUGCUGCACCAGGACUGGCUGAACGGCAAGGAGUACAAG | |
| | UGCAAGGUGAGCAACAAGGCCCUGCCUGCACCUAUCGAGAAGACC | |
| | AUCAGCAAGGCCAAGGGCCAACCUCGAGAGCCUCAGGUGUACACC | |
| | CUGCCUCCUAGCAGAGACGAGCUGACCAAGAACCAGGUGAGCCUG | |
| | ACCUGCCUGGUGAAGGGCUUCUACCCUAGCGACAUCGCCGUGGAG | |
| | UGGGAGAGCAACGGCCAACCAGAGAACAACUACAAGACCACCCCU | |
| | CCUGUGCUGGACAGCGACGGCAGCUUCUUCCUCUACAGCAAGCUG | |
| | ACUGUUGACAAGAGCAGAUGGCAGCAGGGCAACGUGUUCAGCUGC | |
| | AGCGUGAUGCACGAGGCGCUGCACAACCACUACACCCAGAAGAGU | |
| | CUUUCCCUGAGCCCUGGCAAGAUAACGUGUCCGCCUCCUAUGAGC | |
| | GUGGAGCACGCCGACAUCUGGGUGAAGAGUUACAGCCUGUAUUCC | |
| | AGAGAGAGAUACAUCUGCAACAGCGGCUUCAAGAGAAAGGCCGGC | |
| | ACCAGCAGCUUAACCGAGUGCGUCCUUAACAAGGCUACCAACGUG | |
| | GCCCACUGGACAACGCCGAGCCUGAAGUGCAUCCGCGACCCUGCC | |
| | CUGGUGCACCAACGUCCAGCACCUCCGAGUGGCGGCAGUGGUGGC | |
| | GGAGGCUCAGGCGGUGGUAGUGGAGGAGGCGGAAGUCUGCAGAAU | |
| | UGGGUGAACGUGAUCAGCGACCUGAAGAAGAUCGAGGACCUGAUC | |
| | CAGAGCAUGCACAUCGACGCCACCCUGUACACCGAGAGCGACGUG | |
| | CACCCUAGCUGUAAGGUAACCGCCAUGAAGUGCUUCCUGCUGGAG | |
| | CUGCAGGUGAUUAGCCUGGAGAGCGGCGACGCCAGCAUCCACGAC | |
| | ACCGUGGAGAACCUGAUCAUCCUGGCCAACAAUUCUCUCAGUUCU | |
| | AACGGUAACGUGACCGAAUCGGGUUGUAAGGAGUGCGAGGAGCUG | |
| | GAGGAGAAGAACAUCAAGGAGUUCCUGCAGAGCUUCGUGCACAUU | |
| | GUGCAGAUGUUCAUCAACACCAGC | |
| | Fc domain (underline); SushiL (bold | |
| | italics); mature IL15 (bold underline) | |
| Fc-SushiL-IL15 | EPKSCDKTHTCPPCPAPELLGGPSVFLFPPKPKDTLMISRTPEVT | 127 |
| (aa) | CVVVAVSHEDPEVKFNWYVDGVEVHNAKTKPREEQYNSTYRVVSV | |
| | LTVLHQDWLNGKEYKCKVSNKALPAPIEKTISKAKGQPREPQVYT | |
| | LPPSRDELTKNQVSLTCLVKGFYPSDIAVEWESNGQPENNYKTTP | |
| | PVLDSDGSFFLYSKLTVDKSRWQQGNVFSCSVMHEALHNHYTQKS | |
| | LSLSPGKITCPPPMSVEHADIWVKSYSLYSRERYICNSGFKRKAG | |
| | TSSLTECVLNKATNVAHWTTPSLKCIRDPALVHQRPAPPSGGSGG | |
| | GGSGGGSGGGGSLQNWVNVISDLKKIEDLIQSMHIDATLYTESDV | |
| | HPSCKVTAMKCFLLELQVISLESGDASIHDTVENLIILANNSLSS | |
| | NGNVTESGCKECEELEEKNIKEFLQSFVHIVQMFINTS | |
| | Fc domain (underline); SushiL (bold | |
| | italics);mature IL15 (bold underline) | |

1. A messenger RNA (mRNA) comprising an open reading frame (ORF) encoding a fusion protein, wherein the fusion protein comprises from N-terminus to C-terminus:
(i) an apolipoprotein A (ApoA) polypeptide;
(ii) an extended IL15 Receptor alpha (IL15Rα) Sushi polypeptide; and
(iii) an interleukin 15 (IL15) polypeptide,
wherein (i), (ii), and (iii) are operably linked, optionally via a linker.

2. The mRNA of claim 1, wherein:
(a) the extended IL15Rα Sushi polypeptide comprises the Sushi domain of a human IL15Rα ectodomain, wherein the human IL15Rα ectodomain comprises the amino acid sequence of SEQ ID NO: 51 or an amino acid sequence having at least 90% identity to SEQ ID NO: 51;
(b) the extended IL15Rα Sushi polypeptide comprises a contiguous amino acid sequence extending from the N-terminus of the Sushi domain to at least one amino acid residue after the fourth cysteine residue of the Sushi domain of a human IL15Rα ectodomain, wherein the human IL15Rα ectodomain comprises the amino acid sequence of SEQ ID NO: 51;
(c) the extended IL15Rα Sushi polypeptide is at least 62 amino acid residues in length;
(d) the extended IL15Rα Sushi polypeptide is 62-80 amino acid residues in length;
(e) the ApoA polypeptide is a human origin ApoA-1 polypeptide or functional derivative thereof;
(f) the ApoA polypeptide comprises the amino acid sequence of SEQ ID NO: 14, or an amino acid sequence having at least 90% identity to the amino acid sequence of SEQ ID NO: 14;
(g) the IL15 polypeptide is a human IL15 polypeptide or functional derivative thereof;
(h) the ApoA polypeptide is encoded by a nucleotide sequence comprising a nucleotide sequence selected from SEQ ID NOs: 34-37, or a nucleotide sequence having at least 80% sequence identity to a nucleotide sequence selected from SEO ID NOs: 34-37; and/or
(i) the IL15 polypeptide comprises the amino acid sequence of SEQ ID NO: 16, or an amino acid sequence having at least 90% identity to the amino acid sequence of SEQ ID NO: 16.

3-5. (canceled)

6. An mRNA comprising an ORF encoding a fusion protein, wherein:
(a) the fusion protein comprises from N-terminus to C-terminus:
(i) an ApoA polypeptide;
(ii) an extended IL15Rα Sushi polypeptide comprising the amino acid sequence of SEQ ID NO: 17, or an amino acid sequence having at least 90% identity to the amino acid sequence of SEQ ID NO: 17; and
(iii) an IL15 polypeptide,
wherein (i), (ii), and (iii) are operably linked, optionally via a linker, or (b) the fusion protein comprises from N‘ terminus to C’ terminus:
(i) an ApoA polypeptide;
(ii) an extended IL15 Receptor alpha (IL15Rα) Sushi polypeptide comprising the amino acid sequence of SEQ ID NO: 18, or an amino acid sequence having at least 90% identity to the amino acid sequence of SEQ ID NO: 18; and
(iii) an IL15 polypeptide,
wherein (i), (ii), and (iii) are operably linked, optionally via a linker.

7. The mRNA of claim 6, wherein:
(a) the extended IL15Rα Sushi polypeptide is encoded by a nucleotide sequence comprising a nucleotide sequence selected from SEQ ID NOs: 29-31, or a nucleotide sequence having at least 80% identity to a nucleotide sequence selected from SEQ ID NOs: 29-31; or
(b) the extended IL15Rα Sushi polypeptide is encoded by a nucleotide sequence comprising a nucleotide sequence selected from SEQ ID NOs: 32 and 33, or a nucleotide sequence having at least 80% identity to a nucleotide sequence selected from SEO ID NOs: 32 and 33.

8-14. (canceled)

15. The mRNA of claim 2, wherein the IL15 polypeptide is encoded by a nucleotide sequence comprising a nucleotide sequence selected from SEQ ID NOs: 38-42, or a nucleotide sequence having at least 80% identity to a nucleotide sequence selected from SEQ ID NOs: 38-42.

16. The mRNA of claim 1, wherein
(a) the ApoA polypeptide is directly fused to the extended IL15Rα Sushi polypeptide;
(b) the ApoA polypeptide is operably linked to the extended IL15Rα Sushi polypeptide by a linker;
(c) the IL15Rα Sushi polypeptide is directly fused to the IL15 polypeptide;
(d) the IL15Rα Sushi polypeptide is operably linked to the IL15 polypeptide by a linker;
and/or
(e) the linker is a peptide linker.

17-20. (canceled)

21. The mRNA of claim 16, wherein the peptide linker is a GlySer linker, optionally wherein the GlySer linker comprises (GGGS)₃ (SEQ ID NO: 76).

22. An mRNA comprising an ORF encoding a fusion protein, wherein the fusion protein comprises:
(i) the amino acid sequence of SEQ ID NO: 123, or an amino acid sequence having at least 90% identity to the amino acid sequence of SEQ ID NO: 123; or
(ii) the amino acid sequence of SEQ ID NO: 121, or an amino acid sequence having at least 90% identity to the amino acid sequence of SEQ ID NO: 121.

23. The mRNA of claim 22, wherein:
(a) the fusion protein of (i) is encoded by a nucleotide sequence comprising the nucleotide sequence of SEQ ID NO: 122, or a nucleotide sequence having at least 80% identity to the nucleotide sequence of SEQ ID NO: 122, or
(b) the fusion protein of (ii) is encoded by a nucleotide sequence comprising the nucleotide sequence of SEQ ID NO: 120, or a nucleotide sequence having at least 80% identity to the nucleotide sequence of SEQ ID NO: 120.

24. (canceled)

25. The mRNA of claim 1, wherein the fusion protein comprises:
(a) a signal peptide at the N-terminus;
(b) a 5′ untranslated region (UTR);
(b) a 3′UTR;
(c) at least one chemical modification;
(d) a polyA tail; and/or
(e) a 5′Cap, optionally wherein the 5′Cap is a Cap 1 structure.

26. The mRNA of claim 25, wherein:
(a) the signal peptide is a human IgG heavy chain signal peptide;
(b) the signal peptide comprises the amino acid sequence of SEQ ID NO: 13;
(c) the 5′UTR comprises the nucleotide sequence set forth in SEQ ID NO: 19, or a nucleotide sequence having at least 80% identity to the nucleotide sequence of SEQ ID NO: 19;
(d) the 3′UTR comprises the nucleotide sequence set forth in SEQ ID NO: 20, or a nucleotide sequence having at least 80% identity to the nucleotide sequence of SEQ ID NO: 20;
(e) the 3′UTR comprises at least one microRNA (miR) binding site;
(f) the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine:
(g) (i) at least 95% of uridines are chemically modified; (ii) at least 99% of uridines are chemically modified; or (iii) 100% of uridines are chemically modified; and/or
(h) (i) at least 95% of uridines are N1-methylpseudouridine: (ii) at least 99% of uridines are N1-methylpseudouridine; or (iii) 100% of uridines are N1-methylpseudouridine.

27-38. (canceled)

39. A pharmaceutical composition comprising the mRNA of claim 1, and a pharmaceutically acceptable carrier.

40. A lipid nanoparticle (LNP) comprising the mRNA of claim 1.

41. The lipid nanoparticle of claim 40, wherein the lipid nanoparticle comprises:
(a) an ionizable amino lipid, a phospholipid, a structural lipid, and a polyethylene glycol (PEG)-modified lipid;
(b) (i) 40-60% ionizable amino lipid, 8-16% phospholipid, 30-45% sterol, and 1-5% PEG modified lipid, or (ii) a molar ratio of 45-65% ionizable amino lipid, 5-10% phospholipid, 25-40% sterol, and 0.5-5% PEG modified lipid;
(c) a molar ratio of 40-60% ionizable amino lipid, 8-16% phospholipid, 30-45% sterol, and 1-5% PEG modified lipid; or
(d) a molar ratio of 45-65% ionizable amino lipid, 5-10% phospholipid, 25-40% sterol, and 0.5-5% PEG-modified lipid.

42-44. (canceled)

45. The lipid nanoparticle of claim 41, wherein:
(a) the ionizable amino lipid is Compound 1: 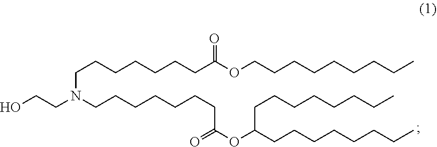 [SMILES: CCCCCCCCCOC(=O)CCCCCCCN(CCO)CCCCCCCC(=O)OC(CCCCCCCC)CCCCCCCC]
(b) the sterol is cholesterol and the PEG-modified lipid is PEG-DMG:
(c) the sterol is cholesterol and the PEG-modified lipid is Compound 2:

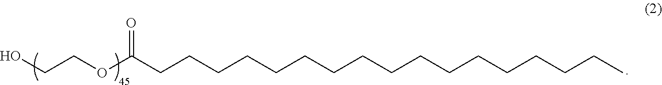

46. (canceled)

47. The lipid nanoparticle of claim 45, wherein the lipid nanoparticle comprises:
(i) about 40-60 mol % Compound 1; about 8-16 mol % DSPC; about 30-45 mol % cholesterol;
and about 1-5 mol % PEG-DMG;
(ii) about 45-65 mol % Compound 1; about 5-10 mol % DSPC; about 25-40 mol % cholesterol; and about 0.5-5 mol % PEG-DMG;
(iii) about 40-60 mol % Compound 1; about 8-16 mol % DSPC: about 30-45 mol % cholesterol; and about 1-5 mol % Compound 2; or
(iv) about 45-65 mol % is Compound 1; about 5-10 mol % DSPC: about 25-40 mol % cholesterol; and about 0.5-5 mol % Compound 2.

48. (canceled)

49. (canceled)

50. The lipid nanoparticle of claim 40, formulated for intravenous delivery.

51. A pharmaceutical composition comprising the lipid nanoparticle of claim 40, and a pharmaceutically acceptable carrier.

52. A method of treating a cancer in a subject, comprising administering to the subject the mRNA of claim 1.

53. A method of reducing or inhibiting tumor growth in a subject, comprising administering to the subject the mRNA of claim 1.

54. The method of claim 52, wherein:
(a) the subject has a disseminated tumor;
(b) the subject has a solid tumor; and/or
(c) the mRNA is administered intravenously.

55. (canceled)

56. (canceled)

57. A method of inducing or enhancing an anti-tumor immune response in a subject, comprising administering to the subject the mRNA of claim 1.

58. (canceled)

59. The method of claim 57, wherein the mRNA-encoded fusion protein is:
(a) expressed in the liver, the spleen, or both;
(b) expressed in hepatocytes; or
(c) expressed in Kupffer cells.

60-62. (canceled)

63. The method of any ene of claim 59, wherein following expression the ApoA polypeptide assembles to form a high-density lipoprotein (HDL) particle comprising the fusion protein.

64. The method of claim 63, wherein:
(a) the HDL particle anchors the IL15 polypeptide and extended IL15Rα Sushi polypeptide for presentation to immune cells; and/or
(b) the HDL particle facilitates trafficking of the fusion protein to the tumor.

65. (canceled)

66. The method of claim 57, wherein:
(a) the anti-tumor immune response comprises increased proliferation of CD8 T cells, NK cells, NKT cells, or a combination thereof; and/or
(b) the anti-tumor immune response comprises increased activation of CD8 T cells, NK cells, NKT cells, or a combination thereof.

67-73. (canceled)

74. A kit comprising a container comprising the mRNA of claim 1, and a package insert comprising instructions for administering the mRNA, or a lipid nanoparticle or a pharmaceutical composition comprising the same for (i treating a cancer in a subject, (ii) reducing or inhibiting tumor growth in a subject, or (iii) inducing or enhancing an anti-tumor immune response in a subject.

75. (canceled)

76. (canceled)

77. The method of any one of claim 52, wherein the subject is a human patient.
